(19) United States
(12) Patent Application Publication
(10) Pub. No.: US 2022/0009920 A1
(43) Pub. Date: Jan. 13, 2022

(54) KINASE INHIBITORS

(71) Applicant: GB005, Inc., San Diego, CA (US)

(72) Inventors: Craig Alan Coburn, San Diego, CA (US); Dange Vijay Kumar, San Diego, CA (US); Luis Antonio Lopez, San Diego, CA (US); Daniel John Buzard, San Diego, CA (US)

(21) Appl. No.: 17/225,984

(22) Filed: Apr. 8, 2021

Related U.S. Application Data
(60) Provisional application No. 63/142,411, filed on Jan. 27, 2021.
(60) Provisional application No. 63/008,531, filed on Apr. 10, 2020.

(51) Int. Cl. C07D 471/04; C07D 401/04; C07D 209/08; C07D 491/113; C07D 491/107; C07D 495/04; C07D 487/04; C07F 7/08

(52) U.S. Cl. CPC C07D 471/04; C07D 401/04; C07D 209/08; C07F 7/0816; C07D 491/107; C07D 495/04; C07D 487/04; C07D 491/113

(57) ABSTRACT

Disclosed herein are protein kinase inhibitors, in particular Bruton's tyrosine kinase (BTK) inhibitors, pharmaceutical compositions comprising them, processes for preparing them and uses of such protein kinase inhibitors to treat or prevent diseases, disorders and conditions associated with kinase function. In particular, the present invention relates to selective BTK inhibitors.

### FIELD OF THE INVENTION

The present invention relates generally to protein kinase inhibitors, in particular Bruton's tyrosine kinase (BTK) inhibitors, pharmaceutical compositions comprising them, processes for preparing them and uses of such inhibitors to treat or prevent diseases, disorders and conditions associated with kinase function.

### BACKGROUND

Protein kinases are a large group of intracellular and transmembrane signaling proteins in eukaryotic cells. These enzymes are responsible for transfer of the terminal (gamma) phosphate from ATP to specific amino acid residues of target proteins.

Phosphorylation of specific amino acid residues in target proteins can modulate their activity leading to profound changes in cellular signaling and metabolism. Protein kinases can be found in the cell membrane, cytosol and organelles such as the nucleus and are responsible for mediating multiple cellular functions including metabolism, cellular growth and differentiation, cellular signaling, modulation of immune responses, and cell death. Serine kinases specifically phosphorylate serine or threonine residues in target proteins. Similarly, tyrosine kinases, including tyrosine receptor kinases, phosphorylate tyrosine residues in target proteins. Tyrosine kinase families include: TEC, SRC, ABL, JAK, CSK, FAK, SYK, FER, ACK and the receptor tyrosine kinase subfamilies including ERBB, FGFR, VEGFR, RET and EPH. Subclass I of the receptor tyrosine kinase superfamily includes the ERBB receptors and comprises four members: ErbB1 (also called epidermal growth factor receptor (EGFR)), ErbB2, ErbB3 and ErbB4.

Kinases exert control on key biological processes related to health and disease. Furthermore, aberrant activation or excessive expression of various protein kinases are implicated in the mechanism of multiple diseases and disorders characterized by benign and malignant proliferation, as well as diseases resulting from inappropriate activation of the immune system. Thus, inhibitors of select kinases or kinase families are considered useful in the treatment of cancer, vascular disease, autoimmune diseases, and inflammatory conditions including, but not limited to: solid tumors, hematological malignancies, thrombus, arthritis, graft versus host disease, lupus erythematosus, psoriasis, colitis, illeitis, multiple sclerosis, uveitis, coronary artery vasculopathy, systemic sclerosis, atherosclerosis, asthma, transplant rejection, allergy, ischemia, dermatomyositis, pemphigus, and the like.

Tec kinases are a family of non-receptor tyrosine kinases predominantly, but not exclusively, expressed in cells of hematopoietic origin. The Tec family includes TEC, Bruton's tyrosine kinase (BTK), inducible T-cell kinase (ITK), resting lymphocyte kinase (RLK/TXK for Tyrosine Protein Kinase), and bone marrow-expressed kinase (BMX/ETK).

BTK is important in B-cell receptor signaling and regulation of B-cell development and activation. Mutation of the gene encoding BTK in humans leads to X-linked agammaglobulinemia which is characterized by reduced immune function, including impaired maturation of B-cells, decreased levels of immunoglobulin and peripheral B cells, and diminished T-cell independent immune response. BTK is activated by Src-family kinases and phosphorylates PLC gamma leading to effects on B-cell function and survival. Additionally, BTK is important for cellular function of mast cells, macrophage and neutrophils indicating that BTK inhibition is effective in treatment of diseases mediated by these and related cells including inflammation, bone disorders, and allergic disease. BTK inhibition is also important in survival of lymphoma cells indicating that inhibition of BTK is useful in the treatment of lymphomas and other cancers. As such, inhibitors of BTK and related kinases are of great interest as anti-inflammatory, as well as anti-cancer, agents. BTK is also important for platelet function and thrombus formation indicating that BTK-selective inhibitors are also useful as antithrombotic agents. Furthermore, BTK is required for inflammasome activation, and inhibition of BTK may be used in treatment of inflammasome-related disorders, including; stroke, gout, type 2 diabetes, obesity-induced insulin resistance, atherosclerosis and Muckle-Wells syndrome. In addition, BTK is expressed in HIV infected T-cells and treatment with BTK inhibitors sensitizes infected cells to apoptotic death and results in decreased virus production. Accordingly, BTK inhibitors are considered useful in the treatment of HIV-AIDS and other viral infections.

Further, BTK is important in neurological function. Specifically targeting BTK in the brain and CNS has the potential to significantly advance the treatment of neurological diseases such as progressive and relapsing forms of MS and primary CNS lymphoma (PCNSL).

PCNSL is a rare brain tumor with an annual incidence in the United States of approximately 1900 new cases each year and constitutes approximately 3% of all newly diagnosed brain tumors.

PCNSL is highly aggressive and unlike other lymphomas outside the CNS, prognosis remains poor despite improvements in treatments in the front-line setting.

High dose methotrexate remains the backbone of treatment and is used in combination with other cytotoxic agents, and more recently the addition of rituximab. From initial diagnosis, 5-year survival has improved from 19% to 30% between 1990 and 2000 but has not improved in the elderly population (>70 years), due to 20% or more of these patients being considered unfit for chemotherapy. Tumor regression is observed in ˜85% of patients regardless of the treatment modality in the front-line setting, however, approximately half of these patients will experience recurrent disease within 10-18 months after initial treatment and most relapses occur within the first 2 years of diagnosis.

Thus, the prognosis for patients with relapsed/refractory PCNSL (R/R PCNSL) remains poor with a median survival of ˜2 months without further treatment. As there is no uniform standard of care for the treatment of R/R PCNSL, participation in clinical trials is encouraged. New safe and effective treatments are urgently needed.

BTK is involved in the signal transduction in the B cell antigen receptor (BCR) signaling pathway and integrates BCR and Toll-like receptor (TLR)-signaling. Genes in these pathways frequently harbor mutations in diffuse large B-cell lymphoma (DLBCL), including CD79B and myeloid differentiation primary response 88 (MyD88). Ibrutinib, a first-generation irreversible selective inhibitor of BTK, has been approved for chronic lymphocytic-leukemia/small cell lymphocytic lymphoma (CLL/SLL), previously treated Mantle Cell lymphoma (MCL) and Marginal Zone Lymphoma (MZL), Waldenström's macroglobulin, and previously treated chronic Graft Versus Host Disease. In clinical studies the recommended dose of Ibrutinib (480 mg/d in CLL or 560 mg/d in MCL) was escalated to 840 mg to achieve adequate brain exposure in primary CNS lymphoma.

Aberrant activation of the NF-κB pathway in PCNSL is emerging as a potential mechanism for more targeted therapy. In particular, activating mutations of CARD 11 as well as of MyD88 (Toll-like receptor pathway) have been implicated. The activating exchange of leucine to proline at position 265 of MyD88, noted to occur in between 38% (11/29) and 50% (7/14) of patients, is the most frequent mutation identified thus far in PCNSL. In addition, the coding region of CD79B, a component of the B-cell receptor signaling pathway, appears to contain mutations in 20% of cases, suggesting that dysregulation of the B-cell receptor and NF-κB pathways contribute to the pathogenesis of PCNSL. These data suggest that BCR pathway mutations and BTK dependence are of particular relevance to PCNSL.

Recently, several clinical studies have reported substantial single-agent clinical activity in the treatment of PCNSL with response rates of 70-77%. The majority of patients, however, discontinued therapy by 9 months. Although Ibrutinib therapy has been reported to be generally well tolerated with manageable adverse events, there are reports of sometimes fatal fungal infections. Of note, escalating doses beyond 560 mg to 840 mg/day have been used to achieve higher brain exposure and these higher doses may be associated with off-target effects mediated by Ibrutinib's kinase selectivity profile. Finally, the combination of high dose Ibrutinib in conjunction with high-dose steroids may contribute to exacerbate the increased fungal infections. Therefore, there remains a need for BTK inhibitors with an improved efficacy and safety profile due to greater brain penetration and BTK inactivation rate with greater kinase selectivity.

There remains a need for compounds that modulate protein kinases generally, as well as compounds that modulate specific protein kinases, such as BTK, as well as compounds that modulate specific protein kinases and selectively cross the blood/brain barrier for related compositions and methods for treating diseases, disorders and conditions that would benefit from such modulation and selectivity.

### BRIEF SUMMARY

In one aspect, compounds are provided having the structure of Formula (I):

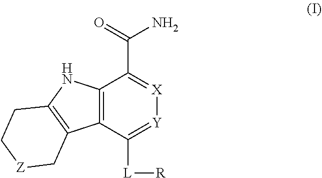

or a pharmaceutically acceptable salt, solvate, hydrate, isomer, tautomer, racemate, or isotope thereof, wherein:

X is CR¹ or N;

Y is CR² or N;

R¹ is H, halo, or C₁₋₃ alkyl;

R² is H, halo, or C₁₋₃ alkyl;

Z is a bond, C(R³R⁴), C(R⁵R⁶)—C(R⁷R⁸), C(R⁹R¹⁰)—N(R¹¹), N(R¹¹)—C(R⁹R¹⁰), O, S, or Si(R¹²R¹³),

R³ and R⁴ are each, independently, H, halo, or C₁₋₃ alkyl, or R³ and R⁴, together with the carbon to which they are attached, form ring A:

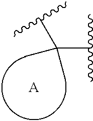

wherein ring A is an optionally substituted carbocycle or an optionally substituted heterocycle;

R⁵, R⁶, R⁷, R⁸, R⁹, and R¹⁰ are each, independently, H, halo, or C₁₋₃ alkyl;

R¹¹, R¹², and R¹³ are each, independently, H or C₁₋₃ alkyl;

L is

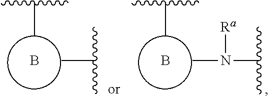

ring B is an optionally substituted carbocycle or an optionally substituted heterocycle;

R^{a} is H or C₁₋₃ alkyl;

R is C(O)R¹⁴; and

R¹⁴ is C₂₋₆ alkenyl substituted with 0-3 R′ or C₂₋₆ alkynyl substituted with 0-3 R′;

R′ is at each occurrence, independently, halo, —OR^{b}, —NR^{b}R^{c}, or optionally substituted carbocycle; and

R^{b} and R^{c} are at each occurrence, independently, H, C₁₋₆ alkyl, or C₁₋₆ haloalkyl; with the proviso that if Z is CH₂ or CF₂, then at least one of X and Y is N.

In one embodiment, a pharmaceutical composition is provided comprising a compound having the structure of any one of Formulas (I), (I′), (I-A), (I-B), (I-C), (I-D), or (II), or a pharmaceutically acceptable salt, solvate, hydrate, isomer, tautomer, racemate, or isotope thereof, and at least one pharmaceutically acceptable excipient.

In one embodiment, a method of modulating a protein kinase is provided comprising contacting the protein kinase with an effective amount of a compound having the structure of any one of Formulas (I), (I′), (I-A), (I-B), (I-C), (I-D), or (II), or a pharmaceutically acceptable salt, solvate, hydrate, isomer, tautomer, racemate, isotope, or pharmaceutical composition thereof. In one embodiment, the protein kinase is BTK.

In one embodiment, a method for treating a BTK dependent condition is provided, comprising administering to a subject in need thereof an effective amount of a compound having the structure of any one of Formulas (I), (I′), (I-A), (I-B), (I-C), (I-D), or (II), or a pharmaceutically acceptable salt, solvate, hydrate, isomer, tautomer, racemate, isotope, or pharmaceutical composition thereof.

In one embodiment, the use of a compound having the structure of any one of Formulas (I), (I′), (I-A), (I-B), (I-C), (I-D), or (II), or a pharmaceutically acceptable salt, solvate, hydrate, isomer, tautomer, racemate, isotope, or pharmaceutical composition thereof is provided, in the manufacture of a medicament.

### DETAILED DESCRIPTION

Unless defined otherwise, all technical and scientific terms used herein have the same meaning as is commonly understood by one of skill in the art to which the claimed subject matter belongs. It is to be understood that the detailed description is exemplary and explanatory only and is not restrictive of any subject matter claimed. In this application, the use of the singular includes the plural unless specifically stated otherwise. It must be noted that, as used in the specification, the singular forms “a,” “an” and “the” include plural referents unless the context clearly dictates otherwise. In this application, the use of “or” means “and/or” unless stated otherwise. Furthermore, use of the term “including” as well as other forms, such as “include”, “includes,” and “included,” is not limiting.

Although various features of the invention may be described in the context of a single embodiment, the features may also be provided separately or in any suitable combination. Conversely, although the invention may be described herein in the context of separate embodiments for clarity, the invention may also be implemented in a single embodiment.

Reference in the specification to “some embodiments”, “an embodiment”, “one embodiment” or “other embodiments” means that a particular feature, structure, or characteristic described in connection with the embodiments is included in at least some embodiments, but not necessarily all embodiments, of the inventions.

As used herein, ranges and amounts can be expressed as “about” a particular value or range. About also includes the exact amount. Hence “about 100 μL” means “about 100 μL” and also “100 μL.” In some embodiments, about means within 5% of the value. Hence, “about 100 μL” means 95-105 μL. Generally, the term “about” includes an amount that would be expected to be within experimental error.

As used herein, “alkyl” means a straight chain or branched saturated hydrocarbon group. “Lower alkyl” means a straight chain or branched alkyl group having from 1 to 8 carbon atoms, in some embodiments from 1 to 6 carbon atoms, in some embodiments from 1 to 4 carbon atoms, and in some embodiments from 1 to 2 carbon atoms. Examples of straight chain lower alkyl groups include, but are not limited to, methyl, ethyl, n-propyl, n-butyl, n-pentyl, n-hexyl, n-heptyl, and n-octyl groups. Examples of branched lower alkyl groups include, but are not limited to, isopropyl, iso-butyl, sec-butyl, t-butyl, neopentyl, isopentyl, and 2,2-dimethylpropyl groups.

“Alkenyl” groups include straight and branched chain and cyclic alkyl groups as defined above, except that at least one double bond exists between two carbon atoms. Thus, alkenyl groups have from 2 to about 20 carbon atoms, and typically from 2 to 12 carbons, or, in some embodiments, from 2 to 8 carbon atoms. Examples include, but are not limited to —CH═CH₂, —CH═CH(CH₃), —CH═C(CH₃)₂, —C(CH₃)═CH₂, —C(CH₃)═CH(CH₃), —C(CH₂CH₃)═CH₂, —CH═CHCH₂CH₃, —CH═CH(CH₂)₂CH₃, —CH═CH(CH₂)₃CH₃, —CH═CH(CH₂)₄CH₃, vinyl, cyclohexenyl, cyclopentenyl, cyclohexadienyl, butadienyl, pentadienyl, and hexadienyl among others.

“Alkynyl” groups include straight and branched chain alkyl groups, except that at least one triple bond exists between two carbon atoms. Thus, alkynyl groups have from 2 to about 20 carbon atoms, and typically from 2 to 12 carbons, or, in some embodiments, from 2 to 8 carbon atoms. Examples include, but are not limited to —C≡CH, —C≡C(CH₃), —C≡C(CH₂CH₃), —CH₂C≡CH, —CH₂C≡C(CH₃), and —CH₂C≡C(CH₂CH₃), among others.

As used herein, “alkylene” means a divalent alkyl group. Examples of straight chain lower alkylene groups include, but are not limited to, methylene (i.e., —CH₂—), ethylene (i.e., —CH₂CH₂—), propylene (i.e., —CH₂CH₂CH₂—), and butylene (i.e., —CH₂CH₂CH₂CH₂—). As used herein, “heteroalkylene” is an alkylene group of which one or more carbon atoms is replaced with a heteroatom such as, but not limited to, N, O, S, or P.

“Alkoxy” refers to an alkyl as defined above joined by way of an oxygen atom (i.e., —O-alkyl). Examples of lower alkoxy groups include, but are not limited to, methoxy, ethoxy, n-propoxy, n-butoxy, isopropoxy, sec-butoxy, tert-butoxy, and the like.

The terms “carbocyclic” and “carbocycle” denote a ring structure wherein the atoms of the ring are carbon. Carbocycles may be monocyclic or polycyclic. Carbocycle encompasses both saturated and unsaturated rings. Carbocycle encompasses both cycloalkyl and aryl groups. In some embodiments, the carbocycle has 3 to 8 ring members, whereas in other embodiments the number of ring carbon atoms is 4, 5, 6, or 7. Unless specifically indicated to the contrary, the carbocyclic ring can be substituted with as many as N substituents wherein N is the size of the carbocyclic ring with for example, alkyl, amino, hydroxy, cyano, carboxy, nitro, thio, alkoxy, and halogen groups.

“Cycloalkyl” groups are alkyl groups forming a ring structure, which can be substituted or unsubstituted. Examples of cycloalkyl include, but are not limited to, cyclopropyl, cyclobutyl, cyclopentyl, cyclohexyl, cycloheptyl, and cyclooctyl groups. In some embodiments, the cycloalkyl group has 3 to 8 ring members, whereas in other embodiments the number of ring carbon atoms range from 3 to 5, 3 to 6, or 3 to 7. Cycloalkyl groups further include polycyclic cycloalkyl groups such as, but not limited to, norbornyl, adamantyl, bornyl, camphenyl, isocamphenyl, and carenyl groups, and fused rings such as, but not limited to, decalinyl, and the like. Cycloalkyl groups also include rings that are substituted with straight or branched chain alkyl groups as defined above. Representative substituted cycloalkyl groups can be mono-substituted or substituted more than once, such as, but not limited to, 2,2-, 2,3-, 2,4-2,5-, or 2,6-disubstituted cyclohexyl groups or mono-, di-, or tri-substituted norbornyl or cycloheptyl groups, which can be substituted with, for example, amino, hydroxy, cyano, carboxy, nitro, thio, alkoxy, and halogen groups.

“Aryl” groups are cyclic aromatic hydrocarbons that do not contain heteroatoms. Thus, aryl groups include, but are not limited to, phenyl, azulenyl, heptalenyl, biphenyl, indacenyl, fluorenyl, phenanthrenyl, triphenylenyl, pyrenyl, naphthacenyl, chrysenyl, biphenylenyl, anthracenyl, and naphthyl groups. In some embodiments, aryl groups contain 6-14 carbons in the ring portions of the groups. The terms “aryl” and “aryl groups” include fused rings wherein at least one ring, but not necessarily all rings, are aromatic, such as fused aromatic-aliphatic ring systems (e.g., indanyl, tetrahydronaphthyl, and the like).

“Carbocyclealkyl” refers to an alkyl as defined above with one or more hydrogen atoms replaced with carbocycle. Examples of carbocyclealkyl groups include, but are not limited to, benzyl and the like.

As used herein, “heterocycle” or “heterocyclyl” groups include aromatic and non-aromatic ring compounds (heterocyclic rings) containing 3 or more ring members, of which one or more is a heteroatom such as, but not limited to, N, O, S, or P. A heterocycle group as defined herein can be a heteroaryl group or a partially or completely saturated cyclic group including at least one ring heteroatom. In some embodiments, heterocycle groups include 3 to 20 ring members, whereas other such groups have 3 to 15 ring members. At least one ring contains a heteroatom, but every ring in a polycyclic system need not contain a heteroatom. For example, a dioxolanyl ring and a benzodioxolanyl ring system (methylenedioxyphenyl ring system) are both heterocycle groups within the meaning herein. A heterocycle group designated as a C₂-heterocycle can be a 5-membered ring with two carbon atoms and three heteroatoms, a 6-membered ring with two carbon atoms and four heteroatoms and so forth. Likewise, a C₄-heterocycle can be a 5-membered ring with one heteroatom, a 6-membered ring with two heteroatoms, and so forth. The number of carbon atoms plus the number of heteroatoms sums up to equal the total number of ring atoms. A saturated heterocyclic ring refers to a heterocyclic ring containing no unsaturated carbon atoms.

“Heteroaryl” groups are aromatic ring compounds containing 5 or more ring members, of which, one or more is a heteroatom such as, but not limited to, N, O, and S. A heteroaryl group designated as a C₂-heteroaryl can be a 5-membered ring with two carbon atoms and three heteroatoms, a 6-membered ring with two carbon atoms and four heteroatoms and so forth. Likewise, a C₄-heteroaryl can be a 5-membered ring with one heteroatom, a 6-membered ring with two heteroatoms, and so forth. The number of carbon atoms plus the number of heteroatoms sums up to equal the total number of ring atoms. Heteroaryl groups include, but are not limited to, groups such as pyrrolyl, pyrazolyl, triazolyl, tetrazolyl, oxazolyl, isoxazolyl, thiazolyl, pyridinyl, thiophenyl, benzothiophenyl, benzofuranyl, indolyl, azaindolyl, indazolyl, benzimidazolyl, azabenzimidazolyl, benzoxazolyl, benzothiazolyl, benzothiadiazolyl, imidazopyridinyl, isoxazolopyridinyl, thianaphthalenyl, purinyl, xanthinyl, adeninyl, guaninyl, quinolinyl, isoquinolinyl, tetrahydroquinolinyl, tetrahydroisoquinolinyl, quinoxalinyl, and quinazolinyl groups. The terms “heteroaryl” and “heteroaryl groups” include fused ring compounds such as wherein at least one ring, but not necessarily all rings, are aromatic, including tetrahydroquinolinyl, tetrahydroisoquinolinyl, indolyl and 2,3-dihydro indolyl.

“Heterocyclealkyl” refers to an alkyl as defined above with one or more hydrogen atoms replaced with heterocycle. Examples of heterocyclealkyl groups include, but are not limited to, morpholinoethyl and the like.

“Halo” or “halogen” refers to fluorine, chlorine, bromine and iodine.

“Haloalkyl” refers to an alkyl as defined above with one or more hydrogen atoms replaced with halogen. Examples of lower haloalkyl groups include, but are not limited to, —CF₃, —CH₂CF₃, and the like.

“Haloalkoxy” refers to an alkoxy as defined above with one or more hydrogen atoms replaced with halogen. Examples of lower haloalkoxy groups include, but are not limited to —OCF₃, —OCH₂CF₃, and the like.

“Hydroxyalkyl” refers to an alkyl as defined above with one or more hydrogen atoms replaced with —OH. Examples of lower hydroxyalkyl groups include, but are not limited to —CH₂OH, —CH₂CH₂OH, and the like.

As used herein, the term “optionally substituted” refers to a group (e.g., an alkyl, carbocycle, or heterocycle) having 0, 1, or more substituents, such as 0-25, 0-20, 0-10, or 0-5 substituents. Substituents include, but are not limited to —OR^{aa}, —NR^{aa}R^{bb}, —S(O)₂R^{aa}, or —S(O)₂OR^{aa}, halogen, cyano, oxo (═O), alkyl, haloalkyl, alkoxy, carbocycle, heterocycle, carbocyclealkyl, or heterocyclealkyl, wherein each R^{aa} and R^{bb} is, independently, H, alkyl, haloalkyl, carbocycle, or heterocycle, or R^{aa} and R^{bb}, together with the atom to which they are attached, form a 3-8 membered carbocycle or heterocycle.

“Isomer” is used herein to encompass all chiral, diastereomeric, or racemic forms of a structure, unless a particular stereochemistry or isomeric form is specifically indicated. Such compounds can be enriched or resolved optical isomers at any or all asymmetric atoms as are apparent from the depictions, at any degree of enrichment. Both racemic and diastereomeric mixtures, as well as the individual optical isomers can be synthesized to be substantially free of their enantiomeric or diastereomeric partners, and these are all within the scope of certain embodiments of the disclosure. The isomers resulting from the presence of a chiral center comprise a pair of non-superimposable isomers that are called “enantiomers.” Single enantiomers of a pure compound are optically active (i.e., they can rotate the plane of plane polarized light and designated R or S).

“Isolated optical isomer” means a compound which has been substantially purified from the corresponding optical isomer(s) of the same formula. For example, the isolated isomer may be at least about 80%, at least 80%, or at least 85% pure. In other embodiments, the isolated isomer is at least 90% pure, at least 98% pure, or at least 99% pure by weight.

“Substantially enantiomerically or diastereomerically pure” means a level of enantiomeric or diastereomeric enrichment of one enantiomer with respect to the other enantiomer or diastereomer of at least about 80%, and more specifically in excess of 80%, 85%, 90%, 95%, 98%, 99%, 99.5%, or 99.9%.

The terms “racemate” and “racemic mixture” refer to an equal mixture of two enantiomers. A racemate is labeled “(±)” because it is not optically active (i.e., will not rotate plane-polarized light in either direction since its constituent enantiomers cancel each other out).

A “hydrate” is a compound that exists in combination with water molecules. The combination can include water in stoichiometric quantities, such as a monohydrate or a dihydrate, or can include water in random amounts. As the term is used herein a “hydrate” refers to a solid form; that is, a compound in a water solution, while it may be hydrated, is not a hydrate as the term is used herein.

A “solvate” is similar to a hydrate except that a solvent other that water is present. For example, methanol or ethanol can form an “alcoholate”, which can again be stoichiometric or non-stoichiometric. As the term is used herein a “solvate” refers to a solid form; that is, a compound in a solvent solution, while it may be solvated, is not a solvate as the term is used herein.

“Isotope” refers to atoms with the same number of protons but a different number of neutrons, and an isotope of a compound of Formulas (I) includes any such compound wherein one or more atoms are replaced by an isotope of that atom. For example, carbon 12, the most common form of carbon, has six protons and six neutrons, whereas carbon 13 has six protons and seven neutrons, and carbon 14 has six protons and eight neutrons. Hydrogen has two stable isotopes, deuterium (one proton and one neutron) and tritium (one proton and two neutrons). While fluorine has several isotopes, fluorine 19 is longest-lived. Thus, an isotope of a compound having the structure of Formulas (I) includes, but not limited to, compounds of Formulas (I) wherein one or more carbon 12 atoms are replaced by carbon-13 and/or carbon-14 atoms, wherein one or more hydrogen atoms are replaced with deuterium and/or tritium, and/or wherein one or more fluorine atoms are replaced by fluorine-19.

“Salt” generally refers to an organic compound, such as a carboxylic acid or an amine, in ionic form, in combination with a counter ion. For example, salts formed between acids in their anionic form and cations are referred to as “acid addition salts”. Conversely, salts formed between bases in the cationic form and anions are referred to as “base addition salts.”

The term “pharmaceutically acceptable” refers an agent that has been approved for human consumption and is generally non-toxic. For example, the term “pharmaceutically acceptable salt” refers to nontoxic inorganic or organic acid and/or base addition salts (see, e.g., Lit et al., Salt Selection for Basic Drugs, Int. J. Pharm., 33, 201-217, 1986) (incorporated by reference herein).

Pharmaceutically acceptable base addition salts of compounds of the disclosure include, for example, metallic salts including alkali metal, alkaline earth metal, and transition metal salts such as, for example, calcium, magnesium, potassium, sodium, and zinc salts. Pharmaceutically acceptable base addition salts also include organic salts made from basic amines such as, for example, N,N′dibenzylethylenediamine, chloroprocaine, choline, diethanolamine, ethylenediamine, meglumine (N-methylglucamine), and procaine.

Pharmaceutically acceptable acid addition salts may be prepared from an inorganic acid or from an organic acid. Examples of inorganic acids include hydrochloric, hydrobromic, hydriodic, nitric, carbonic, sulfuric, and phosphoric acids. Appropriate organic acids may be selected from aliphatic, cycloaliphatic, aromatic, aromatic aliphatic, heterocyclic, carboxylic, and sulfonic classes of organic acids, examples of which include formic, acetic, propionic, succinic, glycolic, gluconic, lactic, malic, tartaric, citric, ascorbic, glucuronic, maleic, fumaric, pyruvic, aspartic, glutamic, benzoic, anthranilic, 4-hydroxybenzoic, phenylacetic, mandelic, hippuric, malonic, oxalic, embonic (pamoic), methanesulfonic, ethanesulfonic, benzenesulfonic, panthothenic, trifluoromethanesulfonic, 2-hydroxyethanesulfonic, p-toluenesulfonic, sulfanilic, cyclohexylaminosulfonic, stearic, alginic, β-hydroxybutyric, salicylic, -galactaric, and galacturonic acid.

Although pharmaceutically unacceptable salts are not generally useful as medicaments, such salts may be useful, for example as intermediates in the synthesis of the compounds described herein, for example in their purification by recrystallization.

In certain embodiments, the disclosure provides a pharmaceutical composition comprising a compound as described herein, or a pharmaceutically acceptable isomer, racemate, hydrate, solvate, isotope, or salt thereof, together with at least one pharmaceutically acceptable carrier, diluent, or excipient. For example, the active compound will usually be mixed with a carrier, or diluted by a carrier, or enclosed within a carrier which can be in the form of an ampoule, capsule, sachet, paper, or other container. When the active compound is mixed with a carrier, or when the carrier serves as a diluent, it can be solid, semi-solid, or liquid material that acts as a vehicle, excipient, or medium for the active compound. The active compound can be adsorbed on a granular solid carrier, for example contained in a sachet. Some examples of suitable carriers are water, salt solutions, alcohols, polyethylene glycols, polyhydroxyethoxylated castor oil, peanut oil, olive oil, gelatin, lactose, terra alba, sucrose, dextrin, magnesium carbonate, sugar, cyclodextrin, amylose, magnesium stearate, talc, gelatin, agar, pectin, acacia, stearic acid, or lower alkyl ethers of cellulose, silicic acid, fatty acids, fatty acid amines, fatty acid monoglycerides and diglycerides, pentaerythritol fatty acid esters, polyoxyethylene, hydroxymethylcellulose, and polyvinylpyrrolidone. Similarly, the carrier or diluent can include any sustained release material known in the art, such as glyceryl monostearate or glyceryl distearate, alone or mixed with a wax.

As used herein, the term “pharmaceutical composition” refers to a composition containing one or more of the compounds described herein, or a pharmaceutically acceptable isomer, racemate, hydrate, solvate, homolog, or salt thereof, formulated with a pharmaceutically acceptable carrier, which can also include other additives, and manufactured or sold with the approval of a governmental regulatory agency as part of a therapeutic regimen for the treatment of disease in a mammal. Pharmaceutical compositions can be formulated, for example, for oral administration in unit dosage form (e.g., a tablet, capsule, caplet, gelcap, or syrup); for topical administration (e.g., as a cream, gel, lotion, or ointment); for intravenous administration (e.g., as a sterile solution free of particulate emboli and in a solvent system suitable for intravenous use); or in any other formulation described herein. Conventional procedures and ingredients for the selection and preparation of suitable formulations are described, for example, in Remington: The Science and Practice of Pharmacy, 21st Ed., Gennaro, Ed., Lippencott Williams & Wilkins (2005) and in The United States Pharmacopeia: The National Formulary (USP 36 NF31), published in 2013.

In other embodiments, there are provided methods of making a composition of a compound described herein including formulating a compound of the disclosure with a pharmaceutically acceptable carrier or diluent. In some embodiments, the pharmaceutically acceptable carrier or diluent is suitable for oral administration. In some such embodiments, the methods can further include the step of formulating the composition into a tablet or capsule. In other embodiments, the pharmaceutically acceptable carrier or diluent is suitable for parenteral administration. In some such embodiments, the methods further include the step of lyophilizing the composition to form a lyophilized preparation.

As used herein, the term “pharmaceutically acceptable carrier” refers to any ingredient other than the disclosed compounds, or a pharmaceutically acceptable isomer, racemate, hydrate, solvate, homolog, or salt thereof (e.g., a carrier capable of suspending or dissolving the active compound) and having the properties of being nontoxic and non-inflammatory in a patient. Excipients may include, for example: antiadherents, antioxidants, binders, coatings, compression aids, disintegrants, dyes (colors), emollients, emulsifiers, fillers (diluents), film formers or coatings, flavors, fragrances, glidants (flow enhancers), lubricants, preservatives, printing inks, sorbents, suspensing or dispersing agents, sweeteners, or waters of hydration. Exemplary excipients include, but are not limited to: butylated hydroxytoluene (BHT), calcium carbonate, calcium phosphate (dibasic), calcium stearate, croscarmellose, crosslinked polyvinyl pyrrolidone, citric acid, crospovidone, cysteine, ethylcellulose, gelatin, hydroxypropyl cellulose, hydroxypropyl methylcellulose, lactose, magnesium stearate, maltitol, mannitol, methionine, methylcellulose, methyl paraben, microcrystalline cellulose, polyethylene glycol, polyvinyl pyrrolidone, povidone, pregelatinized starch, propyl paraben, retinyl palmitate, shellac, silicon dioxide, sodium carboxymethyl cellulose, sodium citrate, sodium starch glycolate, sorbitol, starch (corn), stearic acid, stearic acid, sucrose, talc, titanium dioxide, vitamin A, vitamin E, vitamin C, and xylitol.

The formulations can be mixed with auxiliary agents which do not deleteriously react with the active compounds. Such additives can include wetting agents, emulsifying and suspending agents, salt for influencing osmotic pressure, buffers and/or coloring substances, preserving agents, sweetening agents, or flavoring agents. The compositions can also be sterilized if desired.

The route of administration can be any route which effectively transports the active compound of the disclosure to the appropriate or desired site of action, such as oral, nasal, pulmonary, buccal, subdermal, intradermal, transdermal, or parenteral, e.g., rectal, depot, subcutaneous, intravenous, intraurethral, intramuscular, intranasal, ophthalmic solution, or an ointment, the oral route being preferred.

Dosage forms can be administered once a day, or more than once a day, such as twice or thrice daily. Alternatively, dosage forms can be administered less frequently than daily, such as every other day, or weekly, if found to be advisable by a prescribing physician. Dosing regimens include, for example, dose titration to the extent necessary or useful for the indication to be treated, thus allowing the patient's body to adapt to the treatment and/or to minimize or avoid unwanted side effects associated with the treatment. Other dosage forms include delayed or controlled-release forms. Suitable dosage regimens and/or forms include those set out, for example, in the latest edition of the Physicians' Desk Reference, incorporated herein by reference.

As used herein, the term “administering” or “administration” refers to providing a compound, a pharmaceutical composition comprising the same, to a subject by any acceptable means or route, including (for example) by oral, parenteral (e.g., intravenous), or topical administration.

As used herein, the term “treatment” refers to an intervention that ameliorates a sign or symptom of a disease or pathological condition. As used herein, the terms “treatment”, “treat” and “treating,” with reference to a disease, pathological condition or symptom, also refers to any observable beneficial effect of the treatment. The beneficial effect can be evidenced, for example, by a delayed onset of clinical symptoms of the disease in a susceptible subject, a reduction in severity of some or all clinical symptoms of the disease, a slower progression of the disease, a reduction in the number of relapses of the disease, an improvement in the overall health or well-being of the subject, or by other parameters well known in the art that are specific to the particular disease. A prophylactic treatment is a treatment administered to a subject who does not exhibit signs of a disease or exhibits only early signs, for the purpose of decreasing the risk of developing pathology. A therapeutic treatment is a treatment administered to a subject after signs and symptoms of the disease have developed.

As used herein, the term “subject” refers to an animal (e.g., a mammal, such as a human). A subject to be treated according to the methods described herein may be one who has been diagnosed with a proliferative disease or disorder such as cancer or an oncology indication, an autoimmune disease or disorder, an inflammatory disease or disorder, or a thromboembolic disease or disorder, or one at risk of developing the condition. In an embodiment, the autoimmune disease is multiple sclerosis, rheumatoid arthritis, psoriasis, Sjogren's syndrome, or systemic lupus erythematosus. In another embodiment, the inflammatory disease is urticaria. In another embodiment, the oncology indication is primary CNS lymphoma. Diagnosis may be performed by any method or technique known in the art. One skilled in the art will understand that a subject to be treated according to the present disclosure may have been subjected to standard tests or may have been identified, without examination, as one at risk due to the presence of one or more risk factors associated with the disease or condition.

As used herein, the term “effective amount” refers to a quantity of a specified agent sufficient to achieve a desired effect in a subject being treated with that agent. Ideally, an effective amount of an agent is an amount sufficient to inhibit or treat the disease without causing substantial toxicity in the subject. The effective amount of an agent will be dependent on the subject being treated, the severity of the affliction, and the manner of administration of the pharmaceutical composition. Methods of determining an effective amount of the disclosed compound sufficient to achieve a desired effect in a subject will be understood by those of skill in the art in light of this disclosure.

As used herein, the terms “modulate” or “modulating” refer to the ability to increase or decrease the activity of one or more protein kinases. Accordingly, compounds of the invention can be used in methods of modulating a protein kinase by contacting the protein kinase with any one or more of the compounds or compositions described herein. In some embodiments, the compounds can act as inhibitors of one or more protein kinases. In some embodiments, the compounds can act to stimulate the activity of one or more protein kinases. In further embodiments, the compounds of the invention can be used to modulate activity of a protein kinase in an individual in need of modulation of the receptor by administering a modulating amount of a compound as described herein.

As used herein, the term “BTK-mediated” or “BTK-modulated” or “BTK-dependent” diseases or disorders means any disease or other deleterious condition in which BTK, or a mutant thereof, is known to play a role. Accordingly, another embodiment of the present application relates to treating or lessening the severity of one or more diseases in which BTK, or a mutant thereof, is known to play a role. Specifically, the present application relates to a method of treating or lessening the severity of a disease or condition selected from a proliferative disease or disorder, such as cancer or an oncology indication, an autoimmune disease or disorder, an inflammatory disease or disorder, or a thromboembolic disease or disorder, wherein said method comprises administering to a patient in need thereof a compound of Formula (I), or a pharmaceutically acceptable salt, solvate, hydrate, isomer, tautomer, racemate, or isotope thereof, or a composition according to the present application. In an embodiment, the autoimmune disease or disorder is multiple sclerosis, rheumatoid arthritis, psoriasis, Sjogren's syndrome, or systemic lupus erythematosus. In another embodiment, the inflammatory disease or disorder is urticaria. In another embodiment, the cancer or oncology indication is primary CNS lymphoma.

### Compounds

In one embodiment, a compound is provided having the structure of Formula (I):

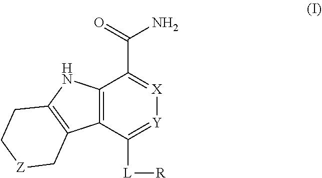

or a pharmaceutically acceptable salt, solvate, hydrate, isomer, tautomer, racemate, or isotope thereof, wherein:

X is CR¹ or N;

Y is CR² or N;

R¹ is H, halo, or C₁₋₃ alkyl;

R² is H, halo, or C₁₋₃ alkyl;

Z is a bond, C(R³R⁴), C(R⁵R⁶)—C(R⁷R⁸), C(R⁹R¹⁰)—N(R¹¹), N(R¹¹)—C(R⁹R¹⁰), O, S, Si(R¹²R¹³),

R³ and R⁴ are each, independently, H, halo, or C₁₋₃ alkyl, or R³ and R⁴, together with the carbon to which they are attached, form ring A:

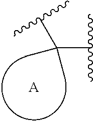

wherein ring A is an optionally substituted carbocycle or an optionally substituted heterocycle;

R⁵, R⁶, R⁷, R⁸, R⁹, and R¹⁰ are each, independently, H, halo, or C₁₋₃ alkyl;

R¹¹, R¹², and R¹³ are each, independently, H or C₁₋₃ alkyl;

L is

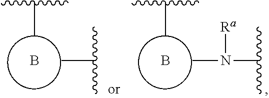

ring B is an optionally substituted carbocycle or an optionally substituted heterocycle;

R^{a} is H or C₁₋₃ alkyl;

R is C(O)R¹⁴; and

R¹⁴ is C₂₋₆ alkenyl substituted with 0-3 R′ or C₂₋₆ alkynyl substituted with 0-3 R′;

R′ is at each occurrence, independently, halo, —OR^{b}, —NR^{b}R^{c}, or optionally substituted carbocycle; and

R^{b} and R^{c} are at each occurrence, independently, H, C₁₋₆ alkyl, or C₁₋₆ haloalkyl; with the proviso that if Z is CH₂ or CF₂, then at least one of X and Y is N.

In some embodiments X is CH. In some embodiments X is CH and Y is CH. In some embodiments X is CH and Y is N. In some embodiments X is CH and Y is CF. In some embodiments X is CH and Y is Y is C(CH₃). In some embodiments X is N. In some embodiments X is N and Y is CH. In some embodiments X is N and Y is N. In some embodiments X is N and Y is CF. In some embodiments X is N and Y is C(CH₃).

In some embodiments Y is CH. In some embodiments Y is N. In some embodiments Y is CF. In some embodiments Y is C(CH₃).

In some embodiments Z is a bond. In some embodiments Z is CH₂ or CF₂. In some embodiments Z is CH₂ or CH₂—CH₂. In some embodiments Z is CH₂. In some embodiments Z is CF₂. In some embodiments Z is CH₂—CH₂. In some embodiments Z is O, S or Si(CH₃)₂. In some embodiments Z is O. In some embodiments Z is S. In some embodiments Z is Si(CH₃)₂. In some embodiments Z is CH₂—N(CH₃).

In some embodiments, Z is C(R³R⁴) and R³ and R⁴ are each, independently, H. In one embodiment, a compound is provided having the structure of Formula (I-A):

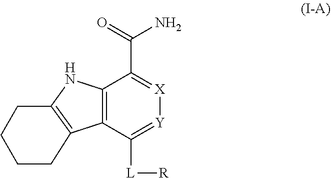

or a pharmaceutically acceptable salt, solvate, hydrate, isomer, tautomer, racemate, or isotope thereof, wherein X, Y, L, and R are as described by Formula (I).

In some embodiments, Z is CH₂—CH₂. In one embodiment, a compound is provided having the structure of Formula (I-B):

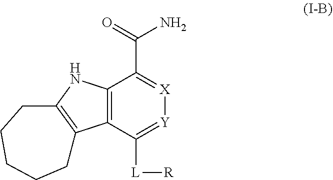

or a pharmaceutically acceptable salt, solvate, hydrate, isomer, tautomer, racemate, or isotope thereof, wherein X, Y, L, and R are as described by Formula (I).

In some embodiments, Z is C(R³R⁴). In one embodiment, R³ is H. In another embodiment, R⁴ is H. In one embodiment, R³ is halo. In a further embodiment, R³ is F or Cl. In another embodiment, R⁴ is halo. In a further embodiment, R⁴ is F or Cl. In one embodiment, R³ is C₁₋₃ alkyl. In one embodiment, R³ is —CH₃. In another embodiment, R⁴ is C₁₋₃ alkyl. In a further embodiment, R⁴ is —CH₃.

In one embodiment, R³ and R⁴ are each, independently, halo. In another embodiment, R³ and R⁴ are each, independently, F. In one embodiment, R³ and R⁴ are each, independently, C₁₋₃ alkyl. In another embodiment, R³ and R⁴ are each, independently, —CH₃.

In another embodiment, R³ and R⁴, together with the carbon to which they are attached, form ring A. In one embodiment, a compound is provided having the structure of Formula (I-C):

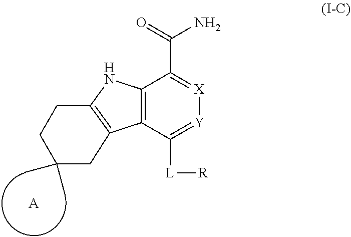

or a pharmaceutically acceptable salt, solvate, hydrate, isomer, tautomer, racemate, or isotope thereof, wherein A, X, Y, L, and R are as described by Formula (I).

In some embodiments, a compound is provided having the structure of Formula (I-D):

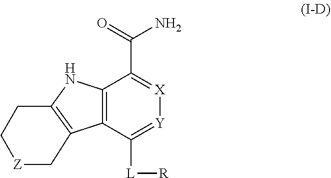

or a pharmaceutically acceptable salt, solvate, hydrate, isomer, tautomer, racemate, or isotope thereof, wherein:

Z is a bond, O, S, Si(CH₃)₂ or CH₂N(CH₃); and

X, Y, L, and R are as described by Formula (I).

In some embodiments, compounds are provided having the structure of Formula (I-A-iii-a), Formula (I-A-iii-b), or Formula (I-A-iv):

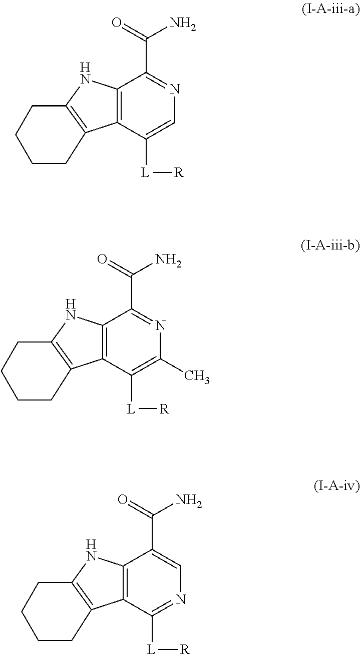

or a pharmaceutically acceptable salt, solvate, hydrate, isomer, tautomer, racemate, or isotope thereof, wherein L and R are as described by Formula (I).

In some embodiments, compounds are provided having the structure of Formula (I-B-i), (I-B-ii), (I-B-iii-a), (I-B-iii-b), or (I-B-iv):

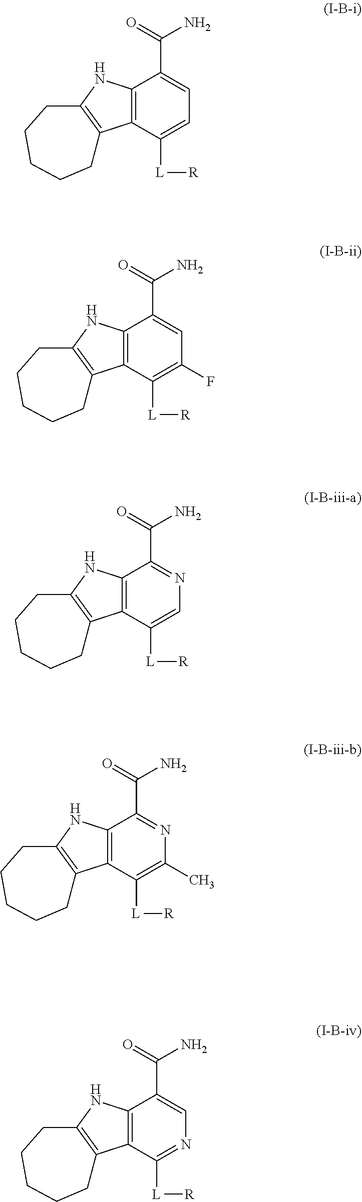

or a pharmaceutically acceptable salt, solvate, hydrate, isomer, tautomer, racemate, or isotope thereof, wherein L and R are as described by Formula (I).

In some embodiments, compounds are provided having the structure of Formula (I-C-i), (I-C-ii), (I-C-iii-a), (I-C-iii-b) or (I-C-iv):

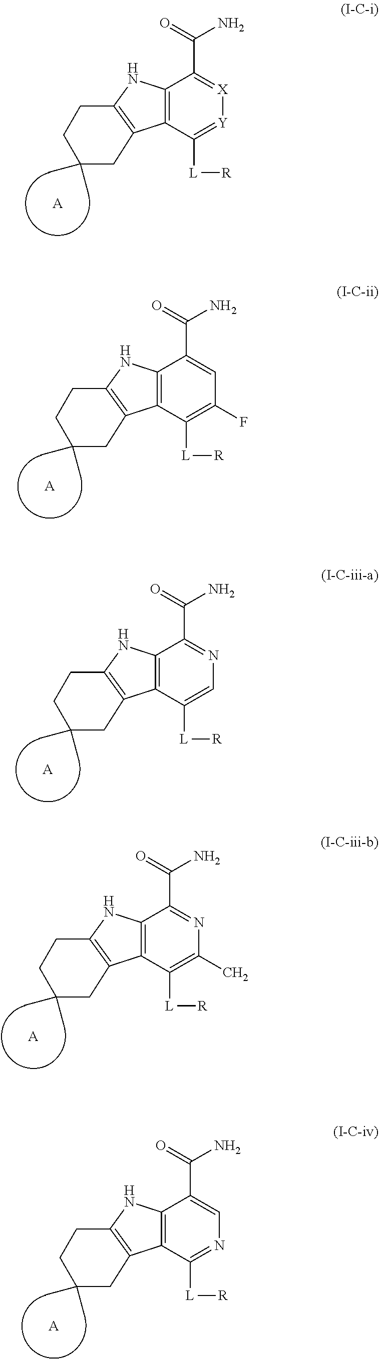

or a pharmaceutically acceptable salt, solvate, hydrate, isomer, tautomer, racemate, or isotope thereof, wherein A, L, and R are as described by Formula (I).

In some embodiments, compounds are provided having the structure of Formula (I), (I-C), (I-C-i), (I-C-ii), (I-C-iii-a), (I-C-iii-b) or (I-C-iv), wherein A is an optionally substituted, 3-, 4-, 5- or 6-membered carbocyclic ring optionally containing 1 or 2 heteroatoms selected from O, S and N in place of a ring carbon atom. In some embodiments A is a substituted 3-, 4-, 5- or 6-membered cycloalkyl ring. In some embodiments A is an unsubstituted 3-, 4-, 5- or 6-membered cycloalkyl ring. In some embodiments A is

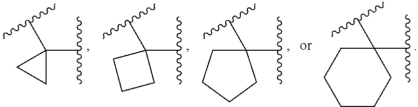

In some embodiments, compounds are provided having the structure of Formula (I), (I-C), (I-C-i), (I-C-ii), (I-C-iii-a), (I-C-iii-b) or (I-C-iv), wherein A contains 1 heteroatom selected from O, S and N. In some embodiments A contains 1 O atom. In some embodiments A contains 1 S atom. In some embodiments A contains 1 N atom. In some embodiments A is a 5-membered ring, containing 1 heteroatom selected from O, S and N. In some embodiments A is substituted. In some embodiments A is unsubstituted. In some embodiments A is a 6-membered ring, containing 1 heteroatom selected from O, S and N. In some embodiments A is substituted. In some embodiments A is unsubstituted. In some embodiments A contains 2 heteroatoms selected from O, S and N. In some embodiments, the two heteroatoms are the same. In some embodiments, the two heteroatoms are the different. In some embodiments A contains 2 N atoms, hi some embodiments A contains 2 O atoms, hi some embodiments A contains 1 N atom and 1 O atom. In some embodiments A is a 5-membered ring, containing 2 heteroatoms selected from O, S and N. In some embodiments A is substituted. In some embodiments A is unsubstituted. In some embodiments A is a 6-membered ring, containing 2 heteroatoms selected from O, S and N. In some embodiments A is substituted. In some embodiments A is unsubstituted. In some embodiments A is a 5-membered ring, containing 1 or 2 N atoms. In some embodiments A is a 6-membered ring, containing 1 or 2 N atoms. In some embodiments A is a 5-membered ring, containing 1 or 2 O atoms. In some embodiments A is a 6-membered ring, containing 1 or 2 O atoms. In some embodiments A is a 5-membered ring, containing 1 N and 1 O atom. In some embodiments A is a 6-membered ring, containing 1 N and 1 O atom.

In some embodiments, compounds are provided having the structure of Formula (I), (I-C), (I-C-i), (I-C-ii), (I-C-iii-a), (I-C-iii-b) or (I-C-iv), wherein A is substituted by 0-3 substituents independently selected from halo, C₁₋₃ alkyl, C₁₋₃ haloalkyl, oxo, or NH₂.

In some embodiments, compounds are provided having the structure of Formula (I), (I-C), (I-C-i), (I-C-ii), (I-C-iii-a), (I-C-iii-b) or (I-C-iv), wherein A is

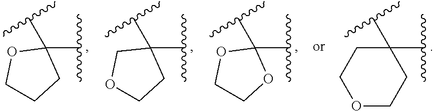

In some embodiments, compounds are provided having the structure of Formula (I), (I-C), (I-C-i), (I-C-ii), (I-C-iii-a), (I-C-iii-b) or (I-C-iv), wherein A is

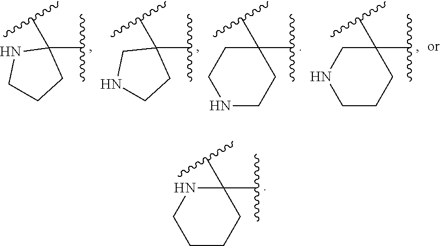

In some embodiments, compounds are provided having the structure of Formula (I), (FC), (I-C-i), (I-C-ii), (I-C-iii-a), (I-C-iii-b) or (I-C-iv), wherein A is

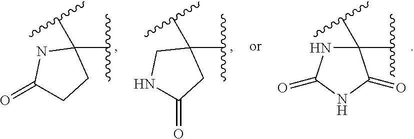 [SMILES: *C1(*)CCC(=O)N1 ; *C1(*)CNC(=O)C1 ; *C1(*)NC(=O)NC1=O]

In some embodiments, compounds are provided having the structure of Formula (I-D-i), (I-D-ii), (I-D-iii-a), (I-D-iii-b) or (I-D-iv):

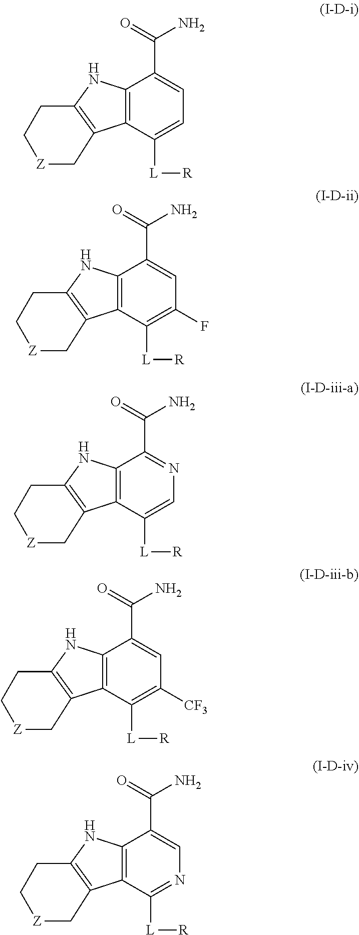

or a pharmaceutically acceptable salt, solvate, hydrate, isomer, tautomer, racemate, or isotope thereof, wherein Z, L, and R are as described by Formula (I-D).

In some embodiments, compounds are provided having the structure of Formula (I-D-v), (I-D-vi), (I-D-vii) or (I-D-viii):

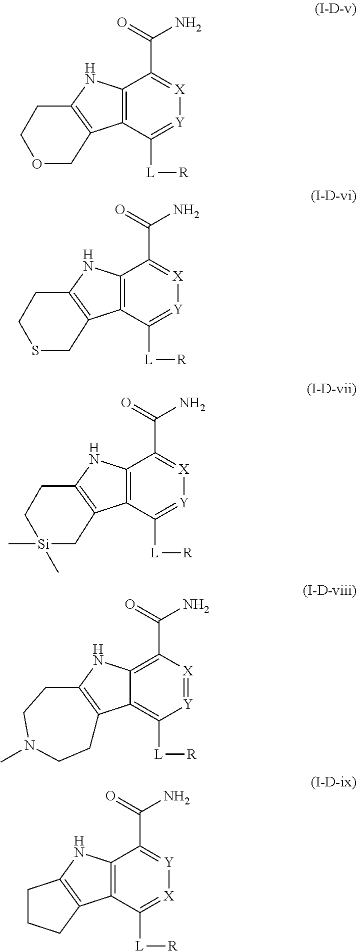

or a pharmaceutically acceptable salt, solvate, hydrate, isomer, tautomer, racemate, or isotope thereof, wherein L, and R are as described by Formula (I).

In one embodiment, a compound having the structure of any one of Formula (I), (I-A), (I-A-iii-a), (I-A-iii-b), (I-A-iv), (I-B), (I-B-i), (I-B-ii), (I-B-iii-a), (I-B-iii-b), (I-B-iv), (I-C), (I-C-i), (I-C-ii), (I-C-iii-a), (I-C-iii-b), (I-C-iv), (I-D), (I-D-i), (I-D-ii), (I-D-iii-a), (I-D-iii-b), (I-D-iv), (I-D-v), (I-D-vi), (I-D-vii), (I-D-viii), or (I-D-ix) is provided, or a pharmaceutically acceptable salt, solvate, hydrate, isomer, tautomer, racemate, or isotope thereof, wherein ring B is optionally substituted with 0-5 R¹⁵, wherein R¹⁵ is at each occurrence, independently, —OH, —CN, halo, C₁₋₆ alkyl, C₁₋₆haloalkyl, C₁₋₆ alkoxy, or C₁₋₆haloalkoxy. In one embodiment, at least one occurrence of R¹⁵ is —OH. In one embodiment, at least one occurrence of R¹⁵ is halo. In one embodiment, at least one occurrence of R¹⁵ is F. In one embodiment, at least one occurrence of R¹⁵ is Cl. In one embodiment, at least one occurrence of R¹⁵ is C₁₋₃ alkyl. In one embodiment, at least one occurrence of R¹⁵ is —CH₃. In one embodiment, at least one occurrence of R¹⁵ is C₁₋₃ haloalkyl. In one embodiment, at least one occurrence of R¹⁵ is —CF₃.

In one embodiment, a compound having the structure of any one of Formula (I), (I-A), (I-A-iii-a), (I-A-iii-b), (I-A-iv), (I-B), (I-B-i), (I-B-ii), (I-B-iii-a), (I-B-iii-b), (I-B-iv), (I-C), (I-C-i), (I-C-ii), (I-C-iii-a), (I-C-iii-b), (I-C-iv), (I-D), (I-D-i), (I-D-ii), (I-D-iii-a), (I-D-iii-b), (I-D-iv), (I-D-v), (I-D-vi), (I-D-vii), (I-D-viii), or (I-D-ix) is provided, or a pharmaceutically acceptable salt, solvate, hydrate, isomer, tautomer, racemate, or isotope thereof, wherein ring B is an optionally substituted carbocycle. In one embodiment, a compound having the structure of any one of Formula (I), (I-A), (I-A-iii-a), (I-A-iii-b), (I-A-iv), (I-B), (I-B-i), (I-B-ii), (I-B-iii-a), (I-B-iii-b), (I-B-iv), (I-C), (I-C-i), (I-C-ii), (I-C-iii-a), (I-C-iii-b), (I-C-iv), (I-D), (I-D-i), (I-D-ii), (I-D-iii-a), (I-D-iii-b), (I-D-iv), (I-D-v), (I-D-vi), (I-D-vii), (I-D-viii), or (I-D-ix) is provided, or a pharmaceutically acceptable salt, solvate, hydrate, isomer, tautomer, racemate, or isotope thereof, wherein ring B is an optionally substituted aromatic carbocycle. In a further embodiment a compound having the structure of any one of Formula (I), (I-A), (I-A-iii-a), (I-A-iii-b), (I-A-iv), (I-B), (I-B-i), (I-B-ii), (I-B-iii-a), (I-B-iii-b), (I-B-iv), (I-C), (I-C-i), (I-C-ii), (I-C-iii-a), (I-C-iii-b), (I-C-iv), (I-D), (I-D-i), (I-D-ii), (I-D-iii-a), (I-D-iii-b), (I-D-iv), (I-D-v), (I-D-vi), (I-D-vii), (I-D-viii), or (I-D-ix) is provided, or a pharmaceutically acceptable salt, solvate, hydrate, isomer, tautomer, racemate, or isotope thereof, wherein L is:

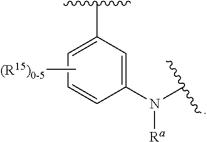

In one embodiment, a compound having the structure of any one of Formula (I), (I-A), (I-A-iii-a), (I-A-iii-b), (I-A-iv), (I-B), (I-B-i), (I-B-ii), (I-B-iii-a), (I-B-iii-b), (I-B-iv), (I-C), (I-C-i), (I-C-ii), (I-C-iii-a), (I-C-iii-b), (I-C-iv), (I-D), (I-D-i), (I-D-ii), (I-D-iii-a), (I-D-iii-b), (I-D-iv), (I-D-v), (I-D-vi), (I-D-vii), (I-D-viii), or (I-D-ix) is provided, or a pharmaceutically acceptable salt, solvate, hydrate, isomer, tautomer, racemate, or isotope thereof, wherein L is

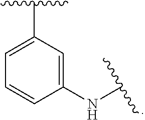 [SMILES: *Nc1cccc(*)c1]

In another embodiment, a compound having the structure of any one of Formula (I), (I-A), (I-A-iii-a), (I-A-iii-b), (I-A-iv), (I-B), (I-B-i), (I-B-ii), (I-B-iii-a), (I-B-iii-b), (I-B-iv), (I-C), (I-C-i), (I-C-ii), (I-C-iii-a), (I-C-iii-b), (I-C-iv), (I-D), (I-D-i), (I-D-ii), (I-D-iii-a), (I-D-iii-b), (I-D-iv), (I-D-v), (I-D-vi), (I-D-vii), (I-D-viii), or (I-D-ix) is provided, or a pharmaceutically acceptable salt, solvate, hydrate, isomer, tautomer, racemate, or isotope thereof, wherein ring B is an optionally substituted non-aromatic carbocycle. In a further embodiment, a compound having the structure of any one of Formula (I), (I-A), (I-A-iii-a), (I-A-iii-b), (I-A-iv), (I-B), (I-B-i), (I-B-ii), (I-B-iii-a), (I-B-iii-b), (I-B-iv), (I-C), (I-C-i), (I-C-ii), (I-C-iii-a), (I-C-iii-b), (I-C-iv), (I-D), (I-D-i), (I-D-ii), (I-D-iii-a), (I-D-iii-b), (I-D-iv), (I-D-v), (I-D-vi), (I-D-vii), (I-D-viii), or (I-D-ix) is provided, or a pharmaceutically acceptable salt, solvate, hydrate, isomer, tautomer, racemate, or isotope thereof, wherein L is

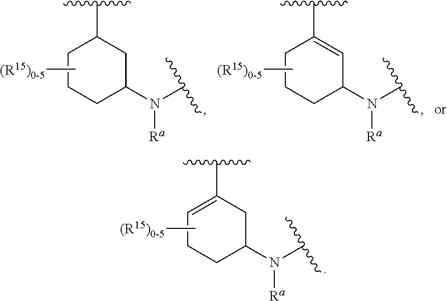

In one embodiment, a compound having the structure of any one of Formula (I), (I-A), (I-A-iii-a), (I-A-iii-b), (I-A-iv), (I-B), (I-B-i), (I-B-ii), (I-B-iii-a), (I-B-iii-b), (I-B-iv), (I-C), (I-C-i), (I-C-ii), (I-C-iii-a), (I-C-iii-b), (I-C-iv), (I-D), (I-D-i), (I-D-ii), (I-D-iii-a), (I-D-iii-b), (I-D-iv), (I-D-v), (I-D-vi), (I-D-vii), (I-D-viii), or (I-D-ix) is provided, or a pharmaceutically acceptable salt, solvate, hydrate, isomer, tautomer, racemate, or isotope thereof, wherein L is

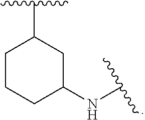 [SMILES: *NC1CCCC(*)C1]

In one embodiment, a compound having the structure of any one of Formula (I), (I-A), (I-A-iii-a), (I-A-iii-b), (I-A-iv), (I-B), (I-B-i), (I-B-ii), (I-B-iii-a), (I-B-iii-b), (I-B-iv), (I-C), (I-C-i), (I-C-ii), (I-C-iii-a), (I-C-iii-b), (I-C-iv), (I-D), (I-D-i), (I-D-ii), (I-D-iii-a), (I-D-iii-b), (I-D-iv), (I-D-v), (I-D-vi), (I-D-vii), (I-D-viii), or (I-D-ix) is provided, or a pharmaceutically acceptable salt, solvate, hydrate, isomer, tautomer, racemate, or isotope thereof, wherein L is

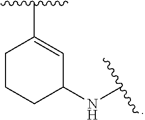 [SMILES: *NC1C=C(*)CCC1]

In one embodiment, a compound having the structure of any one of Formula (I), (I-A), (I-A-iii-a), (I-A-iii-b), (I-A-iv), (I-B), (I-B-i), (I-B-ii), (I-B-iii-a), (I-B-iii-b), (I-B-iv), (I-C), (I-C-i), (I-C-ii), (I-C-iii-a), (I-C-iii-b), (I-C-iv), (I-D), (I-D-i), (I-D-ii), (I-D-iii-a), (I-D-iii-b), (I-D-iv), (I-D-v), (I-D-vi), (I-D-vii), (I-D-viii), or (I-D-ix) is provided, or a pharmaceutically acceptable salt, solvate, hydrate, isomer, tautomer, racemate, or isotope thereof, wherein L is

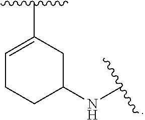 [SMILES: *NC1CCC=C(*)C1]

In one embodiment, a compound having the structure of any one of Formula (I), (I-A), (I-A-iii-a), (I-A-iii-b), (I-A-iv), (I-B), (I-B-i), (I-B-ii), (I-B-iii-a), (I-B-iii-b), (I-B-iv), (I-C), (I-C-i), (I-C-ii), (I-C-iii-a), (I-C-iii-b), (I-C-iv), (I-D), (I-D-i), (I-D-ii), (I-D-iii-a), (I-D-iii-b), (I-D-iv), (I-D-v), (I-D-vi), (I-D-vii), (I-D-viii), or (I-D-ix) is provided, or a pharmaceutically acceptable salt, solvate, hydrate, isomer, tautomer, racemate, or isotope thereof, wherein ring B is an optionally substituted heterocycle. In a further embodiment, a compound having the structure of any one of Formula (I), (I-A), (I-A-iii-a), (I-A-iii-b), (I-A-iv), (I-B), (I-B-i), (I-B-ii), (I-B-iii-a), (I-B-iii-b), (I-B-iv), (I-C), (I-C-i), (I-C-ii), (I-C-iii-a), (I-C-iii-b), (I-C-iv), (I-D), (I-D-i), (I-D-ii), (I-D-iii-a), (I-D-iii-b), (I-D-iv), (I-D-v), (I-D-vi), (I-D-vii), (I-D-viii), or (I-D-ix) is provided, or a pharmaceutically acceptable salt, solvate, hydrate, isomer, tautomer, racemate, or isotope thereof, wherein L is

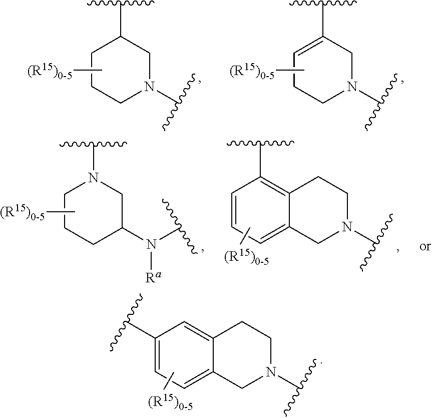

In a further embodiment, a compound having the structure of any one of Formula (I), (I-A), (I-A-iii-a), (I-A-iii-b), (I-A-iv), (I-B), (I-B-i), (I-B-ii), (I-B-iii-a), (I-B-iii-b), (I-B-iv), (I-C), (I-C-i), (I-C-ii), (I-C-iii-a), (I-C-iii-b), (I-C-iv), (I-D), (I-D-i), (I-D-ii), (I-D-iii-a), (I-D-iii-b), (I-D-iv), (I-D-v), (I-D-vi), (I-D-vii), (I-D-viii), or (I-D-ix) is provided, or a pharmaceutically acceptable salt, solvate, hydrate, isomer, tautomer, racemate, or isotope thereof, wherein L is

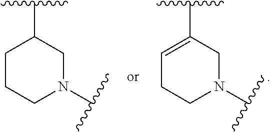 [SMILES: *C1CCCN(*)C1 ; *C1=CCCN(*)C1]

In a further embodiment, a compound having the structure of any one of Formula (I), (I-A), (I-A-iii-a), (I-A-iii-b), (I-A-iv), (I-B), (I-B-i), (I-B-ii), (I-B-iii-a), (I-B-iii-b), (I-B-iv), (I-C), (I-C-i), (I-C-ii), (I-C-iii-a), (I-C-iii-b), (I-C-iv), (I-D), (I-D-i), (I-D-ii), (I-D-iii-a), (I-D-iii-b), (I-D-iv), (I-D-v), (I-D-vi), (I-D-vii), (I-D-viii), or (I-D-ix) is provided, or a pharmaceutically acceptable salt, solvate, hydrate, isomer, tautomer, racemate, or isotope thereof, wherein L is

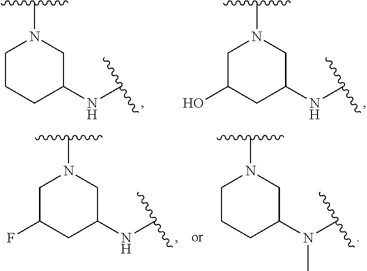

In a further embodiment, a compound having the structure of any one of Formula (I), (I-A), (I-A-iii-a), (I-A-iii-b), (I-A-iv), (I-B), (I-B-i), (I-B-ii), (I-B-iii-a), (I-B-iii-b), (I-B-iv), (I-C), (I-C-i), (I-C-ii), (I-C-iii-a), (I-C-iii-b), (I-C-iv), (I-D), (I-D-i), (I-D-ii), (I-D-iii-a), (I-D-iii-b), (I-D-iv), (I-D-v), (I-D-vi), (I-D-vii), (I-D-viii), or (I-D-ix) is provided, or a pharmaceutically acceptable salt, solvate, hydrate, isomer, tautomer, racemate, or isotope thereof, wherein L is

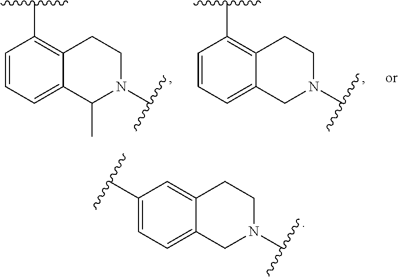 [SMILES: *c1cccc2c1CCN(*)C2C ; *c1cccc2c1CCN(*)C2 ; *c1ccc2c(c1)CCN(*)C2]

In one embodiment, a compound having the structure of any one of Formula (I), (I-A), (I-A-iii-a), (I-A-iii-b), (I-A-iv), (I-B), (I-B-i), (I-B-ii), (I-B-iii-a), (I-B-iii-b), (I-B-iv), (I-C), (I-C-i), (I-C-ii), (I-C-iii-a), (I-C-iii-b), (I-C-iv), (I-D), (I-D-i), (I-D-ii), (I-D-iii-a), (I-D-iii-b), (I-D-iv), (I-D-v), (I-D-vi), (I-D-vii), (I-D-viii), or (I-D-ix) is provided, or a pharmaceutically acceptable salt, solvate, hydrate, isomer, tautomer, racemate, or isotope thereof, wherein R¹⁴ is C₂₋₆ alkenyl substituted with 0-3 R′. In one embodiment, a compound of Formula (I) is provided, wherein R¹⁴ is C₂₋₆ alkenyl substituted with 0 R′.

In one embodiment, a compound having the structure of any one of Formula (I), (I-A), (I-A-iii-a), (I-A-iii-b), (I-A-iv), (I-B), (I-B-i), (I-B-ii), (I-B-iii-a), (I-B-iii-b), (I-B-iv), (I-C), (I-C-i), (I-C-ii), (I-C-iii-a), (I-C-iii-b), (I-C-iv), (I-D), (I-D-i), (I-D-ii), (I-D-iii-a), (I-D-iii-b), (I-D-iv), (I-D-v), (I-D-vi), (I-D-vii), (I-D-viii), or (I-D-ix) is provided, or a pharmaceutically acceptable salt, solvate, hydrate, isomer, tautomer, racemate, or isotope thereof, wherein R¹⁴ is C₂₋₆ alkenyl substituted with 1-3 R′. In one embodiment, at least one R′ is halo. In one embodiment, at least one R′ is —OR^{b}. In one embodiment, at least one R′ is —OH. In another embodiment, at least one R′ is —NR^{b}R^{c}. In a further embodiment, at least one R′ is —NH₂ or —N(CH₃)₂.

In one embodiment, a compound having the structure of any one of Formula (I), (I-A), (I-A-iii-a), (I-A-iii-b), (I-A-iv), (I-B), (I-B-i), (I-B-ii), (I-B-iii-a), (I-B-iii-b), (I-B-iv), (I-C), (I-C-i), (I-C-ii), (I-C-iii-a), (I-C-iii-b), (I-C-iv), (I-D), (I-D-i), (I-D-ii), (I-D-iii-a), (I-D-iii-b), (I-D-iv), (I-D-v), (I-D-vi), (I-D-vii), (I-D-viii), or (I-D-ix) is provided, or a pharmaceutically acceptable salt, solvate, hydrate, isomer, tautomer, racemate, or isotope thereof, wherein R is:

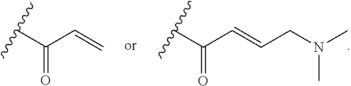

In another embodiment, a compound having the structure of any one of Formula (I), (I-A), (I-A-iii-a), (I-A-iii-b), (I-A-iv), (I-B), (I-B-i), (I-B-ii), (I-B-iii-a), (I-B-iii-b), (I-B-iv), (I-C), (I-C-i), (I-C-ii), (I-C-iii-a), (I-C-iii-b), (I-C-iv), (I-D), (I-D-i), (I-D-ii), (I-D-iii-a), (I-D-iii-b), (I-D-iv), (I-D-v), (I-D-vi), (I-D-vii), (I-D-viii), or (I-D-ix) is provided, or a pharmaceutically acceptable salt, solvate, hydrate, isomer, tautomer, racemate, or isotope thereof, wherein R is:

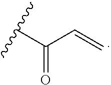 [SMILES: *C(=O)C=C]

In another embodiment, a compound having the structure of any one of Formula (I), (I-A), (I-A-iii-a), (I-A-iii-b), (I-A-iv), (I-B), (I-B-i), (I-B-ii), (I-B-iii-a), (I-B-iii-b), (I-B-iv), (I-C), (I-C-i), (I-C-ii), (I-C-iii-a), (I-C-iii-b), (I-C-iv), (I-D), (I-D-i), (I-D-ii), (I-D-iii-a), (I-D-iii-b), (I-D-iv), (I-D-v), (I-D-vi), (I-D-vii), (I-D-viii), or (I-D-ix) is provided, or a pharmaceutically acceptable salt, solvate, hydrate, isomer, tautomer, racemate, or isotope thereof, wherein R is:

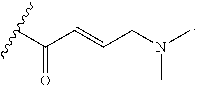 [SMILES: *C(=O)/C=C/CN(C)C]

In one embodiment, a compound having the structure of any one of Formula (I), (I-A), (I-A-iii-a), (I-A-iii-b), (I-A-iv), (I-B), (I-B-i), (I-B-ii), (I-B-iii-a), (I-B-iii-b), (I-B-iv), (I-C), (I-C-i), (I-C-ii), (I-C-iii-a), (I-C-iii-b), (I-C-iv), (I-D), (I-D-i), (I-D-ii), (I-D-iii-a), (I-D-iii-b), (I-D-iv), (I-D-v), (I-D-vi), (I-D-vii), (I-D-viii), or (I-D-ix) is provided, or a pharmaceutically acceptable salt, solvate, hydrate, isomer, tautomer, racemate, or isotope thereof, wherein R¹⁴ is C₂₋₆ alkynyl substituted with 0-3 R′. In one embodiment, a compound of Formula (I) is provided, wherein R¹⁴ is C₂₋₆ alkynyl substituted with 0 R′.

In one embodiment, a compound having the structure of any one of Formula (I), (I-A), (I-A-iii-a), (I-A-iii-b), (I-A-iv), (I-B), (I-B-i), (I-B-ii), (I-B-iii-a), (I-B-iii-b), (I-B-iv), (I-C), (I-C-i), (I-C-ii), (I-C-iii-a), (I-C-iii-b), (I-C-iv), (I-D), (I-D-i), (I-D-ii), (I-D-iii-a), (I-D-iii-b), (I-D-iv), (I-D-v), (I-D-vi), (I-D-vii), (I-D-viii), or (I-D-ix) is provided, or a pharmaceutically acceptable salt, solvate, hydrate, isomer, tautomer, racemate, or isotope thereof, wherein R¹⁴ is C₂₋₆ alkynyl substituted with 1-3 R′. In one embodiment, at least one R′ is halo. In one embodiment, at least one R′ is —OR^{b}. In one embodiment, at least one R′ is —OH. In another embodiment, at least one R′ is —NR^{b}R^{c}. In a further embodiment, at least R′ is —NH₂ or —N(CH₃)₂. In another embodiment, at least one R′ is carbocycle. In another embodiment, at least one R′ is cyclopropyl, cyclobutyl, cyclopentyl, or cyclohexyl.

In one embodiment, a compound having the structure of any one of Formula (I), (I-A), (I-A-iii-a), (I-A-iii-b), (I-A-iv), (I-B), (I-B-i), (I-B-ii), (I-B-iii-a), (I-B-iii-b), (I-B-iv), (I-C), (I-C-i), (I-C-ii), (I-C-iii-a), (I-C-iii-b), (I-C-iv), (I-D), (I-D-i), (I-D-ii), (I-D-iii-a), (I-D-iii-b), (I-D-iv), (I-D-v), (I-D-vi), (I-D-vii), (I-D-viii), or (I-D-ix) is provided, or a pharmaceutically acceptable salt, solvate, hydrate, isomer, tautomer, racemate, or isotope thereof, wherein R is:

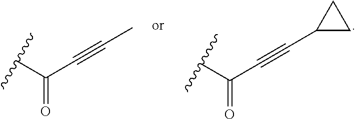

In one embodiment, a compound having the structure of any one of Formula (I), (I-A), (I-A-iii-a), (I-A-iii-b), (I-A-iv), (I-B), (I-B-i), (I-B-ii), (I-B-iii-a), (I-B-iii-b), (I-B-iv), (I-C), (I-C-i), (I-C-ii), (I-C-iii-a), (I-C-iii-b), (I-C-iv), (I-D), (I-D-i), (I-D-ii), (I-D-iii-a), (I-D-iii-b), (I-D-iv), (I-D-v), (I-D-vi), (I-D-vii), (I-D-viii), or (I-D-ix) is provided, or a pharmaceutically acceptable salt, solvate, hydrate, isomer, tautomer, racemate, or isotope thereof, wherein R is:

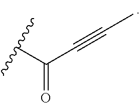 [SMILES: *C(=O)C#CC]

In one embodiment, a compound having the structure of any one of Formula (I), (I-A), (I-A-iii-a), (I-A-iii-b), (I-A-iv), (I-B), (I-B-i), (I-B-ii), (I-B-iii-a), (I-B-iii-b), (I-B-iv), (I-C), (I-C-i), (I-C-ii), (I-C-iii-a), (I-C-iii-b), (I-C-iv), (I-D), (I-D-i), (I-D-ii), (I-D-iii-a), (I-D-iii-b), (I-D-iv), (I-D-v), (I-D-vi), (I-D-vii), (I-D-viii), or (I-D-ix) is provided, or a pharmaceutically acceptable salt, solvate, hydrate, isomer, tautomer, racemate, or isotope thereof, wherein R is:

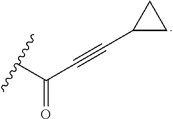 [SMILES: *C(=O)C#CC1CC1]

In one embodiment, a compound is provided having the structure of Formula (I′):

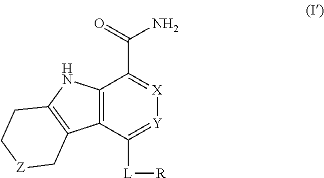

or a pharmaceutically acceptable salt, solvate, hydrate, isomer, tautomer, racemate, or isotope thereof, wherein:

X is CH or N;

Y is CH, N, CF, or C(CH₃);
with the proviso that X and Y cannot both be N,

Z is a bond CH₂, CF₂, CH₂—CH₂, O, S, Si(CH₃)₂, —CH₂—N(CH₃)—, —N(CH₃)—CH₂—, or

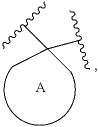

wherein A is an optionally substituted, 3-, 4-, 5- or 6-membered carbocyclic ring optionally containing 1 or 2 heteroatoms selected from O, S, and N in place of 1 or 2 ring carbon atoms,

with the proviso that if Z is CH₂, then at least one of X and Y is N;

L is

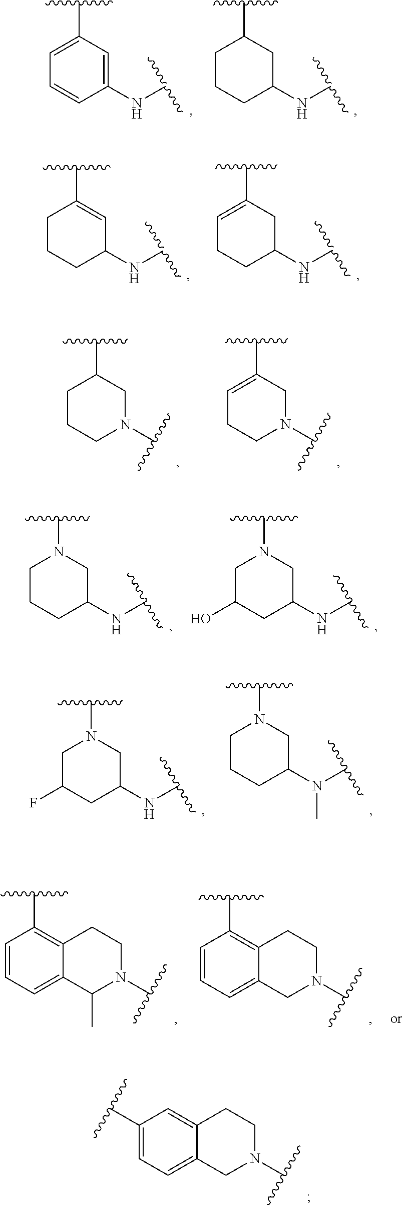

R is

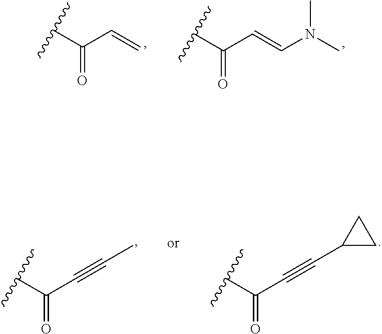

In some embodiments X is CH. In some embodiments X is CH and Y is CH. In some embodiments X is CH and Y is N. In some embodiments X is CH and Y is CF. In some embodiments X is CH and Y is Y is C(CH₃). In some embodiments X is N. In some embodiments X is N and Y is CH. In some embodiments X is N and Y is N. In some embodiments X is N and Y is CF. In some embodiments X is N and Y is C(CH₃).

In some embodiments Y is CH. In some embodiments Y is N. In some embodiments Y is CF. In some embodiments Y is C(CH₃).

In some embodiments Z is a bond. In some embodiments Z is CH₂ or CF₂. In some embodiments Z is CH₂ or CH₂—CH₂. In some embodiments Z is CH₂. In some embodiments Z is CF₂. In some embodiments Z is CH₂—CH₂. In some embodiments Z is O, S or Si(CH₃)₂. In some embodiments Z is O. In some embodiments Z is S. In some embodiments Z is Si(CH₃)₂. In some embodiments Z is CH₂—N(CH₃). In some embodiments Z is

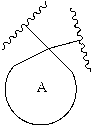

wherein A is an optionally substituted, 3-, 4-, 5- or 6-membered carbocyclic ring optionally containing 1 or 2 heteroatoms selected from O, S and N in place of a ring carbon atom. In some embodiments A is a substituted 3-, 4-, 5- or 6-membered cycloalkyl ring. In some embodiments A is an unsubstituted 3-, 4-, 5- or 6-membered cycloalkyl ring. In some embodiments A is

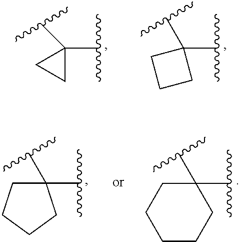 [SMILES: *C1(*)CC1 ; *C1(*)CCC1 ; *C1(*)CCCC1 ; *C1(*)CCCCC1]

In some embodiments A contains 1 heteroatom selected from O, S and N. In some embodiments A contains 1 O atom. In some embodiments A contains 1 S atom. In some embodiments A contains 1 N atom. In some embodiments A is a 5-membered ring, containing 1 heteroatom selected from O, S and N. In some embodiments A is substituted. In some embodiments A is unsubstituted. In some embodiments A is a 6-membered ring, containing 1 heteroatom selected from O, S and N. In some embodiments A is substituted. In some embodiments A is unsubstituted. In some embodiments A contains 2 heteroatoms selected from O, S and N. In some embodiments, the two heteroatoms are the same. In some embodiments, the two heteroatoms are the different. In some embodiments A contains 2 N atoms. In some embodiments A contains 2 O atoms. In some embodiments A contains 1 N atom and 1 O atom. In some embodiments A is a 5-membered ring, containing 2 heteroatoms selected from O, S and N. In some embodiments A is substituted. In some embodiments A is unsubstituted. In some embodiments A is a 6-membered ring, containing 2 heteroatoms selected from O, S and N. In some embodiments A is substituted. In some embodiments A is unsubstituted. In some embodiments A is a 5-membered ring, containing 1 or 2 N atoms. In some embodiments A is a 6-membered ring, containing 1 or 2 N atoms. In some embodiments A is a 5-membered ring, containing 1 or 2 O atoms. In some embodiments A is a 6-membered ring, containing 1 or 2 O atoms. In some embodiments A is a 5-membered ring, containing 1 N and 1 O atom. In some embodiments A is a 6-membered ring, containing 1 N and 1 O atom.

In some embodiments A is

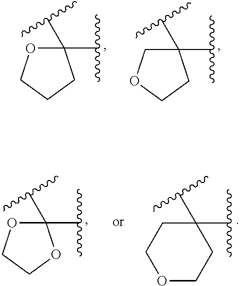 [SMILES: *C1(*)CCCO1 ; *C1(*)CCOC1 ; *C1(*)OCCO1 ; *C1(*)CCOCC1]

In some embodiments A is

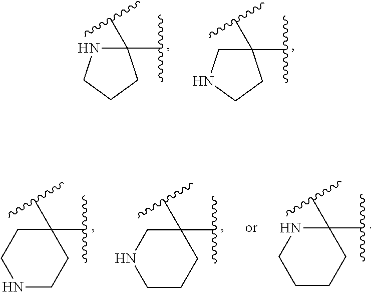 [SMILES: *C1(*)CCCN1 ; *C1(*)CCNC1 ; *C1(*)CCNCC1 ; *C1(*)CCCNC1 ; *C1(*)CCCCN1]

In some embodiments A is

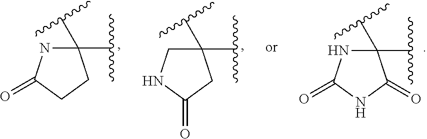 [SMILES: *C1(*)CCC(=O)N1 ; *C1(*)CNC(=O)C1 ; *C1(*)NC(=O)NC1=O]

In some embodiments L is

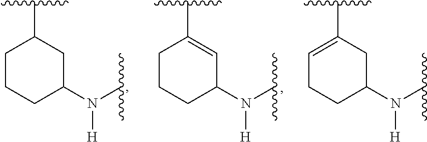

In some embodiments L is

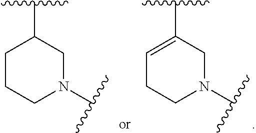 [SMILES: *C1CCCN(*)C1 ; *C1=CCCN(*)C1]

In some embodiments L is

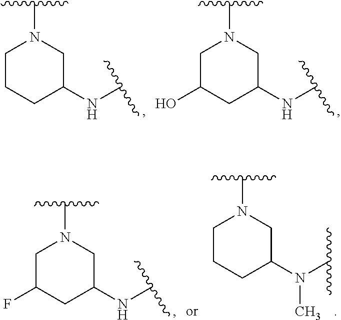

In some embodiments L is

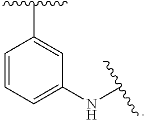 [SMILES: *Nc1cccc(*)c1]

In some embodiments L is

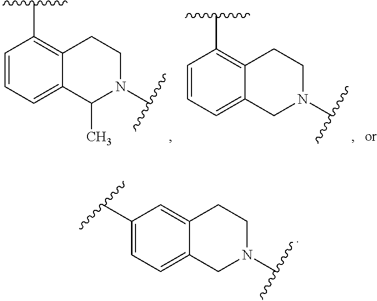

In some embodiments R is

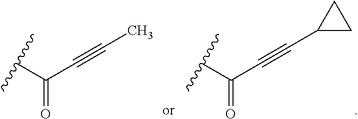

In some embodiments R is

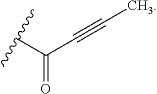 [SMILES: *C(=O)C#CC]

In some embodiments R is

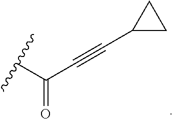 [SMILES: *C(=O)C#CC1CC1]

In one embodiment, a compound is provided having the structure of Formula

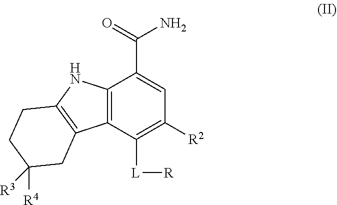

or a pharmaceutically acceptable salt, solvate, hydrate, isomer, tautomer, racemate, or isotope thereof, wherein:

R² is H or C₁₋₃ alkyl;

R³ and R⁴ are each, independently, H, halo, or C₁₋₃ alkyl;

L is

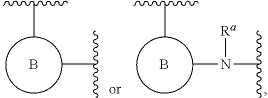

ring B is an optionally substituted non-aromatic carbocycle or an optionally substituted heterocycle;

R^{a} is H or C₁₋₃ alkyl;

R is C(O)R¹⁴; and

R¹⁴ is C₂₋₆ alkenyl substituted with 0-3 R′ or C₂₋₆ alkynyl substituted with 0-3 R′;

R′ is, at each occurrence, independently, halo, —OR^{b}, —NR^{b}R^{c}, or optionally substituted carbocycle; and

R^{b} and R^{c} are at each occurrence, independently, H, C₁₋₆ alkyl, or C₁₋₆ haloalkyl.

In one embodiment, a compound of Formula (II) is provided, wherein R² is H.

In one embodiment, a compound is provided having the structure of Formula (II-A):

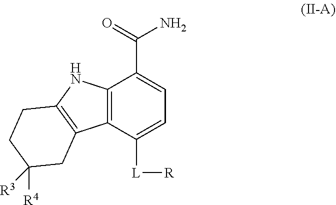

or a pharmaceutically acceptable salt, solvate, hydrate, isomer, tautomer, racemate, or isotope thereof, wherein L, R, R³, and R⁴ are as described by Formula (II).

In one embodiment, a compound of Formula (II) is provided, wherein R² is C₁₋₃ alkyl. In one embodiment, R² is —CH₃.

In one embodiment, a compound of Formula (II) is provided, wherein R³ is H.

In another embodiment, a compound of Formula (II) is provided, wherein R³ is halo. In one embodiment, R³ is F. In another embodiment, R³ is Cl. In one embodiment, a compound of Formula (II) is provided, wherein R³ is C₁₋₃ alkyl. In one embodiment, R³ is —CH₃.

In one embodiment, a compound of Formula (II) is provided, wherein R⁴ is H. In another embodiment, a compound of Formula (II) is provided, wherein R⁴ is halo. In one embodiment, R⁴ is F. In another embodiment, R⁴ is Cl. In one embodiment, a compound of Formula (II) is provided, wherein R⁴ is C₁₋₃ alkyl. In one embodiment, R⁴ is —CH₃.

In one embodiment, a compound of Formula (II) is provided, wherein R³ is H and R⁴ is H. In one embodiment, a compound of Formula (II) is provided, wherein R³ is —CH₃ and R⁴ is —CH₃. In one embodiment, a compound of Formula (II) is provided, wherein R³ is F and R⁴ is F.

In one embodiment, a compound of Formula (II) is provided, wherein R² is H, R³ is H, and R⁴ is H. In one embodiment, a compound is provided having the structure of Formula (II-A-i):

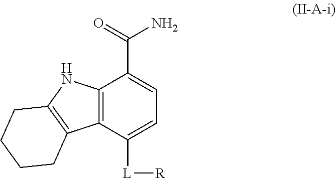

or a pharmaceutically acceptable salt, solvate, hydrate, isomer, tautomer, racemate, or isotope thereof, wherein L and R are as described by Formula (II).

In one embodiment, a compound of Formula (II) is provided, wherein R² is H, R³ is —CH₃, and R⁴ is —CH₃. In one embodiment, a compound of Formula (II) is provided, wherein R² is H, R³ is F, and R⁴ is F.

In one embodiment, a compound of Formula (II) is provided, wherein R² is —CH₃, R³ is H, and R⁴ is H. In one embodiment, a compound of Formula (II) is provided, wherein R² is —CH₃, R³ is —CH₃, and R⁴ is —CH₃. In one embodiment, a compound of Formula (II) is provided, wherein R² is —CH₃, R³ is F, and R⁴ is F.

In one embodiment, a compound of Formula (II), Formula (II-A), or Formula (II-A), or Formula (II-A-i) is provided, wherein ring B is optionally substituted with 0-5 R¹⁵, wherein R¹⁵ is at each occurrence, independently, —OH, —CN, halo, C₁₋₆ alkyl, C₁₋₆haloalkyl, C₁₋₆ alkoxy, or C₁₋₆haloalkoxy. In one embodiment, at least one occurrence of R¹⁵ is —OH. In one embodiment, at least one occurrence of R¹⁵ is halo. In one embodiment, at least one occurrence of R¹⁵ is F. In one embodiment, at least one occurrence of R¹⁵ is Cl. In one embodiment, at least one occurrence of R¹⁵ is C₁₋₃ alkyl. In one embodiment, at least one occurrence of R¹⁵ is —CH₃. In one embodiment, at least one occurrence of R¹⁵ is C₁₋₃ haloalkyl. In one embodiment, at least one occurrence of R¹⁵ is —CF₃.

In one embodiment, a compound of Formula (II), Formula (II-A), or Formula (II-A-i) is provided, wherein ring B is an optionally substituted non-aromatic carbocycle. In another embodiment, a compound of Formula (II), Formula (II-A), or Formula (II-A-i) is provided, wherein L is

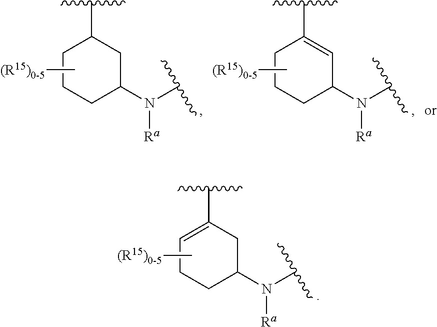

In one embodiment, a compound of Formula (II), Formula (II-A), or Formula (II-A-i) is provided, wherein L is

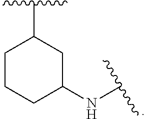 [SMILES: *NC1CCCC(*)C1]

In one embodiment, a compound of Formula (II), Formula (II-A), or Formula (II-A-i) is provided, wherein L is

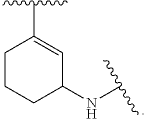 [SMILES: *NC1C=C(*)CCC1]

In one embodiment, a compound of Formula (II), Formula (II-A), or Formula (II-A-i) is provided, wherein L is

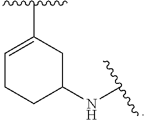 [SMILES: *NC1CCC=C(*)C1]

In one embodiment, a compound of Formula (II), Formula (II-A), or Formula (II-A-i) is provided, wherein ring B is an optionally substituted heterocycle. In a further embodiment, a compound of Formula (II), Formula (II-A), or Formula (II-A-i) is provided, wherein L is

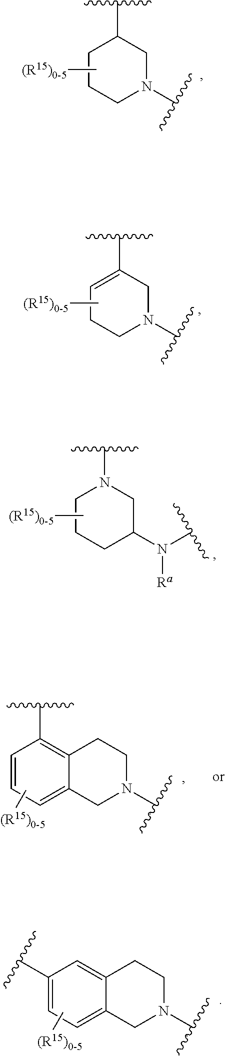

In a further embodiment, a compound of Formula (II), Formula (II-A), or Formula (II-A-i) is provided, wherein L is

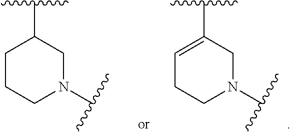 [SMILES: *C1CCCN(*)C1 ; *C1=CCCN(*)C1]

In a further embodiment, a compound of Formula (II), Formula (II-A), or Formula (II-A-i) is provided, wherein L is

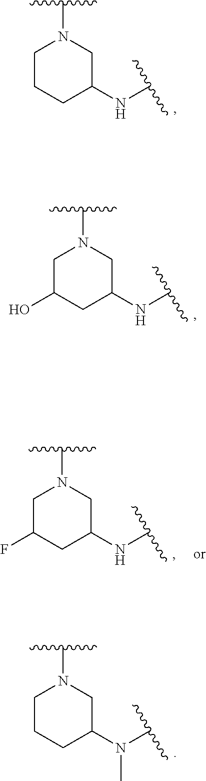

In a further embodiment, a compound of Formula (II), Formula (II-A), or Formula (II-A-i) is provided, wherein L is

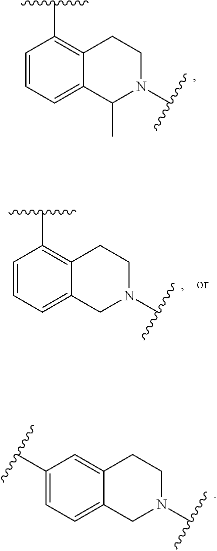 [SMILES: *c1cccc2c1CCN(*)C2C ; *c1cccc2c1CCN(*)C2 ; *c1ccc2c(c1)CCN(*)C2]

In one embodiment, a compound of Formula (II), Formula (II-A), or Formula (II-A-i) is provided, wherein R¹⁴ is C₂₋₆ alkenyl substituted with 0-3 R′. In one embodiment, a compound of Formula (II), Formula (II-A), or Formula (II-A-i) is provided, wherein R¹⁴ is C₂₋₆ alkenyl substituted with 0 R′.

In one embodiment, a compound of Formula (II), Formula (II-A), or Formula (II-A-i) is provided, wherein R¹⁴ is C₂₋₆ alkenyl substituted with 1-3 R′. In one embodiment, at least one R′ is halo. In one embodiment, at least one R′ is —OR^{b}. In one embodiment, at least one R′ is —OH. In another embodiment, at least one R′ is —NR^{b}R^{c}. In a further embodiment, at least one R′ is —NH₂ or —N(CH₃)₂.

In one embodiment, a compound of Formula (II), Formula (II-A), or Formula (II-A-i) is provided, wherein R is:

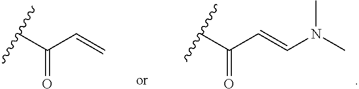

In another embodiment, a compound of Formula (II), Formula (II-A), or Formula (II-A-i) is provided, wherein R is:

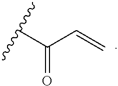 [SMILES: *C(=O)C=C]

In another embodiment, a compound of Formula (II), Formula (II-A), or Formula (II-A-i) is provided, wherein R is:

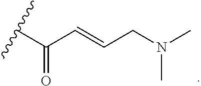 [SMILES: *C(=O)/C=C/CN(C)C]

In one embodiment, a compound of Formula (II), Formula (II-A), or Formula (II-A-i) is provided, wherein R¹⁴ is C₂₋₆ alkynyl substituted with 0-3 R′. In one embodiment, a compound of Formula (II), Formula (II-A), or Formula (II-A-i) is provided, wherein R¹⁴ is C₂₋₆ alkynyl substituted with 0 R′.

In one embodiment, a compound of Formula (II), Formula (II-A), or Formula (II-A-i) is provided, wherein R¹⁴ is C₂₋₆ alkynyl substituted with 1-3 R′. In one embodiment, at least one R′ is halo. In one embodiment, at least one R′ is —OR^{b}. In one embodiment, at least one R′ is —OH. In another embodiment, at least one R′ is —NR^{b}R^{c}. In a further embodiment, at least one R′ is —NH₂ or —N(CH₃)₂. In another embodiment, at least one R′ is carbocycle. In another embodiment, at least one R′ is cyclopropyl, cyclobutyl, cyclopentyl, or cyclohexyl.

In one embodiment, a compound of Formula (II), Formula (II-A), or Formula (II-A-i) is provided, wherein R is:

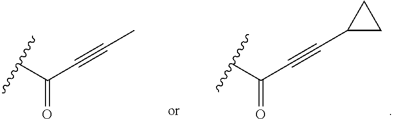

In one embodiment, a compound of Formula (II), Formula (II-A), or Formula (II-A-i) is provided, wherein R is:

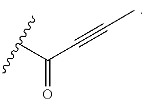 [SMILES: *C(=O)C#CC]

In one embodiment, a compound of Formula (II), Formula (II-A), or Formula (II-A-i) is provided, wherein R is:

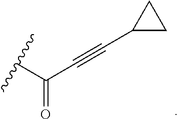 [SMILES: *C(=O)C#CC1CC1]

In one embodiment, a compound is provided, or a pharmaceutically acceptable salt, solvate, hydrate, isomer, tautomer, racemate, or isotope thereof, having the structure of a compound listed in Table 1, below:

TABLE 1
COMPOUNDS OF FORMULA (I)

| Compound | | |
|---|---|---|
| No. | Structure | Name |
| 1-1 | 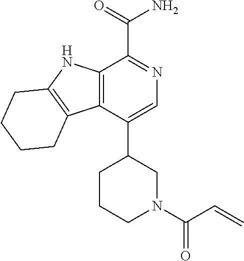 [SMILES: C=CC(=O)N1CCCC(c2cnc(C(N)=O)c3[nH]c4c(c23)CCCC4)C1] | 4-(1-acryloylpiperidin-3-yl)-6,7,8,9-tetrahydro- 5H-pyrido[3,4-b]indole-1-carboxamide |
| 2-1 | 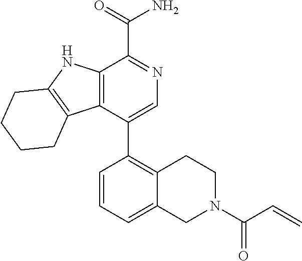 [SMILES: C=CC(=O)N1CCc2c(cccc2-c2cnc(C(N)=O)c3[nH]c4c(c23)CCCC4)C1] | 4-(2-acryloyl-1,2,3,4-tetrahydroisoquinolin-5- yl)-6,7,8,9-tetrahydro-5H-pyrido[3,4-b]indole- 1-carboxamide |
| 2-2 | 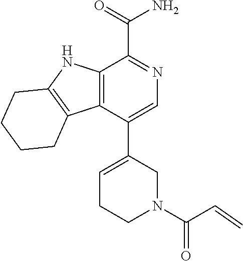 [SMILES: C=CC(=O)N1CCC=C(c2cnc(C(N)=O)c3[nH]c4c(c23)CCCC4)C1] | 4-(2-acryloyl-1,2,3,4-tetrahydroisoquinolin-5- yl)-6,7,8,9-tetrahydro-5H-pyrido[3,4-b]indole- 1-carboxamide |
| 2-3 | 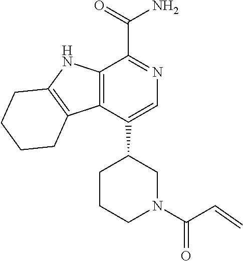 [SMILES: C=CC(=O)N1CCC[C@H](c2cnc(C(N)=O)c3[nH]c4c(c23)CCCC4)C1] | (R)-4-(1-acryloylpiperidin-3-yl)-6,7,8,9- tetrahydro-5H-pyrido[3,4-b]indole-1- carboxamide |
| 2-4 | 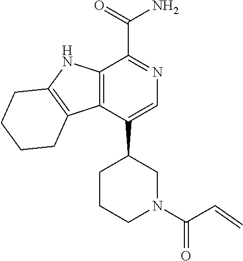 [SMILES: C=CC(=O)N1CCC[C@@H](c2cnc(C(N)=O)c3[nH]c4c(c23)CCCC4)C1] | (S)-4-(1-acryloylpiperidin-3-yl)-6,7,8,9- tetrahydro-5H-pyrido[3,4-b]indole-1- carboxamide |
| 2-5 | 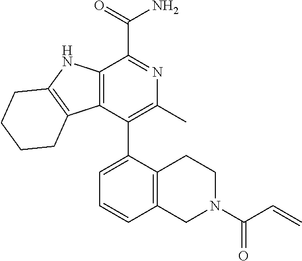 [SMILES: C=CC(=O)N1CCc2c(cccc2-c2c(C)nc(C(N)=O)c3[nH]c4c(c23)CCCC4)C1] | 4-(2-acryloyl-1,2,3,4-tetrahydroisoquinolin-5- yl)-3-methyl-6,7,8,9-tetrahydro-5H-pyrido[3,4- b]indole-1-carboxamide |
| 2-6 | 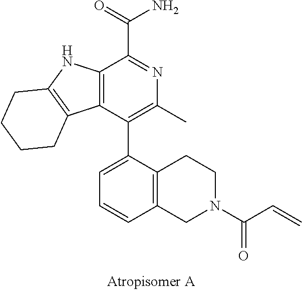 [SMILES: C=CC(=O)N1CCc2c(cccc2-c2c(C)nc(C(N)=O)c3[nH]c4c(c23)CCCC4)C1] | 4-(2-acryloyl-1,2,3,4-tetrahydroisoquinolin-5- yl)-3-methyl-6,7,8,9-tetrahydro-5H-pyrido[3,4- b]indole-1-carboxamide |
| 2-7 | 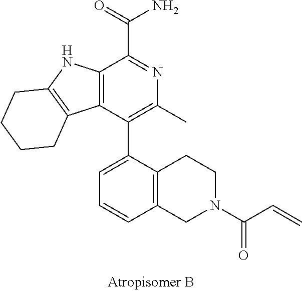 [SMILES: C=CC(=O)N1CCc2c(cccc2-c2c(C)nc(C(N)=O)c3[nH]c4c(c23)CCCC4)C1] | 4-(2-acryloyl-1,2,3,4-tetrahydroisoquinolin-5- yl)-3-methyl-6,7,8,9-tetrahydro-5H-pyrido[3,4- b]indole-1-carboxamide |
| 2-8 | 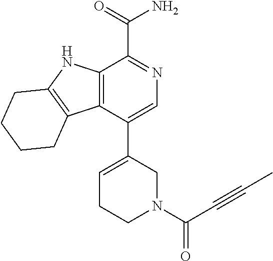 [SMILES: CC#CC(=O)N1CCC=C(c2cnc(C(N)=O)c3[nH]c4c(c23)CCCC4)C1] | 4-(1-(but-2-ynoyl)-1,2,5,6-tetrahydropyridin-3- yl)-6,7,8,9-tetrahydro-5H-pyrido[3,4-b]indole- 1-carboxamide |
| 2-9 | 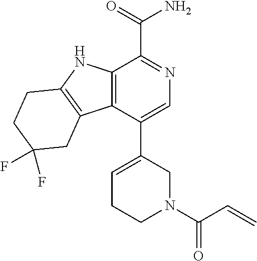 [SMILES: C=CC(=O)N1CCC=C(c2cnc(C(N)=O)c3[nH]c4c(c23)CC(F)(F)CC4)C1] | 4-(1-acryloyl-1,2,5,6-tetrahydropyridin-3-yl)- 6,6-difluoro-6,7,8,9-tetrahydro-5H-pyrido[3,4- b]indole-1-carboxamide |
| 2-10 | 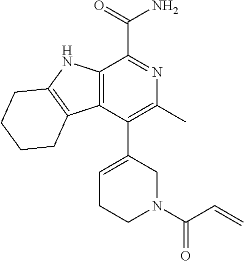 [SMILES: C=CC(=O)N1CCC=C(c2c(C)nc(C(N)=O)c3[nH]c4c(c23)CCCC4)C1] | 4-(1-acryloyl-1,2,5,6-tetrahydropyridin-3-yl)- 3-methyl-6,7,8,9-tetrahydro-5H-pyrido[3,4- b]indole-1-carboxamide |
| 3-1 | 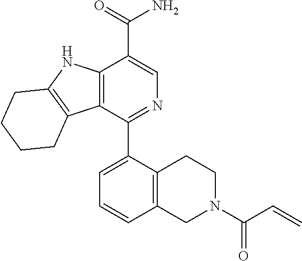 [SMILES: C=CC(=O)N1CCc2c(cccc2-c2ncc(C(N)=O)c3[nH]c4c(c23)CCCC4)C1] | 1-(2-acryloyl-1,2,3,4-tetrahydroisoquinolin-5- yl)-6,7,8,9-tetrahydro-5H-pyrido[4,3-b]indole- 4-carboxamide |
| 3-2 | 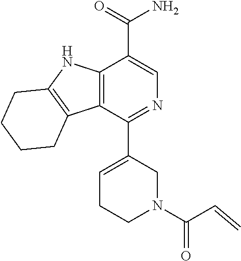 [SMILES: C=CC(=O)N1CCC=C(c2ncc(C(N)=O)c3[nH]c4c(c23)CCCC4)C1] | 1-(1-acryloyl-1,2,5,6-tetrahydropyridin-3-yl)- 6,7,8,9-tetrahydro-5H-pyrido[4,3-b]indole-4- carboxamide |
| 3-3 | 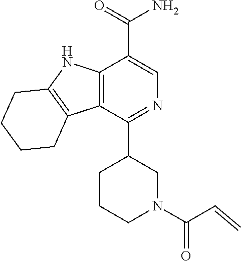 [SMILES: C=CC(=O)N1CCCC(c2ncc(C(N)=O)c3[nH]c4c(c23)CCCC4)C1] | 1-(1-acryloylpiperidin-3-yl)-6,7,8,9-tetrahydro- 5H-pyrido[4,3-b]indole-4-carboxamide |
| 3-4 | 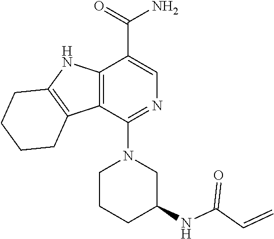 [SMILES: C=CC(=O)N[C@H]1CCCN(c2ncc(C(N)=O)c3[nH]c4c(c23)CCCC4)C1] | (S)-1-(3-acrylamidopiperidin-1-yl)-6,7,8,9- tetrahydro-5H-pyrido[4,3-b]indole-4- carboxamide |
| 4-1 | 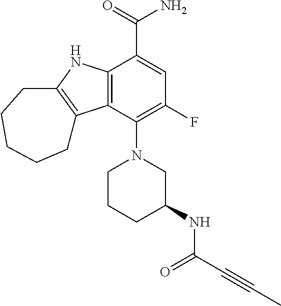 [SMILES: CC#CC(=O)N[C@H]1CCCN(c2c(F)cc(C(N)=O)c3[nH]c4c(c23)CCCCC4)C1] | (S)-1-(3-acrylamidopiperidin-1-yl)-6,7,8,9- tetrahydro-5H-pyrido[4,3-b]indole-4- carboxamide |
| 4-2 | 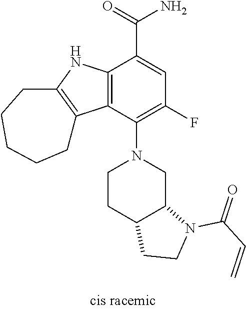 | 1-((3aS,7aR)-1-acryloyloctahydro-6H- pyrrolo[2,3-c]pyridin-6-yl)-2-fluoro- 5,6,7,8,9,10-hexahydrocyclohepta[b]indole-4- carboxamide |
| 4-3 | 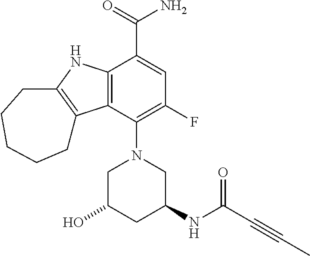 [SMILES: CC#CC(=O)N[C@H]1C[C@H](O)CN(c2c(F)cc(C(N)=O)c3[nH]c4c(c23)CCCCC4)C1] | 1-((3S,5S)-3-(but-2-ynamido)-5- hydroxypiperidin-1-yl)-2-fluoro-5,6,7,8,9,10- hexahydrocyclohepta[b]indole-4-carboxamide |
| 4-4 | 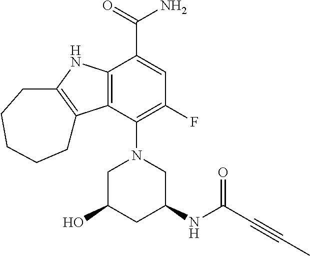 [SMILES: CC#CC(=O)N[C@H]1C[C@@H](O)CN(c2c(F)cc(C(N)=O)c3[nH]c4c(c23)CCCCC4)C1] | 1-((3S,5R)-3-(but-2-ynamido)-5- hydroxypiperidin-1-yl)-2-fluoro-5,6,7,8,9,10- hexahydrocyclohepta[b]indole-4-carboxamide |
| 5-1 | 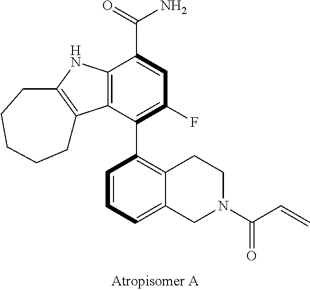 | (R)-1-(2-acryloyl-1,2,3,4- tetrahydroisoquinolin-5-yl)-2-fluoro- 5,6,7,8,9,10-hexahydrocyclohepta[b]indole-4- carboxamide |
| 5-2 | 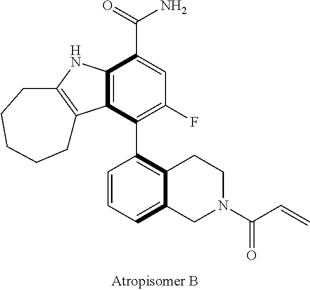 | (S)-1-(2-acryloyl-1,2,3,4- tetrahydroisoquinolin-5-yl)-2-fluoro- 5,6,7,8,9,10-hexahydrocyclohepta[b]indole-4- carboxamide |
| 5-3 | 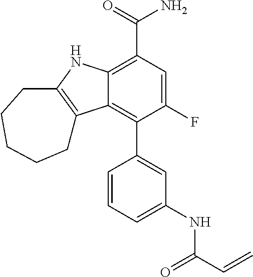 [SMILES: C=CC(=O)Nc1cccc(-c2c(F)cc(C(N)=O)c3[nH]c4c(c23)CCCCC4)c1] | 1-(3-acrylamidophenyl)-2-fluoro-5,6,7,8,9,10- hexahydrocyclohepta[b]indole-4-carboxamide |
| 5-4 | 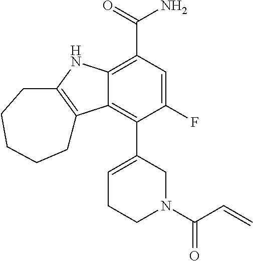 [SMILES: C=CC(=O)N1CCC=C(c2c(F)cc(C(N)=O)c3[nH]c4c(c23)CCCCC4)C1] | 1-(1-acryloyl-1,2,5,6-tetrahydropyridin-3-yl)- 2-fluoro-5,6,7,8,9,10- hexahydrocyclohepta[b]indole-4-carboxamide |
| 5-5 | 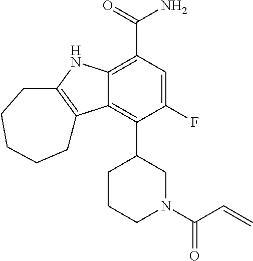 [SMILES: C=CC(=O)N1CCCC(c2c(F)cc(C(N)=O)c3[nH]c4c(c23)CCCCC4)C1] | 1-(1-acryloylpiperidin-3-yl)-2-fluoro- 5,6,7,8,9,10-hexahydrocyclohepta[b]indole-4- carboxamide |
| 5-6 | 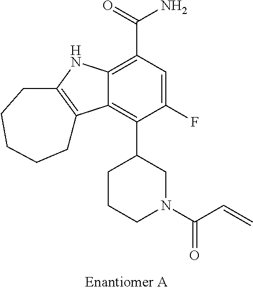 [SMILES: C=CC(=O)N1CCCC(c2c(F)cc(C(N)=O)c3[nH]c4c(c23)CCCCC4)C1] | 1-(1-acryloylpiperidin-3-yl)-2-fluoro- 5,6,7,8,9,10-hexahydrocyclohepta[b]indole-4- carboxamide |
| 5-7 | 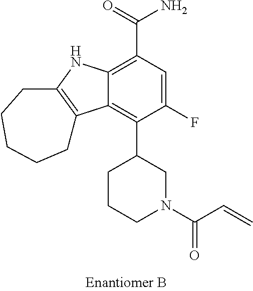 [SMILES: C=CC(=O)N1CCCC(c2c(F)cc(C(N)=O)c3[nH]c4c(c23)CCCCC4)C1] | 1-(1-acryloylpiperidin-3-yl)-2-fluoro- 5,6,7,8,9,10-hexahydrocyclohepta[b]indole-4- carboxamide |
| 5-8 | 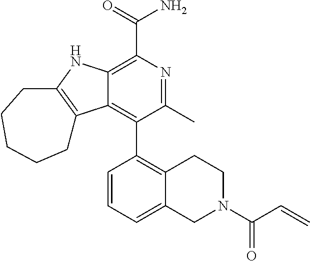 [SMILES: C=CC(=O)N1CCc2c(cccc2-c2c(C)nc(C(N)=O)c3[nH]c4c(c23)CCCCC4)C1] | 4-(2-acryloyl-1,2,3,4-tetrahydroisoquinolin-5- yl)-3-methyl-5,6,7,8,9,10- hexahydrocyclohepta[4,5]pyrrolo[2,3- c]pyridine-1-carboxamide |
| 6-1 | 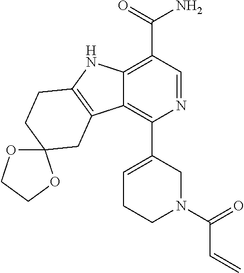 [SMILES: C=CC(=O)N1CCC=C(c2ncc(C(N)=O)c3[nH]c4c(c23)CC2(CC4)OCCO2)C1] | 5-(1-acryloyl-1,2,5,6-tetrahydropyridin-3-yl)- 1,2,4,9-tetrahydrospiro[carbazole-3,2′- [1,3]dioxolane]-8-carboxamide |
| 6-2 | 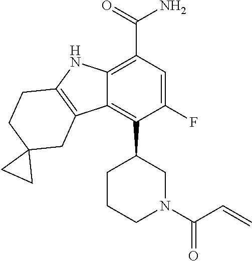 [SMILES: C=CC(=O)N1CCC[C@@H](c2c(F)cc(C(N)=O)c3[nH]c4c(c23)CC2(CC4)CC2)C1] | (S)-5-(1-acryloylpiperidin-3-yl)-6-fluoro- 1,2,4,9-tetrahydrospiro[carbazole-3,1′- cyclopropane]-8-carboxamide |
| 6-3 | 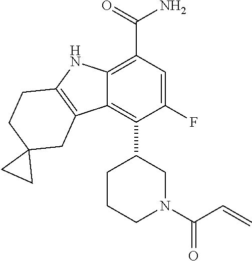 | (R)-5-(1-acryloylpiperidin-3-yl)-6-fluoro- 1,2,4,9-tetrahydrospiro[carbazole-3,1′- cyclopropane]-8-carboxamide |
| 7-1 | 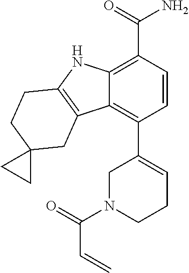 [SMILES: C=CC(=O)N1CCC=C(c2ccc(C(N)=O)c3[nH]c4c(c23)CC2(CC4)CC2)C1] | 5-(1-acryloyl-1,2,5,6-tetrahydropyridin-3-yl)- 1,2,4,9-tetrahydrospiro[carbazole-3,1′- cyclopropane]-8-carboxamide |
| 7-2 | 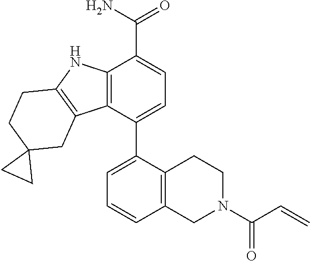 [SMILES: C=CC(=O)N1CCc2c(cccc2-c2ccc(C(N)=O)c3[nH]c4c(c23)CC2(CC4)CC2)C1] | 5-(2-acryloyl-1,2,3,4-tetrahydroisoquinolin-5- yl)-1,2,4,9-tetrahydrospiro[carbazole-3,1′- cyclopropane]-8-carboxamide |
| 7-3 | 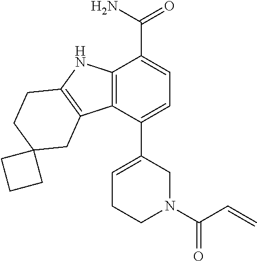 [SMILES: C=CC(=O)N1CCC=C(c2ccc(C(N)=O)c3[nH]c4c(c23)CC2(CCC2)CC4)C1] | 5-(1-acryloyl-1,2,5,6-tetrahydropyridin-3-yl)- 1,2,4,9-tetrahydrospiro[carbazole-3,1′- cyclobutane]-8-carboxamide |
| 7-4 | 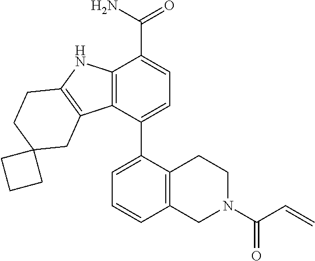 [SMILES: C=CC(=O)N1CCc2c(cccc2-c2ccc(C(N)=O)c3[nH]c4c(c23)CC2(CCC2)CC4)C1] | 5-(2-acryloyl-1,2,3,4-tetrahydroisoquinolin-5- yl)-1,2,4,9-tetrahydrospiro[carbazole-3,1′- cyclobutane]-8-carboxamide |
| 7-5 | 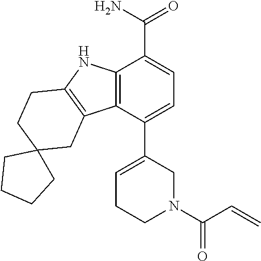 [SMILES: C=CC(=O)N1CCC=C(c2ccc(C(N)=O)c3[nH]c4c(c23)CC2(CCCC2)CC4)C1] | 5-(1-acryloyl-1,2,5,6-tetrahydropyridin-3-yl)- 1,2,4,9-tetrahydrospiro[carbazole-3,1′- cyclopentane]-8-carboxamide |
| 7-6 | 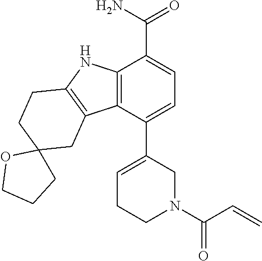 [SMILES: C=CC(=O)N1CCC=C(c2ccc(C(N)=O)c3[nH]c4c(c23)CC2(CCCO2)CC4)C1] | 5-(1-acryloyl-1,2,5,6-tetrahydropyridin-3-yl)- 1,2,4,4′,5′,9-hexahydro-3′H-spiro[carbazole- 3,2′-furan]-8-carboxamide |
| 7-7 | 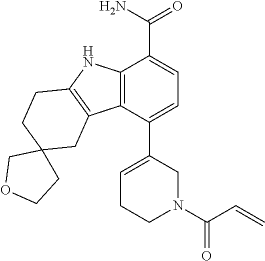 [SMILES: C=CC(=O)N1CCC=C(c2ccc(C(N)=O)c3[nH]c4c(c23)CC2(CCOC2)CC4)C1] | 5-(1-acryloyl-1,2,5,6-tetrahydropyridin-3-yl)- 1,2,4,4′,5′,9-hexahydro-2′H-spiro[carbazole- 3,3′-furan]-8-carboxamide |
| 7-8 | 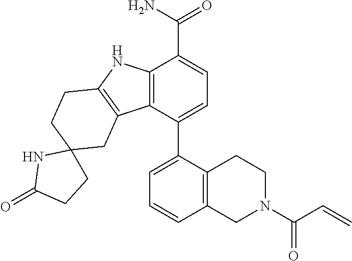 [SMILES: C=CC(=O)N1CCc2c(cccc2-c2ccc(C(N)=O)c3[nH]c4c(c23)CC2(CCC(=O)N2)CC4)C1] | 5-(2-acryloyl-1,2,3,4-tetrahydroisoquinolin-5- yl)-5′-oxo-1,2,4,9-tetrahydrospiro[carbazole- 3,2′-pyrrolidine]-8-carboxamide |
| 7-9 | 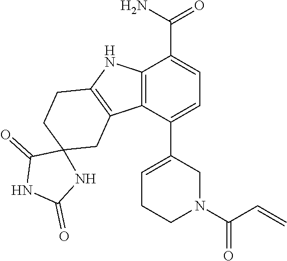 [SMILES: C=CC(=O)N1CCC=C(c2ccc(C(N)=O)c3[nH]c4c(c23)CC2(CC4)NC(=O)NC2=O)C1] | 5-(1-acryloyl-1,2,5,6-tetrahydropyridin-3-yl)- 2′,5′-dioxo-1,2,4,9-tetrahydrospiro[carbazole- 3,4′-imidazolidine]-8-carboxamide |
| 7-10 | 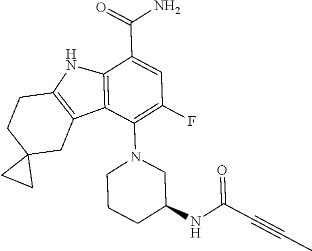 [SMILES: CC#CC(=O)N[C@H]1CCCN(c2c(F)cc(C(N)=O)c3[nH]c4c(c23)CC2(CC4)CC2)C1] | (S)-5-(3-(but-2-ynamido)piperidin-1-yl)-6- fluoro-1,2,4,9-tetrahydrospiro[carbazole-3,1′- cyclopropane]-8-carboxamide |
| 7-11 | 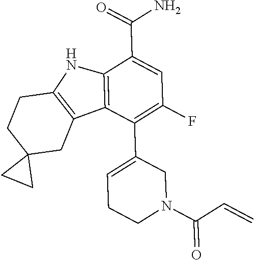 [SMILES: C=CC(=O)N1CCC=C(c2c(F)cc(C(N)=O)c3[nH]c4c(c23)CC2(CC4)CC2)C1] | 5-(1-acryloyl-1,2,5,6-tetrahydropyridin-3-yl)- 6-fluoro-1,2,4,9-tetrahydrospiro[carbazole-3,1′- cyclopropane]-8-carboxamide |
| 7-12 | 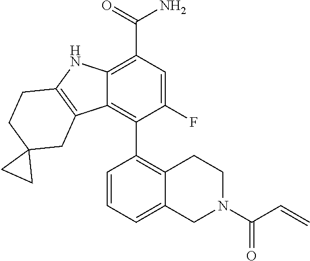 [SMILES: C=CC(=O)N1CCc2c(cccc2-c2c(F)cc(C(N)=O)c3[nH]c4c(c23)CC2(CC4)CC2)C1] | 5-(2-acryloyl-1,2,3,4-tetrahydroisoquinolin-5- yl)-6-fluoro-1,2,4,9-tetrahydrospiro[carbazole- 3,1′-cyclopropane]-8-carboxamide |
| 7-13 | 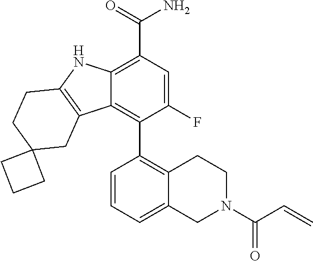 [SMILES: C=CC(=O)N1CCc2c(cccc2-c2c(F)cc(C(N)=O)c3[nH]c4c(c23)CC2(CCC2)CC4)C1] | 5-(2-acryloyl-1,2,3,4-tetrahydroisoquinolin-5- yl)-6-fluoro-1,2,4,9-tetrahydrospiro[carbazole- 3,1′-cyclobutane]-8-carboxamide |
| 7-14 | 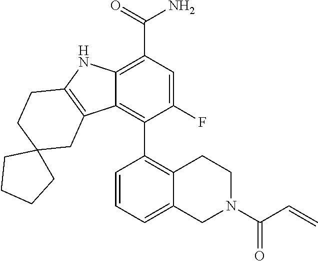 [SMILES: C=CC(=O)N1CCc2c(cccc2-c2c(F)cc(C(N)=O)c3[nH]c4c(c23)CC2(CCCC2)CC4)C1] | 5-(2-acryloyl-1,2,3,4-tetrahydroisoquinolin-5- yl)-6-fluoro-1,2,4,9-tetrahydrospiro[carbazole- 3,1′-cyclopentane]-8-carboxamide |
| 7-15 | 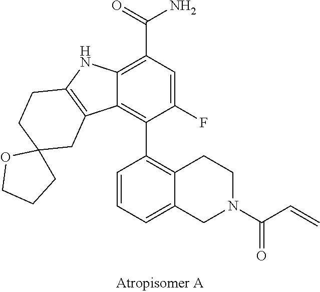 [SMILES: C=CC(=O)N1CCc2c(cccc2-c2c(F)cc(C(N)=O)c3[nH]c4c(c23)CC2(CCCO2)CC4)C1] | 5-(2-acryloyl-1,2,3,4-tetrahydroisoquinolin-5- yl)-6-fluoro-1,2,4,4′,5′,9-hexahydro-3′H- spiro[carbazole-3,2′-furan]-8-carboxamide |
| 7-16 | 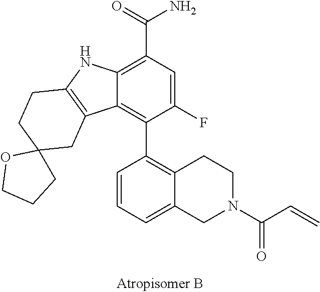 [SMILES: C=CC(=O)N1CCc2c(cccc2-c2c(F)cc(C(N)=O)c3[nH]c4c(c23)CC2(CCCO2)CC4)C1] | 5-(2-acryloyl-1,2,3,4-tetrahydroisoquinolin-5- yl)-6-fluoro-1,2,4,4′,5′,9-hexahydro-3′H- spiro[carbazole-3,2′-furan]-8-carboxamide |
| 7-17 | 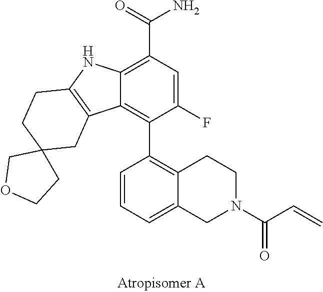 [SMILES: C=CC(=O)N1CCc2c(cccc2-c2c(F)cc(C(N)=O)c3[nH]c4c(c23)CC2(CCOC2)CC4)C1] | 5-(2-acryloyl-1,2,3,4-tetrahydroisoquinolin-5- yl)-6-fluoro-1,2,4,4′,5′,9-hexahydro-2′H- spiro[carbazole-3,3′-furan]-8-carboxamide |
| 7-18 | 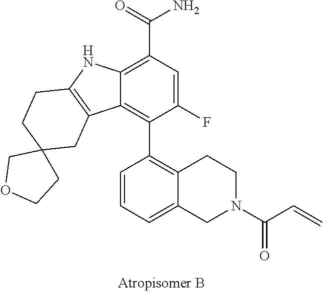 [SMILES: C=CC(=O)N1CCc2c(cccc2-c2c(F)cc(C(N)=O)c3[nH]c4c(c23)CC2(CCOC2)CC4)C1] | 5-(2-acryloyl-1,2,3,4-tetrahydroisoquinolin-5- yl)-6-fluoro-1,2,4,4′,5′,9-hexahydro-2′H- spiro[carbazole-3,3′-furan]-8-carboxamide |
| 7-19 | 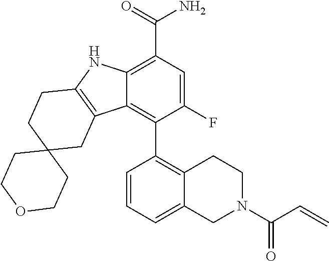 [SMILES: C=CC(=O)N1CCc2c(cccc2-c2c(F)cc(C(N)=O)c3[nH]c4c(c23)CC2(CCOCC2)CC4)C1] | 5-(2-acryloyl-1,2,3,4-tetrahydroisoquinolin-5- yl)-6-fluoro-1,2,2′,3′,4,5′,6′,9- octahydrospiro[carbazole-3,4′-pyran]-8- carboxamide |
| 7-20 | 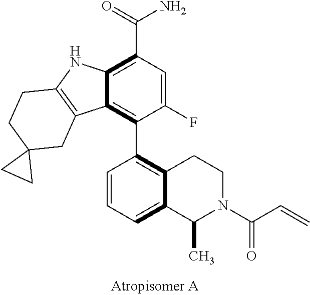 | 5-((S)-2-acryloyl-1-methyl-1,2,3,4- tetrahydroisoquinolin-5-yl)-6-fluoro-1,2,4,9- tetrahydrospiro[carbazole-3,1′-cyclopropane]- 8-carboxamide |
| 7-21 | 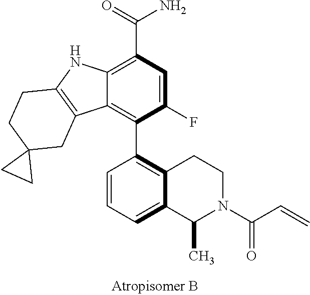 | (R)-5-((S)-2-acryloyl-1-methyl-1,2,3,4- tetrahydroisoquinolin-5-yl)-6-fluoro-1,2,4,9- tetrahydrospiro[carbazole-3,1′-cyclopropane]- 8-carboxamide |
| 7-22 | 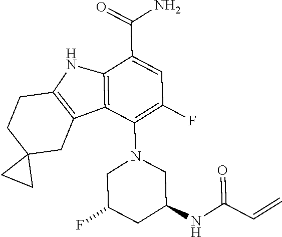 [SMILES: C=CC(=O)N[C@H]1C[C@H](F)CN(c2c(F)cc(C(N)=O)c3[nH]c4c(c23)CC2(CC4)CC2)C1] | 5-((3S,5S)-3-acrylamido-5-fluoropiperidin-1- yl)-6-fluoro-1,2,4,9-tetrahydrospiro[carbazole- 3,1′-cyclopropane]-8-carboxamide |
| 7-23 | 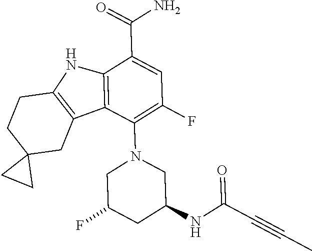 [SMILES: CC#CC(=O)N[C@H]1C[C@H](F)CN(c2c(F)cc(C(N)=O)c3[nH]c4c(c23)CC2(CC4)CC2)C1] | 5-((3S,5S)-3-(but-2-ynamido)-5- fluoropiperidin-1-yl)-6-fluoro-1,2,4,9- tetrahydrospiro[carbazole-3,1′-cyclopropane]- 8-carboxamide |
| 7-24 | 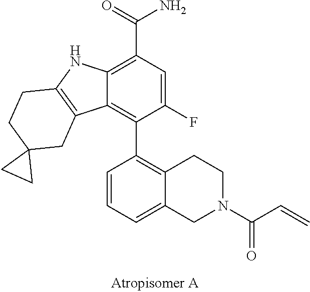 [SMILES: C=CC(=O)N1CCc2c(cccc2-c2c(F)cc(C(N)=O)c3[nH]c4c(c23)CC2(CC4)CC2)C1] | 5-(2-acryloyl-1,2,3,4-tetrahydroisoquinolin-5- yl)-6-fluoro-1,2,4,9-tetrahydrospiro[carbazole- 3,1′-cyclopropane]-8-carboxamide |
| 7-25 | 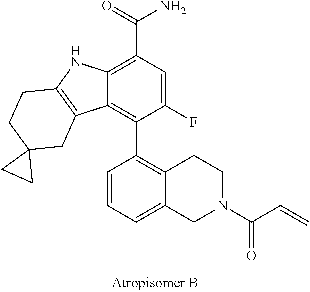 [SMILES: C=CC(=O)N1CCc2c(cccc2-c2c(F)cc(C(N)=O)c3[nH]c4c(c23)CC2(CC4)CC2)C1] | 5-(2-acryloyl-1,2,3,4-tetrahydroisoquinolin-5- yl)-6-fluoro-1,2,4,9-tetrahydrospiro[carbazole- 3,1′-cyclopropane]-8-carboxamide |
| 7-26 | 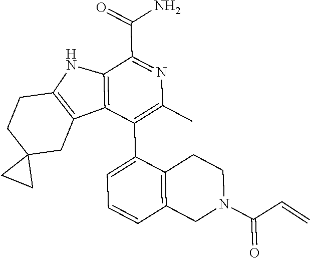 [SMILES: C=CC(=O)N1CCc2c(cccc2-c2c(C)nc(C(N)=O)c3[nH]c4c(c23)CC2(CC4)CC2)C1] | 4′-(2-acryloyl-1,2,3,4-tetrahydroisoquinolin-5- yl)-3′-methyl-5′,7′,8′,9′- tetrahydrospiro[cyclopropane-1,6′-pyrido[3,4- b]indole]-1′-carboxamide |
| 7-27 | 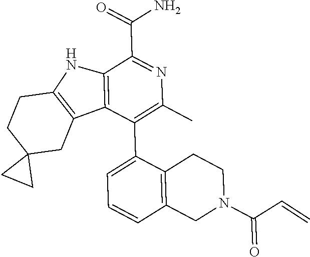 [SMILES: C=CC(=O)N1CCc2c(cccc2-c2c(C)nc(C(N)=O)c3[nH]c4c(c23)CC2(CC4)CC2)C1] | 4′-(2-acryloyl-1,2,3,4-tetrahydroisoquinolin-5- yl)-3′-methyl-5′,7′,8′,9′- tetrahydrospiro[cyclopropane-1,6′-pyrido[3,4- b]indole]-1′-carboxamide |
| 7-28 | 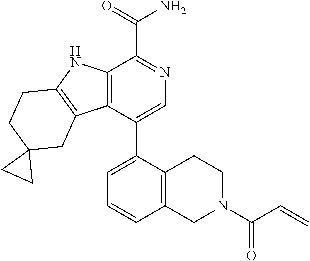 [SMILES: C=CC(=O)N1CCc2c(cccc2-c2cnc(C(N)=O)c3[nH]c4c(c23)CC2(CC4)CC2)C1] | 4′-(2-acryloyl-1,2,3,4-tetrahydroisoquinolin-5- yl)-5′,7′,8′,9′-tetrahydrospiro[cyclopropane- 1,6′-pyrido[3,4-b]indole]-1′-carboxamide |
| 7-29 | 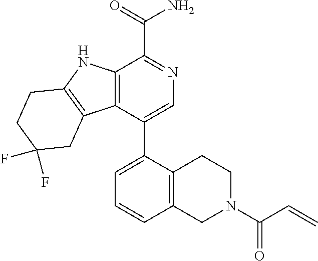 [SMILES: C=CC(=O)N1CCc2c(cccc2-c2cnc(C(N)=O)c3[nH]c4c(c23)CC(F)(F)CC4)C1] | 4-(2-acryloyl-1,2,3,4-tetrahydroisoquinolin-5- yl)-6,6-difluoro-6,7,8,9-tetrahydro-5H- pyrido[3,4-b]indole-1-carboxamide |
| 7-30 | 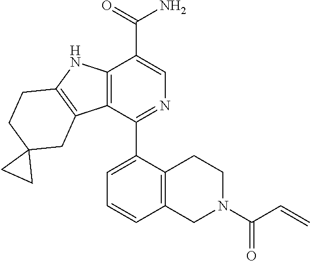 [SMILES: C=CC(=O)N1CCc2c(cccc2-c2ncc(C(N)=O)c3[nH]c4c(c23)CC2(CC4)CC2)C1] | 1′-(2-acryloyl-1,2,3,4-tetrahydroisoquinolin-5- yl)-5′,6′,7′,9′-tetrahydrospiro[cyclopropane- 1,8′-pyrido[4,3-b]indole]-4′-carboxamide |
| 8-1 | 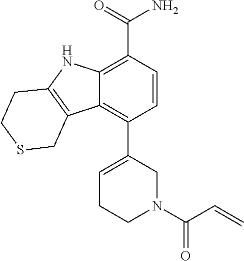 [SMILES: C=CC(=O)N1CCC=C(c2ccc(C(N)=O)c3[nH]c4c(c23)CSCC4)C1] | 9-(1-acryloyl-1,2,5,6-tetrahydropyridin-3-yl)- 1,3,4,5-tetrahydrothiopyrano[4,3-b]indole-6- carboxamide |
| 9-1 | 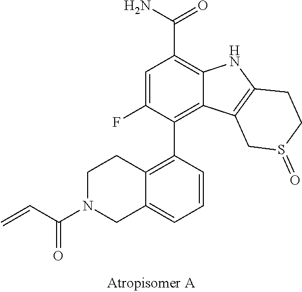 [SMILES: C=CC(=O)N1CCc2c(cccc2-c2c(F)cc(C(N)=O)c3[nH]c4c(c23)CS(=O)CC4)C1] | 9-(2-acryloyl-1,2,3,4-tetrahydroisoquinolin-5- yl)-8-fluoro-1,3,4,5-tetrahydrothiopyrano[4,3- b]indole-6-carboxamide 2-oxide |
| 9-2 | 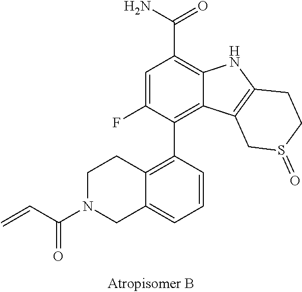 [SMILES: C=CC(=O)N1CCc2c(cccc2-c2c(F)cc(C(N)=O)c3[nH]c4c(c23)CS(=O)CC4)C1] | 9-(2-acryloyl-1,2,3,4-tetrahydroisoquinolin-5- yl)-8-fluoro-1,3,4,5-tetrahydrothiopyrano[4,3- b]indole-6-carboxamide 2-oxide |
| 9-3 | 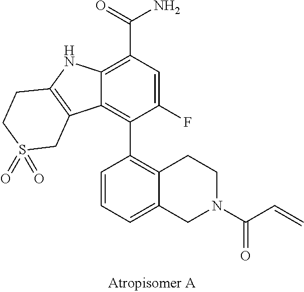 [SMILES: C=CC(=O)N1CCc2c(cccc2-c2c(F)cc(C(N)=O)c3[nH]c4c(c23)CS(=O)(=O)CC4)C1] | 9-(2-acryloyl-1,2,3,4-tetrahydroisoquinolin-5- yl)-8-fluoro-1,3,4,5-tetrahydrothiopyrano[4,3- b]indole-6-carboxamide 2,2-dioxide |
| 9-4 | 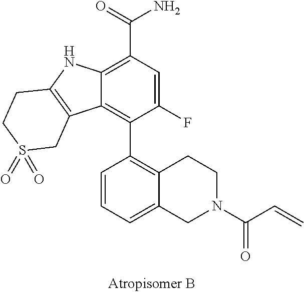 [SMILES: C=CC(=O)N1CCc2c(cccc2-c2c(F)cc(C(N)=O)c3[nH]c4c(c23)CS(=O)(=O)CC4)C1] | 9-(2-acryloyl-1,2,3,4-tetrahydroisoquinolin-5- yl)-8-fluoro-1,3,4,5-tetrahydrothiopyrano[4,3- b]indole-6-carboxamide 2,2-dioxide |
| 9-5 | 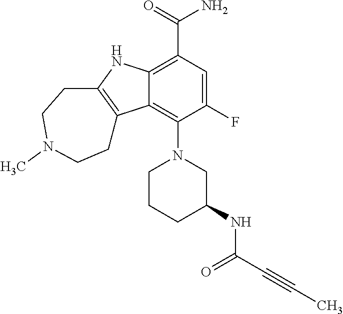 [SMILES: CC#CC(=O)N[C@H]1CCCN(c2c(F)cc(C(N)=O)c3[nH]c4c(c23)CCN(C)CC4)C1] | (S)-10-(3-(but-2-ynamido)piperidin-1-yl)-9- fluoro-3-methyl-1,2,3,4,5,6- hexahydroazepino[4,5-b]indole-7-carboxamide |
| 9-6 | 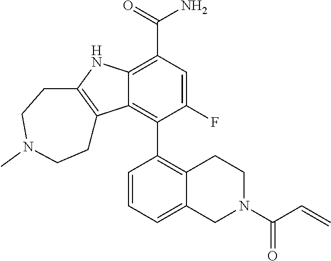 [SMILES: C=CC(=O)N1CCc2c(cccc2-c2c(F)cc(C(N)=O)c3[nH]c4c(c23)CCN(C)CC4)C1] | 10-(2-acryloyl-1,2,3,4-tetrahydroisoquinolin-5- yl)-9-fluoro-3-methyl-1,2,3,4,5,6- hexahydroazepino[4,5-b]indole-7-carboxamide |
| 9-7 | 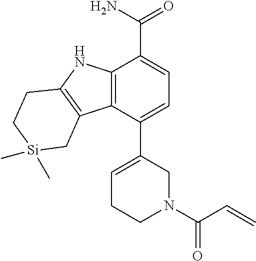 [SMILES: C=CC(=O)N1CCC=C(c2ccc(C(N)=O)c3[nH]c4c(c23)C[Si](C)(C)CC4)C1] | 9-(1-acryloyl-1,2,5,6-tetrahydropyridin-3-yl)- 2,2-dimethyl-2,3,4,5-tetrahydro-1H-silino[4,3- b]indole-6-carboxamide |
| 10-1 | 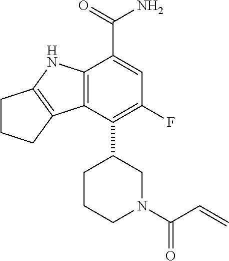 | (R)-8-(1-acryloylpiperidin-3-yl)-7-fluoro- 1,2,3,4-tetrahydrocyclopenta[b]-indole-5- carboxamide |
| 10-2 | 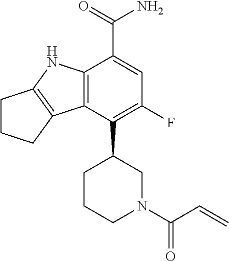 [SMILES: C=CC(=O)N1CCC[C@@H](c2c(F)cc(C(N)=O)c3[nH]c4c(c23)CCC4)C1] | (S)-8-(1-acryloylpiperidin-3-yl)-7-fluoro- 1,2,3,4-tetrahydrocyclo-penta[b]indole-5- carboxamide |
| 10-3 | 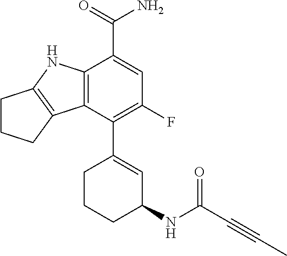 [SMILES: CC#CC(=O)N[C@@H]1C=C(c2c(F)cc(C(N)=O)c3[nH]c4c(c23)CCC4)CCC1] | 8-[(3S)-3-(but-2-ynoylamino)cyclohexen-1- yl]-7-fluoro-1,2,3,4- tetrahydrocyclopenta[b]indole-5-carboxamide |
| 10-4 | 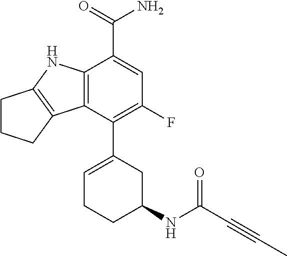 [SMILES: CC#CC(=O)N[C@H]1CCC=C(c2c(F)cc(C(N)=O)c3[nH]c4c(c23)CCC4)C1] | 8-[(5S)-5-(but-2-ynoylamino)cyclohexen-1- yl]-7-fluoro-1,2,3,4- tetrahydrocyclopenta[b]indole-5-carboxamide |
| 10-5 | 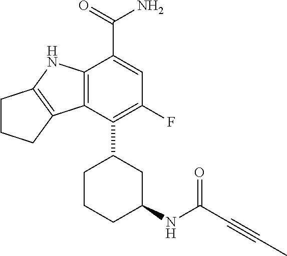 | 8-[(1S,3S)-3-(but-2-ynoylamino)cyclohexyl]- 7-fluoro-1,2,3,4- tetrahydrocyclopenta[b]indole-5-carboxamide |
| 10-6 | 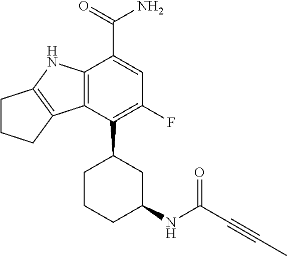 [SMILES: CC#CC(=O)N[C@H]1CCC[C@@H](c2c(F)cc(C(N)=O)c3[nH]c4c(c23)CCC4)C1] | 8-[(1R,3S)-3-(but-2-ynoylamino)cyclohexyl]- 7-fluoro-1,2,3,4-tetrahydro- cyclopenta[b]indole-5-carboxamide |
| 10-7 | 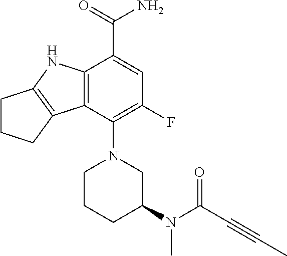 [SMILES: CC#CC(=O)N(C)[C@H]1CCCN(c2c(F)cc(C(N)=O)c3[nH]c4c(c23)CCC4)C1] | 8-[(3S)-3-[but-2-ynoyl(methyl)amino]-1- piperidyl]-7-fluoro-1,2,3,4- tetrahydrocyclopenta[b]indole-5-carboxamide |
| 10-8 | 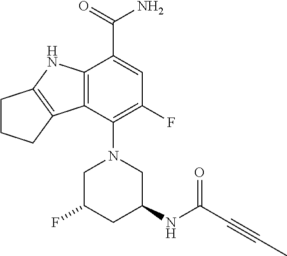 [SMILES: CC#CC(=O)N[C@H]1C[C@H](F)CN(c2c(F)cc(C(N)=O)c3[nH]c4c(c23)CCC4)C1] | 8-((3S,5S)-3-(but-2-ynamido)-5- fluoropiperidin-1-yl)-7-fluoro-1,2,3,4- tetrahydrocyclopenta[b]indole-5-carboxamide |
| 10-9 | 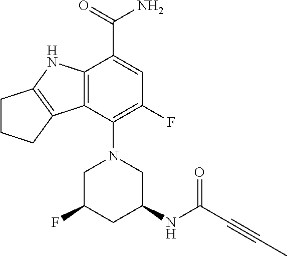 [SMILES: CC#CC(=O)N[C@H]1C[C@@H](F)CN(c2c(F)cc(C(N)=O)c3[nH]c4c(c23)CCC4)C1] | 8-((3S,5R)-3-(but-2-ynamido)-5- fluoropiperidin-1-yl)-7-fluoro-1,2,3,4- tetrahydrocyclopenta[b]indole-5-carboxamide |

In some embodiments are compounds having the structure of any one of Formulas (I), (I′), (I-A), (I-B), (I-C), (I-D), or (II) or of Table 1. In some embodiments are pharmaceutically acceptable salts of compounds having the structure of any one of Formulas (I), (I′), (I-A), (I-B), (I-C), (I-D), or (II) or of Table 1. In some embodiments are solvates of compounds having the structure of any one of Formulas (I), (I′), (I-A), (I-B), (I-C), (I-D), or (II) or of Table 1. In some embodiments are hydrates of compounds having the structure of any one of Formulas (I), (I′), (I-A), (I-B), (I-C), (I-D), or (II) or of Table 1. In some embodiments are isomers of compounds having the structure of any one of Formulas (I), (I′), (I-A), (I-B), (I-C), (I-D), or (II) or of Table 1. In some embodiments are atropisomers of compounds having the structure of any one of Formulas (I), (F), (I-A), (I-B), (I-C), (I-D), or (II) or of Table 1. In some embodiments are tautomers of compounds having the structure of any one of Formulas (I), (I′), (I-A), (I-B), (I-C), (I-D), or (II) or of Table 1. In some embodiments are racemates of compounds having the structure of any one of Formulas (I), (I′), (I-A), (I-B), (I-C), (I-D), or (II) or of Table 1. In some embodiments are isotopic forms of compounds having the structure of any one of Formulas (I), (I′), (I-A), (I-B), (I-C), (I-D), or (II) or of Table 1.

In further embodiments, pharmaceutical compositions are provided comprising a compound having the structure of any one of Formulas (I), (I′), (I-A), (I-B), (I-C), (I-D), or (II) or of Table 1, or a pharmaceutically acceptable salt, solvate, hydrate, isomer, tautomer, racemate or isotope, and at least one pharmaceutically acceptable excipient.

In yet further embodiments, methods of inhibiting a protein kinase are provided comprising contacting the protein kinase with an effective amount of a compound having the structure of any one of Formulas (I), (I′), (I-A), (I-B), (I-C), (I-D), or (II) or of Table 1, or a pharmaceutically acceptable salt, solvate, hydrate, isomer, tautomer, racemate, isotope, or pharmaceutical composition thereof. In some embodiments the protein kinase is BTK.

In some embodiments, are methods for treating a BTK dependent condition, comprising administering to a subject in need thereof, an effective amount of a compound having the structure of any one of Formulas (I), (I′), (I-A), (I-B), (I-C), (I-D), or (II) or of Table 1, or a pharmaceutically acceptable salt, solvate, hydrate, isomer, tautomer, racemate, isotope, or pharmaceutical composition thereof.

In some embodiments the BTK dependent condition is cancer, an autoimmune disease, an inflammatory disease, or a thromboembolic disease. In some embodiments the autoimmune disease is multiple sclerosis, rheumatoid arthritis, psoriasis, Sjogren's syndrome, or systemic lupus erythematosus. In some embodiments the inflammatory disease is urticaria. In some embodiments the BTK dependent condition is cancer. In some embodiments the BTK dependent condition is an autoimmune disease. In some embodiments the BTK dependent condition is an inflammatory disease. In some embodiments the BTK dependent condition is a thromboembolic disease. In some embodiments the BTK dependent condition is multiple sclerosis. In some embodiments the BTK dependent condition is rheumatoid arthritis. In some embodiments the BTK dependent condition is psoriasis. In some embodiments the BTK dependent condition is Sjogren's syndrome. In some embodiments the BTK dependent condition is systemic lupus erythematosus. In some embodiments the BTK dependent condition is urticaria. In some embodiments the BTK dependent condition is primary CNS lymphoma.

In some embodiments, disclosed are uses of a compound of any one of Formulas (I), (I′), (I-A), (I-B), (I-C), (I-D), or (II) or of Table 1, or a pharmaceutically acceptable salt, solvate, hydrate, isomer, tautomer, racemate, isotope, or pharmaceutical composition thereof in the manufacture of a medicament. In some embodiments the medicament is for the treatment of cancer. In some embodiments the medicament is for the treatment of an autoimmune disease. In some embodiments the medicament is for the treatment of an inflammatory disease. In some embodiments the medicament is for the treatment of a thromboembolic disease. In some embodiments the medicament is for the treatment of multiple sclerosis. In some embodiments the medicament is for the treatment of rheumatoid arthritis. In some embodiments the medicament is for the treatment of psoriasis. In some embodiments the medicament is for the treatment of Sjogren's syndrome. In some embodiments the medicament is for the treatment of systemic lupus erythematosus. In some embodiments the medicament is for the treatment of urticaria. In some embodiments the BTK dependent condition is primary CNS lymphoma.

### Diseases

Described herein are methods of treating a disease treatable by inhibition of BTK in a mammal in need thereof comprising administering to the mammal, a therapeutically effective amount of a compound of any one of Formulas (I), (I′), (I-A), (I-B), (I-C), (I-D), or (II) or of Table 1, or a pharmaceutically acceptable salt, solvate, hydrate, isomer, tautomer, racemate or isotope thereof. In some embodiments the disease or disorder is a proliferative disease or disorder such as cancer or an oncology indication, an autoimmune disease or disorder, an inflammatory disease or disorder, or a thromboembolic disease or disorder.

Inhibition of BTK activity can be useful for the treatment of allergic disorders and/or autoimmune diseases and/or inflammatory diseases including, but not limited to: SLE, rheumatoid arthritis, multiple vasculitides, idiopathic thrombocytopenic purpura (ITP), myasthenia gravis, allergic rhinitis, multiple sclerosis (MS), transplant rejection, type I diabetes, membranous nephritis, inflammatory bowel dis-ease, autoimmune hemolytic anemia, autoimmune thyroid-itis, cold and warm agglutinin diseases, Evans syndrome, hemolytic uremic syndrome/thrombotic thrombocytopenic purpura, sarcoidosis, Sjogren's syndrome, peripheral neuropathies (e.g., Guillain-Barre syndrome), pemphigus vulgaris, and asthma.

BTK has been reported to play a role in controlling B-cell survival in certain B-cell cancers. For example, BTK has been shown to be important for the survival of BCR-Abl-positive B-cell acute lymphoblastic leukemia cells. Thus, inhibition of BTK activity can be useful for the treatment of B-cell lymphoma and leukemia. Further, it has been reported that dysregulation of the B-cell receptor and NF-κB pathways contribute to the pathogenesis of primary CNS lymphoma.

The compounds described herein, or a pharmaceutically acceptable salt, solvate, hydrate, isomer, tautomer, racemate, or isotope thereof, may be useful for the treatment of the above listed diseases. In one embodiment is provided a method of treating a BTK dependent condition, comprising administering to a subject in need thereof, an effective amount of a compound of Formula (I), (I′), (I-A), (I-B), (I-C), (I-D), or (II) or of Table 1, or a pharmaceutically acceptable salt, solvate, hydrate, isomer, tautomer, racemate, isotope, or pharmaceutical composition thereof. In one embodiment is provided a method of treating cancer, an autoimmune disease or disorder, an inflammatory disease or disorder, or a thromboembolic disease or disorder, comprising administering to a subject in need thereof, an effective amount of a compound of Formula (I), (I′), (I-A), (I-B), (I-C), (I-D), or (II) or of Table 1, or a pharmaceutically acceptable salt, solvate, hydrate, isomer, tautomer, racemate, isotope, or pharmaceutical composition thereof.

In one embodiment is provided a method of treating an autoimmune disease or disorder, comprising administering to a subject in need thereof, an effective amount of a compound of Formula (I), (I′), (I-A), (I-B), (I-C), (I-D), or (II) or of Table 1, or a pharmaceutically acceptable salt, solvate, hydrate, isomer, tautomer, racemate, isotope, or pharmaceutical composition thereof. In one embodiment, the autoimmune disease or disorder is multiple sclerosis, rheumatoid arthritis, psoriasis, Sjogren's syndrome, or systemic lupus erythematosus.

In one embodiment is provided a method of treating an inflammatory disease or disorder, comprising administering to a subject in need thereof, an effective amount of a compound of Formula (I), (I′), (I-A), (I-B), (I-C), (I-D), or (II) or of Table 1, or a pharmaceutically acceptable salt, solvate, hydrate, isomer, tautomer, racemate, isotope, or pharmaceutical composition thereof. In one embodiment, the inflammatory disease or disorder is urticaria.

In one embodiment is provided a method of treating a proliferative disease or disorder comprising administering to a subject in need thereof, an effective amount of a compound of Formula (I), (I′), (I-A), (I-B), (I-C), (I-D), or (II) or of Table 1, or a pharmaceutically acceptable salt, solvate, hydrate, isomer, tautomer, racemate, isotope, or pharmaceutical composition thereof. In one embodiment, the proliferative disease or disorder is cancer or an oncology indication. In another embodiment, the cancer or oncology indication is primary CNS lymphoma.

In one embodiment, the treatment of the above listed diseases is provided comprising administering to a subject in need thereof, an effective amount of a compound of Formula (I), (I′), (I-A), (I-B), (I-C), (I-D), (II), (II-A), or (II-A-i) or of Table 1, or a pharmaceutically acceptable salt, solvate, hydrate, isomer, tautomer, racemate, isotope, or pharmaceutical composition thereof optionally in combination with a corticosteroid, noncorticosteroidal, immunosuppressive, and/or antiinflammatory agents. In one embodiment, the immunosuppressive agent is selected from interferon alpha, interferon gamma, cyclophosphamide, tacrolimus, mycophenolate mofetil, methotrexate, dapsone, sulfasalazine, azathioprine, an anti-CD20 agent (such as rituximab, ofatumumab, obinutuzumab, or veltuzumab, or a biosimilar version thereof), anti-TNFalpha agent (such as entanercept, infliximab, golilumab, adalimumab, or certolizumab pegol or a biosimilar version thereof), anti-IL6 agent toward ligand or its receptors (such as tocilizumab, sarilumab, olokizumab, elsililumab, or siltuximab), anti-IL17 agent to ligand or its receptors (such as secukinumab, ustekinumab, brodalumab, or ixekizumab), anti-IL1 agent to ligand or its receptors (such as with rilonacept, canakinumab, or anakinra), anti-IL2 agent to ligand or its receptors (such as basiliximab or daclizumab), anti-CD2 agent such as alefacept, anti-CD3 agent such as muromonab-cd3, anti-CD80/86 agent such as abatacept or belatacept, anti-sphingosine-1-phosphate receptor agent such as fingolimod, anti-C5 agent such as eculizumab, anti-integrin alpha4 agent such as natalizumab, anti-α₄β₇ agent such as vedolizumab, anti-mTOR agent such as sirolimus or everolimus, anti-calcineurin agent such as tacrolimus, and anti-BAFF/BlyS agent (such as belimumab, VAY736, or blisibimod), leflunomide and teriflunomide. Preferably, the immunosuppressive agent is rituximab, ofatumumab, obinutuzumab, or veltuzumab, or a biosimilar version thereof.

### Atropisomers

Many organic compounds exist in optically active forms, i.e., they have the ability to rotate plane-polarized light. The prefixes d and l or (+) and (−) are employed to designate the sign of rotation of plane-polarized light by the compound, with (−) or l meaning that the compound is levorotatory. A compound prefixed with (+) or d is dextrorotatory. For a given chemical structure, these compounds, called stereoisomers, are identical except that they are mirror images of one another. Stereoisomers that are mirror images of one another may also be referred to as enantiomers, and a mixture of such isomers is often called an enantiomeric mixture. A 50:50 mixture of enantiomers is referred to as a racemic mixture or a racemate. Atropisomers are stereoisomers arising due to hindered rotation about a single bond, where energy differences create a barrier to rotation high enough to allow for isolation of individual conformers. Thus, atropisomers exist in a thermally controlled equilibrium, differing from most other types of chiral structures, where interconversion requires chemical isomerization (i.e. breaking covalent bonds).

Compounds of Formula (I), (F), (I-A), (I-B), (I-C), (I-D), (II), (II-A), or (II-A-i) or of Table 1 may exist as atropisomers, which may be present as isolated single enantiomers, or as racemic mixtures of both enantiomers, wherein the mixtures may comprise equal or unequal amounts of each enantiomer.

The energy barrier to thermal racemization of atropisomers may be determined by the steric hindrance to free rotation of one or more bonds forming a chiral axis. Certain biaryl compounds exhibit atropisomerism where rotation around an interannular bond lacking C2 symmetry is restricted. The free energy barrier for isomerization (enantiomerization) is a measure of the stability of the interannular bond with respect to rotation. Optical and thermal excitation can promote racemization of such isomers, dependent on electronic and steric factors.

Ortho-substituted biphenyl compounds may exhibit this type of conformational, rotational isomerism. Such biphenyls are enantiomeric, chiral atropisomers where the sp2-sp2 carbon-carbon, interannular bond between the phenyl rings has a sufficiently high energy barrier to prevent free rotation, and where substituents A≠B and A′≠B′ render the molecule asymmetric.

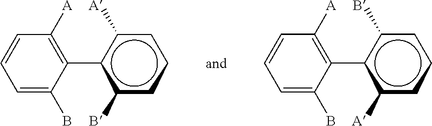

The steric interaction between A: A′, B:B′, and/or A:B′, B:A′ is large enough to make the planar conformation an energy maximum. Two non-planar, axially chiral enantiomers then exist as atropisomers when their interconversion is slow enough such that they can be isolated free of each other. By one definition, atropisomerism is defined to exist where the isomers have a half-life, t_{1/2}, of at least 1,000 seconds, which is a free energy barrier of 22.3 kcal mol⁻¹ (93.3 kJ mol⁻¹) at 300K (Oki, M. “Recent Advances in Atropisomerism,” Topics in Stereochemistry, 1983, 14, 1). Bold lines and dashed lines in the figures shown above indicate those moieties, or portions of the molecule, which are sterically restricted due to a rotational energy barrier. Bolded moieties exist orthogonally above the plane of the page, and dashed moieties exist orthogonally below the plane of the page. The “flat” part of the molecule (the left ring in each of the two depicted biphenyls) is in the plane of the page. Compounds with axial chirality, such as chiral biphenyl rings, can be described using configurational nomenclature. Atropisomers often, though not always, have substituents ortho to the aryl-aryl bond that cause significant steric repulsion thereby hindering the rotation. Factors influencing the stability of individual atropisomers include: repulsive interactions (e.g. steric bulk) of substituents near the axis of rotation; the length and rigidity of the aryl-aryl bond; and whether there are pathways, other than thermal, to induce rotation.

### Stereochemical Assignment

Determining the axial stereochemistry of biaryl atropisomers can be accomplished by analysis of a Newman projection along the axis of hindered rotation. The ortho substituents are assigned priority according to Cahn-Ingold-Prelog priority rules. Starting with the substituent of highest priority in the closest ring and moving along the shortest 90° path to the substituent of highest priority in the other ring (A to A′ in scheme 1 below), the absolute configuration is assigned P (plus) for clockwise and M (minus) for counterclockwise. In the example below, A has priority over A′ and B has priority over B′

For a review of atropisomers, including their nomenclature, see “Directed Synthesis of Chiral Biaryl Compounds” by Bringmann et. al. in Angew. Chem. Int. Ed. 2005, 44, 5384-5427. Alternate methods for assigning absolute axial configuration have been contemplated; see for example U.S. Pat. No. 8,440,677, columns 8 and 9.

### General Synthetic Schemes

Compounds having the structure of Formulas (I), as well as Formulas (I-A), (I-B), (I-C) and (I-D) and Table 1, can be synthesized using standard synthetic techniques known to those of skill in the art.

To this end, the reactions, processes and synthetic methods described herein are not limited to the specific conditions described in the following experimental section, but rather are intended as a guide to one with suitable skill in this field. For example, reactions may be carried out in any suitable solvent, or other reagents to perform the transformation[s] necessary. Generally, suitable solvents are protic or aprotic solvents which are substantially non-reactive with the reactants, the intermediates or products at the temperatures at which the reactions are carried out (i.e., temperatures which may range from the freezing to boiling temperatures). A given reaction may be carried out in one solvent or a mixture of more than one solvent. Depending on the particular reaction, suitable solvents for a particular work-up following the reaction may be employed. Unless otherwise indicated, conventional methods of mass spectroscopy (MS), liquid chromatography-mass spectroscopy (LCMS), NMR, HPLC, protein chemistry, biochemistry, recombinant DNA techniques, and pharmacology are employed.

Compounds are prepared using standard organic chemistry techniques such as those described in, for example, March's Advanced Organic Chemistry, 7th Edition, John Wiley and Sons, Inc. (2013). Alternate reaction conditions for the synthetic transformations described herein may be employed such as variation of solvent, reaction temperature, reaction time, as well as different chemical reagents and other reaction conditions. As necessary, the use of appropriate protecting groups may be required. The incorporation and cleavage of such groups may be carried out using standard methods described in Peter G. M. Wuts and Theodora W. Green, Protecting Groups in Organic Synthesis, 4th Edition, Wiley-Interscience. (2006). All starting materials and reagents are commercially available or readily prepared.

### Preparation of Compounds of Formula (I-A-iii)

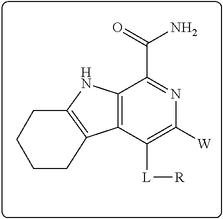

Compounds of Formula (I-A-iii) are prepared according to general Scheme 1 shown below:

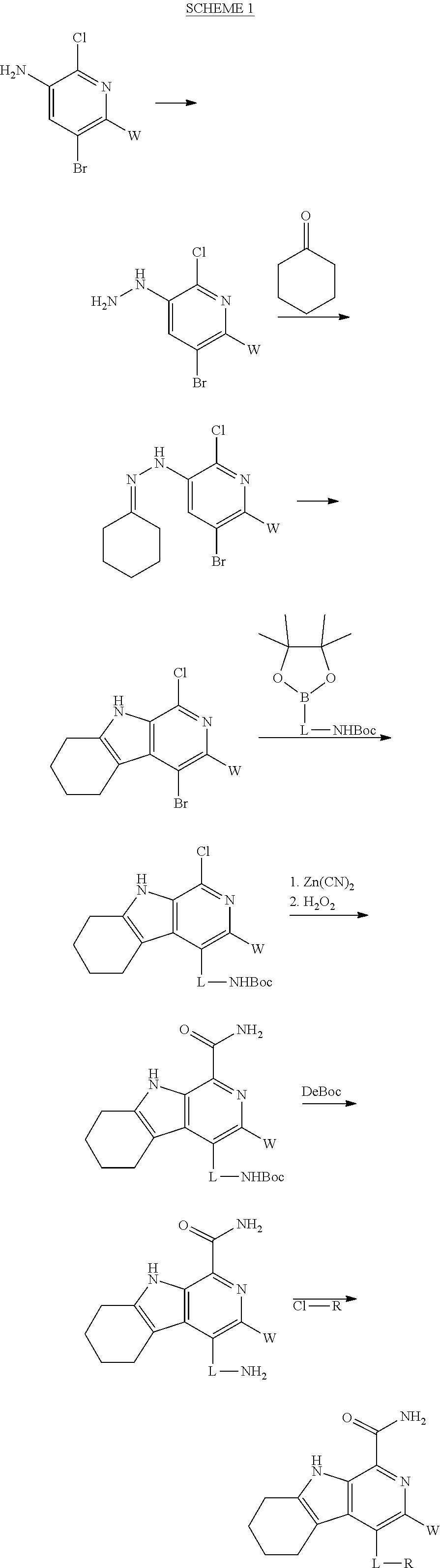

Formula (I-A-iii-a) when W is H:

5-bromo-2-chloropyridin-3-amine can be converted to the corresponding hydrazine using sodium nitrite in water in the presence of an acid such as HCl followed by the addition of a reducing agent such as stannous chloride to provide 5-bromo-2-chloro-3-hydrazineylpyridine. The hydrazine can be reacted with a ketone such as cyclohexanone in the presence of acid to form 5-bromo-2-chloro-3-(2-cyclohexylidenehydrazineyl)pyridine which can then be heated to form 4-bromo-1-chloro-6,7,8,9-tetrahydro-5H-pyrido[3,4-b]indole. Palladium catalyzed cross coupling of the bromoindole with a Boc protected amine-containing boronic acid or cyclic boronate ester can afford the coupled chloropyridine adduct which can be converted to the primary amide using a two-step procedure of cyanation and hydrolysis using known conditions. Deprotection then acylation with a carboxylic acid or carboxylic acid chloride provides target compounds of Formula (I-A-iii-a).

Formula (I-A-iii-b) when W is CH₃:

5-bromo-2-chloro-6-methylpyridin-3-amine can be converted to the corresponding hydrazine using sodium nitrite in water in the presence of an acid such as HCl followed by the addition of a reducing agent such as stannous chloride to provide 5-bromo-2-chloro-3-hydrazineyl-6-methylpyridine. The hydrazine can be reacted with a ketone such as cyclohexanone in the presence of acid to form 5-bromo-2-chloro-3-(2-cyclohexylidenehydrazineyl)-6-methylpyridine which can then be heated to form 4-bromo-1-chloro-3-methyl-6,7,8,9-tetrahydro-5H-pyrido[3,4-b]indole. Palladium catalyzed cross coupling of the bromoindole with a Boc protected amine-containing boronic acid or cyclic boronate ester can afford the coupled chloropyridine adduct which can be converted to the primary amide using a two-step procedure of cyanation and hydrolysis using known conditions. Deprotection then acylation with a carboxylic acid or carboxylic acid chloride provides target compounds of Formula (I-A-iii-b).

### Preparation of Compounds of Formula (I-A-iv)

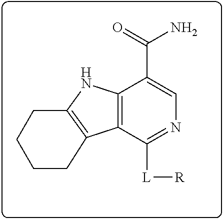

Compounds of Formula (I-A-iv) are prepared according to general Scheme 2 shown below:

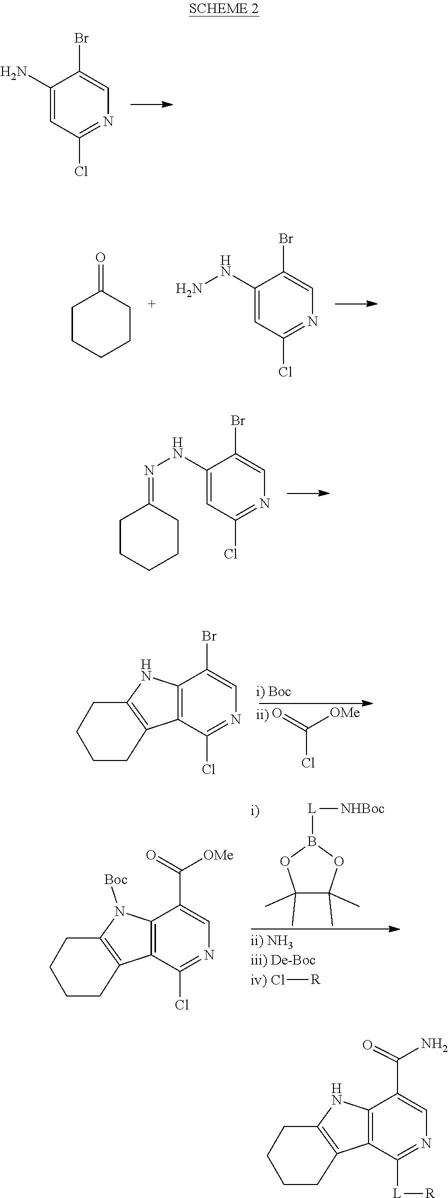

5-bromo-2-chloropyridin-4-amine can be converted to the corresponding hydrazine using sodium nitrite in water in the presence of an acid such as HCl followed by the addition of a reducing agent such as stannous chloride to provide 5-bromo-2-chloro-4-hydrazineylpyridine. The hydrazine can be reacted with a ketone such as cyclohexanone in the presence of acid to form 5-bromo-2-chloro-4-(2-cyclohexylidenehydrazineyl)pyridine which can then be heated to form 4-bromo-1-chloro-6,7,8,9-tetrahydro-5H-pyrido[4,3-b]indole. Protection of the indole nitrogen can be achieved using di-tert-butyl dicarbonate and metal halogen exchange followed by quench with methyl chloroformate can provide the desired ester intermediate. Cross coupling of the chloroindole with a Boc protected amine-containing boronic acid or cyclic boronate ester followed by conversion of the methyl ester to primary amide and deprotection of the Boc protecting groups can afford the penultimate intermediate amine. Acylation with a carboxylic acid or carboxylic acid chloride provides target compounds of Formula (I-A-iv).

### Preparation of Compounds of Formula (I-B-ii)

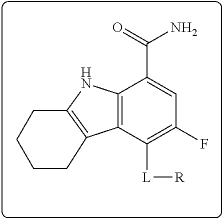

Compounds of Formula (I-B-ii) are prepared according to one of general Schemes 3 and 4, shown below:

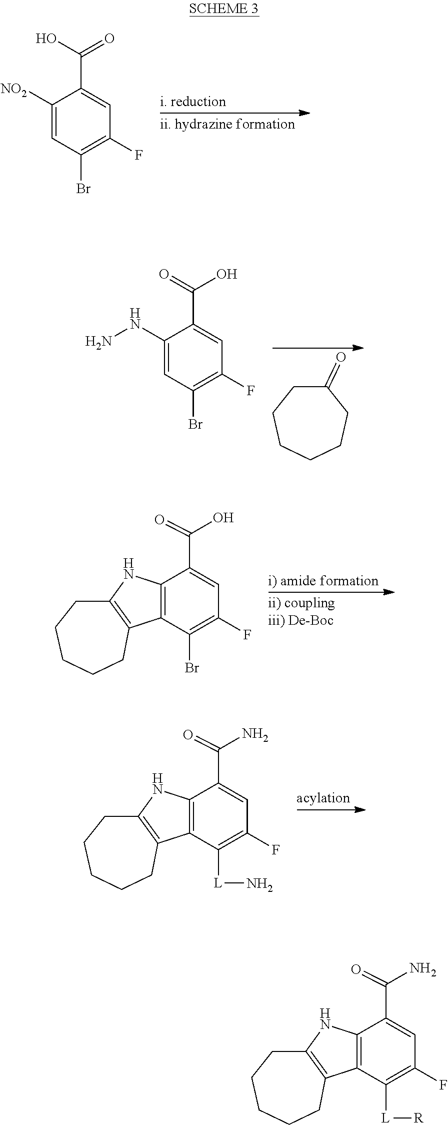

4-bromo-5-fluoro-2-nitrobenzoic acid can be converted to the corresponding hydrazine using a two-step procedure by first reducing the nitro group with a reagent such as tin chloride to provide the corresponding aniline which was reacted with sodium nitrite in water in the presence of an acid such as HCl followed by the addition of a reducing agent such as stannous chloride to provide 4-bromo-5-fluoro-2-hydrazineylbenzoic acid. The hydrazine can be reacted with a ketone such as cycloheptanone in the presence of acetic acid to form l-bromo-2-fluoro-5,6,7,8,9,10-hexahydrocyclohepta[b]indole-4-carboxylic acid. The pendant carboxylic acid group could be converted to the corresponding primary carboxamide using standard methodologies such as conversion to the acid chloride then quenching with ammonium hydroxide. Palladium catalyzed cross coupling of the tricyclic bromoindole with a Boc protected amine-containing boronic acid or cyclic boronate ester can afford the desired cross-coupling adduct. Deprotection then acylation with a carboxylic acid or carboxylic acid chloride provides target compounds of Formula (I-B-ii).

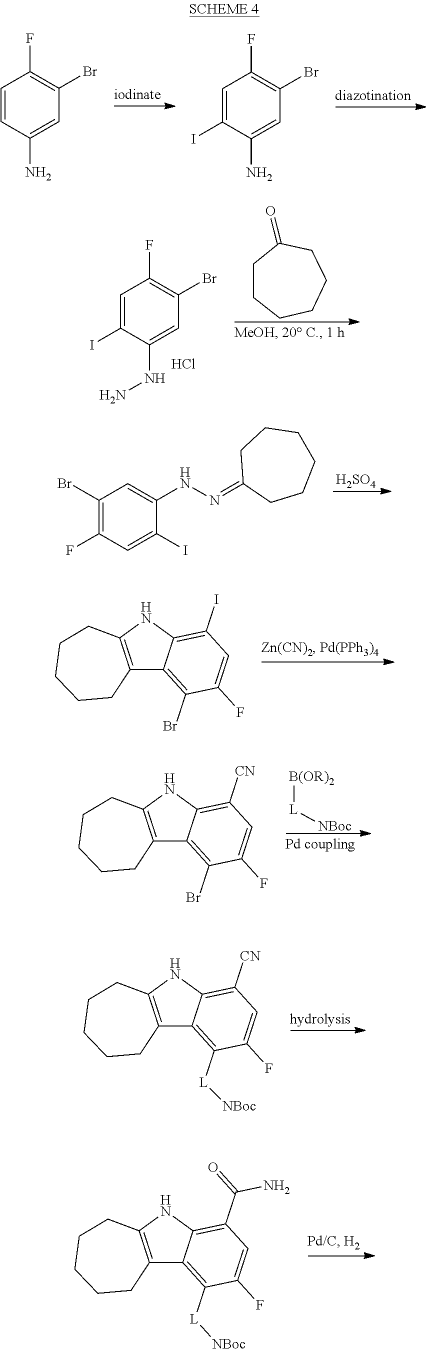

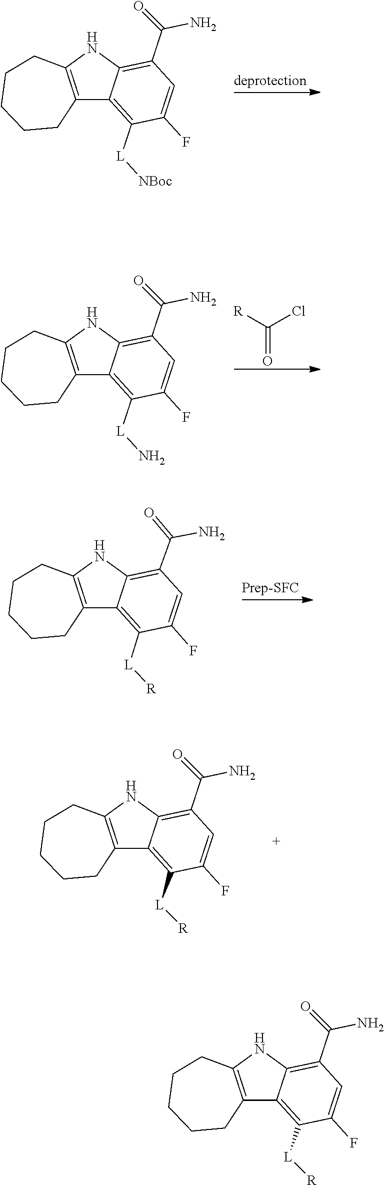

3-bromo-4-fluoroaniline can be reacted with an iodinating reagent such as N-iodosuccinimide to provide 2-iodo-4-fluoro-5-bromoaniline which can be converted to the corresponding hydrazine by reacting with sodium nitrite in water in the presence of an acid such as HCl followed by the addition of a reducing agent such as stannous chloride. The hydrazine can be reacted with a ketone such as cycloheptanone in a two-step manner by stirring in methanol then heating in the presence of catalytic amount of sulfuric acid to form the tricyclic indole intermediate. The iodo substituent can react with zinc cyanide in the presence of a palladium catalyst to afford 1-bromo-2-fluoro-5,6,7,8,9,10-hexahydrocyclohepta[b]indole-4-carbonitrile. Palladium catalyzed cross coupling of the tricyclic compound with a Boc protected amine-containing boronic acid or cyclic boronate ester can afford the desired cross-coupling adduct. The coupled product could be converted to the corresponding primary carboxamide using standard methodologies such as hydrido(dimethylphosphinous acid-kP)[hydrogenbis(dimethylphosphinito-kP)]platinum(II). Reduction of the olefin using hydrogen then deprotection then acylation with a carboxylic acid or carboxylic acid chloride provides target compounds of Formula (I-B-ii). The racemic end product could be separated into the pure single enantiomers using chiral chromatography.

### Preparation of Compounds of Formula (I-C-i)

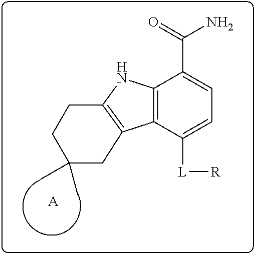

Compounds of Formula (I-C-i) are prepared according to one of general Schemes 5 and 6, shown below:

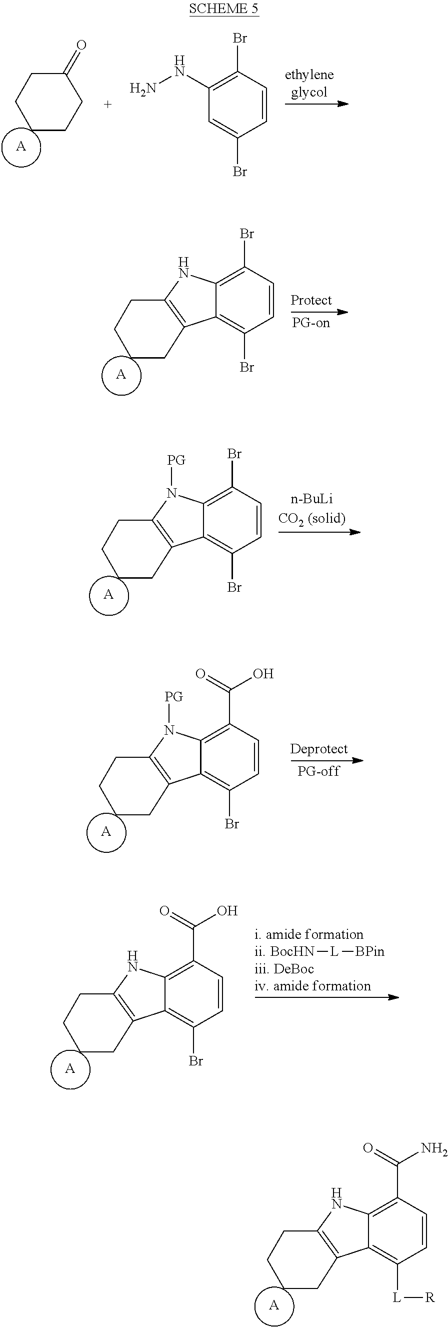

2-5-dibromphenylhydrazine can react with a spirocyclic ketone under standard conditions used for Fischer indole synthesis to provide the tricyclic indoles. The indole NH can be protected with either a Boc group or a SEM group and the ortho bromo group can be metallated using n-BuLi and quenched with carbon dioxide to provide the desired carboxylic acid. The Boc group could then be removed using a strong acid such as TFA or HCl (for Boc protection) or TBAF (for SEM protection) and the pendant carboxylic acid group could be converted to the corresponding primary carboxamide using standard methodologies such as conversion to the acid chloride then quenching with ammonium hydroxide. Palladium catalyzed cross coupling of the tricyclic bromoindole with a Boc protected amine-containing boronic acid or cyclic boronate ester can afford the desired cross-coupling adduct. Deprotection then acylation with a carboxylic acid or carboxylic acid chloride provides target compounds of Formula (I-C-i).

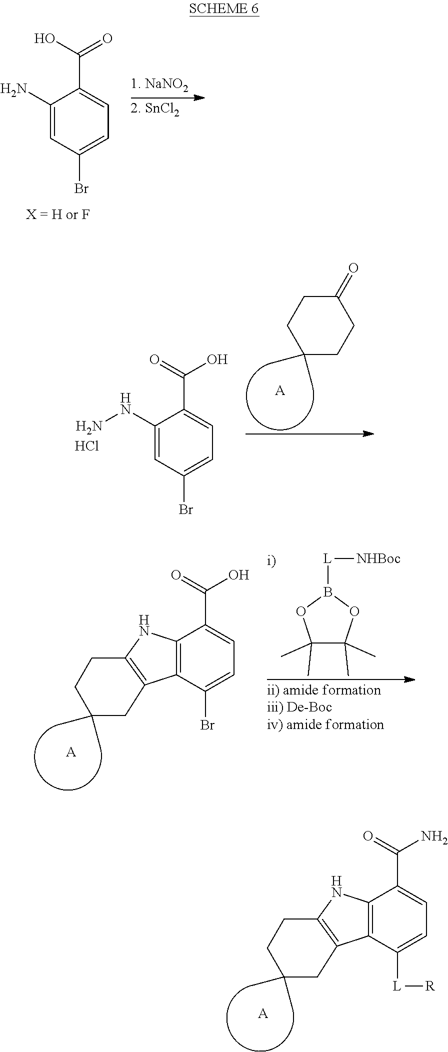

5-bromoanthranilic acid is be converted to the corresponding hydrazine using sodium nitrite in water in the presence of an acid such as HCl followed by the addition of a reducing agent such as stannous chloride. The hydrazine can be reacted with a spirocyclic ketone in the presence of acid to form the desired tricyclic indole. Palladium catalyzed cross coupling of the bromoindole with a Boc protected amine-containing boronic acid or cyclic boronate ester can afford the coupled chloropyridine adduct which can be converted to the primary amide using known conditions. Deprotection and acylation with a carboxylic acid or carboxylic acid chloride provides target compounds of Formula (I-C-i).

### Compounds of Formula (I-D)

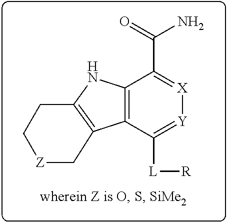

Compounds of Formula (I-D) are prepared according to general Scheme 7 shown below:

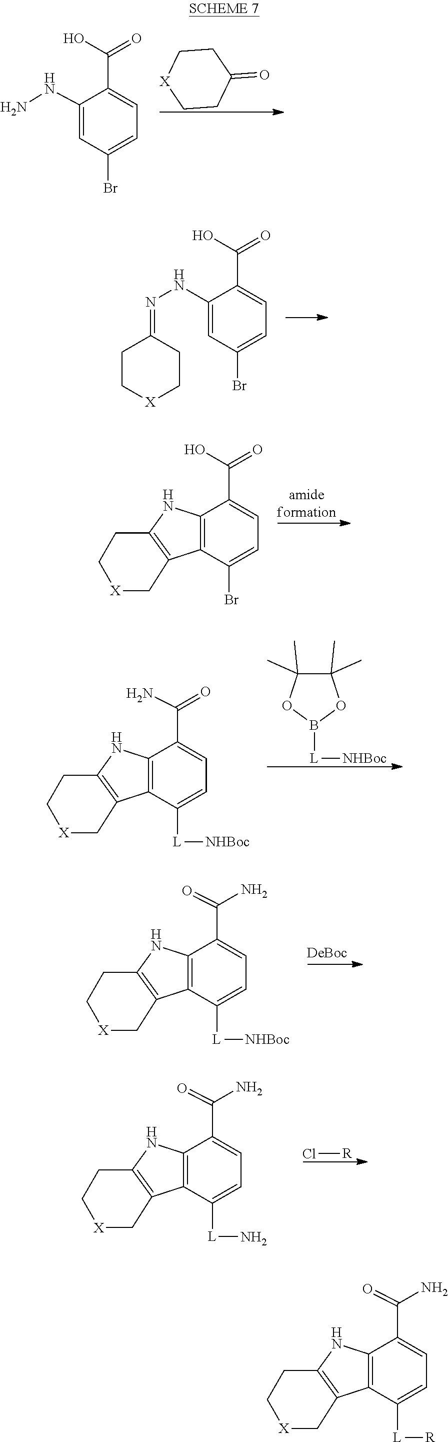

Arylhydrazine described above can be reacted with a spirocyclic ketone in the presence of acid to form the desired tricyclic indole. Conversion of the carboxylic acid to a carboxamide can be achieved using standard conditions for amide bond formation. Palladium catalyzed cross coupling of the bromoindole with a Boc protected amine-containing boronic acid or cyclic boronate ester can afford the coupled chloropyridine adduct which can be converted to the primary amide using known conditions. Deprotection and acylation with a carboxylic acid or carboxylic acid chloride provides target compounds of Formula (I-D).

### Preparation of Compounds of Formula (I-D-ix)

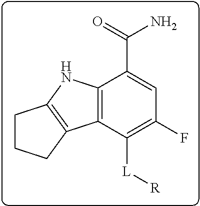

Compounds of Formula (I-D-ix) are prepared according to general Scheme 8, shown below:

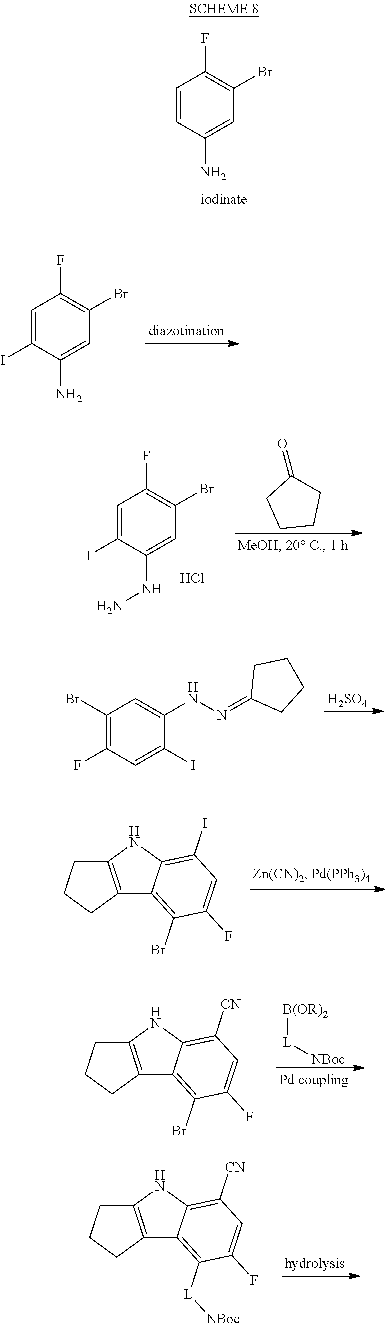

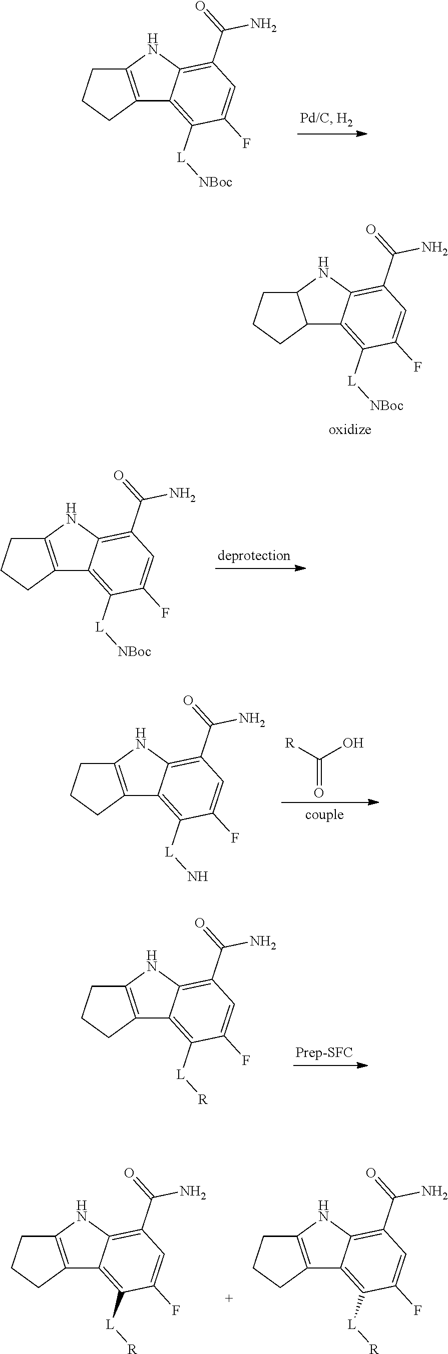

3-bromo-4-fluoroaniline can be reacted with an iodinating reagent such as N-iodosuccinimide to provide 2-iodo-4-fluoro-5-bromoaniline which can be converted to the corresponding hydrazine by reacting with sodium nitrite in water in the presence of an acid such as HCl followed by the addition of a reducing agent such as stannous chloride. The hydrazine can be reacted with a ketone such as cycloheptanone in a two-step manner by stirring in methanol then heating in the presence of catalytic amount of sulfuric acid to form the tricyclic indole intermediate. The iodo substituent can react with zinc cyanide in the presence of a palladium catalyst to afford 1-bromo-2-fluoro-5,6,7,8,9,10-hexahydrocyclohepta[b]indole-4-carbonitrile. Palladium catalyzed cross coupling of the tricyclic compound with a Boc protected amine-containing boronic acid or cyclic boronate ester can afford the desired cross-coupling adduct. The coupled product could be converted to the corresponding primary carboxamide using standard methodologies such as hydrido(dimethylphosphinous acid-kP)[hydrogenbis(dimethylphosphinito-kP)]platinum(II). Reduction of the olefin using hydrogen then reoxidation of the indole followed by deprotection then acylation with a carboxylic acid and an amide coupling agent provides target compounds of Formula (I-D-ix). The racemic end products could be separated into the pure single enantiomers using chiral chromatography.

### EXAMPLES

The following examples are provided for illustrative purposes only and not to limit the scope of the claims provided herein. While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

### Example 1-1

### Preparation of 4-(1-acryloylpiperidin-3-yl)-6,7,8,9-tetrahydro-5H-pyrido[3,4-b]indole-1-carboxamide (Compound 1-1)

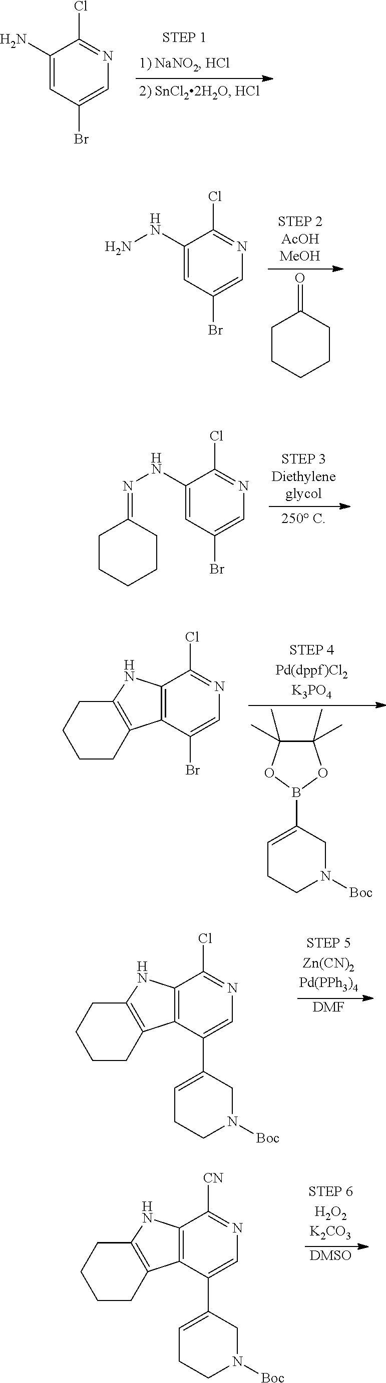

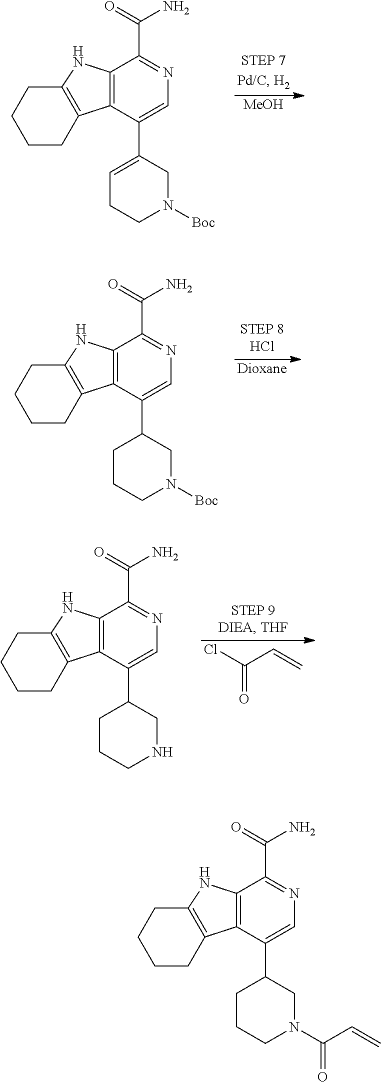

### Step 1: Preparation of 5-bromo-2-chloro-3-hydrazineylpyridine

A solution of sodium nitrite (6.65 g, 96.41 mmol) in water (70 mL) at 0° C. was added dropwise to a stirred mixture of 5-bromo-2-chloro-pyridin-3-amine (20.0 g, 96.4 mmol) in hydrochloric acid (70 mL, 36-38%). After stirring for 30 min at this temperature, the mixture was added dropwise to a solution of stannous chloride dihydrate (43.51 g, 192.81 mmol) in hydrochloric acid (50 mL, 36-38%) at 0° C. The reaction mixture was stirred for 1 h at 20° C. and then filtered. The isolated solid was washed with hydrochloric acid (50 mL, 36-38%) and dissolved in MeOH (100 mL). The resulting solution was neutralized with saturated aqueous sodium bicarbonate and extracted with ethyl acetate (3×300 mL). The combined organic layers were washed with brine (300 mL), dried over anhydrous sodium sulfate and concentrated under vacuum to give 5-bromo-2-chloro-3-hydrazineylpyridine (16.00 g, 70%) as a yellow solid. ESI-MS [M+H]⁺ calculated for (C₅H₅BrClN₃)=221.94, 223.94, 225.94; found: 221.95, 223.95, 225.95. ¹H NMR (300 MHz, DMSO-d₆) δ 7.66 (d, J=2.3 Hz, 1H), 7.59 (d, J=2.3 Hz, 1H), 7.20 (s, 1H), 4.33 (s, 2H)

### Step 2: Preparation of 5-bromo-2-chloro-3-(2-cyclohexylidenehydrazineyl) pyridine

To a stirred solution of 5-bromo-2-chloro-3-hydrazineylpyridine (16.0 g, 71.9 mmol) and cyclohexanone (8.47 g, 86.3 mmol) in methanol (200 mL) was added acetic acid (2 mL, 34.97 mmol) at 20° C. The reaction mixture was stirred for 12 h at 20° C. The reaction mixture was quenched with saturated aqueous sodium bicarbonate (400 mL) and extracted with ethyl acetate (3×300 mL). The combined organic layers were washed with brine (300 mL), dried over anhydrous sodium sulfate and concentrated under vacuum. The residue was purified by column chromatography on silica gel eluting with ethyl acetate in petroleum ether (10%) to give 5-bromo-2-chloro-3-(2-cyclohexylidenehydrazineyl)pyridine (20.0 g, 92%) as a yellow solid. ESI-MS [M+H]⁺ calculated for (C₁₁H₁₃BrClN₃)=302.00, 304.00, 306.00; found: 302.10, 304.10, 306.10. ¹H NMR (300 MHz, DMSO-d₆) δ 8.47 (s, 1H), 7.88 (d, J=2.3 Hz, 1H), 7.78 (d, J=2.3 Hz, 1H), 2.43 (t, J=5.8 Hz, 2H), 2.32 (t, J=5.9 Hz, 2H), 1.71-1.47 (m, 6H).

### Step 3: Preparation of 4-bromo-1-chloro-6,7,8,9-tetrahydro-5H-pyrido[3,4-b]indole

5-bromo-2-chloro-3-(2-cyclohexylidenehydrazineyl)pyridine (20.00 g, 66.09 mmol) in diethylene glycol (150 mL) was heated for 30 min at 250° C. The cooled reaction mixture was quenched with water (300 mL) and extracted with ethyl acetate (3×200 mL). The combined organic layers were washed with brine (300 mL), dried over anhydrous sodium sulfate and concentrated under vacuum. The residue was purified by column chromatography on silica gel eluting with ethyl acetate in petroleum ether (23%) to give 4-bromo-1-chloro-6,7,8,9-tetrahydro-5H-pyrido[3,4-b]indole (8.50 g, 43%) as a yellow solid. ESI-MS [M+H]⁺ calculated for (C₁₁H₁₀BrClN₂)=284.97, 286.97, 287.97; found: 284.95, 286.95, 288.95. ¹H NMR (300 MHz, DMSO-d₆) δ 11.92 (s, 1H), 7.91 (s, 1H), 2.92 (t, J=6.0 Hz, 2H), 2.76 (t, J=5.7 Hz, 2H), 1.88-1.75 (m, 4H).

### Step 4: Preparation of tert-butyl 5-(1-chloro-6,7,8,9-tetrahydro-5H-pyrido[3,4-b]indol-4-yl)-3,6-dihydropyridine-1(2H)-carboxylate

A mixture of 4-bromo-1-chloro-6,7,8,9-tetrahydro-5H-pyrido[3,4-b]indole (2.00 g, 7.00 mmol), tert-butyl 5-(4,4,5,5-tetramethyl-1,3,2-dioxaborolan-2-yl)-3,6-dihydropyridine-1(2H)-carboxylate (2.60 g, 8.40 mmol), potassium phosphate (4.46 g, 21.01 mmol) and Pd(dppf)Cl₂ (512 mg, 0.70 mmol) in water (5 mL) and THF (20 mL) was degassed and backfilled with nitrogen (×5). The reaction mixture was heated for 12 h at 60° C. under nitrogen atmosphere. The cooled mixture was diluted with water (100 mL) and extracted with ethyl acetate (3×60 mL). The combined organic layers were washed with brine (150 mL), dried over anhydrous sodium sulfate and concentrated under vacuum. The residue was purified by column chromatography on silica gel eluting with ethyl acetate in petroleum ether (22%) to give tert-butyl-5-(1-chloro-6,7,8,9-tetrahydro-5H-pyrido[3,4-b]indol-4-yl)-3,6-dihydropyridine-1(2H)-carboxylate (850 mg, 31%) as a white solid. ESI-MS [M+H]⁺ calculated for (C₂₁H₂₆ClN₃O₂)=388.17, 390.17; found: 388.25, 390.25. ¹H NMR (300 MHz, DMSO-d₆) δ 11.63 (s, 1H), 7.59 (s, 1H), 5.84-5.75 (m, 1H), 4.09-3.99 (m, 2H), 3.52 (t, J=5.9 Hz, 2H), 2.77 (t, J=5.9 Hz, 2H), 2.62 (t, J=5.3 Hz, 2H), 2.31-2.22 (m, 2H), 1.86-1.70 (m, 4H), 1.42 (s, 9H).

### Step 5: Preparation of tert-butyl 5-(1-cyano-6,7,8,9-tetrahydro-5H-pyrido[3,4-b]indol-4-yl)-3,6-dihydropyridine-1(2H)-carboxylate

A mixture of tert-butyl 5-(1-chloro-6,7,8,9-tetrahydro-5H-pyrido[3,4-b]indol-4-yl)-3,6-dihydropyridine-1(2H)-carboxylate (850 mg, 2.19 mmol), zinc cyanide (257 mg, 2.19 mmol) and tetrakis(triphenylphosphine)palladium (253 mg, 0.22 mmol) in DMF (10 mL) was degassed and backfilled with nitrogen(×5). The reaction mixture was heated for 1 h at 100° C. under nitrogen atmosphere. The cooled reaction mixture was diluted with water (50 mL) and extracted with ethyl acetate (3×50 mL). The combined organic layers were washed with brine (2×50 mL), dried over anhydrous sodium sulfate and concentrated under vacuum. The residue was purified by column chromatography on silica gel eluting with ethyl acetate in petroleum ether (24%) to give tert-butyl-5-(1-cyano-6,7,8,9-tetrahydro-5H-pyrido[3,4-b]indol-4-yl)-3,6-dihydropyridine-1(2H)-carboxylate (550 mg, 65%) as a yellow solid. ESI-MS [M+H]⁺ calculated for (C₂₂H₂₆N₄O₂)=379.21; found=379.30. ¹H NMR (300 MHz, DMSO-d₆) δ 12.20 (s, 1H), 7.98 (s, 1H), 5.89-5.81 (m, 1H), 4.09-3.99 (m, 2H), 3.53 (t, J=5.7 Hz, 2H), 2.80 (t, J=6.0 Hz, 2H), 2.63 (t, J=6.2 Hz, 2H), 2.32-2.24 (m, 2H), 1.90-1.68 (m, 4H), 1.42 (s, 9H)

### Step 6: Preparation of tert-butyl 5-(1-carbamoyl-6,7,8,9-tetrahydro-5H-pyrido[3,4-b]indol-4-yl)-3,6-dihydropyridine-1(2H)-carboxylate

Hydrogen peroxide (1.65 g, 14.53 mmol, 30%) was added to a stirred mixture of tert-butyl 5-(1-cyano-6,7,8,9-tetrahydro-5H-pyrido[3,4-b]indol-4-yl)-3,6-dihydropyridine-1(2H)-carboxylate (550 mg, 1.45 mmol) and potassium carbonate (602 mg, 4.36 mmol) in DMSO (10 mL) at 20° C. The reaction mixture was heated for 0.5 h at 60° C. The cooled reaction mixture was quenched by the addition of water (50 mL) and extracted with ethyl acetate (3×40 mL). The combined organic layers were washed with aqueous Na₂S₂O₃ (2×30 mL) and brine (30 mL), dried over anhydrous sodium sulfate and concentrated under vacuum to give tert-butyl-5-(1-carbamoyl-6,7,8,9-tetrahydro-5H-pyrido[3,4-b]indol-4-yl)-3,6-dihydro-2H-pyridine-1-carboxylate (500 mg, 85%) as a yellow solid. ESI-MS [M+H]⁺ calculated for (C₂₂H₂₈N₄O₃)=397.22; found=397.30. ¹H NMR (300 MHz, DMSO-d₆) δ 11.24 (s, 1H), 8.13 (s, 1H), 7.85 (s, 1H), 7.59 (s, 1H), 5.86-5.77 (m, 1H), 4.15-4.05 (m, 2H), 3.54 (t, J=5.8 Hz, 2H), 2.81 (t, J=5.9 Hz, 2H), 2.64 (t, J=5.3 Hz, 2H), 2.31-2.25 (m, 2H), 1.88-1.69 (m, 4H), 1.42 (s, 9H).

### Step 7: Preparation of tert-butyl 3-(1-carbamoyl-6,7,8,9-tetrahydro-5H-pyrido[3,4-b]indol-4-yl)piperidine-1-carboxylate

A mixture of tert-butyl 5-(1-carbamoyl-6,7,8,9-tetrahydro-5H-pyrido[3,4-b]indol-4-yl)-3,6-dihydro-2H-pyridine-1-carboxylate (350 mg, 0.88 mmol) and 10% palladium on carbon (100 mg) in methanol (10 mL) was stirred for 48 h at 20° C. under hydrogen (2-3 atm). The mixture was filtered, and the filtrate concentrated under vacuum to give tert-butyl 3-(1-carbamoyl-6,7,8,9-tetrahydro-5H-pyrido[3,4-b]indol-4-yl)piperidine-1-carboxylate (330 mg, crude) as a yellow solid. ESI-MS [M+H]⁺ calculated for (C₂₂H₃₀N₄O₃)=399.23; found=399.30.

### Step 8: Preparation of 4-(piperidin-3-yl)-6,7,8,9-tetrahydro-5H-pyrido[3,4-b]indole-1-carboxamide hydrochloride

A mixture of tert-butyl 3-(1-carbamoyl-6,7,8,9-tetrahydro-5H-pyrido[3,4-b]indol-4-yl)piperidine-1-carboxylate (330 mg, 0.83 mmol) and HCl (4 M) in 1,4-dioxane, (5 mL) was stirred for 1 h at 20° C. The resulting mixture was concentrated under vacuum to give 4-(3-piperidyl)-6,7,8,9-tetrahydro-5H-pyrido[3,4-b]indole-1-carboxamide hydrochloride (360 mg, crude) as a yellow solid. ESI-MS [M+H]⁺ calculated for (C₁₇H₂₂N₄O)=299.18; found=299.15.

### Step 9: Preparation of 4-(1-acryloylpiperidin-3-yl)-6,7,8,9-tetrahydro-5H-pyrido[3,4-b]indole-1-carboxamide (Compound 1-1)

To a stirred mixture of 4-(3-piperidyl)-6,7,8,9-tetrahydro-5H-pyrido[3,4-b]indole-1-carboxamide hydrochloride (360 mg, crude) and N,N-diisopropylethylamine (1.39 g, 10.75 mmol) in THF (10 mL) was added dropwise acryloyl chloride (97.31 mg, 1.08 mmol) at −78° C. The reaction mixture was stirred for 1 h at −78° C. The mixture was quenched with water (20 mL) and extracted with ethyl acetate (3×15 mL). The combined organic layers were washed with brine (20 mL), dried over sodium sulfate and concentrated under vacuum. The residue was purified by Prep-HPLC (Column: Xselect CSH OBD Column 30×150 mm 5 um; Mobile Phase A: Water (0.1% FA), Mobile Phase B: Acetonitrile; Flow rate: 60 mL/min; Gradient: 17% B to 39% B in 7 min; 220 nm) to give 4-(1-prop-2-enoyl-3-piperidyl)-6,7,8,9-tetrahydro-5H-pyrido[3,4-b]indole-1-carboxamide (54.2 mg) as yellow solid. ESI-MS [M+H]⁺ calculated for (C₂₄H₂₄N₄O₂)=353.19; found=353.15. ¹H NMR (300 MHz, DMSO-d₆) δ 11.14 (s, 1H), 8.07 (s, 1H), 8.03 (s, 1H), 7.54 (s, 1H), 6.95-6.75 (m, 1H), 6.19-6.04 (m, 1H), 5.74-5.59 (m, 1H), 4.65-4.54 (m, 1H), 4.20-4.09 (m, 1H), 3.32-3.07 (m, 2H), 2.99-2.73 (m, 5H), 2.05-1.91 (m, 2H), 1.86-1.72 (m, 5H), 1.63-1.42 (m, 1H).

### Example 2-1

### Preparation of 4-(2-acryloyl-1,2,3,4-tetrahydroisoquinolin-5-yl)-6,7,8,9-tetrahydro-5H-pyrido[3,4-b]indole-1-carboxamide (Compound 2-1)

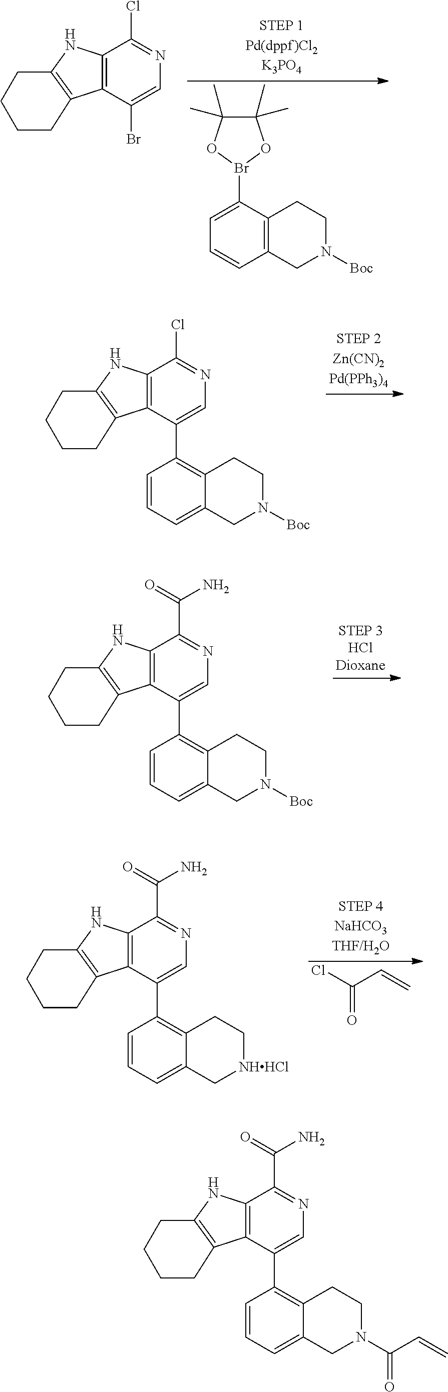

### Step 1: Preparation of tert-butyl 5-(1-chloro-6,7,8,9-tetrahydro-5H-pyrido[3,4-b]indol-4-yl)-3,4-dihydroisoquinoline-2(1H)-carboxylate

A mixture of 4-bromo-1-chloro-6,7,8,9-tetrahydro-5H-pyrido[3,4-b]indole (3.10 g, 10.86 mmol), tert-butyl 5-(4,4,5,5-tetramethyl-1,3,2-dioxaborolan-2-yl)-3,4-dihydro-1H-isoquinoline-2-carboxylate (4.68 g, 13.0 mmol), [1,1′-bis(diphenylphosphino) ferrocene]dichloropalladium(II) (794 mg, 1.09 mmol) and potassium phosphate (6.91 g, 32.5 mmol) in THF (25 mL) and water (2 mL) was degassed and backfilled with nitrogen (×5). The reaction mixture was heated overnight at 60° C. under nitrogen atmosphere. The cooled mixture was diluted with water (100 mL) and extracted with ethyl acetate (50 mL×3). The combined organic layers were washed with water (50 mL) and brine (50 mL), dried over anhydrous sodium sulfate and concentrated under vacuum. The residue was purified by column chromatography on silica gel eluting with ethyl acetate in petroleum ether (0 to 20%) to give tert-butyl 5-(1-chloro-6,7,8,9-tetrahydro-5H-pyrido[3,4-b]indol-4-yl)-3,4-dihydroisoquinoline-2(1H)-carboxylate (4.5 g, 97%) as an off-white solid. ¹H NMR (400 MHz, DMSO-d₆) δ 11.69 (s, 1H), 7.59 (s, 1H), 7.30-7.21 (m, 2H), 7.12-7.06 (m, 1H), 4.67-4.46 (m, 2H), 3.43 (t, J=6.2 Hz, 2H), 2.92-2.64 (m, 2H), 2.40-2.24 (m, 2H), 1.90-1.78 (m, 2H), 1.77-1.65 (m, 2H), 1.60-1.46 (m, 2H), 1.39 (s, 9H).

### Step 2: Preparation of tert-butyl 5-(1-carbamoyl-6,7,8,9-tetrahydro-5H-pyrido[3,4-b]indol-4-yl)-3,4-dihydroisoquinoline-2(1H)-carboxylate

A mixture of tert-butyl 5-(1-chloro-6,7,8,9-tetrahydro-5H-pyrido[3,4-b]indol-4-yl)-3,4-dihydroisoquinoline-2(1H)-carboxylate (4.50 g, 10.27 mmol), zinc cyanide (664 mg, 5.65 mmol) and tetrakis(triphenylphosphine)palladium (1.19 g, 1.03 mmol) in DMF (35 mL) was degassed and backfilled with nitrogen (×5). The reaction mixture was heated for 48 h at 120° C. under nitrogen atmosphere. The cooled mixture was diluted with water (200 mL) and extracted with ethyl acetate (80 mL×3). The combined organic layers were washed with water (50 mL×3) and brine (80 mL), dried over anhydrous sodium sulfate and concentrated under vacuum. The residue was purified by column chromatography on silica gel eluting with ethyl acetate in petroleum ether (0 to 45%) to give tert-butyl 5-(1-carbamoyl-6,7,8,9-tetrahydro-5H-pyrido[3,4-b]indol-4-yl)-3,4-dihydroisoquinoline-2(1H)-carboxylate (670 mg, 15%) as a yellow solid. ¹H NMR (300 MHz, DMSO-d₆) δ 11.30 (s, 1H), 8.19 (s, 1H), 7.86 (s, 1H), 7.63 (s, 1H), 7.34-7.23 (m, 2H), 7.18-7.09 (m, 1H), 4.71-4.48 (m, 2H), 3.45 (t, J=6.0 Hz, 2H), 2.80 (t, J=6.9 Hz, 2H), 2.34 (t, J=6.0 Hz, 2H), 1.94-1.83 (m, 2H), 1.78-1.66 (m, 2H), 1.59-1.48 (m, 2H), 1.41 (s, 9H).

### Step 3: Preparation of 4-(1,2,3,4-tetrahydroisoquinolin-5-yl)-6,7,8,9-tetrahydro-5H-pyrido[3,4-b]indole-1-carboxamide hydrochloride

A mixture of tert-butyl 5-(1-carbamoyl-6,7,8,9-tetrahydro-5H-pyrido[3,4-b]indol-4-yl)-3,4-dihydroisoquinoline-2(1H)-carboxylate (330 mg, 739 μmol) and hydrogen chloride (4 M in dioxane, 15 mL) was stirred for 2 h at 20° C. The mixture was evaporated to dryness under vacuum to give 4-(1,2,3,4-tetrahydroisoquinolin-5-yl)-6,7,8,9-tetrahydro-5H-pyrido[3,4-b]indole-1-carboxamide hydrochloride (256 mg, crude) as a yellow solid. ESI-MS [M+H]⁺ calculated for (C₂₁H₂₂N₄O)=347.18; found=347.30.

### Step 4: Preparation of 4-(2-acryloyl-1,2,3,4-tetrahydroisoquinolin-5-yl)-6,7,8,9-tetrahydro-5H-pyrido[3,4-b]indole-1-carboxamide (Compound 2-1)

To a stirred mixture of 4-(1,2,3,4-tetrahydroisoquinolin-5-yl)-6,7,8,9-tetrahydro-5H-pyrido[3,4-b]indole-1-carboxamide hydrochloride (256 mg, crude) and sodium bicarbonate (562 mg, 6.69 mmol) in THF (10 mL) and water (2 mL) was added acryloyl chloride (61 mg, 0.67 mmol) at 0° C. The reaction mixture was stirred for 1 h at 0° C. The mixture was diluted with water (50 mL) and extracted with ethyl acetate (50 mL×3). The combined organic layers were washed with water (30 mL×2) and brine (30 mL), dried over anhydrous sodium sulfate and concentrated under vacuum. The residue was purified by Prep-HPLC (Column: XBridge Prep OBD C18 Column, 30×150 mm 5 um; Mobile Phase A: water (10 mmol/L NH₄HCO₃+0.1% NH₃.H₂O), Mobile Phase B: Acetonitrile; Flow rate: 60 mL/min; Gradient: 25% B to 60% B in 7 min; 220 nm) to give 4-(2-acryloyl-1,2,3,4-tetrahydroisoquinolin-5-yl)-6,7,8,9-tetrahydro-5H-pyrido[3,4-b]indole-1-carboxamide (57.4 mg, 22%) as an off-white solid. ESI-MS [M+H]⁺ calculated for (C₂₄H₂₄N₄O₂)=401.19; found=401.30. ¹H NMR (300 MHz, DMSO-d₆) δ 11.29 (s, 1H), 8.18 (d, J=2.9 Hz, 1H), 7.85 (s, H), 7.62 (d, J=3.1 Hz, 1H), 7.35-7.25 (m, 2H), 7.18-7.09 (m, 1H), 7.00-6.71 (m, 1H), 6.14 (dd, J=16.8, 2.7 Hz, 1H), 5.78-5.62 (m, 1H), 4.95-4.66 (m, 2H), 3.79-3.49 (m, 2H), 2.78 (t, J=6.3 Hz, 2H), 2.48-2.29 (m, 2H), 1.97-1.79 (m, 2H), 1.77-1.62 (m, 2H), 1.60-1.44 (m, 2H).

### Examples 2-2 to 2-10

Compounds 2-2 through 2-10 in Table 2, were prepared in a similar manner to compounds 1-1 and 2-1, as described in examples 1 and 2.

TABLE 2
COMPOUNDS 2-2 THROUGH 2-10

| Compound | | |
|---|---|---|
| No. | Structure | ¹H NMR |
| 2-2 | 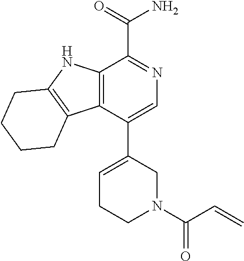 [SMILES: C=CC(=O)N1CCC=C(c2cnc(C(N)=O)c3[nH]c4c(c23)CCCC4)C1] | (300 MHz, DMSO-d⁶) δ 11.23 (s, 1H), 8.13 (s, 1H), 7.95-7.84 (m, 1H), 7.59 (s, 1H), 6.99-6.76 (m, 1H), 6.29-6.06 (m, 1H), 5.88-5.82 (m, 1H), 5.78-5.63 (m, 1H), 4.41-4.20 (m, 2H), 3.82-3.69 (m, 2H), 2.81 (t, J = 5.8 Hz, 2H), 2.70-2.53 (m, 2H), 2.45-2.20 (m, 2H), 1.88-1.63 (m, 4H). |
| 2-3 | 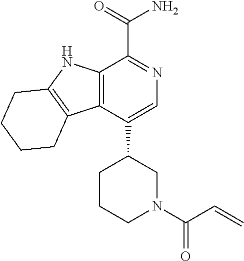 [SMILES: C=CC(=O)N1CCC[C@H](c2cnc(C(N)=O)c3[nH]c4c(c23)CCCC4)C1] | (300 MHz, CD₃OD) δ 8.07 (s, 1H), 6.95-6.68 (m, 1H), 6.22 (d, J = 16.5 Hz, 1H), 5.82-5.69 (m, 1H), 4.80-4.62 (m, 1H), 4.33-4.15 (m, 1H), 3.57-3.37 (m, 2H), 3.27-3.04 (m, 1H), 2.97-2.78 (m, 4H), 2.22-2.10 (m, 1H), 2.09-1.81 (m, 6H), 1.78-1.63 (m, 1H). |
| 2-4 | 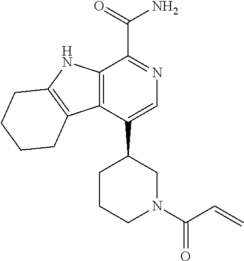 [SMILES: C=CC(=O)N1CCC[C@@H](c2cnc(C(N)=O)c3[nH]c4c(c23)CCCC4)C1] | (300 MHz, CD₃OD) δ 8.07 (s, 1H), 6.93-6.71 (m, 1H), 6.22 (d, J = 16.8 Hz, 1H), 5.84-5.68 (m, 1H), 4.82-4.62 (m, 1H), 4.31-4.14 (m, 1H), 3.58-3.35 (m, 2H), 3.27-3.04 (m, 1H), 3.01-2.79 (m, 4H), 2.22-2.10 (m, 1H), 2.09-1.85 (m, 6H), 1.79-1.60 (m, 1H). |
| 2-5 | 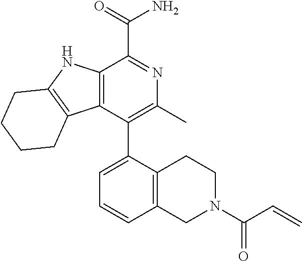 [SMILES: C=CC(=O)N1CCc2c(cccc2-c2c(C)nc(C(N)=O)c3[nH]c4c(c23)CCCC4)C1] | (300 MHz, DMSO-d⁶) δ 11.04 (s, 1H), 8.03 (s, 1H), 7.57 (s, 1H), 7.36-7.24 (m, 2H), 7.13-7.06 (m, 1H), 7.03-6.68 (m, 1H), 6.13 (d, J = 16.6 Hz, 1H), 5.77- 5.59 (m, 1H), 4.96-4.64 (m, 2H), 3.78-3.50 (m, 2H), 2.73 (t, J = 5.9 Hz, 2H), 2.35-2.12 (m, 5H), 1.82-1.57 (m, 4H), 1.56-1.34 (m, 2H). |
| 2-6 | 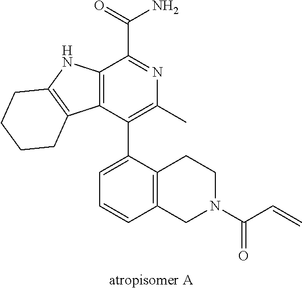 [SMILES: C=CC(=O)N1CCc2c(cccc2-c2c(C)nc(C(N)=O)c3[nH]c4c(c23)CCCC4)C1] | (400 MHz, DMSO-d⁶) δ 11.08 (s, 1H), 8.05 (s, 1H), 7.61 (s, 1H), 7.36-7.24 (m, 2H), 7.13-7.06 (m, 1H), 7.03-6.68 (m, 1H), 6.13 (d, J = 16.6 Hz, 1H), 5.77- 5.59 (m, 1H), 4.96-4.64 (m, 2H), 3.78-3.50 (m, 2H), 2.73 (t, J = 5.9 Hz, 2H), 2.35-2.12 (m, 5H), 1.82-1.57 (m, 4H), 1.56-1.34 (m, 2H). |
| 2-7 | 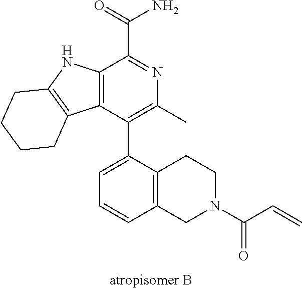 [SMILES: C=CC(=O)N1CCc2c(cccc2-c2c(C)nc(C(N)=O)c3[nH]c4c(c23)CCCC4)C1] | (400 MHz, DMSO-d⁶) δ 11.04 (s, 1H), 8.03 (s, 1H), 7.57 (s, 1H), 7.36-7.24 (m, 2H), 7.13-7.06 (m, 1H), 7.03-6.68 (m, 1H), 6.13 (d, J = 16.6 Hz, 1H), 5.77- 5.59 (m, 1H), 4.96-4.64 (m, 2H), 3.78-3.50 (m, 2H), 2.73 (t, J = 5.9 Hz, 2H), 2.35-2.12 (m, 5H), 1.82-1.57 (m, 4H), 1.56-1.34 (m, 2H). |
| 2-8 | 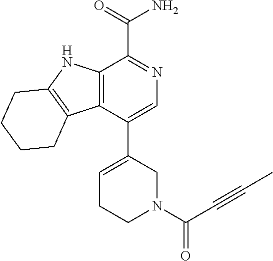 [SMILES: CC#CC(=O)N1CCC=C(c2cnc(C(N)=O)c3[nH]c4c(c23)CCCC4)C1] | (400 MHz, DMSO-d₆) δ 11.2 (s, 1H), 8.06 (s, 1H), 7.80 (d, J = 8.4 Hz, 1H), 7.52 (s, 1H), 5.77 (m, 1H), 4.37-4.22 (m, 2H), 3.81 (t, J = 5.6 Hz, 1H), 3.63 (t, J = 5.6 Hz, 1H), 2.81 (t, J = 5.8 Hz, 2H), 2.70-2.10 (m, 4H), 2.00 (s, 3H), 1.91-1.73 (m, 4H). |
| 2-9 | 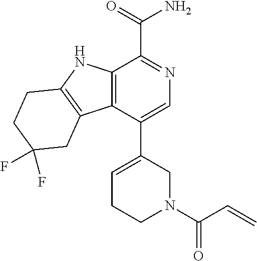 [SMILES: C=CC(=O)N1CCC=C(c2cnc(C(N)=O)c3[nH]c4c(c23)CC(F)(F)CC4)C1] | (400 MHz, DMSO-d₆) δ 11.6 (s, 1H), 8.17 (s, 1H), 7.95 (m, 1H), 7.65 (s, 1H), 6.89 (m, 1H), 6.16 (m, 1H), 5.86 (m, 1H), 5.73 (dd, J = 1.2, 5.2 Hz, 1H), 4.37- 4.22 (m, 2H), 3.77 (t, J = 5.6 Hz, 1H), 3.12-3.01 (m, 4H), 2.81 (t, J = 5.8 Hz, 2H), 2.23 (m, 4H). |
| 2-10 | 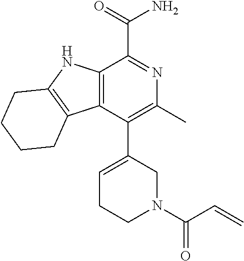 [SMILES: C=CC(=O)N1CCC=C(c2c(C)nc(C(N)=O)c3[nH]c4c(c23)CCCC4)C1] | (300 MHz, DMSO-d₆) δ 10.9 (s, 1H), 7.96 (s, 1H), 7.54 (s, 1H), 6.93-6.71 (m, 1H), 6.22-6.12 (m, 1H), 5.84-5.68 (m, 2H), 4.22-4.12 (m, 2H), 3.85-3.65 (m, 1H), 3.58-3.35 (m, 2H), 2.85-2.75 (m, 4H), 2.09-1.85 (m, 6H), 1.85-1.75 (m, 4H). |

### Example 3-1

### Preparation of 1-(2-acryloyl-1,2,3,4-tetrahydroisoquinolin-5-yl)-6,7,8,9-tetrahydro-5H-pyrido[4,3-b]indole-4-carboxamide (Compound 3-1)

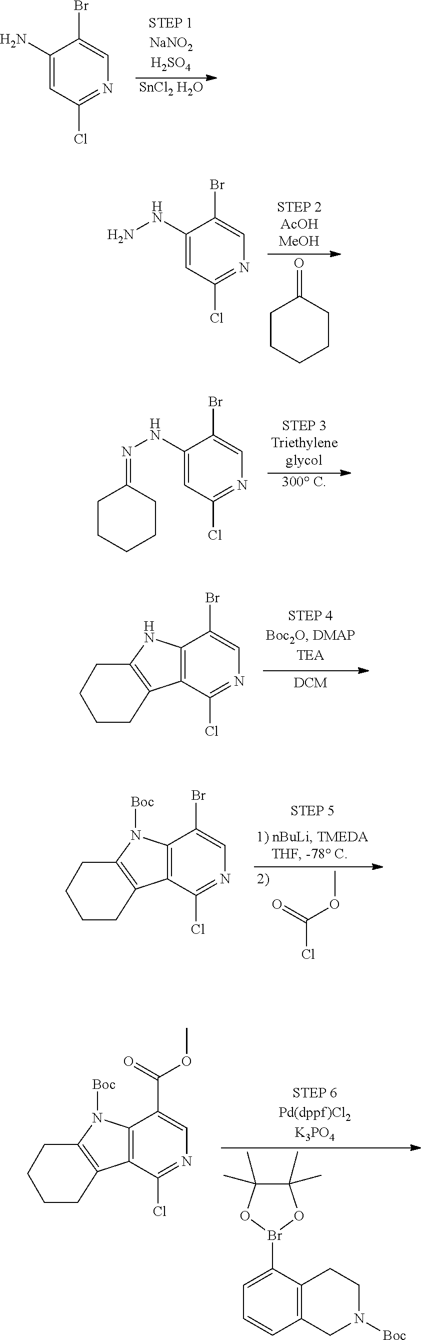

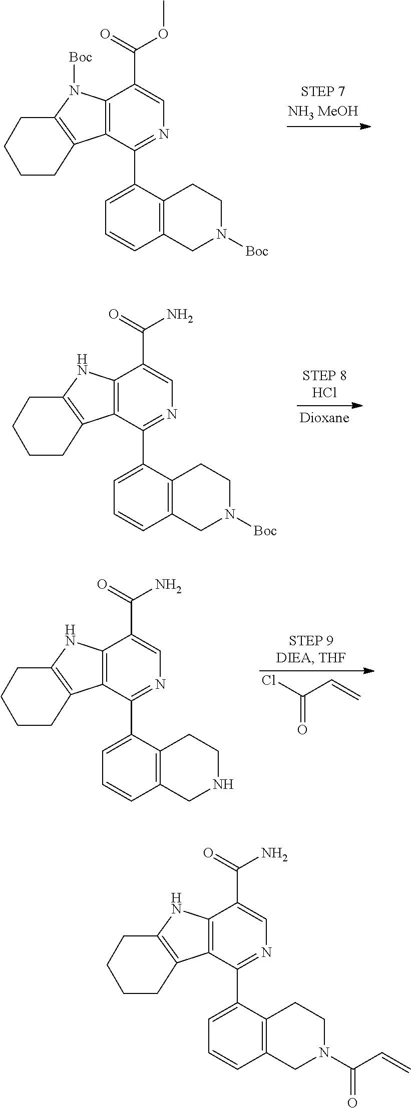

### Step 1: Preparation of 5-bromo-2-chloro-4-hydrazineylpyridine

To a solution of 5-bromo-2-chloro-pyridin-4-amine (20.00 g, 96.41 mmol) in 40% H₂SO₄ (150 mL) was added dropwise a solution of sodium nitrite (7.98 g, 115.69 mmol) in water (8 mL) at 0° C. After stirring for 2 h at 0° C., the resulting mixture was added to a stirred solution of stannous chloride dihydrate (43.51 g, 192.81 mmol) in 40% H₂SO₄ (150 mL) at 0° C. and stirred for 1 h at this temperature. The reaction mixture was basified with ammonium hydroxide and extracted with dichloromethane and methanol (9:1, 1 L×5). The combined organic layers were washed with water (1 L×2) and brine (1 L), dried over anhydrous magnesium sulfate and concentrated under vacuum to give 5-bromo-2-chloro-4-pydrazineylpyridine (12.60 g) as a white solid. ¹H NMR (400 MHz, DMSO-d₆) δ 8.00 (s, 1H), 7.80 (s, 1H), 6.94 (s, 1H), 4.45 (s, 2H).

### Step 2: Preparation of 5-bromo-2-chloro-4-(2-cyclohexylidenehydrazineyl)pyridine

To a suspension of 5-bromo-2-chloro-4-pydrazineylpyridine (8.00 g, 35.96 mmol) and cyclohexanone (5.29 g, 53.94 mmol) in methanol (50 mL) was added acetic acid (216 mg, 3.60 mmol) at 20° C. The reaction mixture was stirred for 2 h at this temperature. The resulting mixture was filtered, the isolated solid washed with methanol (10 mL) and dried under reduced pressure to give 5-bromo-2-chloro-4-(2-cyclohexylidenehydrazineyl)pyridine (7.50 g) as a white solid. ¹H NMR (300 MHz, DMSO-d₆) δ 8.73 (s, 1H), 8.22 (s, 1H), 7.15 (s, 1H), 2.42 (t, J=5.6 Hz, 2H), 2.33 (t, J=5.8 Hz, 2H), 1.67-1.58 (m, 6H).

### Step 3: Preparation of 4-bromo-1-chloro-6,7,8,9-tetrahydro-5H-pyrido[4,3-b]indole

5-bromo-2-chloro-4-(2-cyclohexylidenehydrazineyl)pyridine (6.50 g, 21.48 mmol) in triethylene glycol (20 mL) was stirred for 30 min at 300° C. The cooled mixture was diluted with ethyl acetate (300 mL), washed with water (100 mL×2) and brine (100 mL), dried over anhydrous sodium sulfate and concentrated under vacuum. The residue was purified by column chromatography on silica gel eluting with ethyl acetate in petroleum ether (0 to 11%) to give 4-bromo-1-chloro-6,7,8,9-tetrahydro-5H-pyrido[4,3-b]indole (1.00 g) as a brown solid. ¹H NMR (DMSO-d₆) δ 11.89 (s, 1H), 7.99 (s, 1H), 2.88 (t, J=4.9 Hz, 2H), 2.74 (t, J=4.6 Hz, 2H), 1.87-1.74 (m, 4H).

### Step 4: Preparation of tert-butyl 4-bromo-1-chloro-6,7,8,9-tetrahydro-5H-pyrido[4,3-b]indole-5-carboxylate

To a solution of 4-bromo-1-chloro-6,7,8,9-tetrahydro-5H-pyrido[4,3-b]indole (1.00 g, 3.50 mmol) in dichloromethane (10 mL) were added N,N-dimethyl-4-aminopyridine (43 mg, 0.35 mmol) and triethylamine (710 mg, 7.02 mmol) followed by the addition of di-tert-butyl dicarbonate (1.15 g, 5.27 mmol) at 20° C. The reaction mixture was stirred for 0.5 h at 20° C., diluted with dichloromethane (100 mL) and washed with water (50 mL×2) and brine (50 mL). The organic layer was dried over anhydrous magnesium sulfate and concentrated under vacuum. The residue was purified by column chromatography on silica gel eluting with ethyl acetate in petroleum ether (0 to 9%) to give tert-butyl 4-bromo-1-chloro-6,7,8,9-tetrahydro-5H-pyrido[4,3-b]indole-5-carboxylate (1.12 g) as a light yellow solid. ¹H NMR (DMSO-d₆) δ 8.23 (s, 1H), 2.86 (t, J=6.1 Hz, 2H), 2.77 (t, J=6.2 Hz, 2H), 1.88-1.72 (m, 4H), 1.62 (s, 9H).

### Step 5: Preparation of 5-(tert-butyl) 4-methyl 1-chloro-6,7,8,9-tetrahydro-5H-pyrido[4,3-b]indole-4,5-dicarboxylate

To a stirred mixture of tert-butyl 4-bromo-1-chloro-6,7,8,9-tetrahydro-5H-pyrido[4,3-b]indole-5-carboxylate (1.12 g, 2.90 mmol) and TMEDA (371 mg, 3.19 mmol) in THF (15 mL) was added nBuLi (1.28 mL, 2.5 M in hexane, 3.20 mmol) at −78° C. under nitrogen atmosphere. After stirring for 1 h at −78° C., methyl chloroformate (411 mg, 4.35 mmol) was added dropwise. The reaction mixture was stirred for 1 h at −78° C., quenched with saturated aqueous ammonium chloride (100 mL) and extracted with ethyl acetate (50 mL×3). The combined organic layers were washed with water (50 mL×2) and brine (50 mL), dried over anhydrous sodium sulfate and concentrated under vacuum. The residue was purified by column chromatography on silica gel eluting with ethyl acetate in petroleum ether (0 to 15%) to give 5-(tert-butyl) 4-methyl 1-chloro-6,7,8,9-tetrahydro-5H-pyrido[4,3-b]indole-4,5-dicarboxylate (780 mg) as a light yellow solid. ¹H NMR (400 MHz, DMSO-d₆) δ 8.32 (s, 1H), 3.83 (s, 3H), 2.94-2.83 (m, 4H), 1.89-1.73 (m, 4H), 1.57 (s, 9H).

### Step 6: Preparation of 5-(tert-butyl) 4-methyl 1-(2-(tert-butoxycarbonyl)-1,2,3,4-tetrahydroisoquinolin-5-yl)-6,7,8,9-tetrahydro-5H-pyrido[4,3-b]indole-4,5-dicarboxylate

A mixture of 5-(tert-butyl) 4-methyl 1-chloro-6,7,8,9-tetrahydro-5H-pyrido[4,3-b]indole-4,5-dicarboxylate (200 mg, 0.55 mmol), tert-butyl 5-(4,4,5,5-tetramethyl-1,3,2-dioxaborolan-2-yl)-3,4-dihydro-1H-isoquinoline-2-carboxylate (217 mg, 0.60 mmol), potassium phosphate (349 mg, 1.64 mmol) and [1,1′-bis(diphenylphosphino)-ferrocene]dichloropalladium(II) (40 mg, 0.05 mmol) in THF (5 mL) and water (0.5 mL) was degassed and backfilled with nitrogen (×5). The reaction mixture was heated for 12 h at 60° C. under nitrogen atmosphere. The cooled mixture was diluted with water (50 mL) and extracted with ethyl acetate (50 mL×3). The combined organic layers were washed with water (100 mL) and brine (50 mL), dried over anhydrous sodium sulfate and concentrated under vacuum. The residue was purified by column chromatography on silica gel eluting with ethyl acetate in petroleum ether (0 to 20%) to give 5-(tert-butyl) 4-methyl 1-(2-(tert-butoxycarbonyl)-1,2,3,4-tetrahydroisoquinolin-5-yl)-6,7,8,9-tetrahydro-5H-pyrido[4,3-b]indole-4,5-dicarboxylate (290 mg, 94%) as a white solid. ¹H NMR (300 MHz, DMSO-d₆) δ 8.56 (s, 1H), 7.32-7.28 (m, 2H), 7.14-7.08 (m, 1H), 4.67-4.52 (m, 2H), 3.86 (s, 3H), 3.55-3.37 (m, 2H), 2.91-2.79 (m, 2H), 2.47-2.37 (m, 1H), 2.29-2.15 (m, 1H), 1.79-1.70 (m, 4H), 1.59 (s, 9H), 1.56-1.43 (m, 2H), 1.41 (s, 9H).

### Step 7: Preparation of tert-butyl 5-(4-carbamoyl-6,7,8,9-tetrahydro-5H-pyrido[4,3-b]indol-1-yl)-3,4-dihydroisoquinoline-2(1H)-carboxylate

A mixture of 5-(tert-butyl) 4-methyl 1-(2-(tert-butoxycarbonyl)-1,2,3,4-tetrahydroisoquinolin-5-yl)-6,7,8,9-tetrahydro-5H-pyrido[4,3-b]indole-4,5-dicarboxylate (290 mg, 0.52 mmol) and ammonia (10 mL, 7.0 M in methanol) was heated in a sealed tube for 36 h at 100° C. The cooled reaction mixture was concentrated under vacuum and the isolated residue purified by column chromatography on silica gel eluting with ethyl acetate in petroleum ether (0 to 50%) to give tert-butyl 5-(4-carbamoyl-6,7,8,9-tetrahydro-5H-pyrido[4,3-b]indol-1-yl)-3,4-dihydro-1H-isoquinoline-2-carboxylate (170 mg, 74%) as an off-white solid. ¹H NMR (300 MHz, DMSO-d₆) δ 11.23 (s, 1H), 8.66 (s, 1H), 8.15 (s, 1H), 7.50 (s, 1H), 7.33-7.22 (m, 2H), 7.12-7.05 (m, 1H), 4.67-4.51 (m, 2H), 3.52-3.37 (m, 2H), 2.80-2.67 (m, 2H), 2.48-2.40 (m, 1H), 2.32-2.18 (m, 1H), 2.02-1.86 (m, 1H), 1.83-1.65 (m, 3H), 1.60-1.47 (m, 2H), 1.41 (s, 9H).

### Step 8: Preparation of 1-(1,2,3,4-tetrahydroisoquinolin-5-yl)-6,7,8,9-tetrahydro-5H-pyrido[4,3-b]indole-4-carboxamide hydrochloride

A mixture of tert-butyl 5-(4-carbamoyl-6,7,8,9-tetrahydro-5H-pyrido[4,3-b]indol-1-yl)-3,4-dihydro-1H-isoquinoline-2-carboxylate (170 mg, 0.38 mmol) and hydrogen chloride (4 M in dioxane, 5 mL) was stirred for 2 h at 20° C. The reaction mixture was concentrated under vacuum to give 1-(1,2,3,4-tetrahydroisoquinolin-5-yl)-6,7,8,9-tetrahydro-5H-pyrido[4,3-b]indole-4-carboxamide hydrochloride (200 mg, crude) as a yellow solid. ESI-MS [M+H]⁺ calculated for (C₂₁H₂₂N₄O) 347.18, found: 347.15.

### Step 9: Preparation of 1-(2-acryloyl-1,2,3,4-tetrahydroisoquinolin-5-yl)-6,7,8,9-tetrahydro-5H-pyrido[4,3-b]indole-4-carboxamide (Compound 3-1)

To a stirred mixture of 1-(1,2,3,4-tetrahydroisoquinolin-5-yl)-6,7,8,9-tetrahydro-5H-pyrido[4,3-b]indole-4-carboxamide hydrochloride (200 mg, crude) and N-ethyl-N-isopropyl-propan-2-amine (675 mg, 5.22 mmol) in THF (8 mL) was added dropwise acryloyl chloride (47 mg, 0.52 mmol) at −78° C. The reaction mixture was stirred for 1 h −78° C. The mixture was quenched with saturated aqueous ammonium chloride (50 mL) and extracted with ethyl acetate (80 mL×2). The combined organic layers were washed with water (100 mL×2) and brine (100 mL), dried over anhydrous sodium sulfate and concentrated under vacuum. The residue was purified by Prep-HPLC (Column: XBridge Prep OBD C18 Column, 30×150 mm 5 um; Mobile Phase A: Water (10 mmol/L NH₄HCO₃+0.1% NH₃.H₂O), Mobile Phase B: Acetonitrile; Flow rate: 60 mL/min; Gradient: 25% B to 45% B in 7 min; 220 nm) to give 1-(2-acryloyl-1,2,3,4-tetrahydroisoquinolin-5-yl)-6,7,8,9-tetrahydro-5H-pyrido[4,3-b]indole-4-carboxamide (11 mg) as an off-white solid. ESI-MS [M+H]⁺ calculated for (C₂₄H₂₄N₄O₂)=401.19; found=401.30. ¹H NMR (300 MHz, DMSO-d₆) δ 11.23 (s, 1H), 8.65 (s, 1H), 8.15 (s, 1H), 7.50 (s, 1H), 7.33-7.25 (m, 2H), 7.15-7.06 (m, 1H), 6.99-6.73 (m, 1H), 6.14 (d, J=16.7 Hz, 1H), 5.77-5.62 (m, 1H), 4.93-4.70 (m, 2H), 3.79-3.45 (m, 2H), 2.84-2.67 (m, 2H), 2.66-2.57 (m, 1H), 2.37-2.16 (m, 1H), 2.03-1.87 (m, 1H), 1.85-1.63 (m, 3H), 1.61-1.39 (m, 2H).

### Examples 3-2, 3-3 & 3-4

Compounds 3-2, 3-3 and 3-4, in Table 3, were prepared in a similar manner to compound 3-1, as described in example 3.

TABLE 3
COMPOUNDS 3-2, 3-3 AND 3-4

| Compound | | |
|---|---|---|
| No. | Structure | ¹H NMR |
| 3-2 | 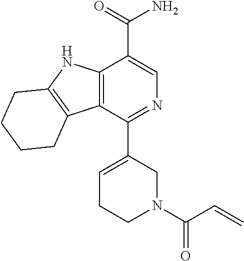 [SMILES: C=CC(=O)N1CCC=C(c2ncc(C(N)=O)c3[nH]c4c(c23)CCCC4)C1] | (300 MHz, DMSO-d₆) δ 11.19 (s, 1H), 8.58 (s, 1H), 8.11 (s, 1H), 7.48 (s, 1H), 7.01-6.71 (m, 1H), 6.15 (dd, J = 16.7, 2.4 Hz, 1H), 6.01-5.90 (m, 1H), 5.72 (dd, J = 10.4, 2.4 Hz, 1H), 4.63-4.44 (m, 2H), 3.82-3.70 (m, 2H) 2.76 (t, J = 6.4 Hz, 2H), 2.57 (t, J = 5.7 Hz, 2H), 2.44-2.25 (m, 2H), 1.90-1.63 (m, 4H). |
| 3-3 | 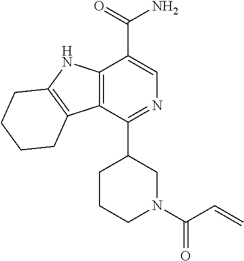 [SMILES: C=CC(=O)N1CCCC(c2ncc(C(N)=O)c3[nH]c4c(c23)CCCC4)C1] | (300 MHz, DMSO-d₆) δ 11.12 (s, 1H), 8.54 (s, 1H), 8.07 (s, 1H), 7.45 (s, 1H), 6.95-6.76 (m, 1H), 6.18-6.03 (m, 1H), 5.73-5.56 (m, 1H), 4.56 (t, J = 12.8 Hz, 1H), 4.23- 4.07 (m, 1H), 3.54 (t, J = 12.2 Hz, 0.5H), 3.32-3.30 (m, 1H), 3.10 (t, J = 12.8 Hz, 0.5H), 3.01-2.86 (m, 1H), 2.84- 2.60 (m, 4H), 2.10-1.89 (m, 2H), 1.88-1.67 (m, 5H), 1.64- 1.42 (m, 1H). |
| 3-4 | 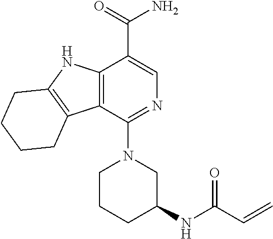 [SMILES: C=CC(=O)N[C@H]1CCCN(c2ncc(C(N)=O)c3[nH]c4c(c23)CCCC4)C1] | (400 MHz, DMSO-d₆) δ 10.9 (s, 1H), 8.26 (s, 1H), 8.07 (d, J = 7.6 Hz, 1H), 7.88 (br s, 1H), 7.21 (br s, 1H), 6.18- 6.03 (m, 1H), 6.07 (d, J = 12.0 Hz, 1H), 5.58 (dd, J = 12.0, 2.0 Hz, 1H), 3.95 (m, 1H), 3.64-3.53 (m, 2H), 3.32-3.30 (m, 1H), 3.01-2.66 (m, 6H), 2.04-1.60 (m, 8H). |

### Example 4-1

### Preparation of (S)-1-(3-(but-2-ynamido) piperidin-1-yl)-2-fluoro-5,6,7,8,9,10-hexahydro cyclohepta[b]indole-4-carboxamide (Compound 4-1)

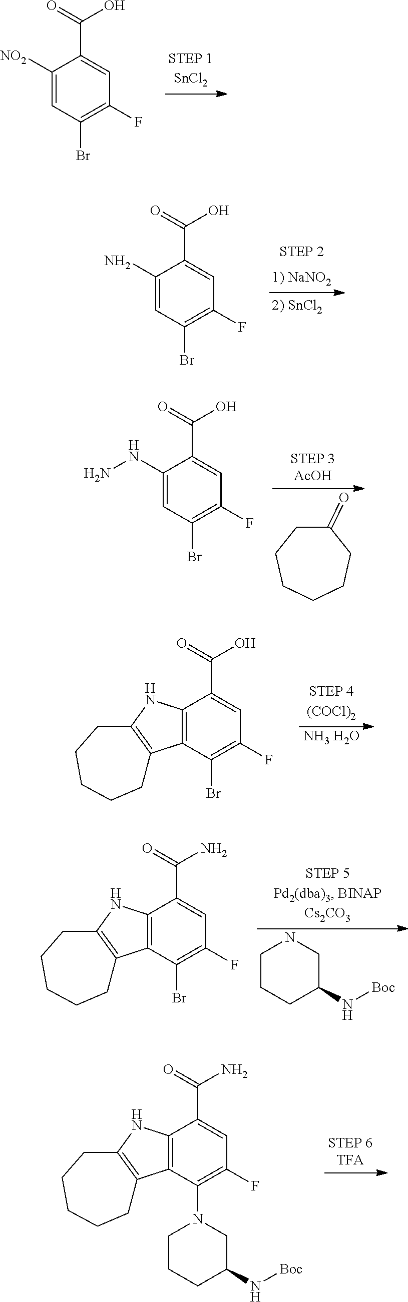

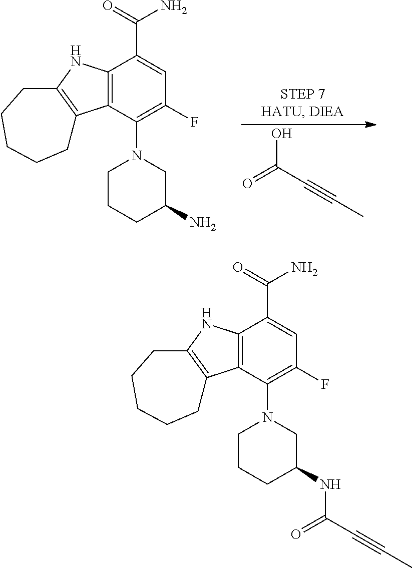

### Step 1: Preparation of 2-amino-4-bromo-5-fluorobenzoic acid

To a solution of 4-bromo-5-fluoro-2-nitrobenzoic acid (20.0 g, 75.76 mmol) in 18% HCl aq (140 mL) was added SnCl₂.2H₂O (51.0 g, 226 mmol). The reaction mixture was stirred at 90° C. for 3 h. The reaction mixture was filtered, the filter cake washed with water and dried under vacuum to give the desired product 2-amino-4-bromo-5-fluorobenzoic acid (17.0 g, 96% yield) as a white solid. LCMS (ESI) calculated for C₇H₅BrFNO₂⁺ [M+H]⁺ m/z 232.95, found 234.0.

### Step 2: Preparation of 4-bromo-5-fluoro-2-hydrazinylbenzoic acid

To a solution of 2-amino-4-bromo-5-fluorobenzoic acid (17.0 g, 72.6 mmol) in 36% HCl aq (120 mL) was added dropwise a solution of NaNO₂ (6.6 g, 95.7 mmol) in water (50 mL) at 0° C. The mixture was stirred at 0° C. for 0.5 h, and then a solution of SnCl₂.2H₂O (45 g, 199.4 mmol) in 18% HCl(aq) (40 mL) was added at 0° C. The mixture was stirred at 0° C. for 0.5 h, and the filter cake washed with water and dried under vacuum to give the desired product 4-bromo-5-fluoro-2-hydrazinylbenzoic acid (12.3 g, 69% yield) as a light brown solid. LCMS (ESI) calculated for C₇H₆BrFN₂O₂⁺ [M+H]⁺ m/z=247.96; found=249.0.

### Step 3: Preparation of 1-bromo-2-fluoro-5,6,7,8,9,10-hexahydrocyclohepta[b]indole-4-carboxylic acid

To a solution of 4-bromo-5-fluoro-2-hydrazinylbenzoic acid (8.0 g, 32.13 mmol) in acetic acid (100 mL) was added cycloheptanone (4.0 g, 35.71 mmol). The reaction was stirred at 130° C. for 16 h. The acetic acid was removed under vacuum, and water (50 mL) added to the residue and stirred at rt for 0.5 h. The mixture was filtered, and the filter cake dried under vacuum to give the desired product 1-bromo-2-fluoro-5,6,7,8,9,10-hexahydrocyclohepta[b]indole-4-carboxylic acid (5.0 g, 48% yield) as a brown solid. LCMS (ESI) calculated for C₁₄H₁₃BrFNO₂⁺ [M+H]⁺ m/z=325.01; found=326.0.

### Step 4: Preparation of 1-bromo-2-fluoro-5,6,7,8,9,10-hexahydrocyclohepta[b]indole-4-carboxamide

Oxalyl chloride (2.6 mL, 30.80 mmol) was added dropwise to a solution of 1-bromo-2-fluoro-5,6,7,8,9,10-hexahydrocyclohepta[b]indole-4-carboxylic acid (5.0 g, 15.33 mmol) in dichloromethane (50 mL) at 0° C. DMF (0.1 mL) was the added and the mixture stirred at 0° C. for another 0.5 h. The reaction mixture was then added dropwise to an ammonium hydroxide solution (25-28%; 20 mL) at 0° C., and the solution stirred at 0° C. for 0.5 h. The solution was then washed with brine dried over Na₂SO₄, filtered and concentrated. The resulting residue was purified by CombiFlash (from 100% petroleum ether to 50% ethyl acetate in petroleum ether in 25 min, 25 ml/min) to give the desired product 1-bromo-2-fluoro-5,6,7,8,9,10-hexahydro-cyclohepta[b]indole-4-carboxamide (610 mg) as a yellow solid. HRMS (ESI) calculated for C₁₄H₁₄BrFN₂O [M+H]⁺ m/z=324.03; found=325.0.

### Step 5: Preparation of tert-butyl (S)-(1-(4-carbamoyl-2-fluoro-5,6,7,8,9,10-hexahydro-cyclohepta[b] indol-1-yl) piperidin-3-yl) carbamate

To a solution of 1-bromo-2-fluoro-5,6,7,8,9,10-hexahydrocyclohepta[b]indole-4-carboxamide (200 mg, 0.62 mmol) in dioxane (2 mL) was added tert-butyl (S)-piperidin-3-ylcarbamate (148 mg, 0.74 mmol), BINAP (80 mg, 0.13 mmol), Pd₂(dba)₃ (56 mg, 0.06 mmol) and Cs₂CO₃ (240 mg, 0.73 mmol). The reaction mixture was stirred at 120° C. under N₂ for 16 h. The reaction mixture was filtered, concentrated, and the resulting residue purified by CombiFlash (from 100% petroleum ether to 80% ethyl acetate in petroleum ether in 25 min, 25 ml/min) to afford the desired product tert-butyl (S)-(1-(4-carbamoyl-2-fluoro-5,6,7,8,9,10-hexahydrocyclohepta[b]indol-1-yl)piperidin-3-yl)carbamate (80 mg, 29% yield) as a yellow solid. HRMS (ESI) calculated for C₂₄H₃₃FN₄O₃ [M+H]⁺ m/z 444.25, found 445.2.

### Step 6: Preparation of (S)-1-(3-aminopiperidin-1-yl)-2-fluoro-5,6,7,8,9,10-hexa-hydrocyclohepta [b]indole-4-carboxamide

To a solution tert-butyl (S)-(1-(4-carbamoyl-2-fluoro-5,6,7,8,9,10-hexahydrocyclohepta[b]indol-1-yl) piperidin-3-yl) carbamate (70 mg, 0.16 mmol) in DCM (2 mL) was added TFA (2 mL). The reaction solution was stirred at rt for 1 h, and then evaporated under vacuum to give the desired product (S)-1-(3-aminopiperidin-1-yl)-2-fluoro-5,6,7,8,9,10-hexahydrocyclohepta[b]indole-4-carboxamide 9 (60 mg, 100% yield) as a yellow solid. HRMS (ESI) calculated for C₁₉H₂₅FN₄O⁺ [M+H]⁺ m/z=344.20, found=345.2.

### Step 7: Preparation of (S)-1-(3-(but-2-ynamido) piperidin-1-yl)-2-fluoro-5,6,7,8,9,10-hexahydro cyclohepta[b]indole-4-carboxamide (Compound 4-1)

A solution of (S)-1-(3-aminopiperidin-1-yl)-2-fluoro-5,6,7,8,9,10-hexahydro-cyclohepta[b]indole-4-carboxamide (60 mg, 0.135 mmol) and DIEA (0.24 mL, 1.4 mmol) in DMF (0.5 mL) was added to a solution of but-2-ynoic acid (11.3 mg, 0.14 mmol) and HATU (56 mg, 0.15 mmol) in DMF (0.5 ml). The reaction solution was stirred at rt for 1 h, and then purified by prep-HPLC (Chromatographic columns: Kromasil-C18 100×21.2 mm 5 um; Mobile Phase ACN-H₂O (0.05% NH₃), Gradient: 45-55), to give (S)-1-(3-(but-2-ynamido) piperidin-1-yl)-2-fluoro-5,6,7,8,9,10-hexahydrocyclohepta[b]indole-4-carboxamide (15.5 mg) as a white solid. HRMS (ESI) calculated for C₂₃H₂₇FN₄O₂⁺ [M+H]⁺ m/z=411.22; found=412.2. ¹H NMR (400 MHz, DMSO-d₆) δ 10.61 (s, 1H), 8.46 (d, J=8.0 Hz, 1H), 7.88 (br s, 1H), 7.35 (d, J=14.4 Hz, 1H), 7.29 (br s, 1H), 3.82 (s, 1H), 3.12-3.10 (m, 3H), 2.80-2.79 (m, 5H), 1.90 (s, 3H), 1.84-1.74 (m, 3H), 1.70-1.54 (m, 7H).

### Examples 4-2 to 4-4

Compounds 4-2 through 4-4, shown in Table 4, were prepared in a manner similar to that described for Compound 4-1 above.

TABLE 4
COMPOUNDS 4-2 THROUGH 4-6

| Compound | | |
|---|---|---|
| No. | Structure | ¹H NMR |
| 4-2 | 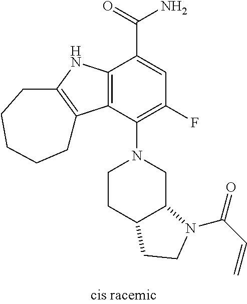 | (400 MHz, DMSO-d₆) δ 10.63-10.60 (m, 1H), 7.93 (s, 1H), 7.44-7.33(m, 2H), 6.55 (m, 1H), 6.08 (d, J = 6.0 Hz, 1H), 5.66-5.62 (m, 1H), 4.55-4.54 (m, 0.58H), 4.39 (s, 0.67H), 4.30-4.21 (m, 1H), 3.78-3.55 (m, 2H), 3.42-3.00 (m, 4H), 2.99-2.75 (m, 4H), 2.20- 2.05 (m, 2H), 1.90-1.60 (m, 8H). |
| 4-3 | 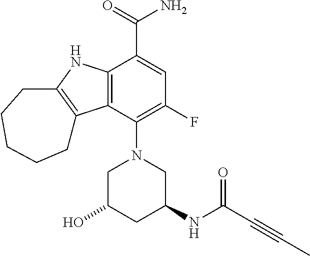 [SMILES: CC#CC(=O)N[C@H]1C[C@H](O)CN(c2c(F)cc(C(N)=O)c3[nH]c4c(c23)CCCCC4)C1] | (400 MHz, DMSO-d₆) δ 10.63-10.60 (m, 1H), 8.34 (d, J = 8.3 Hz, 0.6H), 8.15-8.13 (m, 0.4H), 7.92 (s, 1H), 7.44-7.24 (m, 2H), 4.76-4.75 (m, 0.5H), 4.55- 4.54 (m, 0.58H), 4.39 (s, 0.67H), 4.08 (s, 0.44H), 3.97-3.94 (m, 1H), 3.53-3.47 (m, 1H), 3.18-3.15 (m, 2H), 3.08-2.98 (m, 1H), 2.92-285 (m, 2H), 2.73-2.65 (m, 1H), 2.12-1.98 (m, 1H), 1.93 (s, 3H), 1.83-1.80 (m, 3H), 1.67-1.50 (s, 4H), 1.50- 1.37 (m, 1H). |
| 4-4 | 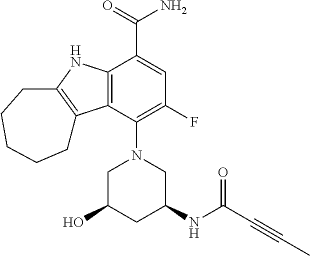 [SMILES: CC#CC(=O)N[C@H]1C[C@@H](O)CN(c2c(F)cc(C(N)=O)c3[nH]c4c(c23)CCCCC4)C1] | (400 MHz, DMSO-d₆) δ 10.67 (s, 1H), 8.54 (d, J = 7.9 Hz, 1H), 7.93 (s, 1H), 7.41-7.35 (m, 2H), 4.96 (d, J = 4.8 Hz, 1H), 3.92-3.91 (m, 1H), 3.76-3.67 (m, 1H), 3.18-3.03 (m, 4H), 2.89 (s, 2H), 2.76- 2.64 (m, 2H), 2.09-2.05 (m, 1H), 1.94 (s, 3H), 1.84- 1.77 (m, 2H), 1.73-1.50 (m, 5H) |

### Examples 5-1 & 5-2

### Preparation of 2-fluoro-1-[2-(prop-2-enoyl)-3,4-dihydro-1H-isoquinolin-5-yl]-5H,6H,7H,8H,-9H,10H-cyclohepta[b]indole-4-carboxamide (Compound 5-1 and 5-2)

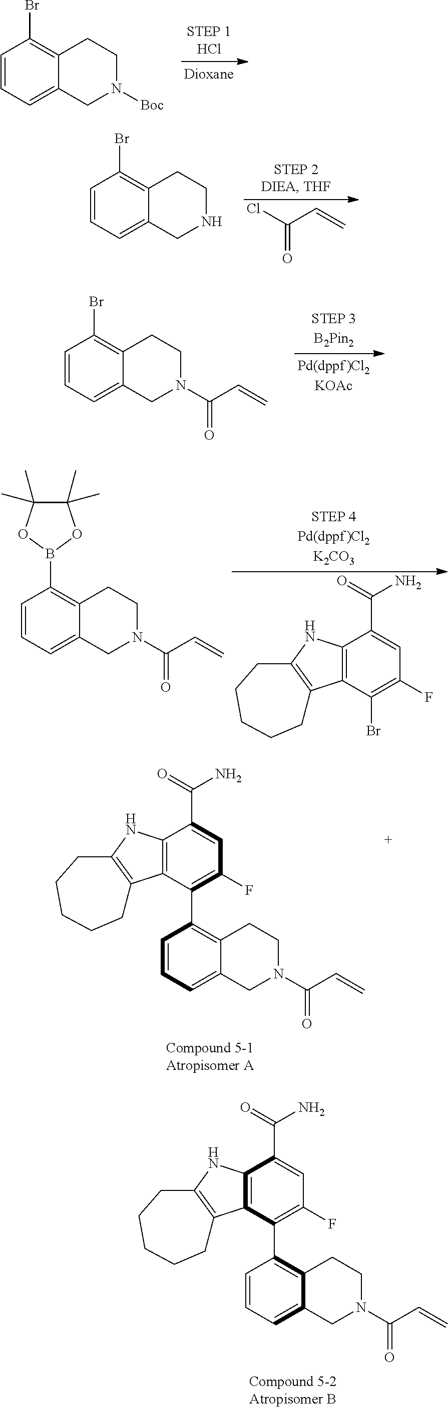

### Step 1: Preparation of 5-bromo-1,2,3,4-tetrahydroisoquinoline 2

A solution of tert-butyl 5-bromo-3,4-dihydro-1H-isoquinoline-2-carboxylate (5.0 g, 16.0 mmol) in HCl/dioxane (50 mL) was stirred at 25° C. for 1 h. The mixture was concentrated to give 5-bromo-1,2,3,4-tetrahydroisoquinoline (3.17 g, yield 89%) as a white solid, which was used directly without any purification. LCMS (ESI) calculated for C₉H₁₁BrN⁺ [M+H]⁺ m/z 212.0, found 212.1.

### Step 2: Preparation of 1-(5-bromo-3,4-dihydro-1H-isoquinolin-2-yl) prop-2-en-1-one 3

To a solution of 5-bromo-1,2,3,4-tetrahydroisoquinoline (3.10 g, 14.62 mmol) in THF (20 mL) was added dropwise prop-2-enoyl chloride (1.33 g, 14.62 mmol) and DIEA (5.67 g, 43.86 mmol) at −78° C. The reaction mixture was stirred at −78° C. for 1 h. The reaction mixture was quenched with water (100 mL) and extracted with ethyl acetate (3×50 mL). The combined organic layers were washed with brine (80 mL), dried over Na₂SO₄, filtered and concentrated. The residue was purified by silica gel (eluent MeOH in DCM=0-5%) to give 1-(5-bromo-3,4-dihydro-1H-isoquinolin-2-yl) prop-2-en-1-one (3.17 g, yield 77%) as a light-yellow oil. LCMS (ESI) calculated for C₁₂H₁₃BrNO⁺ [M+H]⁺ m/z 266.0, found 266.1.

### Step 3: Preparation of 1-[5-(4,4,5,5-tetramethyl-1,3,2-dioxaborolan-2-yl)-3,4-dihydro-1H-isoquinolin-2-yl] prop-2-en-1-one

To a solution of 1-(5-bromo-3,4-dihydro-1H-isoquinolin-2-yl)prop-2-en-1-one (3.17 g, 11.90 mmol) in dioxane (10 mL) was added 4,4,5,5-tetramethyl-2-(4,4,5,5-tetramethyl-1,3,2-dioxaborolan-2-yl)-1,3,2-dioxaborolane (4.53 g, 17.85 mmol), [1,1′-bis(diphenylphosphino)-ferrocene]dichloropalladium(II) (870 mg, 1.19 mmol) and potassium acetate (3.50 g, 35.7 mmol), then the mixture was stirred at 100° C. for 2 h under microwave. The reaction mixture was quenched with water (100 mL), extracted with ethyl acetate (3×50 mL), washed with brine (50 mL), dried over Na₂SO₄, filtrated and evaporated in vacuo. The crude product was purified by reverse flash column (ACN in H₂O=0-95%) to afford 1-[5-(4,4,5,5-tetramethyl-1,3,2-dioxaborolan-2-yl)-3,4-dihydro-1H-isoquinolin-2-yl]prop-2-en-1-one (2.01 g) as a light yellow solid. LCMS (ESI) calculated for C₁₈H₂₅BNO₃⁺[M+H]⁺ m/z 314.2, found 314.2.

### Step 4: Preparation of 2-fluoro-1-[2-(prop-2-enoyl)-3,4-dihydro-1H-isoquinolin-5-yl]-5H,6H,7H,8H,9H,10H-cyclohepta[b]indole-4-carboxamide

To a solution of 1-bromo-2-fluoro-5H,6H,7H,8H,9H,10H-cyclohepta[b]indole-4-carboxamide (279 mg, 0.86 mmol) and N-[3-(4,4,5,5-tetramethyl-1,3,2-dioxaborolan-2-yl)phenyl]prop-2-enamide (270 mg, 0.86 mmol) in 1,4-dioxane (10 mL) and water (1 mL) was added potassium carbonate (178.3 mg, 1.29 mmol) and [1,1-bis(diphenylphosphino)ferrocene]-dichloropalladium(II) (63 mg, 0.086 mmol). The mixture was stirred at 80° C. under nitrogen atmosphere under microwave for 2 h. The mixture was concentrated, purified by silica gel column (ethyl acetate in petroleum ether from 0% to 60%) to afford the crude product, and then further purified by prep-HPLC (mobile phase: acetonitrile-water (0.05% NH₃) to afford the 2-fluoro-1-[2-(prop-2-enoyl)-3,4-dihydro-1H-isoquinolin-5-yl]-5H,6H,7H,8H,9H,10H-cyclohepta[b]indole-4-carboxamide (58 mg). The atropisomers were then separated by supercritical fluid chromatography (SFC) (mobile Phase: CO₂/MeOH (0.2% NH₃.H₂O)=52/48) to afford:

(i) Compound 5-1 (peak A) at t_{R}=2.535 min (19.5 mg) as a light yellow solid; ¹H NMR (400 MHz, CD₃OD) δ 7.40 (d, J=10.4 Hz, 1H), 7.36-7.23 (m, 2H), 7.19-7.09 (m, 1H), 6.92-6.73 (m, 1H), 6.26-6.22 (m, 1H), 5.84-5.70 (m, 1H), 4.90-4.88 (m, 2H), 3.78-3.71 (m, 2H), 2.97-2.76 (m, 2H), 2.55-2.49 (m, 2H), 2.04-1.98 (m, 2H), 1.71-1.67 (m, 4H), 1.34-1.29 (m, 2H). LCMS (ESI) calculated for C₂₆H₂₆FN₃O₂⁺ [M+H]⁺+ m/z=431.20, found=432.2.

(ii) Compound 5-2 (peak B) at t_{R}=3.053 min (19.4 mg) as a light yellow solid. ¹H NMR (400 MHz, CD₃OD) δ 7.40 (d, J=10.4 Hz, 1H), 7.35-7.23 (m, 2H), 7.15-7.13 (m, 1H), 6.91-6.75 (m, 1H), 6.26-6.22 (m, 1H), 5.82-5.70 (m, 1H), 4.90-4.88 (m, 2H), 3.78-3.71 (m, 2H), 2.88-2.86 (m, 2H), 2.61-2.45 (m, 2H), 2.04-1.98 (m, 2H), 1.74-1.69 (m, 4H), 1.34-1.30 (m, 2H).

LCMS (ESI) calculated for C₂₆H₂₆FN₃O₂⁺ [M+H]+ m/z=431.20; found=432.2.

### Examples 5-3 to 5-4

Compounds 5-3 through 5-4, shown in Table 5, were prepared in a manner similar to that described in Example 5.

TABLE 5
COMPOUNDS 5-3 THROUGH 5-4

| Compound | | |
|---|---|---|
| No. | Structure | ¹H NMR |
| 5-3 | 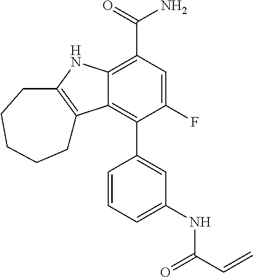 [SMILES: C=CC(=O)Nc1cccc(-c2c(F)cc(C(N)=O)c3[nH]c4c(c23)CCCCC4)c1] | (400 MHz, DMSO-d₆) δ 10.85 (s, 1H), 10.22 (s, 1H), 8.07 (s, 1H), 7.71 (d, J = 11.4 Hz, 2H), 7.46 (m, 3H), 7.05 (d, J = 7.6 Hz, 1H), 6.44 (dd, J = 16.9, 10.1 Hz, 1H), 6.25 (dd, J = 17.0, 2.0 Hz, 1H), 5.76 (dd, J = 10.2, 2.0 Hz, 1H), 2.91 (s, 2H), 2.07 (d, J = 4.6 Hz, 2H), 1.65 (d, J = 31.1 Hz, 4H), 1.36 (s, 2H). |
| 5-4 | 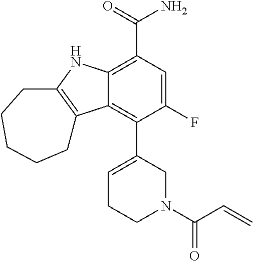 [SMILES: C=CC(=O)N1CCC=C(c2c(F)cc(C(N)=O)c3[nH]c4c(c23)CCCCC4)C1] | (400 MHz, CD₃OD) δ 7.33 (d, J = 10.6 Hz, 1H), 6.91-6.67 (m, 1H), 6.24 (dd, J = 16.8, 1.9 Hz, 1H), 5.91-5.86 (m, 1H), 5.81-5.72(m, 1H), 4.45-4.12 (m, 2H), 4.11-3.90 (m, 1H), 3.81-3.64 (m, 1H), 3.09-2.67 (m, 4H), 2.51- 2.33 (m, 2H), 1.95-1.81 (m, 2H), 1.77-1.70 (m, 2H), 1.69-1.56 (m, 2H). |

### Examples 5-5, 5-6, 5-7

### Preparation of rac-1-(1-acryloylpiperidin-3-yl)-2-fluoro-5,6,7,8,9,10-hexahydrocyclo-hepta[b]indole-4-carboxamide (Compound 5-5), (S)-1-(1-acryloylpiperidin-3-yl)-2-fluoro-5,6,7,8,9,10-hexahydrocyclohepta[b]indole-4-carboxamide (Compound 5-6) and (R)-1-(1-acryloylpiperidin-3-yl)-2-fluoro-5,6,7,8,9,10-hexahydrocyclohepta[b]indole-4-carboxamide (Compound 5-7)

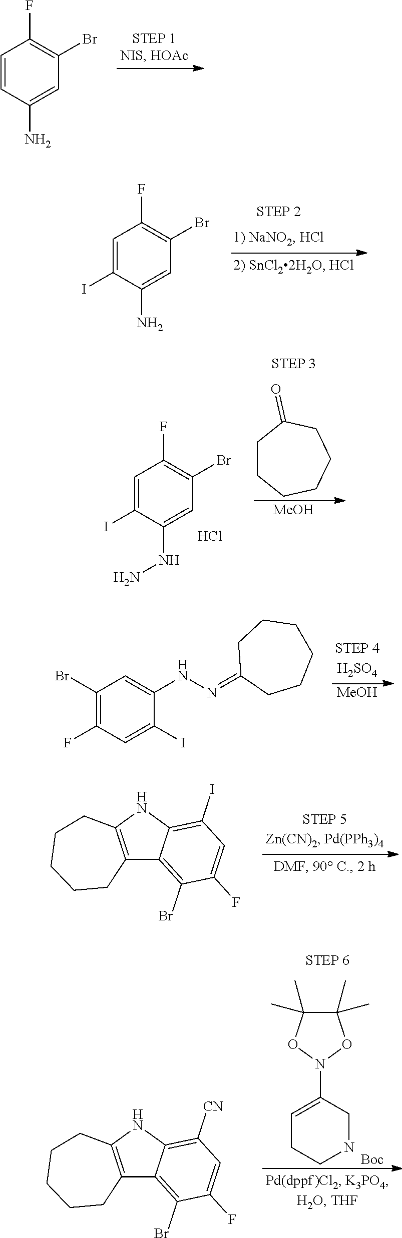

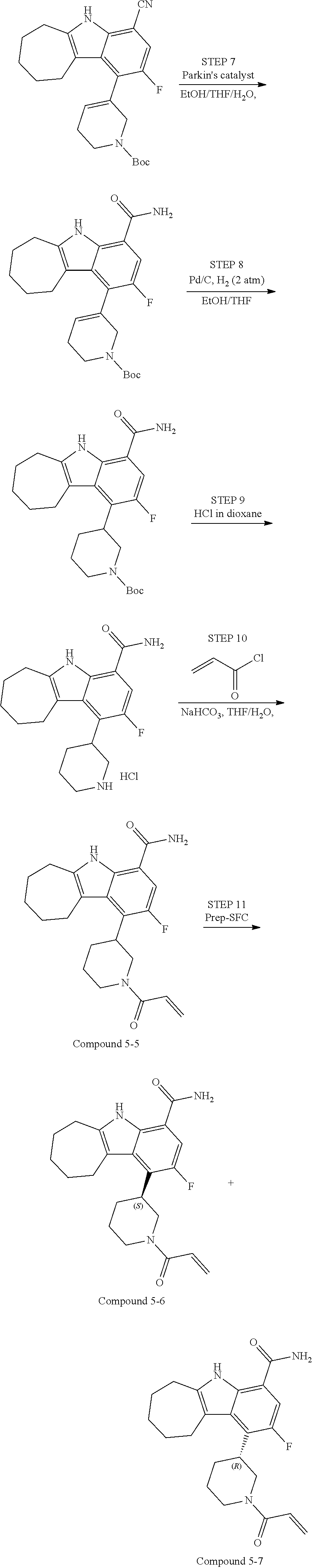

### Step 1: 5-bromo-4-fluoro-2-iodoaniline

To a solution of 3-bromo-4-fluoroaniline (100.0 g, 526.3 mmol) in acetic acid (500 mL) was added N-iodosuccinimide (124.3 g, 552.5 mmol) in portions at 25° C. The reaction mixture was stirred for 2 hours at 25° C. The mixture was concentrated under vacuum. The residue was diluted with saturated aqueous sodium carbonate (500 mL) and extracted with ethyl acetate (500 mL×3). The combined organic layers were washed with water (500 mL) and brine (500 mL), dried over anhydrous sodium sulfate and concentrated under vacuum. The residue was triturated with mixed solvents of ethyl acetate and petroleum ether (300 mL, 1:4, v/v) and filtered. The solid was washed with mixed solvents of ethyl acetate and petroleum ether (50 mL×2, 1:4, v/v) and dried under reduced pressure to give 5-bromo-4-fluoro-2-iodoaniline (88.6 g, 53%) as a light blue solid. ¹H NMR (300 MHz, DMSO-d₆) δ 7.55 (d, J=8.1 Hz, 1H), 6.98 (d, J=6.3 Hz, 1H), 5.27 (brs, 2H).

### Step 2: (5-bromo-4-fluoro-2-iodophenyl)hydrazine hydrochloride

To a stirred suspension of 5-bromo-4-fluoro-2-iodoaniline (88.6 g, 280.5 mmol) in concentrated hydrochloric acid (443 mL) was added dropwise a solution of sodium nitrite (23.22 g, 337.0 mmol) in water (90 mL) at 0° C. After stirring for 1 hour at 0° C., the resulting mixture was added dropwise to a solution of stannous chloride dihydrate (126.61 g, 561.1 mmol) in concentrated hydrochloric acid (295 mL) at 0° C. and stirred for 1 hour at this temperature. The precipitate was collected by filtration, washed with concentrated hydrochloric acid (150 mL×5) and ethyl acetate (300 mL), dried under reduced pressure to give (5-bromo-4-fluoro-2-iodophenyl)hydrazine hydrochloride (100.3 g, crude) as a light yellow solid. ¹H NMR (400 MHz, DMSO-d₆) δ 10.23 (brs, 3H), 7.89 (d, J=8.0 Hz, 1H), 7.82 (brs, 1H), 7.31-7.22 (m, 1H).

### Step 3: 1-(5-bromo-4-fluoro-2-iodophenyl)-2-cycloheptylidenehydrazine

To a solution of (5-bromo-4-fluoro-2-iodophenyl)hydrazine hydrochloride (80.0 g, 217.6 mmol) in methanol (400 mL) was added cycloheptanone (24.40 g, 217.6 mmol) at 20° C. The reaction mixture was stirred for 1 hour at 20° C. The precipitate was collected by filtration and dried under reduced pressure to give 1-(5-bromo-4-fluoro-2-iodophenyl)-2-cycloheptylidenehydrazine (72.0 g, 78%) as an off-white solid. ¹H NMR (400 MHz, DMSO-d₆) δ 7.77 (d, J=8.0 Hz, 1H), 7.44 (d, J=6.8 Hz, 1H), 7.39 (brs, 1H), 2.50-2.44 (m, 4H), 1.80-1.67 (m, 2H), 1.64-1.48 (m, 6H).

### Step 4: 1-bromo-2-fluoro-4-iodo-5,6,7,8,9,10-hexahydrocyclohepta[b]indole

A mixture of 1-(5-bromo-4-fluoro-2-iodophenyl)-2-cycloheptylidenehydrazine (72.0 g, 169.4 mmol) and concentrated sulfuric acid (18 mL) in methanol (360 mL) was stirred for 16 hours at 80° C. The methanol was removed under reduced pressure. The residue was basified with saturated aqueous sodium carbonate until pH=10 and extracted with ethyl acetate (600 mL×3). The combined organic layers were washed with water (500 mL×2) and brine (500 mL), dried over anhydrous sodium sulfate and filtered. The filtrate was concentrated under vacuum to give 1-bromo-2-fluoro-4-iodo-5,6,7,8,9,10-hexahydrocyclohepta[b]indole (43.0 g, 80% purity, 50%) as a brown solid. ¹H NMR (300 MHz, DMSO-d₆) δ 10.95 (s, 1H), 7.37 (d, J=8.7 Hz, 1H), 3.23-3.15 (m, 2H), 2.94-2.85 (m, 2H), 1.89-1.76 (m, 2H), 1.72-1.58 (m, 4H).

### Step 5: 1-bromo-2-fluoro-5,6,7,8,9,10-hexahydrocyclohepta[b]indole-4-carbonitrile

A mixture of 1-bromo-2-fluoro-4-iodo-5,6,7,8,9,10-hexahydrocyclohepta[b]indole (43.0 g, 80% purity, 84.3 mmol), zinc cyanide (4.95 g, 42.2 mmol) and tetrakis(triphenylphosphine)palladium (9.74 g, 8.4 mmol) in N,N-dimethylformamide (215 mL) was degassed and backfilled with nitrogen for three times. The reaction mixture was stirred under nitrogen at 90° C. for 2 hours. The cooled reaction mixture was diluted with water (1 L) and extracted with ethyl acetate (800 mL×3). The combined organic layers were washed with water (500 mL×3) and brine (800 mL), dried over anhydrous sodium sulfate, filtered and concentrated under vacuum. The residue was triturated with acetonitrile (100 mL) and filtered. The solid was washed with acetonitrile (30 mL×2) and dried under reduced pressure to give 1-bromo-2-fluoro-5,6,7,8,9,10-hexahydrocyclohepta[b]indole-4-carbonitrile (25.5 g, 94%) as a light yellow solid. ESI-MS [M−H]⁻ calculated for (C₁₄H₁₂BrFN₂) 305.02, 307.02, found: 304.95, 306.95. ¹H NMR (300 MHz, DMSO-d₆) δ 11.99 (s, 1H), 7.58 (d, J=9.0 Hz, 1H), 3.24-3.17 (m, 2H), 2.91-2.85 (m, 2H), 1.87-1.78 (m, 2H), 1.70-1.61 (m, 4H).

### Step 6: Tert-butyl 5-(4-cyano-2-fluoro-5,6,7,8,9,10-hexahydrocyclohepta[b]indol-1-yl)-3,6-dihydropyridine-1(2H)-carboxylate

A mixture of 1-bromo-2-fluoro-5,6,7,8,9,10-hexahydrocyclohepta[b]indole-4-carbonitrile (25.0 g, 81.4 mmol), tert-butyl 5-(4,4,5,5-tetramethyl-1,3,2-dioxaborolan-2-yl)-3,6-dihydropyridine-1(2H)-carboxylate (30.2 g, 97.7 mmol), [1,1′-bis(diphenylphosphino)ferrocene]dichloro-palladium(II) (5.96 g, 8.1 mmol) and potassium phosphate (51.8 g, 244.2 mmol) in tetrahydrofuran (125 mL) and water (31 mL) was degassed and backfilled with nitrogen for three times and stirred for 2 hours at 60° C. under nitrogen atmosphere. The cooled mixture was diluted with water (600 mL) and extracted with ethyl acetate (500 mL×3). The combined organic layers was washed with water (500 mL×2) and brine (500 mL), dried over anhydrous sodium sulfate, filtered and concentrated under vacuum to give tert-butyl 5-(4-cyano-2-fluoro-5,6,7,8,9,10-hexahydrocyclohepta[b]indol-1-yl)-3,6-dihydropyridine-1(2H)-carboxylate (45 g, crude) as a brown solid, which was used directly in next step without purification. ESI-MS [M+H-tBu]⁺ calculated for (C₂₄H₂₈FN₃O₂) 354.22, found: 354.05.

### Step 7: Tert-butyl 5-(4-carbamoyl-2-fluoro-5,6,7,8,9,10-hexahydrocyclohepta[b]indol-1-yl)-3,6-dihydropyridine-1(2H)-carboxylate

To a mixture of 5-(4-cyano-2-fluoro-5,6,7,8,9,10-hexahydrocyclohepta[b]indol-1-yl)-3,6-dihydro-pyridine-1(2H)-carboxylate (45 g, crude) in ethanol (100 mL), tetrahydrofuran (100 mL) and water (100 mL) was added Parkin's catalyst (2.0 g, 4.68 mmol). The reaction mixture was stirred for 16 hours at 90° C. The cooled mixture was diluted with water (500 mL) and extracted with ethyl acetate (500 mL×3). The combined organic layers were washed with water (500 mL×2) and brine (500 mL), dried over anhydrous sodium sulfate, filtered and concentrated under vacuum. The residue was purified by column chromatography on silica gel eluting with ethyl acetate in petroleum ether (0 to 60%) to give tert-butyl 5-(4-carbamoyl-2-fluoro-5,6,7,8,9,10-hexahydrocyclohepta[b]indol-1-yl)-3,6-dihydropyridine-1(2H)-carboxylate (20.0 g, 57% over two steps) as a light yellow solid. ESI-MS [M+H]⁺ calculated for (C₂₄H₃₀FN₃O₃) 428.23, found: 428.15. ¹H NMR (400 MHz, DMSO-d₆) δ 10.77 (s, 1H), 8.02 (s, 1H), 7.46-7.38 (m, 2H), 5.79 (s, 1H), 4.10-3.97 (m, 1H), 3.95-3.83 (m, 1H), 3.80-3.57 (m, 1H), 3.51-3.23 (m, 1H), 2.99-2.85 (m, 2H), 2.82-2.69 (m, 2H), 2.30-2.21 (m, 2H), 1.86-1.72 (m, 2H), 1.70-1.50 (m, 4H), 1.41 (s, 9H).

### Step 8: Tert-butyl 3-(4-carbamoyl-2-fluoro-5,6,7,8,9,10-hexahydrocyclohepta[b]indol-1-yl)piperidine-1-carboxylate

To a solution of tert-butyl 5-(4-carbamoyl-2-fluoro-5,6,7,8,9,10-hexahydrocyclohepta[b]indol-1-yl)-3,6-dihydropyridine-1(2H)-carboxylate (20 g, 46.8 mmol) in ethanol (300 mL) and tetrahydrofuran (300 mL) was added 10% Pd/C (15.0 g) under nitrogen atmosphere. The reaction mixture was degassed and backfilled with hydrogen for three times and stirred for 4 days at 50° C. under hydrogen (2 atm). The cooled mixture was filtered. The filtrate was concentrated under vacuum. The residue was recrystallized with tetrahydrofuran (100 mL) and petroleum ether (100 mL) to give tert-butyl 3-(4-carbamoyl-2-fluoro-5,6,7,8,9,10-hexahydrocyclohepta[b]indol-1-yl)piperidine-1-carboxylate (12.1 g, 60%) as an off-white solid. ESI-MS [M+H]⁺ calculated for (C₂₄H₃₂FN₃O₃) 430.24, found: 430.25. ¹H NMR (400 MHz, DMSO-d₆) δ 10.75 (s, 1H), 8.00 (s, 1H), 7.46-7.35 (m, 2H), 4.17-3.86 (m, 2H), 3.55-3.43 (m, 1H), 3.31-3.10 (m, 1H), 3.08-2.63 (m, 5H), 2.14-1.96 (m, 1H), 1.93-1.60 (m, 9H), 1.39 (s, 9H).

### Step 9: 2-fluoro-1-(piperidin-3-yl)-5,6,7,8,9,10-hexahydrocyclohepta[b]indole-4-carboxamide hydrochloride

Tert-butyl 3-(4-carbamoyl-2-fluoro-5,6,7,8,9,10-hexahydrocyclohepta[b]indol-1-yl)piperidine-1-carboxylate (12.1 g, 28.2 mmol) was dissolved in hydrogen chloride (150 mL, 4 M in 1,4-dioxane) and the solution was stirred for 2 hours at 25° C. The mixture was concentrated under vacuum to give 2-fluoro-1-(piperidin-3-yl)-5,6,7,8,9,10-hexahydrocyclohepta[b]indole-4-carboxamide hydrochloride (13.4 g, crude) as a yellow solid. ESI-MS [M+H]⁺ calculated for (C₁₉H₂₄FN₃O) 330.19, found: 330.10.

### Step 10: 1-(1-acryloylpiperidin-3-yl)-2-fluoro-5,6,7,8,9,10-hexahydrocyclohepta[b]indole-4-carboxamide

To a mixture of 2-fluoro-1-(piperidin-3-yl)-5,6,7,8,9,10-hexahydrocyclohepta[b]indole-4-carboxamide hydrochloride (13.4 g, crude) and sodium bicarbonate (23.7 g, 282.0 mmol) in tetrahydrofuran (300 mL) and water (150 mL) was added acryloyl chloride (2.81 g, 31.0 mmol) at 0° C. After stirring for 1 hour at 0° C., the mixture was diluted with water (500 mL) and extracted with ethyl acetate (400 mL×3). The combined organic layers were washed with water (500 mL×2) and brine (500 mL), dried over anhydrous sodium sulfate, filtered and concentrated under vacuum. The residue was recrystallized with tetrahydrofuran (290 mL), methanol (48 mL) and petroleum ether (330 mL) to give 1-(1-acryloylpiperidin-3-yl)-2-fluoro-5,6,7,8,9,10-hexahydrocyclohepta[b]indole-4-carboxamide (6.0 g, 56% over two steps) as a white solid. ESI-MS [M+H]⁺ calculated for (C₂₂H₂₆FN₃O₂) 384.20, found: 384.15.

### Step 11: (S)-1-(1-acryloylpiperidin-3-yl)-2-fluoro-5,6,7,8,9,10-hexahydrocyclohepta[b]indole-4-carboxamide and (R)-1-(1-acryloylpiperidin-3-yl)-2-fluoro-5,6,7,8,9,10-hexahydrocyclohepta[b]-indole-4-carboxamide

1-(1-acryloylpiperidin-3-yl)-2-fluoro-5,6,7,8,9,10-hexahydrocyclohepta[b]indole-4-carboxamide (6.0 g) was separated by Prep-SFC with the following conditions: Column: (R,R)-Whelk-01, 2.12×25 cm, 5 um; Mobile Phase A: CO₂, Mobile Phase B: IPA/DCM=5:1; Flow rate: 200 mL/min; Gradient: 50% B; 220 nm; Injection Volume: 19 mL; Number Of Runs: 29; RT1: 4.97 min to afford assumed (S)-1-(1-acryloylpiperidin-3-yl)-2-fluoro-5,6,7,8,9,10-hexahydrocyclohepta[b]indole-4-carboxamide (2.55 g, 43%) as an off-white solid and RT2: 8.2 min to afford assumed (R)-1-(1-acryloylpiperidin-3-yl)-2-fluoro-5,6,7,8,9,10-hexahydrocyclohepta[b]indole-4-carboxamide (2.63 g, 44%) as an off-white solid.

Compound 5-6

ESI-MS [M+H]⁺ calculated for (C₂₂H₂₆FN₃O₂) 384.20, found: 384.20. ¹H NMR (300 MHz, DMSO-d₆) δ 10.75 (s, 1H), 8.00 (s, 1H), 7.49-7.31 (m, 2H), 6.93-6.72 (m, 1H), 6.18-6.02 (m, 1H), 5.73-5.56 (m, 1H), 4.67-4.42 (m, 1H), 4.27-4.05 (m, 1H), 3.63-3.41 (m, 1.5H), 3.19-3.02 (m, 1H), 3.00-2.79 (m, 4H), 2.70-2.62 (m, 0.5H), 2.21-2.02 (m, 1H), 2.01-1.87 (m, 1H), 1.86-1.61 (m, 7H), 1.57-1.37 (m, 1H).

Compound 5-7

ESI-MS [M+H]⁺ calculated for (C₂₂H₂₆FN₃O₂) 384.20, found: 384.20. ¹H NMR (300 MHz, DMSO-d₆) δ 10.74 (s, 1H), 8.00 (s, 1H), 7.47-7.33 (m, 2H), 6.93-6.72 (m, 1H), 6.18-6.03 (m, 1H), 5.75-5.55 (m, 1H), 4.64-4.44 (m, 1H), 4.22-4.04 (m, 1H), 3.64-3.39 (m, 1.5H), 3.19-3.01 (m, 1H), 3.00-2.79 (m, 4H), 2.70-2.62 (m, 0.5H), 2.23-2.02 (m, 1H), 2.01-1.87 (m, 1H), 1.86-1.59 (m, 7H), 1.58-1.37 (m, 1H).

### Example 5-8 & 5-9

### Preparation of: (S)-1-(5-(but-2-ynamido)cyclohex-1-en-1-yl)-2-fluoro-5,6,7,8,9,10-hexahydrocyclohepta [b]indole-4-carboxamide (Compound 5-8) and (S)-1-(3-(but-2-ynamido)cyclohex-1-en-1-yl)-2-fluoro-5,6,7,8,9,10-hexahydrocyclohepta [b]indole-4-carboxamide (Compound (5-9)

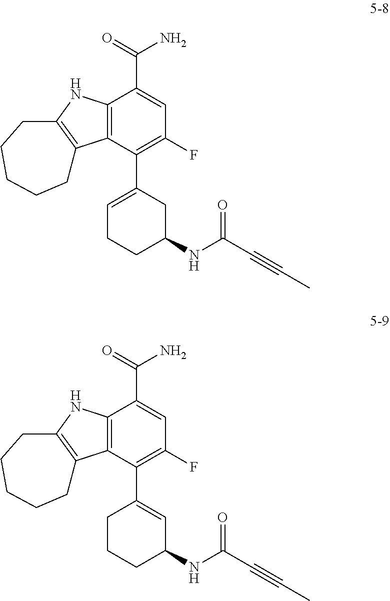 [SMILES: CC#CC(=O)N[C@H]1CCC=C(c2c(F)cc(C(N)=O)c3[nH]c4c(c23)CCCCC4)C1 ; CC#CC(=O)N[C@@H]1C=C(c2c(F)cc(C(N)=O)c3[nH]c4c(c23)CCCCC4)CCC1]

### Step 1: Preparation of tert-butyl N-[3-(4-cyano-2-fluoro-5,6,7,8,9,10-hexahydro-cyclohepta[b]indol-1-yl)cyclohex-3-en-1-yl]carbamate

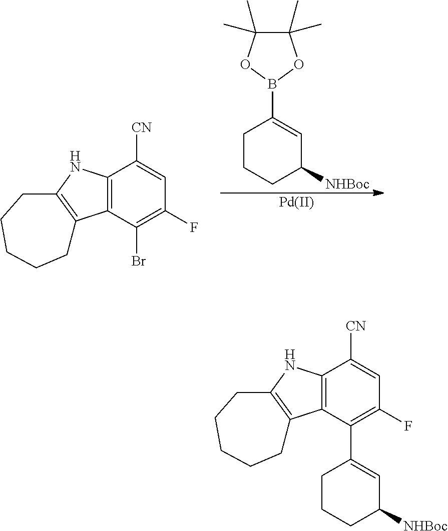

To a mixture of 1-bromo-2-fluoro-5,6,7,8,9,10-hexahydrocyclohepta[b]indole-4-carbonitrile (300 mg, 976 μmol), tert-butyl N-[3-(4,4,5,5-tetramethyl-1,3,2-dioxaborolan-2-yl)cyclohex-3-en-1-yl]carbamate (473.55 mg, 1.47 mmol) and K₃PO₄ (621.95 mg, 2.93 mmol) in 1,4-dioxane (6 mL) and water (2 mL) was added 1,1-bis(diphenylphosphino)-ferrocenedichloropalladium(II) (71.46 mg, 97.67 μmol) under nitrogen atmosphere. The reaction mixture was evacuated and flushed three times with nitrogen atmosphere and stirred at 90° C. for 2 h. The reaction mixture was quenched with water (20 mL), extracted with ethyl acetate (20 mL×3). The combined organic extracts were washed with brine (30 mL), dried over anhydrous sodium sulfate and filtered. The filtrate was concentrated under reduced pressure to afford tert-butyl N-[3-(4-cyano-2-fluoro-5,6,7,8,9,10-hexahydro-cyclohepta[b]indol-1-yl)cyclohex-3-en-1-yl]carbamate (600 mg, crude) as a brown solid.

### Step 2: Preparation of (S)-1-(3-aminocyclohex-1-en-1-yl)-2-fluoro-5,6,7,8,9,10-hexahydrocyclo hepta[b]indole-4-carboxamide hydrochloride

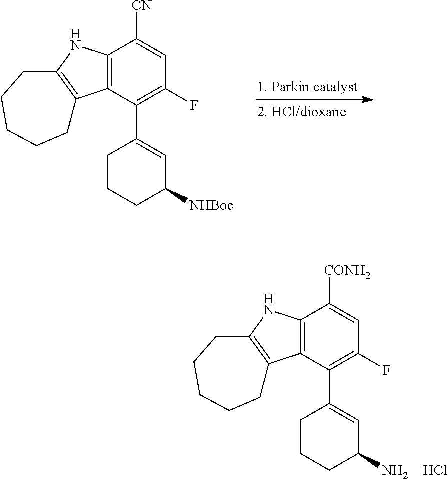 [SMILES: CC(C)(C)OC(=O)N[C@@H]1C=C(c2c(F)cc(C#N)c3[nH]c4c(c23)CCCCC4)CCC1 ; NC(=O)c1cc(F)c(C2=C[C@@H](N)CCC2)c2c3c([nH]c12)CCCCC3 ; Cl]

To a mixture of tert-butyl N-[(1S)-3-(4-cyano-2-fluoro-5,6,7,8,9,10-hexahydrocyclohepta[b]indol-1-yl)cyclohex-3-en-1-yl]carbamate (600 mg, 1.42 mmol) in EtOH (6 mL) and water (2 mL) was added Parkin's catalyst (30.26 mg, 70.83 μmol). The reaction mixture was stirred at 90° C. for 12 h. The reaction mixture was cooled and quenched with water (50 ml), extracted with ethyl acetate (30 ml×3). The combined organic extracts were washed with brine (50 ml), dried over anhydrous sodium sulfate, filtered and concentrated under vacuum. The residue was purified by column chromatography onto silica gel, eluting with ethyl acetate in petroleum ether (0-60%) to afford tert-butyl N-[(1S)-3-(4-carbamoyl-2-fluoro-5,6,7,8,9,10-hexahydrocyclohepta[b]-indol-1-yl)cyclohex-3-en-1-yl]carbamate (480 mg) which was dissolved in 4M HCl (4.30 mL) was stirred at 20° C. for 2 h. The reaction solvent was concentrated under reduced pressure to afford 1-[(5S)-5-aminocyclohexen-1-yl]-2-fluoro-5,6,7,8,9,10-hexahydrocyclohepta[b]indole-4-carboxamide HCl salt (480 mg) as a yellow solid.

### Step 3: Preparation of 1-[(5S)-5-(but-2-ynoylamino)cyclohexen-1-yl]-2-fluoro-5,6,7,8,9,10-hexahydrocyclo-hepta[b]indole-4-carboxamide and 1-[(3S)-3-(but-2-ynoylamino)cyclohexen-1-yl]-2-fluoro-5,6,7,8,9,10-hexahydrocyclohepta[b]indole-4-carboxamide

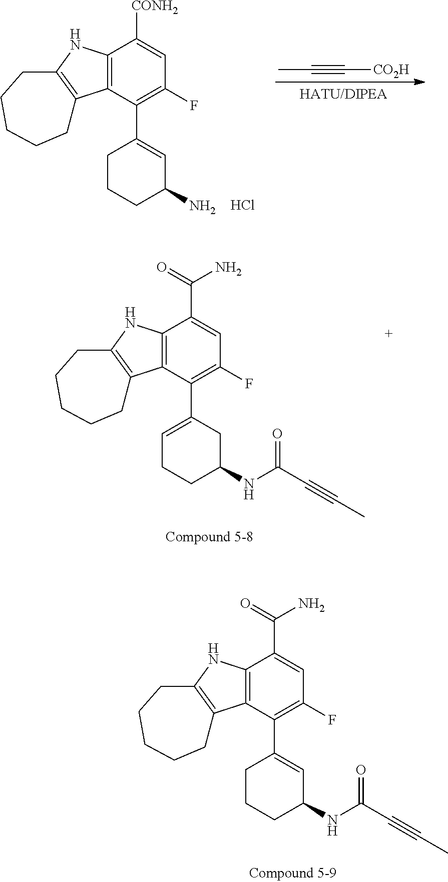

To a solution of 1-[(5S)-5-aminocyclohexen-1-yl]-2-fluoro-5,6,7,8,9,10-hexahydrocyclohepta[b]-indole-4-carboxamide (200 mg, 585 μmol) and but-2-ynoic acid (59.10 mg, 702.94 μmol) in DMF (5 mL) was added HATU (311.83 mg, 820 μmol) then DIPEA (227.12 mg, 1.76 mmol, 306.09 μL). The reaction was stirred at 20° C. for 2 h. then quenched with water (15 mL), extracted with EtOAc (20 mL×3).

The organic extracts were washed with brine (40 ml), dried over anhydrous sodium sulfate, filtered and concentrated under vacuum. The residue was purified by prep-achiral-SFC with the flowing condition: Column: GreenSep Basic, 30*150 mm 5 um; Mobile Phase A: CO₂, Mobile Phase B: MeOH (0.1% 2M NH₃-MeOH); Flow rate: 60 mL/min; Gradient: 30% B; 254 nm.

Compound 5-8

RT1: (5.10 min) 1-[(5S)-5-(but-2-ynoylamino)cyclohexen-1-yl]-2-fluoro-5,6,7,8,9,10-hexahydrocyclo-hepta[b]indole-4-carboxamide (48.6 mg) as a light-yellow solid. ¹H NMR (400 MHz, DMSO-d₆) δ 10.71 (d, J=5.2 Hz, 1H), 8.56 (dd, J=13.6, 7.6 Hz, 1H), 7.99 (s, 1H), 7.43-7.35 (m, 2H), 5.61 (d, J=10.0 Hz, 1H), 4.03-3.93 (m, 1H), 2.95-2.64 (m, 4H), 2.38-2.17 (m, 4H), 1.94 (s, 3H), 1.90-1.75 (m, 3H), 1.67-1.47 (m, 5H). ESI-MS [M+H]⁺ calculated for (C₂₄H₂₆FN₃O₂) 408.20 found: 408.20.

Compound 5-9

RT2: (5.60 min) 1-[(3S)-3-(but-2-ynoylamino)cyclohexen-1-yl]-2-fluoro-5,6,7,8,9,10-hexahydrocyclohepta[b]indole-4-carboxamide (6.6 mg) as a light-yellow solid. ¹H NMR (400 MHz, DMSO-d₆) δ 10.71 (d, J=3.2 Hz, 1H), 8.72 (dd, J=10.2, 3.6 Hz, 1H), 7.99 (s, 1H), 7.42-7.37 (m, 2H), 5.46 (d, J=17.2 Hz, 1H), 4.49-4.38 (m, 1H), 2.96-2.71 (m, 4H), 2.30-2.02 (m, 2H), 1.96-1.51 (m, 13H). ESI-MS [M+H]⁺ calculated for (C₂₄H₂₆FN₃O₂) 408.20 found: 408.20.

### Example 6-1

### Preparation of 5-bromo-1,2,4,9-tetrahydrospiro[carbazole-3,2′-[1,3]dioxolane]-8-carboxylic acid (Compound 6-1)

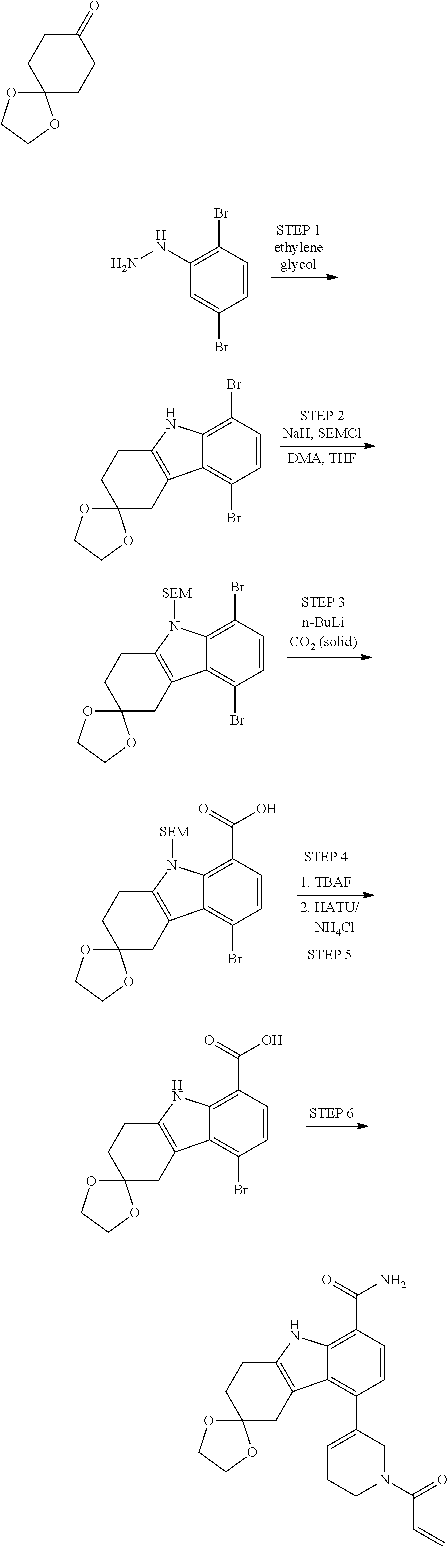

### Step 1: 5, 8-dibromospiro [1,2,4,9-tetrahydrocarbazole-3,2′-1,3-dioxolane]

A solution of (2,5-dibromophenyl)hydrazine (4.00 g, 15.0 mmol, 1.00 eq) and 1,4-dioxaspiro[4.5]decan-8-one (2.47 g, 15.7 mmol, 1.05 eq) in ethylene glycol (34.0 mL) were stirred at 140-190° C. under N₂ for 48 h. The mixture was cooled and poured into water (100 mL). The solids were isolated by filtration and the residue purified by column chromatography (SiO₂, petroleum ether/ethyl acetate=1/0 to 0/1), to provide 5,8-dibromospiro [1,2,4,9-tetrahydrocarbazole-3,2′-1,3-dioxolane] (1.90 g, 4.91 mmol) as a white solid.

### Step 2: 2-[(5′,8′-dibromospiro [1,3-dioxolane-2,3′-2,4-dihydro-1H-carbazole]-9′-yl) methoxy]ethyltrimethyl silane

NaH (215 mg, 5.40 mmol, 60% purity, 1.10 eq) was added portion-wise to a solution of 5,8-dibromospiro[1,2,4,9-tetrahydrocarbazole-3,2′-1,3-dioxolane] (1.90 g, 4.91 mmol, 1.00 eq) and DMA (6 mL) in THF (9 mL) at 25° C., and the mixture stirred for 10 mins. 2-(trimethylsilyl)-ethoxymethyl chloride (SEM-Cl, 900 mg, 5.40 mmol, 955 μL, 1.10 eq) was then added dropwise over 5 mins and the mixture stirred at 25° C. for 16 h. The mixture was then slowly poured into saturated NH₄Cl solution (50 mL) and extracted with MTBE (2×30 mL). The combined organic extracts were evaporated to provide an oil that was purified by column chromatography (SiO₂, petroleum ether/ethyl acetate=1/0 to 0/1). 2-[(5′,8′-dibromospiro [1,3-dioxolane-2,3′-2,4-dihydro-1H-carbazole]-9′-yl) methoxy]ethyl-trimethyl-silane (2.25 g, 4.35 mmol, 88.6% yield) was obtained as a yellow oil.

### Step 3: 4′-bromo-9′-(2-trimethylsilylethoxymethyl)spiro[1,3-dioxolane-2,6′-7,8-dihydro-5H-carbazole]-1′-carboxylic acid

n-BuLi (2.50 M, 1.83 mL, 1.05 eq) was added dropwise over 5 mins to a solution of 2-[(5′,8′-dibromospiro[1,3-dioxolane-2,3′-2,4-dihydro-1H-carbazole]-9′-yl) methoxy]ethyl-trimethyl-silane (2.25 g, 4.35 mmol, 1.00 eq) in THF (23 mL) under N₂ at −60° C., and the mixture stirred at −60° C. for 15 mins. Solid carbon dioxide (1.91 g, 43.4 mmol, 10.0 eq) was added in one portion and the mixture stirred at −60° C.˜20° C. for 3 h. Maintaining the temperature at 0° C., the pH was adjusted to pH=5 by addition of HCl (1N). Water (10 mL) was added and the organic phase removed. The aqueous phase was extracted with ethyl acetate (20 mL) and the combined organic extracts concentrated. The resulting residue was purified by prep-HPLC (column: Agela DuraShell C18 250*25 mm*10 um; mobile phase: [water (10 mM NH₄HCO₃)-ACN]; B %: 15%-40%, 20 min) to provide 4′-bromo-9′-(2-trimethylsilylethoxymethyl)spiro[1,3-dioxolane-2,6′-7,8-dihydro-5H-carbazole]-1′-carboxylic acid (1.50 g, 3.11 mmol, 71.4% yield) as a white solid. ¹H NMR (400 MHz, DMSO-d₆) δ 7.43-7.31 (m, 2H), 5.78 (s, 2H), 4.09 (s, 4H), 3.33 (br t, J=3.7 Hz, 4H), 3.01 (br t, J=6.0 Hz, 2H), 2.08 (br t, J=6.3 Hz, 2H), 0.81 (t, J=8.0 Hz, 2H), 0.00 (s, 9H)

### Step 4: 4′-bromospiro[1,3-dioxolane-2,6′-5,7,8,9-tetrahydrocarbazole]-1′-carboxylic acid

TBAF (1 M, 11.4 mL, 5.00 eq) was added to a solution of 4′-bromo-9′-(2-trimethylsilylethoxymethyl)spiro[1,3-dioxolane-2,6′-7,8-dihydro-5H-carbazole]-1′-carboxylic acid (1.10 g, 2.28 mmol, 1.00 eq) in THF (100 mL) and the mixture stirred at 80° C. for 4 days. The mixture was concentrated, water added (30 mL), solids removed by filtration, acetonitrile (5 mL) added and the mixture stirred at 25° C. for 1 hour. This suspension was filtered and the isolated solids washed with DCM and dried to provide 4′-bromospiro[1,3-dioxolane-2,6′-5,7,8,9-tetrahydrocarbazole]-1′-carboxylic acid (400 mg, 1.14 mmol, 49.8% yield, 100% purity) as a white solid. ¹H NMR (400 MHz, DMSO-d₆) δ 13.10 (br s, 1H), 11.04 (s, 1H), 7.49 (d, J=8.2 Hz, 1H), 7.19 (d, J=7.9 Hz, 1H), 4.02-3.88 (m, 4H), 3.15 (s, 2H), 2.86 (br t, J=6.3 Hz, 2H), 1.90 (t, J=6.5 Hz, 2H).

### Step 5: 4′-bromospiro[1,3-dioxolane-2,6′-5,7,8,9-tetrahydrocarbazole]-1′-carboxamide

To a mixture of 4′-bromospiro[1,3-dioxolane-2,6′-5,7,8,9-tetrahydrocarbazole]-1′-carboxylic acid (0.60 mmol), ammonium chloride (137 mg, 2.56 mmol) in DMA (2 mL) was added HATU (269.38 mg, 708.46 μmol) and the mixture was allowed to continue stirring at RT for 30 min. The mixture was diluted with EtOAc and water, the organic layer was separated, and the aqueous layer was extracted with EtOAc. The combined organic layer was washed with brine, dried over Na₂SO₄, filtered and concentrated. The residue obtained was purified by silica gel chromatography using a gradient of 5%-60% EtOAc in heptane to give 9-bromo-1,3,4,5-tetrahydrothiopyrano[4,3-b]indole-6-carboxamide as an off-white solid which was used in the next step without further purification.

### Step 6: 4′-(1-prop-2-enoyl-3,6-dihydro-2H-pyridin-5-yl)spiro[1,3-dioxolane-2,6′-5,7,8,9-tetrahydrocarbazole]-1′-carboxamide

To a solution of 4′-bromospiro[1,3-dioxolane-2,6′-5,7,8,9-tetrahydrocarbazole]-1′-carboxamide (70 mg, 199.32 μmol) in THF (1 mL), Methanol (0.5 mL) and water (0.5 mL),1-[5-(4,4,5,5-tetramethyl-1,3,2-dioxaborolan-2-yl)-3,6-dihydro-2H-pyridin-1-yl]prop-2-en-1-one (62.9 mg, 0.24 mmol), PdCl₂(dppf) (66.2 mg, 0.091 mmol) and Na₂CO₃ (144 mg, 1.358 mmol) were added. The reaction mixture was heated in the microwave at 70° C. for 1 h. The mixture was cooled to rt and diluted with EtOAc and water. The organic layer was separated and the aq layer was extracted with EtOAc twice more. The combined organic layer was washed with brine, dried over Na₂SO₄, filtered and concentrated under reduced pressure. The residue obtained was purified by reverse phase chromatography using a gradient of 10%-95% MeCN in water w/0.10% Formic Acid. The like fractions were combined and freeze dried on the lyophilizer to give as a 4′-(1-prop-2-enoyl-3,6-dihydro-2H-pyridin-5-yl)spiro[1,3-dioxolane-2,6′-5,7,8,9-tetrahydro-carbazole]-1′-carboxamide (34.7 mg) as an off-white solid. ¹H NMR (DMSO-d₆) δ: 10.85 (s, 1H), 7.98 (br s, 1H), 7.55 (d, J=7.7 Hz, 1H), 7.30 (br s, 1H), 6.71-7.00 (m, 2H), 6.07-6.21 (m, 1H), 5.60-5.78 (m, 2H), 4.27 (br d, J=12.0 Hz, 2H), 3.90 (s, 4H), 3.66-3.79 (m, 2H), 2.87 (br t, J=1.0 Hz, 2H), 2.73 (br s, 2H), 2.30 (br d, J=15.7 Hz, 2H), 1.89 (br t, J=6.3 Hz, 2H).

### Examples 6-2 & 6-3

### Preparation of: (S)-5-(1-acryloylpiperidin-3-yl)-6-fluoro-1,2,4,9-tetrahydrospiro[carbazole-3,1′-cyclopropane]-8-carboxamide (Compound 6-2); and (R)-5-(1-acryloylpiperidin-3-yl)-6-fluoro-1,2,4,9-tetrahydrospiro[carbazole-3,1′-cyclopropane]-8-carboxamide (Compound 6-3)

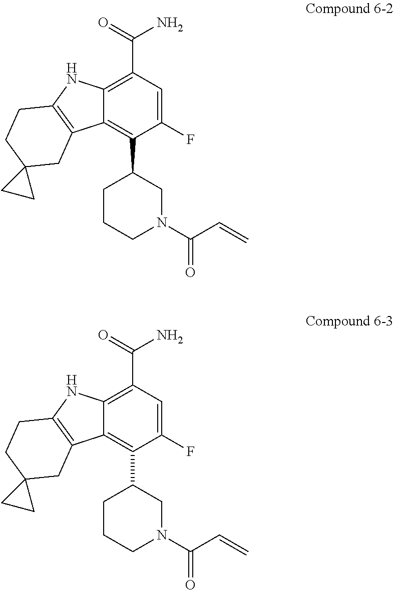

### Step 1: 8-methylene-1,4-dioxaspiro[4.5]decane

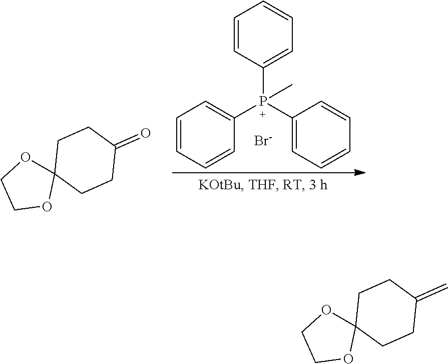

Potassium tert-butoxide (499 mL, 499 mmol, 1 M in THF) was added dropwise to a mixture of methyltriphenylphosphonium bromide (178 g, 499.43 mmol) in THF (450 mL) at 0° C. After stirring at this temperature for 1 h, 1,4-dioxaspiro[4.5]decan-8-one (26 g, 166.48 mmol) was added at 0° C. The reaction mixture warmed to room temperature and was stirred for 3 h. The reaction mixture was poured into saturated aqueous ammonium chloride (500 mL) and extracted with diethyl ether (300 mL×4).

The combined organic extracts were washed with brine (400 mL), dried over Na₂SO₄, filtered and concentrated under vacuum. The residue was purified by column chromatography on silica gel eluting with ethyl acetate in petroleum ether (11%) to give 8-methylene-1,4-dioxa-spiro[4.5]decane (18.0 g) as a colorless oil. ¹H NMR (400 MHz, CDCl₃) δ 4.67 (s, 2H), 3.96 (s, 4H), 2.28 (t, J=6.8 Hz, 4H), 1.70 (t, J=6.8 Hz, 4H).

### Step 2: 7,10-dioxadispiro[2.2.4⁶.2³]dodecane

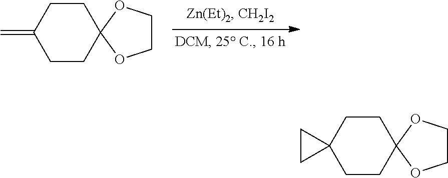

To a stirred solution of 8-methylene-1,4-dioxaspiro[4.5]decane (47.0 g, 305 mmol) and diiodomethane (278 g, 1.04 mol) in dichloromethane (500 mL) was added dropwise diethylzinc (1.0 M n-hexane, 518.14 mL) under nitrogen at 25° C. After the addition, the reaction mixture was stirred at 25° C. for 16 h. The reaction mixture was quenched with saturated aqueous ammonium chloride (1 L) and extracted with dichloromethane (600 mL×3). The combined organic extracts were washed with brine (800 mL), dried over sodium sulfate and concentrated under vacuum. The residue was purified by column chromatography on silica gel eluting with ethyl acetate in petroleum ether (0 to 9%) to give the desired compound (49.5 g) as a colorless oil. ¹H NMR (400 MHz, CDCl₃) δ 3.93 (s, 4H), 1.66 (t, J=6.4 Hz, 4H), 1.39 (t, J=6.0 Hz, 4H), 0.25 (s, 4H).

### Step 3: 1-(5-bromo-4-fluoro-2-iodophenyl)-2-(spiro[2.5]octan-6-ylidene)hydrazine

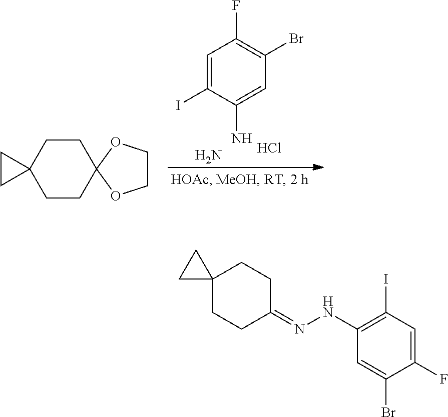

To a solution of (5-bromo-4-fluoro-2-iodophenyl)hydrazine hydrochloride (60.0 g, 163.32 mmol) in methanol (500 mL) were added 7,10-dioxadispiro[2.2.4⁶.2³]dodecane (30.22 g, 179.65 mmol) and acetic acid (29.42 g, 489.95 mmol). The reaction mixture was stirred at room temperature for 2 h. The reaction mixture was concentrated under vacuum. The residue was washed with ethyl acetate, filtered and dried under reduced pressure to give 1-(5-bromo-4-fluoro-2-iodophenyl)-2-(spiro[2.5]octan-6-ylidene)hydrazine (66.0 g) as a white solid. ¹H NMR (300 MHz, DMSO-d₆) δ 7.75 (d, J=8.1 Hz, 1H), 7.45 (d, J=6.6 Hz, 1H), 2.50-2.26 (m, 4H), 1.64-1.41 (m, 4H), 0.38 (s, 4H). ESI-MS [M+H]⁺ calculated for (C₁₄H₁₅BrFIN₂) 436.94, 438.94, found: 436.95, 438.95.

### Step 4: 5-bromo-6-fluoro-8-iodo-1,2,4,9-tetrahydrospiro[carbazole-3,1′-cyclopropane]

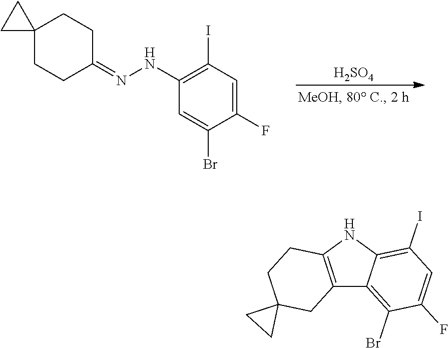 [SMILES: Fc1cc(I)c(NN=C2CCC3(CC2)CC3)cc1Br ; "H2SO4" ; Fc1cc(I)c2[nH]c3c(c2c1Br)CC1(CC3)CC1]

To a mixture of 1-(5-bromo-4-fluoro-2-iodophenyl)-2-(spiro[2.5]octan-6-ylidene)hydrazine (66.0 g, 151.0 mmol) in methanol (600 mL) was added concentrated sulfuric acid (12 mL). The reaction mixture was heated at 80° C. for 2 h. The cooled reaction mixture was concentrated under vacuum. The residue was diluted with saturated aqueous sodium bicarbonate and extracted with ethyl acetate (300 mL×4). The combined organic layers was washed with water (400 mL) and brine (400 mL), dried over anhydrous sodium sulfate, filtered and concentrated under vacuum. The residue was purified by column chromatography on silica gel eluting with ethyl acetate in petroleum ether (0 to 15%) to give 5-bromo-6-fluoro-8-iodo-1,2,4,9-tetrahydrospiro[carbazole-3,1′-cyclopropane] (27.0 g) as a yellow solid. ¹H NMR (400 MHz, DMSO-d₆) δ 11.03 (s, 1H), 7.36 (d, J=8.8 Hz, 1H), 2.82 (s, 2H), 2.77 (t, J=6.4 Hz, 2H), 1.56 (t, J=6.4 Hz, 2H), 0.46-0.34 (m, 4H). ESI-MS [M−H]⁻ calculated for (C₁₄H₁₂BrFIN) 417.92, 419.92, found: 417.80, 419.80.

### Step 5: 5-bromo-6-fluoro-1,2,4,9-tetrahydrospiro[carbazole-3,1′-cyclopropane]-8-carbonitrile

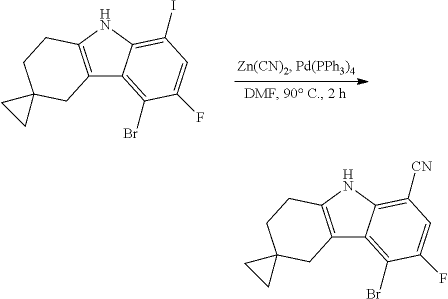 [SMILES: Fc1cc(I)c2[nH]c3c(c2c1Br)CC1(CC3)CC1 ; N#Cc1cc(F)c(Br)c2c3c([nH]c12)CCC1(CC1)C3]

To a solution of 5-bromo-6-fluoro-8-iodo-1,2,4,9-tetrahydrospiro[carbazole-3,1′-cyclopropane] (26.6 g, 63.32 mmol) in N,N-dimethylformamide (250 mL) were added zinc cyanide (15.37 g, 31.66 mmol) and tetrakis(triphenylphosphine)palladium (7.32 g, 6.33 mmol). The mixture was degassed and backfilled with nitrogen for five times and stirred for 2 h at 90° C. The cooled mixture was diluted with water (600 mL) and extracted with ethyl acetate (400 mL×3). The combined organic extracts were washed with water (400 mL) and brine (400 mL), dried over anhydrous sodium sulfate, filtered and concentrated under vacuum. The residue was purified by column chromatography on silica gel eluting with ethyl acetate in petroleum ether (0 to 30%) to give 5-bromo-6-fluoro-1,2,4,9-tetrahydrospiro[carbazole-3,1′-cyclopropane]-8-carbonitrile (18.0 g) as a yellow solid. ¹H NMR (400 MHz, DMSO-d₆) δ 12.01 (s, 1H), 7.53 (d, J=9.2 Hz, 1H), 2.80 (s, 2H), 2.76 (t, J=6.4 Hz, 2H), 1.57 (t, J=6.0 Hz, 2H), 0.46-0.33 (m, 4H). ESI-MS [M−H]⁻ calculated for (C₁₅H₁₂BrFN₂) 317.02, 319.02, found: 317.10, 319.10.

### Step 6: tert-butyl 5-(8-cyano-6-fluoro-1,2,4,9-tetrahydrospiro[carbazole-3,1′-cyclopropan]-5-yl)-3,6-dihydropyridine-1(2H)-carboxylate

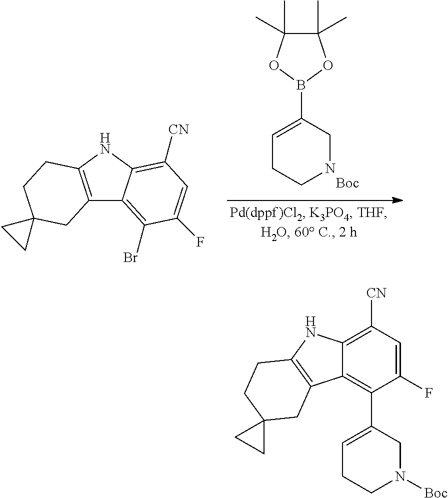

A mixture of 5-bromo-6-fluoro-1,2,4,9-tetrahydrospiro[carbazole-3,1′-cyclopropane]-8-carbonitrile (17.5 g, 54.83 mmol), tert-butyl 5-(4,4,5,5-tetramethyl-1,3,2-dioxaborolan-2-yl)-3,6-dihydropyridine-1(2H)-carboxylate (20.34 g, 65.80 mmol), potassium phosphate (34.92 g, 164.49 mmol) and Pd(dppf)Cl₂ (4.01 g, 5.48 mmol) in tetrahydrofuran (200 mL) and water (50 mL) was degassed and backfilled with nitrogen for five times. The reaction mixture was stirred under nitrogen at 60° C. for 2 h. The cooled reaction mixture was diluted with water (300 mL) and extracted with ethyl acetate (300 mL×3). The combined organic layers was washed with brine (300 mL), dried over sodium sulfate and concentrated under vacuum to give tert-butyl 5-(8-cyano-6-fluoro-1,2,4,9-tetrahydrospiro[carbazole-3,1′-cyclopropan]-5-yl)-3,6-dihydropyridine-1(2H)-carboxylate (40.0 g, crude) as a brown solid. ESI-MS [M−H]⁻ calculated for (C₂₅H₂₈FN₃O₂) 420.22, found: 420.20.

### Step 7: tert-butyl 5-(8-carbamoyl-6-fluoro-1,2,4,9-tetrahydrospiro-[carbazole-3,1′-cyclopropan]-5-yl)-3,6-dihydropyridine-1(2H)-carboxylate

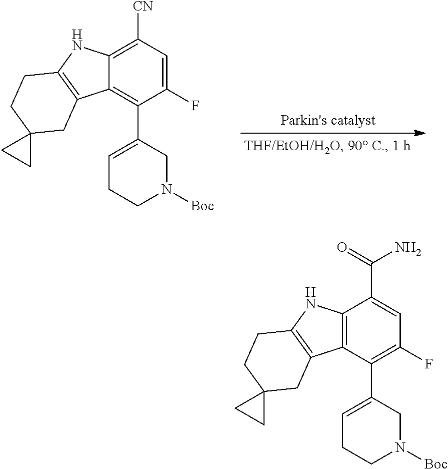 [SMILES: CC(C)(C)OC(=O)N1CCC=C(c2c(F)cc(C#N)c3[nH]c4c(c23)CC2(CC4)CC2)C1 ; CC(C)(C)OC(=O)N1CCC=C(c2c(F)cc(C(N)=O)c3[nH]c4c(c23)CC2(CC4)CC2)C1]

To a mixture of tert-butyl 5-(8-cyano-6-fluoro-1,2,4,9-tetrahydrospiro[carbazole-3,1′-cyclopropan]-5-yl)-3,6-dihydropyridine-1 (2H)-carboxylate (40.0 g, crude) in ethanol (100 mL), water (100 mL) and tetrahydrofuran (100 mL) was added Parkin's catalyst (2.33 g, 5.46 mmol). The reaction mixture was heated at 90° C. for 2 h. The cooled reaction mixture was diluted with water (400 mL) and extracted with ethyl acetate (300 ml×3). The combined organic extracts were washed with brine (300 mL), dried over anhydrous sodium sulfate, filtered and concentrated under vacuum. The residue was purified by column chromatography on silica gel eluting with ethyl acetate in petroleum ether (50%) to give tert-butyl 5-(8-carbamoyl-6-fluoro-1,2,4,9-tetrahydrospiro [carbazole-3,1′-cyclopropan]-5-yl)-3,6-dihydropyridine-1(2H)-carboxylate (22.0 g, 91% over two steps) as a yellow solid. ESI-MS [M+H]⁺ calculated for (C₂₅H₃₀FN₃O₃) 440.23, found: 440.20.

### Step 8: tert-butyl 3-(8-carbamoyl-6-fluoro-1,2,4,9-tetrahydrospiro[carbazole-3,1′-cyclopropan]-5-yl)piperidine-1-carboxylate

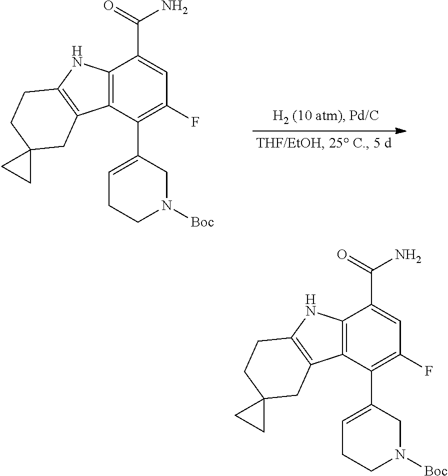 [SMILES: CC(C)(C)OC(=O)N1CCC=C(c2c(F)cc(C(N)=O)c3[nH]c4c(c23)CC2(CC4)CC2)C1 ; CC(C)(C)OC(=O)N1CCC=C(c2c(F)cc(C(N)=O)c3[nH]c4c(c23)CC2(CC4)CC2)C1]

A mixture of tert-butyl 5-(8-carbamoyl-6-fluoro-1,2,4,9-tetrahydrospiro-[carbazole-3,1′-cyclopropan]-5-yl)-3,6-dihydropyridine-1(2H)-carboxylate (18.0 g, 40.95 mmol) and 10% palladium on carbon (18.0 g) in ethanol (150 mL) and tetrahydrofuran (150 mL) was stirred under hydrogen (10 atm) at 25° C. for 5 d. The reaction mixture was filtered. The filtrate was concentrated under vacuum. The residue was purified by column chromatography on silica gel eluting with tetrahydrofuran in petroleum ether (15%) to give tert-butyl 3-(8-carbamoyl-6-fluoro-1,2,4,9-tetrahydrospiro[carbazole-3,1′-cyclopropan]-5-yl)piperidine-1-carboxylate (12.0 g) as a white solid. ¹H NMR (300 MHz, DMSO-d₆) δ 10.83 (s, 1H), 7.99 (s, 1H), 7.51-7.30 (m, 2H), 4.20-3.84 (m, 2H), 3.30-3.20 (m, 1H), 2.90-2.60 (m, 5H), 2.12-1.82 (m, 2H), 1.71-1.48 (m, 3H), 1.39 (s, 9H), 1.30-0.92 (m, 2H), 0.52-0.26 (m, 4H). ESI-MS [M+H]⁺ calculated for (C₂₅H₃₂FN₃O₃) 442.24, found: 442.20.

### Step 9: 6-fluoro-5-(piperidin-3-yl)-1,2,4,9-tetrahydrospiro[carbazole-3,1′-cyclopropane]-8-carboxamide 2,2,2-trifluoroacetate

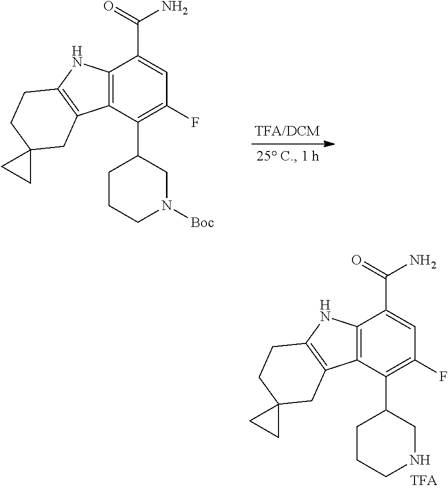 [SMILES: CC(C)(C)OC(=O)N1CCCC(c2c(F)cc(C(N)=O)c3[nH]c4c(c23)CC2(CC4)CC2)C1 ; NC(=O)c1cc(F)c(C2CCCNC2)c2c3c([nH]c12)CCC1(CC1)C3]

To a solution of tert-butyl 3-(8-carbamoyl-6-fluoro-1,2,4,9-tetrahydrospiro[carbazole-3,1′-cyclopropan]-5-yl)piperidine-1-carboxylate (12.0 g, 27.18 mmol) in dichloromethane (120 mL) was added trifluoroacetic acid (20 mL). The reaction mixture was stirred for 1 h at 25° C. The reaction mixture was concentrated under vacuum to give 6-fluoro-5-(piperidin-3-yl)-1,2,4,9-tetrahydrospiro[carbazole-3,1′-cyclopropane]-8-carboxamide 2,2,2-trifluoroacetate (12.3 g, crude) as a brown oil. ESI-MS [M+H]⁺ calculated for (C₂₀H₂₄FN₃O) 342.19, found: 342.20.

### Step 10: 5-(1-acryloylpiperidin-3-yl)-6-fluoro-1,2,4,9-tetrahydrospiro-[carbazole-3,1′-cyclopropane]-8-carboxamide

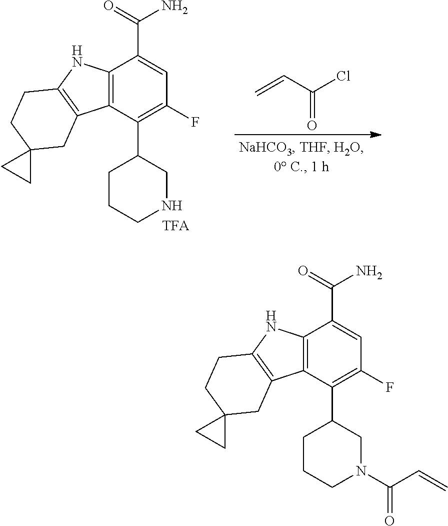

To a mixture of 7-fluoro-8-(piperidin-3-yl)-1,2,3,4-tetrahydrocyclo-penta[b]indole-5-carboxamide 2,2,2-trifluoroacetate (12 g, crude) in tetrahydrofuran (100 mL) and water (25 mL) was added sodium bicarbonate (22.13 g, 263.48 mmol). After stirring for 10 min, acryloyl chloride (2.86 g, 31.62 mmol) was added at 0° C. The reaction mixture was stirred at 0° C. for 1 h. The reaction mixture was diluted with water (300 ml) and extracted with ethyl acetate (300 ml×3). The combined organic extracts were washed with brine (300 ml), dried over anhydrous sodium sulfate, filtered and concentrated under vacuum. The residue was purified by Prep-FLASH with the flowing conditions: Column: XB C18 50×250 mm, 10 um; Mobile Phase A: Water (10 mmol/L NH₄HCO₃), Mobile Phase B: Acetonitrile; Flow rate: 100 mL/min; Gradient: 30% B to 55% B in 40 min; 254 nm; Rt: 27.0 min to give 5-(1-acryloylpiperidin-3-yl)-6-fluoro-1,2,4,9-tetrahydrospiro[carbazole-3,1′-cyclopropane]-8-carboxamide (5.4 g, 52% over two steps) as an off-white solid. ESI-MS [M+H]⁺ calculated for (C₂₃H₂₆FN₃O₂) 396.20 found: 396.25.

### Step 11: (S)-5-(1-acryloylpiperidin-3-yl)-6-fluoro-1,2,4,9-tetrahydrospiro[carbazole-3,1′-cyclopropane]-8-carboxamide (Compound 6-2) and (R)-5-(1-acryloylpiperidin-3-yl)-6-fluoro-1,2,4,9-tetrahydrospiro[carbazole-3,1′-cyclopropane]-8-carboxamide (Compound 6-3)

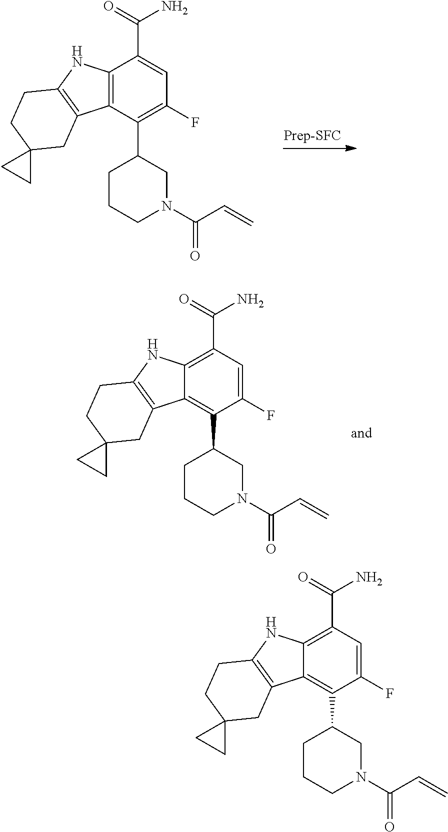

5-(1-acryloylpiperidin-3-yl)-6-fluoro-1,2,4,9-tetrahydrospiro[carbazole-3,1′-cyclopropane]-8-carboxamide (5.4 g) was separated by Prep-SFC with the following conditions: Column: CHIRALPAK AD-H SFC, 5×25 cm, 5 um; Mobile Phase A: CO₂, Mobile Phase B: iPrOH (0.5% 2 M NH₃-MeOH); Flow rate: 180 mL/min; Gradient: 50% B; 220 nm:

RT1: 3.32 min:

Compound 6-2 (S)-5-(1-acryloylpiperidin-3-yl)-6-fluoro-1,2,4,9-tetrahydrospiro[carbazole-3,1′-cyclopropane]-8-carboxamide (2.4 g, 92% pure) and

RT2: 5.42 min

Compound 6-3 (R)-5-(1-acryloylpiperidin-3-yl)-6-fluoro-1,2,4,9-tetrahydrospiro[carbazole-3,1′-cyclopropane]-8-carboxamide (2.24 g, 41%) as a light-yellow solid.

¹H NMR (300 MHz, DMSO-d₆) δ 10.84 (s, 1H), 8.01 (s, 1H), 7.53-7.35 (m, 2H), 6.89-6.79 (m, 1H), 6.20-6.01 (m, 1H), 5.77-5.52 (m, 1H), 4.52 (t, J=12.0 Hz, 1H), 4.20-4.00 (m, 1H), 3.61-3.40 (m, 0.5H), 3.32-2.90 (m, 2H), 2.88-2.53 (m, 4.5H), 2.20-2.00 (m, 1H), 2.00-1.71 (m, 2H), 1.67-1.34 (m, 3H), 0.49-0.23 (m, 4H). ESI-MS [M+H]⁺ calculated for (C₂₃H₂₆FN₃O₂) 396.20 found: 396.35.

Compound 6-2 was further purified by Prep-Achiral-SFC with the following conditions: column: DAICEL DCpak P4VP, 20 mm×250 mm, 5 um; Mobile Phase A: CO₂, Mobile Phase B: MeOH (0.1% 2 M NH₃-MeOH); Flow rate: 50 mL/min; Gradient: 25% B; 254 nm; RT: 4.22 min to give (S)-5-(1-acryloylpiperidin-3-yl)-6-fluoro-1,2,4,9-tetrahydrospiro[carbazole-3,1′-cyclopropane]-8-carboxamide (1.99 g, 37%) as a light-yellow solid. ¹H NMR (300 MHz, DMSO-d₆) δ 10.84 (s, 1H), 8.01 (s, 1H), 7.53-7.35 (m, 2H), 6.89-6.79 (m, 1H), 6.20-6.01 (m, 1H), 5.77-5.52 (m, 1H), 4.52 (t, J=12.9 Hz, 1H), 4.20-4.00 (m, 1H), 3.61-3.40 (m, 0.5H), 3.32-2.90 (m, 2H), 2.88-2.53 (m, 4.5H), 2.20-2.00 (m, 1H), 2.00-1.71 (m, 2H), 1.67-1.34 (m, 3H), 0.50-0.25 (m, 4H). ESI-MS [M+H]⁺ calculated for (C₂₃H₂₆FN₃O₂) 396.20 found: 396.35.

### Example 7-1

### Preparation of 4-(1-prop-2-enoyl-3,6-dihydro-2H-pyridin-5-yl)spiro[5,7,8,9-tetrahydro-carbazole-6,1′-cyclopropane]-1-carboxamide (Compound 7-1)

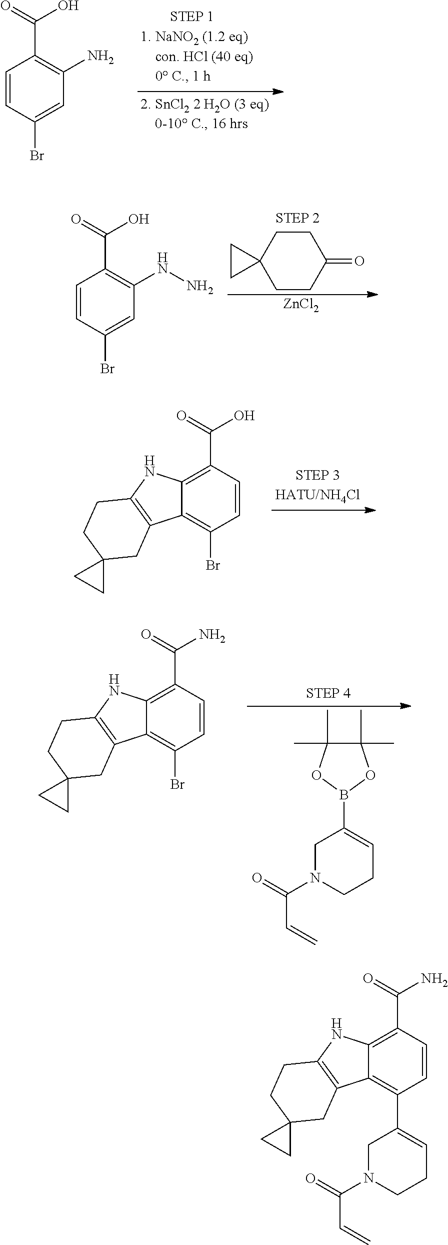

### Step 1: 4-bromo-2-hydrazineylbenzoic acid

A solution of sodium nitrite (9.58 g, 138 mmol, 1.20 eq) in water (˜1 mol/L) was added dropwise to a suspension of 2-amino-4-bromobenzoic acid (25.0 g, 115 mmol, 1.00 eq) in HCl (12 M, 385 mL, 40.0 eq) at 0° C. and the mixture stirred at 0° C. for 1 hr. A solution of SnCl₂.2H₂O (78.3 g, 347 mmol, 3.00 eq) in HCl (12 M) (˜2 mol/L) was then added dropwise at 0° C. and the mixture stirred at 0-10° C. for 16 hrs. The mixture was filtered and freeze-dried to produce a yellow solid. Dichloromethane (200 mL) and acetonitrile (300 mL) were added to the solid and the resulting mixture stirred at 20° C. for 2 h and then filtrated. The isolated solids were washed with acetonitrile and then dried to provide 4-bromo-2-hydrazineylbenzoic acid hydrochloride (15.0 g, 56.0 mmol) as a white solid.

### Step 2: 5-bromo-1,2,4,9-tetrahydrospiro[carbazole-3,1′-cyclopropane]-8-carboxylic acid

4-bromo-2-hydrazineylbenzoic acid hydrochloride was coupled with a spiro cyclic ketone in the presence of ZnCl₂ in either isopropyl alcohol or diethyl ether, as follows: ZnCl₂ (1.50 eq) in IPA (20 mL) or DME (30 mL) was added to a suspension of 4-bromo-2-hydrazineylbenzoic acid hydrochloride (500 mg, 1.00 eq) and cyclic ketone (1.05 eq), and the mixture stirred at 90° C. under N₂ for 1-7 days. Solvents were removed by evaporation to provide an oil that was purified by prep-HPLC.

### Step 3: 4-bromospiro[5,7,8,9-tetrahydrocarbazole-6,1′-cyclopropane]-1-carboxamide

To a mixture of 4-bromospiro[5,7,8,9-tetrahydrocarbazole-6,1′-cyclopropane]-1-carboxylic acid (100 mg, 312.32 μmol), ammonium chloride (66.83 mg, 1.25 mmol) in DMA (1 mL) was added HATU (131.32 mg, 345.38 μmol), mixture was allowed to continue stirring at RT for 30 min. The mixture was diluted with EtOAc and water, the organic layer was separated, and the aqueous layer was extracted with EtOAc twice more. The combined organic layer was washed with brine, dried over Na₂SO₄, filtered and concentrated. The residue obtained was purified by silica gel chromatography using a gradient of 5%-60% EtOAc in heptane to give 4-bromospiro[5,7,8,9-tetrahydrocarbazole-6,1′-cyclopropane]-1-carboxamide (80.5 mg) as a white solid.

### Step 4: 4-(1-prop-2-enoyl-3,6-dihydro-2H-pyridin-5-yl)spiro[5,7,8,9-tetrahydrocarbazole-6,1′-cyclopropane]-1-carboxamide (Compound 6-1)

To a solution of 4-bromospiro[5,7,8,9-tetrahydrocarbazole-6,1′-cyclopropane]-1-carboxamide (75 mg, 234.97 μmol) in THF (1 mL), methanol (0.5 mL) and water (0.5 mL), 1-[5-(4,4,5,5-tetramethyl-1,3,2-dioxaborolan-2-yl)-3,6-dihydro-2H-pyridin-1-yl]prop-2-en-1-one (74.19 mg, 281.96 μmol), PdCl₂(dppf) (66.2 mg, 0.091 mmol) and Na₂CO₃ (144 mg, 1.358 mmol) were added. The reaction mixture was heated at about 70° C. for 16 h. The mixture was cooled to rt and diluted with EtOAc and water. The organic layer was separated and the aq layer was extracted with EtOAc twice more. The combined organic layer was washed with brine, dried over Na₂SO₄, filtered and concentrated under reduced pressure. The residue obtained was purified by reverse phase chromatography using a gradient of 10%-95% MeCN in water w/0.10% Formic Acid. The like fractions were combined and freeze dried on the lyophilizer to give 4-(1-prop-2-enoyl-3,6-dihydro-2H-pyridin-5-yl)spiro[5,7,8,9-tetrahydrocarbazole-6,1′-cyclopropane]-1-carboxamide (42 mg) as a light yellow solid. ¹H NMR (DMSO-d₆) δ: 10.80 (s, 1H), 7.96 (br s, 1H), 7.53 (d, J=7.7 Hz, 1H), 7.29 (br s, 1H), 6.69-6.96 (m, 2H), 6.13 (br d, J=16.5 Hz, 1H), 5.60-5.76 (m, 2H), 4.23 (br d, J=12.2 Hz, 2H), 3.62-3.79 (m, 2H), 2.80 (br t, J=5.0 Hz, 2H), 2.42 (br d, J=6.3 Hz, 2H), 2.18-2.35 (m, 2H), 1.56 (br t, J=6.0 Hz, 2H), 0.22-0.43 (m, 4H).

The tricyclic indoles shown in Table 6 were prepared using a procedure similar to that described in either Examples 6 or 7.

TABLE 6
TRICYCLIC INDOLES

| Spiroketone | Product | ¹H NMR |
|---|---|---|
| 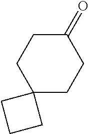 | 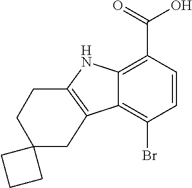 [SMILES: O=C(O)c1ccc(Br)c2c3c([nH]c12)CCC1(CCC1)C3] | (400 MHz, DMSO-d⁶) d 13.1 (s, 1H), 11.0 (s, 1H), 7.46 (d, J = 8.0 Hz, 2H), 7.18 (d, J = 8.0 Hz, 2H), 2.86 (s, 2H), 2.75 (t, J = 7.6 Hz, 2H), 1.90- 1.77 (m, 6H). |
| 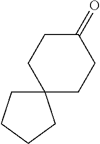 [SMILES: O=C1CCC2(CCCC2)CC1] | 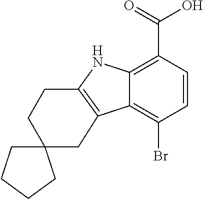 [SMILES: O=C(O)c1ccc(Br)c2c3c([nH]c12)CCC1(CCCC1)C3] | (400 MHz, DMSO-d⁶) d 13.1 (s, 1H), 11.0 (s, 1H), 7.46 (d, J = 8.0 Hz, 2H), 7.15 (d, J = 8.0 Hz, 2H), 3.07 (s, 2H), 2.77 (t, J = 7.6 Hz, 2H), 1.65 (m, 6H), 1.42 (m, 4H). |
| 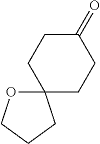 [SMILES: O=C1CCC2(CCCO2)CC1] | 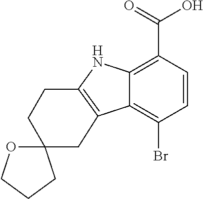 [SMILES: O=C(O)c1ccc(Br)c2c3c([nH]c12)CCC1(CCCO1)C3] | (400 MHz, DMSO-d⁶) d 13.1 (s, 1H), 11.0 (s, 1H), 7.47 (d, J = 8.0 Hz, 2H), 7.16 (d, J = 8.0 Hz, 2H), 3.83 (m, 2H), 3.05 (s, 2H), 2.77 (m, 2H), 1.94-1.73 (m, 6H). |
| 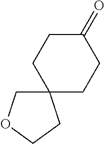 [SMILES: O=C1CCC2(CCOC2)CC1] | 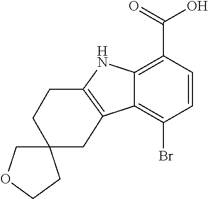 [SMILES: O=C(O)c1ccc(Br)c2c3c([nH]c12)CCC1(CCOC1)C3] | (400 MHz, DMSO-d⁶) d 13.1 (bs, 1H), 11.0 (s, 1H), 7.48 (d, J = 8.0 Hz, 2H), 7.17 (d, J = 8.0 Hz, 2H), 3.80 (t, J = 7.6 Hz, 2H), 3.49 (q, J = 7.6 Hz, 2H), 2.95 (s, 2H), 2.77 (m, 2H), 1.82-1.69 (m, 4H). |
| 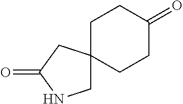 [SMILES: O=C1CCC2(CC1)CNC(=O)C2] | 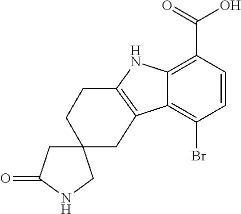 [SMILES: O=C1CC2(CCc3[nH]c4c(C(=O)O)ccc(Br)c4c3C2)CN1] | (400 MHz, DMSO-d⁶) d 13.1 (s, 1H), 11.0 (s, 1H), 7.52 (s, 1H), 7.49 (d, J = 8.0 Hz, 2H), 7.18 (d, J = 8.0 Hz, 2H), 3.13-3.03 (m, 3H), 2.80 (m, 2H), 2.17 (d, J = 16.4 Hz, 1H), 1.97 (d, J = 16.4 Hz, 1H), 1.83-1.79 (m, 2H). |
| 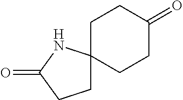 [SMILES: O=C1CCC2(CC1)CCC(=O)N2] | 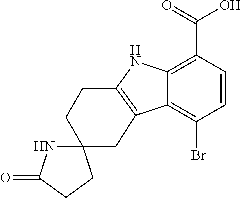 [SMILES: O=C1CCC2(CCc3[nH]c4c(C(=O)O)ccc(Br)c4c3C2)N1] | (400 MHz, DMSO-d⁶) d 13.1 (s, 1H), 11.0 (s, 1H), 7.99 (s, 1H), 7.49 (d, J = 8.0 Hz, 2H), 7.18 (d, J = 8.0 Hz, 2H), 3.14 (d, J = 16.4 Hz, 1H), 3.02 (d, J = 16.4 Hz, 1H), 2.87 (m, 2H), 2.25 (m, 2H), 1.91-1.79 (m, 4H). |
| 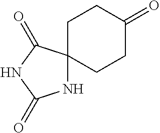 [SMILES: O=C1CCC2(CC1)NC(=O)NC2=O] | 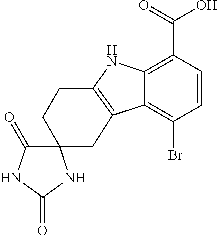 [SMILES: O=C1NC(=O)C2(CCc3[nH]c4c(C(=O)O)ccc(Br)c4c3C2)N1] | (400 MHz, DMSO-d⁶) d 13.1 (bs, 1H), 11.1 (s, 1H), 10.6 (s, 1H), 8.33 (s, 1H), 7.51 (d, J = 8.0 Hz, 2H), 7.20 (d, J = 8.0 Hz, 2H), 3.21-2.88 (m, 4H), 1.99-1.87 (m, 2H). |
| 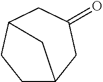 | 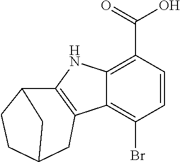 [SMILES: O=C(O)c1ccc(Br)c2c3c([nH]c12)C1CCC(C3)C1] | (400 MHz, DMSO-d⁶) d 13.0 (s, 1H), 11.0 (s, 1H), 7.45 (d, J = 8.0 Hz, 2H), 7.17 (d, J = 8.0 Hz, 2H), 3.90 (s, 1H), 2.98 (dd, J = 3.6, 13.2 Hz,1H), 2.66-2.55 (m, 2H), 1.89-1.68 (m, 5H), 1.31 (m, 1H). |

From the intermediates shown in Table 6, Compounds 7-2 through 7-9, as shown in Table 7, of Formula (I-C-i) were prepared.

TABLE 7
COMPOUNDS 7-2 THROUGH 7-9

| Compound | | |
|---|---|---|
| No. | Structure/Name | ¹H NMR |
| 7-2 | 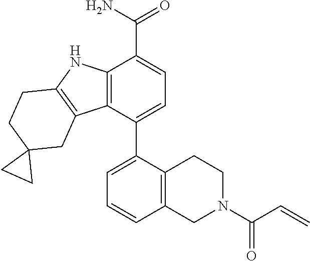 [SMILES: C=CC(=O)N1CCc2c(cccc2-c2ccc(C(N)=O)c3[nH]c4c(c23)CC2(CC4)CC2)C1] | (DMSO-d₆) δ: 10.85 (s, 1H), 8.01 (br s, 1H), 7.45- 7.64 (m, 2H), 7.34 (br s, 1H), 7.24 (br d, J = 5.9 Hz, 1H), 7.02-7.11 (m, 1H), 6.76-6.98 (m, 1H), 6.71 (d, J = 7.7 Hz, 1H), 6.13 (dd, J = 16.6, 2.4 Hz, 1H), 5.60- 5.80 (m, 1H), 4.60-4.92 (m, 2H), 3.44-3.72 (m, 2H), 2.77 (br t, J = 5.8 Hz, 2H), 2.23-2.41 (m, 2H), 1.67-1.82 (m, 1H), 1.31-1.62 (m, 3H), 0.26 (br s, 2H), −0.10-0.04 (m, 2H) |
| 7-3 | 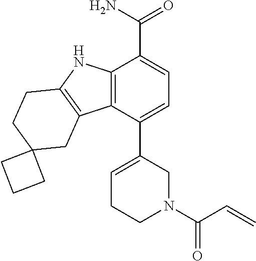 [SMILES: C=CC(=O)N1CCC=C(c2ccc(C(N)=O)c3[nH]c4c(c23)CC2(CCC2)CC4)C1] | (DMSO-d₆) δ: 10.73 (br s, 1H), 7.95 (br s, 1H), 7.52 (br d, J = 7.6 Hz, 1H), 7.27 (br s, 1H), 6.69-7.01 (m, 2H), 6.16 (br d, J = 17.3 Hz, 1H), 5.73 (br d, J = 9.1 Hz, 2H), 4.28 (br d, J = 6.6 Hz, 2H), 3.70-3.87 (m, 2H), 2.76 (br t, J = 5.4 Hz, 2H), 2.62 (br d, J = 8.3 Hz, 2H), 2.22-2.41 (m, 2H), 1.72-1.95 (m, 6H), 1.64 (br d, J = 7.1 Hz, 2H) |
| 7-4 | 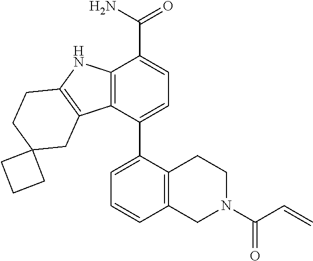 [SMILES: C=CC(=O)N1CCc2c(cccc2-c2ccc(C(N)=O)c3[nH]c4c(c23)CC2(CCC2)CC4)C1] | (DMSO-d₆) δ: 10.77 (s, 1H), 7.99 (br s, 1H), 7.59 (d, J = 7.6 Hz, 1H), 7.30 (br d, J = 6.0 Hz, 3H), 7.03- 7.14 (m, 1H), 6.75-7.00 (m, 1H), 6.72 (d, J = 7.6 Hz, 1H), 6.06-6.20 (m, 1H), 5.58-5.78 (m, 1H), 4.58- 5.01 (m, 2H), 3.42-3.83 (m, 2H), 2.72 (br s, 2H), 2.24-2.41 (m, 2H), 1.53-1.87 (m, 8H), 1.36 (br d, J = 3.7 Hz, 2H) |
| 7-5 | 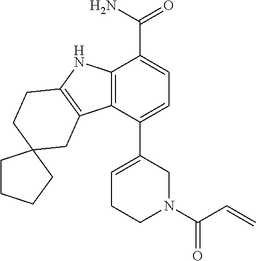 [SMILES: C=CC(=O)N1CCC=C(c2ccc(C(N)=O)c3[nH]c4c(c23)CC2(CCCC2)CC4)C1] | (400 MHz, DMSO-d₆) δ: 10.75 (br s, 1H), 7.97 (br s, 1H), 7.52 (br d, J = 7.8 Hz, 1H), 7.30 (br s, 1H), 6.68-6.99 (m, 2H), 6.14 (br d, J = 16.5 Hz, 1H), 5.71 (br d, J = 11.0 Hz, 2H), 4.25 (br d, J = 11.9 Hz, 2H), 3.66-3.82 (m, 2H), 2.70-2.84 (m, 2H), 2.46 (br s, 2H), 2.19-2.38 (m, 2H), 1.51-1.74 (m, 6H), 1.24- 1.48 (m, 4H) |
| 7-6 | 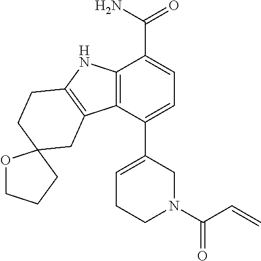 [SMILES: C=CC(=O)N1CCC=C(c2ccc(C(N)=O)c3[nH]c4c(c23)CC2(CCCO2)CC4)C1] | (400 MHz, DMSO-d₆) δ: 10.80 (s, 1H), 7.97 (br s, 1H), 7.53 (d, J = 7.7 Hz, 1H), 7.30 (br s, 1H), 6.71- 6.99 (m, 2H), 6.15 (br d, J = 17.3 Hz, 1H), 5.62-5.78 (m, 2H), 4.12-4.37 (m, 2H), 3.71 (br t, J = 6.7 Hz, 3H), 2.54-2.94 (m, 5H), 2.30 (br d, J = 14.4 Hz, 2H), 1.51-1.96 (m, 6H) |
| 7-7 | 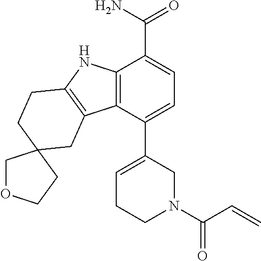 [SMILES: C=CC(=O)N1CCC=C(c2ccc(C(N)=O)c3[nH]c4c(c23)CC2(CCOC2)CC4)C1] | (400 MHz, DMSO-d₆) δ: 10.81 (s, 1H), 7.96 (br s, 1H), 7.54 (d, J = 7.7 Hz, 1H), 7.29 (br s, 1H), 6.69- 6.99 (m, 2H), 6.14 (br dd, J = 15.9, 5.0 Hz, 1H), 5.61-5.78 (m, 2H), 4.13-4.36 (m, 2H), 3.65-3.86 (m, 4H), 3.50 (d, J = 8.2 Hz, 1H), 3.28-3.38 (m, J = 18.8 Hz, 1H), 2.76-2.84 (m, 2H), 2.58 (br d, J = 6.7 Hz, 2H), 2.30 (br d, J = 13.8 Hz, 2H), 1.69- 1.83 (m, 3H), 1.52-1.66 (m, 1H) |
| 7-8 | 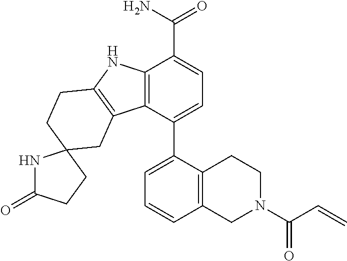 [SMILES: C=CC(=O)N1CCc2c(cccc2-c2ccc(C(N)=O)c3[nH]c4c(c23)CC2(CCC(=O)N2)CC4)C1] | (400 MHz, CD₃OD) δ: 7.62 (d, J = 7.7 Hz, 1H), 7.26-7.36 (m, 2H), 7.16 (dd, J = 6.4, 2.4 Hz, 1H), 6.68-6.98 (m, 2H), 6.19-6.32 (m, 1H), 5.69-5.86 (m, 1H), 4.90-5.07 (m, 2H), 3.51-3.94 (m, 2H), 2.95 (br t, J = 6.5 Hz, 2H), 2.45-2.58 (m, 2H), 2.28- 2.42 (m, 1H), 2.11-2.26 (m, 1H), 1.90-2.10 (m, 4H), 1.76-1.89 (m, 1H), 1.61-1.75 (m, 1H) |
| 7-9 | 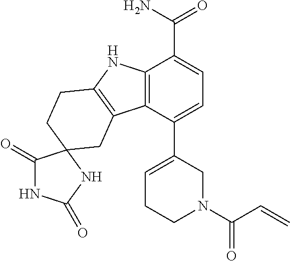 [SMILES: C=CC(=O)N1CCC=C(c2ccc(C(N)=O)c3[nH]c4c(c23)CC2(CC4)NC(=O)NC2=O)C1] | (400 MHz, DMSO-d₆) δ: 10.96 (s, 1H), 10.61 (br s, 1H), 8.22 (s, 1H), 7.99 (br s, 1H), 7.58 (d, J = 7.7 Hz, 1H), 7.30 (br s, 1H), 6.74-6.99 (m, 2H), 6.03- 6.20 (m, 1H), 5.58-5.78 (m, 2H), 4.34 (br d, J = 14.7 Hz, 1H), 4.08-4.26 (m, 1H), 3.67-3.77 (m, 2H), 2.94-3.07 (m, 2H), 2.59-2.78 (m, 2H), 2.27 (br t, J = 8.5 Hz, 2H), 1.80-2.07 (m, 2H) |

According to similar procedures, Compounds 7-10 through 7-25, as shown in Table 8, of Formula (I-C-ii) were prepared.

TABLE 8
COMPOUNDS 7-10 THROUGH 7-25

| Compound | | |
|---|---|---|
| No. | Structure/Name | ¹H NMR |
| 7-10 | 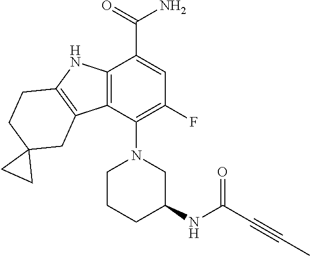 [SMILES: CC#CC(=O)N[C@H]1CCCN(c2c(F)cc(C(N)=O)c3[nH]c4c(c23)CC2(CC4)CC2)C1] | (400 MHz, DMSO-d₆) δ 10.71 (s, 1H), 8.45 (s, 1H), 7.92 (s, 1H), 7.41 (d, J = 14.2 Hz, 1H), 7.36-7.25 (m, 1H), 3.89-3.71 (m, 1H), 3.22-3.07 (m, 1H), 3.06-2.89 (m, 2H), 2.86-2.68 (m, 5H), 1.94 (s, 3H), 1.89-1.43 (m, 5H), 1.40-1.19 (m, 1H), 0.48-0.33 (m, 4H). |
| 7-11 | 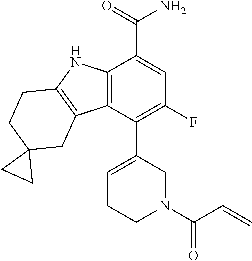 [SMILES: C=CC(=O)N1CCC=C(c2c(F)cc(C(N)=O)c3[nH]c4c(c23)CC2(CC4)CC2)C1] | (400 MHz, DMSO-d₆) δ 10.85 (s, 1H), 8.04 (s, 1H), 7.57-7.36 (m, 2H), 7.01-6.65 (m, 1H), 6.13 (d, J = 16.5 Hz, 1H), 5.84-5.65 (m, 2H), 4.38- 4.01 (m, 2H), 3.84-3.61 (m, 2H), 2.91-2.70 (m, 2H), 2.41-1.95 (m, 4H), 1.85-1.26 (m, 2H), 0.51- 0.17 (m, 4H). |
| 7-12 | 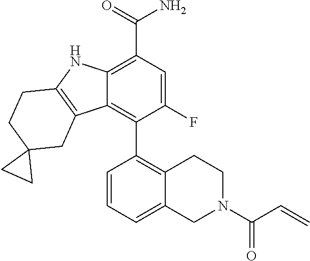 [SMILES: C=CC(=O)N1CCc2c(cccc2-c2c(F)cc(C(N)=O)c3[nH]c4c(c23)CC2(CC4)CC2)C1] | (400 MHz, DMSO-d₆) δ 10.91 (s, 1H), 8.08 (s, 1H), 7.54 (d, J = 10.8 Hz, 1H), 7.49 (s, 1H), 7.23- 7.32 (m, 2H), 7.07-7.12 (m, 1H), 6.85 (ddd, J = 44.8, 16.7, 10.6 Hz, 1H), 6.14 (dd, J = 16.7, 2.3 Hz, 1H), 5.70 (dd, J = 18.5, 10.6 Hz, 1H), 4.67-4.86 (m, 2H), 3.59-3.74 (m, 2H), 2.77 (s, 2H), 2.34 (dd, J = 14.2, 6.5 Hz, 2H), 1.76 (d, J = 15.9 Hz, 1H), 1.49-1.58 (m, 1H), 1.32- 1.47 (m, 2H), 0.26 (s, 2H), −0.03 (d, J = 8.6 Hz, 2H). |
| 7-13 | 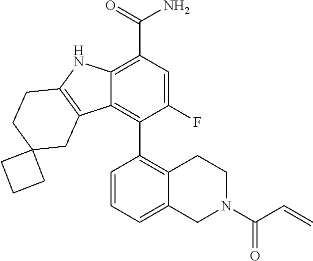 [SMILES: C=CC(=O)N1CCc2c(cccc2-c2c(F)cc(C(N)=O)c3[nH]c4c(c23)CC2(CCC2)CC4)C1] | (400 MHz, DMSO-d₆) δ 10.85 (s, 1H), 8.07 (s, 1H), 7.54 (d, J = 10.7 Hz, 1H), 7.45 (d, J = 17.0 Hz, 1H), 7.37-7.30 (m, 2H), 7.17-7.09 (m, 1H), 6.97-6.76 (m, 1H), 6.13 (dd, J = 16.6, 4.3 Hz, 1H), 5.75-5.63 (m, 1H), 4.97-4.66 (m, 2H), 3.78-3.58 (m, 2H), 2.72 (s, 2H), 2.36 (d, J = 5.6 Hz, 2H), 1.86-1.56 (m, 8H), 1.33 (s, 2H). |
| 7-14 | 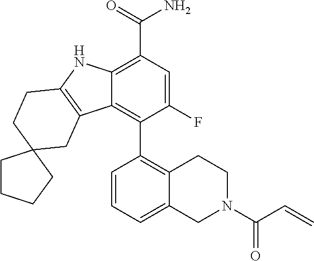 [SMILES: C=CC(=O)N1CCc2c(cccc2-c2c(F)cc(C(N)=O)c3[nH]c4c(c23)CC2(CCCC2)CC4)C1] | (400 MHz, DMSO-d₆) δ 10.84 (s, 1H), 8.07 (s, 1H), 7.53 (d, J = 10.7 Hz, 1H), 7.47 (s, 1H), 7.25- 7.35 (m, 2H), 7.08-7.14 (m, 1H), 6.74-6.93 (m, 1H), 6.13 (dd, J = 16.7, 2.0 Hz, 1H), 5.64- 5.75 (m, 1H), 4.61-4.92 (m, 2H), 3.54-3.76 (m, 2H), 2.66-2.77 (m, 2H), 2.27-2.36 (m, 2H), 1.41-1.68 (m, 6H), 1.20-1.36 (m, 4H), 1.06 (d, J = 5.2 Hz, 2H). |
| 7-15 | 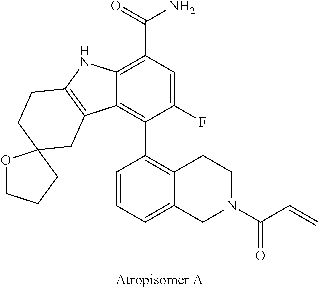 [SMILES: C=CC(=O)N1CCc2c(cccc2-c2c(F)cc(C(N)=O)c3[nH]c4c(c23)CC2(CCCO2)CC4)C1] | (400 MHz, DMSO-d₆) δ 10.91 (s, 1H), 8.08 (s, 1H), 7.55 (d, J = 10.7 Hz, 1H), 7.49 (s, 1H), 7.28- 7.36 (m, 2H), 7.12 (d, J = 8.7 Hz, 1H), 6.80 (dd, J = 16.6, 10.5 Hz, 1H), 6.10-6.18 (m, 1H), 5.65-5.75 (m, 1H), 4.89 (dt, J = 34.9, 12.5 Hz, 2H), 3.62-3.78 (m, 2H), 3.46-3.59 (m, 2H), 2.79-2.95 (m, 2H), 2.69 (dd, J = 17.0, 4.9 Hz, 2H), 2.33 (s, 2H), 1.81 (s, 4H), 1.54-1.68 (m, 4H). |
| 7-16 | 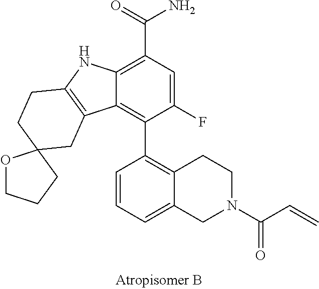 [SMILES: C=CC(=O)N1CCc2c(cccc2-c2c(F)cc(C(N)=O)c3[nH]c4c(c23)CC2(CCCO2)CC4)C1] | (400 MHz, DMSO-d₆) δ 10.90 (s, 1H), 8.09 (s, 1H), 7.54 (d, J = 10.7 Hz, 1H), 7.49 (s, 1H), 7.27- 7.36 (m, 2H), 7.09-7.15 (m, 1H), 6.73-6.93 (m, 1H), 6.09-6.19 (m, 1H), 5.70 (dd, J = 21.6, 11.3 Hz, 1H), 4.83 (dd, J = 42.1, 25.5 Hz, 2H), 3.49-3.72 (m, 4H), 2.71-2.86 (m, 2H), 2.37 (s, 2H), 1.56-1.91 (m, 8H). |
| 7-17 | 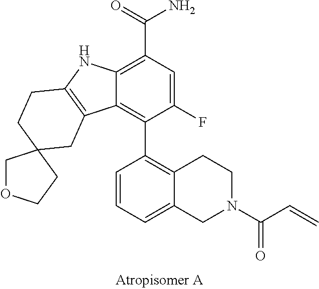 [SMILES: C=CC(=O)N1CCc2c(cccc2-c2c(F)cc(C(N)=O)c3[nH]c4c(c23)CC2(CCOC2)CC4)C1] | (400 MHz, DMSO-d₆) δ 10.93 (s, 1H), 8.08 (s, 2H), 7.55 (d, J = 10.7 Hz, 1H), 7.48 (d, J = 7.4 Hz, 2H), 7.30 (dd, J = 12.1, 6.1 Hz, 2H), 7.12 (d, J = 8.7 Hz, 1H), 6.79 (dd, J = 16.7, 10.6 Hz, 1H), 6.14 (d, J = 16.6 Hz, 1H), 5.64-5.73 (m, 2H), 4.89 (dd, J = 29.3, 12.2 Hz, 2H), 4.66 (d, J = 17.0 Hz, 1H), 3.78-3.86 (m, 1H), 3.62-3.73 (m, 2H), 3.53 (d, J = 8.3 Hz, 1H), 3.41-3.48 (m, 1H), 3.07 (d, J = 8.2 Hz, 1H), 2.77 (s, 2H), 2.33 (d, J = 1.8 Hz, 2H), 1.74-1.84 (m, 2H), 1.63 (dd, J = 13.5, 6.7 Hz, 3H). |
| 7-18 | 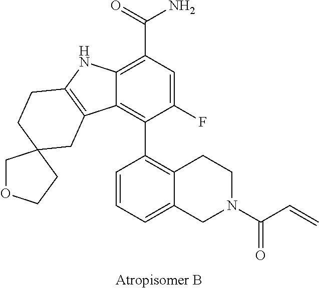 [SMILES: C=CC(=O)N1CCc2c(cccc2-c2c(F)cc(C(N)=O)c3[nH]c4c(c23)CC2(CCOC2)CC4)C1] | (400 MHz, DMSO-d₆) δ 10.93 (s, 1H), 8.07 (d, J = 13.7 Hz, 1H), 7.55 (d, J = 10.7 Hz, 1H), 7.49 (s, 1H), 7.32 (dd, J = 11.4, 7.0 Hz, 2H), 7.12 (d, J = 8.6 Hz, 1H), 6.78 (dd, J = 16.6, 10.5 Hz, 1H), 6.13 (d, J = 16.6 Hz, 1H), 5.70 (dd, J = 24.7, 10.3 Hz, 1H), 4.81 (d, J = 42.0 Hz, 2H), 3.61- 3.77 (m, 3H), 3.45-3.57(m, 2H), 3.04-3.11 (m, 1H), 2.78 (t, J = 12.3 Hz, 2H), 2.31-2.40 (m, 2H), 1.79 (d, J = 15.6 Hz, 1H), 1.57-1.72 (m, 4H). |
| 7-19 | 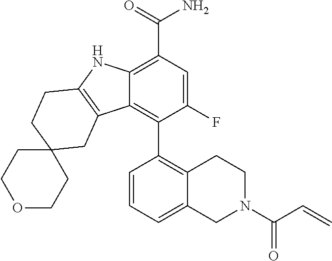 [SMILES: C=CC(=O)N1CCc2c(cccc2-c2c(F)cc(C(N)=O)c3[nH]c4c(c23)CC2(CCOCC2)CC4)C1] | (400 MHz, DMSO-d₆) δ 10.88 (s, 1H), 8.09 (s, 1H), 7.55 (d, J = 10.7 Hz, 1H), 7.49 (s, 1H), 7.33 (t, J = 6.8 Hz, 2H), 7.11-7.16 (m, 1H), 6.79 (dd, J = 16.5, 10.4 Hz, 1H), 6.14 (d, J = 16.8 Hz, 1H), 5.70 (dd, J = 19.3, 11.1 Hz, 1H), 4.82- 4.96 (m, 2H), 4.69 (d, J = 17.2 Hz, 1H), 3.71 (d, J = 5.5 Hz, 2H), 3.11 (d, J = 10.9 Hz, 2H), 2.70 (s, 2H), 2.33 (s, 3H), 1.73 (d, J = 16.0 Hz, 2H), 1.57 (d, J = 15.6 Hz, 2H), 1.27 (d, J = 8.3 Hz, 2H), 1.08 (s, 2H). |
| 7-20 | 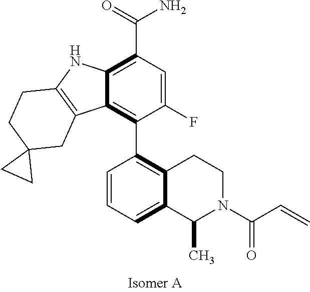 | (400 MHz, DMSO-d₆) δ: 10.9 (s, 1H), 8.01 (br s, 1H), 7.55 (d, J = 8.0 Hz, 1H), 7.34-7.24 (m, 2H), 7.12 (m, 1H), 6.76-6.98 (m, 1H), 6.71 (d, J = 7.7 Hz, 1H), 6.13 (dd, J = 16.6, 2.4 Hz, 1H), 5.60- 5.80 (m, 2H), 4.12 (m, 1H), 3.14-3.02 (m, 2H), 2.77 (br t, J = 5.8 Hz, 2H), 2.23-2.41 (m, 2H), 1.67-1.82 (m, 1H), 1.31-1.62 (m, 6H), 0.26 (br s, 2H), −0.10-0.04 (m, 2H). |
| 7-21 | 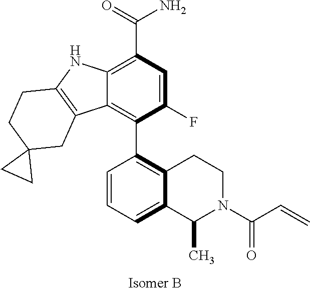 | (400 MHz, DMSO-d₆) δ: 10.9 (s, 1H), 8.01 (br s, 1H), 7.55 (d, J = 8.0 Hz, 1H), 7.34-7.24 (m, 2H), 7.12 (m, 1H), 6.76-6.98 (m, 1H), 6.71 (d, J = 7.7 Hz, 1H), 6.13 (dd, J = 16.6, 2.4 Hz, 1H), 5.60- 5.80 (m, 2H), 4.12 (m, 1H), 3.14-3.02 (m, 2H), 2.77 (br t, J = 5.8 Hz, 2H), 2.23-2.41 (m, 2H), 1.67-1.82 (m, 1H), 1.31-1.62 (m, 6H), 0.26 (br s, 2H), −0.10-0.04 (m, 2H). |
| 7-22 | 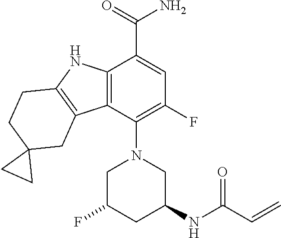 [SMILES: C=CC(=O)N[C@H]1C[C@H](F)CN(c2c(F)cc(C(N)=O)c3[nH]c4c(c23)CC2(CC4)CC2)C1] | (400 MHz, DMSO-d₆) δ 10.73 (s, 1H), 8.06 (d, J = 8.0 Hz, 1H), 7.95 (s, 1H), 7.35-7.25 (m, 2H), 6.17-6.00 (m, 2H), 5.59 (d, J = 7.6 Hz, 1H), 5.00 (m, 1H), 4.15 (m, 1H), 3.15-3.08 (m, 3H), 3.00-2.75 (m, 5H), 2.11 (m, 1H), 1.71-1.54 (m, 3H), 0.42-0.41 (m, 2H), 0.28-0.27 (m, 2H). |
| 7-23 | 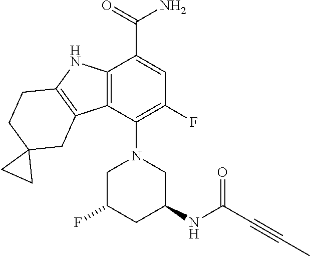 [SMILES: CC#CC(=O)N[C@H]1C[C@H](F)CN(c2c(F)cc(C(N)=O)c3[nH]c4c(c23)CC2(CC4)CC2)C1] | (400 MHz, DMSO-d₆) δ 10.73 (s, 1H), 8.49 (d, J = 8.0 Hz, 1H), 7.95 (s, 1H), 7.44-7.38 (m, 2H), 4.99 (m, 1H), 4.21 (m, 1H), 3.29-3.12 (m, 2H), 3.00-2.75 (m, 6H), 2.11 (m, 1H), 1.94 (s, 3H), 1.71-1.54 (m, 3H), 0.38-0.29 (m, 4H). |
| 7-24 | 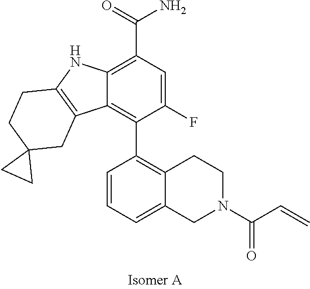 [SMILES: C=CC(=O)N1CCc2c(cccc2-c2c(F)cc(C(N)=O)c3[nH]c4c(c23)CC2(CC4)CC2)C1] | (300 MHz, DMSO-d₆) δ 7.65 (d, J = 8.0 Hz, 1H), 7.34-7.24 (m, 2H), 7.18-7.10 (m, 1H), 6.88-6.75 (m, 2H), 6.22 (dd, J = 16.8, 2.1 Hz, 1H), 5.80- 5.60 (m, 1H), 4.82-4.78 (m, 2H), 3.79-3.65 (m, 2H), 2.80 (t, J = 5.8 Hz, 2H), 2.43-2.41 (m, 2H), 1.87-1.52 (m, 4H), 0.26 (m, 2H), 0.11-0.05 (m, 2H). |
| 7-25 | 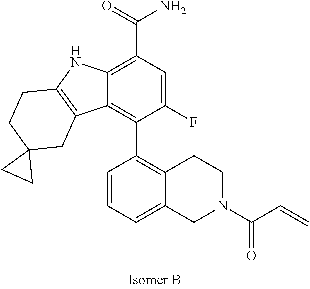 [SMILES: C=CC(=O)N1CCc2c(cccc2-c2c(F)cc(C(N)=O)c3[nH]c4c(c23)CC2(CC4)CC2)C1] | (300 MHz, DMSO-d₆) δ 7.65 (d, J = 8.0 Hz, 1H), 7.34-7.24 (m, 2H), 7.18-7.10 (m, 1H), 6.88-6.75 (m, 2H), 6.22 (dd, J = 16.8, 2.1 Hz, 1H), 5.80- 5.60 (m, 1H), 4.82-4.78 (m, 2H), 3.79-3.65 (m, 2H), 2.80 (t, J = 5.8 Hz, 2H), 2.43-2.41 (m, 2H), 1.87-1.52 (m, 4H), 0.26 (m, 2H), 0.11-0.05 (m, 2H). |

According to similar procedures, Compounds 7-26 through 7-29, as shown in Table 9, of Formula (I-C-iii) were prepared.

TABLE 9
COMPOUNDS 7-26 THROUGH 7-29

| Compound | | |
|---|---|---|
| No. | Structure/Name | ¹H NMR |
| 7-26 | 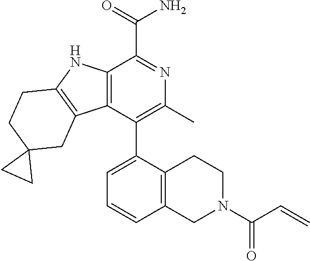 [SMILES: C=CC(=O)N1CCc2c(cccc2-c2c(C)nc(C(N)=O)c3[nH]c4c(c23)CC2(CC4)CC2)C1] | (400 MHz, DMSO-d₆) δ 11.08 (s, 1H), 8.06 (s, 1H), 7.60 (s, 1H), 7.38-7.22 (m, 2H), 7.12-7.00 (m, 1H), 6.99-6.69 (m, 1H), 6.13 (dd, J = 2.4, 16.6 Hz, 1H), 5.78-5.61 (m, 1H), 4.96-4.60 (m, 2H), 3.79-3.45 (m, 2H), 2.79 (t, J = 6.2 Hz, 2H), 2.32-2.12 (m, 5H), 1.71-1.27 (m, 4H), 0.36-0.17 (m, 2H), −0.02-0.14 (m, 2H). |
| 7-27 | 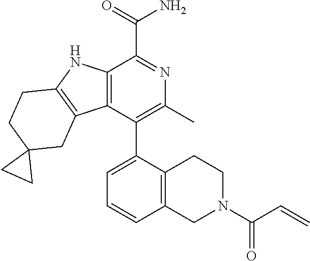 [SMILES: C=CC(=O)N1CCc2c(cccc2-c2c(C)nc(C(N)=O)c3[nH]c4c(c23)CC2(CC4)CC2)C1] | (400 MHz, DMSO-d₆) δ 11.08 (s, 1H), 8.04 (s, 1H), 7.60 (s, 1H), 7.38-7.22 (m, 2H), 7.12-7.00 (m, 1H), 6.99-6.69 (m, 1H), 6.13 (dd, J = 2.4, 16.6 Hz, 1H), 5.78-5.61 (m, 1H), 4.96-4.60 (m, 2H), 3.79-3.45 (m, 2H), 2.79 (t, J = 6.2 Hz, 2H), 2.32-2.12 (m, 5H), 1.71-1.27 (m, 4H), 0.36-0.17 (m, 2H), −0.02-0.14 (m, 2H). |
| 7-28 | 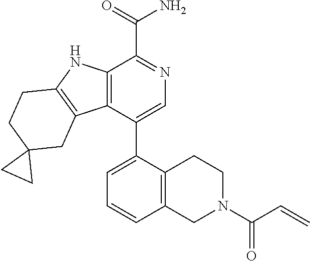 [SMILES: C=CC(=O)N1CCc2c(cccc2-c2cnc(C(N)=O)c3[nH]c4c(c23)CC2(CC4)CC2)C1] | (400 MHz, DMSO-d₆) δ 11.35 (s, 1H), 8.17 (s, 1H), 7.84 (s, 1H), 7.61 (s, 1H), 7.37-7.25 (m, 2H), 7.18- 7.09 (m, 1H), 6.98-6.71 (m, 1H), 6.14 (dd, J = 2.5, 16.6 Hz, 1H), 5.77-5.62 (m, 1H), 4.95-4.60 (m, 2H), 3.78-3.46 (m, 2H), 2.89-2.79 (m, 2H), 2.43-2.24 (m, 2H), 1.83 (d, J = 15.8 Hz, 1H), 1.64-1.35 (m, 3H), 0.40-0.20 (m, 2H), 0.12-−0.09 (m, 2H). |
| 7-29 | 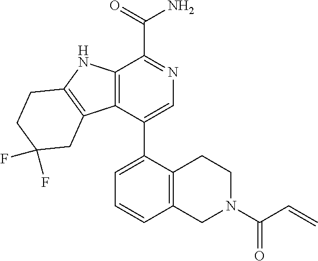 [SMILES: C=CC(=O)N1CCc2c(cccc2-c2cnc(C(N)=O)c3[nH]c4c(c23)CC(F)(F)CC4)C1] | (400 MHz, DMSO-d₆) δ 11.63 (s, 1H), 8.24 (s, 1H), 7.93 (s, 1H), 7.70 (s, 1H), 7.42-7.30 (m, 2H), 7.22- 7.12 (m, 1H), 6.99-6.71 (m, 1H), 6.13 (dd, J = 16.8, 2.0 Hz, 1H), 5.78-5.62 (m, 1H), 4.94-4.68 (m, 2H), 3.80-3.49 (m, 2H), 3.10-2.90 (m, 2H), 2.55-2.23 (m, 6H). |
| 7-30 | 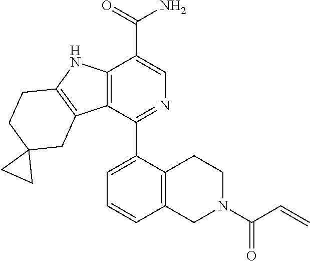 [SMILES: C=CC(=O)N1CCc2c(cccc2-c2ncc(C(N)=O)c3[nH]c4c(c23)CC2(CC4)CC2)C1] | ¹H NMR (400 MHz, DMSO-d₆) δ 11.32 (s, 1H), 8.64 (s, 1H), 8.15 (s, 1H), 7.50 (s, 1H), 7.34-7.18 (m, 2H), 7.12-7.02 (m, 1H), 6.95-6.71 (m, 1H), 6.12 (dd, J = 16.7, 2.5 Hz, 1H), 5.78-5.57 (m, 1H), 4.91-4.63 (m, 2H), 3.73-3.50 (m, 2H), 2.83-2.71 (m, 2H), 2.49- 2.38 (m, 1H), 2.34-2.15 (m, 1H), 1.92 (d, J = 16.1 Hz, 1H), 1.67-1.29 (m, 3H), 0.38-0.17 (m, 2H), 0.09-−0.10 (m, 2H). |

### Example 8-1

### Preparation of 9-(1-acryloyl-1,2,5,6-tetrahydropyridin-3-yl)-1,3,4,5-tetrahydrothiopyrano-[4,3-b]indole-6-carboxamide (Compound 8-1)

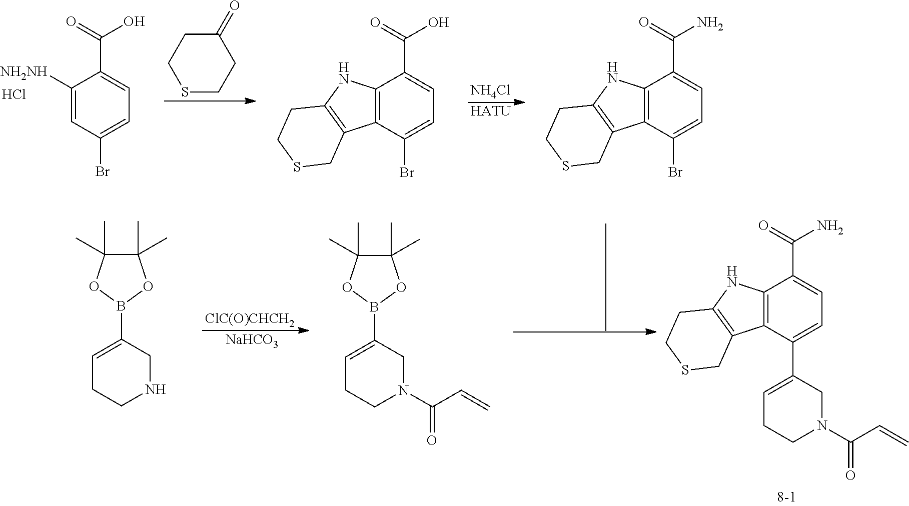

### Step 1: 1-[5-(4,4,5,5-tetramethyl-1,3,2-dioxaborolan-2-yl)-3,6-dihydro-2H-pyridin-1-yl]prop-2-en-1-one

To a solution of 5-(4,4,5,5-tetramethyl-1,3,2-dioxaborolan-2-yl)-1,2,3,6-tetrahydropyridine (2.0 g, 9.57 mmol) in THF (20 mL):Water (5 mL) at 0° C. was added NaHCO₃ (3.21 g, 38.26 mmol, 1.49 mL), mixture was stirred for 5 min. To this was added prop-2-enoyl chloride (952.30 mg, 10.52 mmol, 857.93 μL) and the reaction was allowed to continue at 0° C. for 15 min. The reaction mixture was diluted with water and extracted with ethyl acetate. The organic layer was washed with sat NaHCO₃(aq), brine, dried over Na₂SO₄ and concentrated under reduced pressure to give 1-[5-(4,4,5,5-tetramethyl-1,3,2-dioxaborolan-2-yl)-3,6-dihydro-2H-pyridin-1-yl]prop-2-en-1-one (700 mg) as a white solid.

### Step 2: 5-bromo-1,2,4,9-tetrahydrospiro[carbazole-3,1′-cyclopropane]-8-carboxylic acid

ZnCl₂ (1.50 eq) in IPA (20 mL) or DME (30 mL) was added to a suspension of 4-bromo-2-hydrazineylbenzoic acid hydrochloride (500 mg, 1.00 eq) and thiopyran (1.05 eq), and the mixture stirred at 90° C. under N₂ for 7 days. Solvents were removed by evaporation to provide an oil that was purified by prep-HPLC. ¹H NMR (400 MHz, DMSO-d⁶) d 13.1 (s, 1H), 11.1 (s, 1H), 7.51 (d, J=8.4 Hz, 2H), 7.21 (d, J=8.4 Hz, 2H), 4.13 (s, 2H), 3.00 (m, 2H), 2.90 (m, 2H).

### Step 3: 5-bromo-1,2,4,9-tetrahydrospiro[carbazole-3,1′-cyclopropane]-8-carboxylic acid

To a mixture of 9-bromo-1,3,4,5-tetrahydrothiopyrano[4,3-b]indole-6-carboxamide (200 mg, 640.65 μmol), ammonium chloride (137.08 mg, 2.56 mmol) in DMA (2 mL) was added HATU (269.38 mg, 708.46 μmol) and the mixture was allowed to continue stirring at RT for 30 min. The mixture was diluted with EtOAc and water, the organic layer was separated and the aqueous layer was extracted with EtOAc. The combined organic layer was washed with brine, dried over Na₂SO₄, filtered and concentrated. The residue obtained was purified by silica gel chromatography using a gradient of 5%-60% EtOAc in heptane to give 9-bromo-1,3,4,5-tetrahydrothiopyrano[4,3-b]indole-6-carboxamide as an off-white solid which was used in the next step without further purification.

### Step 4: 9-(1-prop-2-enoyl-3,6-dihydro-2H-pyridin-5-yl)-1,3,4,5-tetrahydrothiopyrano[4,3-b]indole-6-carboxamide (Compound 8-1)

To a solution of 9-bromo-1,3,4,5-tetrahydrothiopyrano[4,3-b]indole-6-carboxamide (100 mg, 321.34 μmol) in THF (1 mL), methanol (0.5 mL) and water (0.5 mL), 1-[5-(4,4,5,5-tetramethyl-1,3,2-dioxaborolan-2-yl)-3,6-dihydro-2H-pyridin-1-yl]prop-2-en-1-one (101.47 mg, 385.61 μmol), PdCl₂(dppf) (66.2 mg, 0.091 mmol) and Na₂CO₃ (144 mg, 1.358 mmol) were added. The reaction mixture was heated at 70° C. for 16 h. The mixture was cooled to rt and diluted with EtOAc and water. The organic layer was separated, and the aqueous layer was extracted with EtOAc twice more. The combined organic layer was washed with brine, dried over Na₂SO₄, filtered and concentrated under reduced pressure. The residue obtained was purified by reverse phase chromatography using a gradient of 10%-95% MeCN in water w/0.10% Formic Acid. The like fractions were combined and freeze dried on the lyophilizer to give as a 9-(1-prop-2-enoyl-3,6-dihydro-2H-pyridin-5-yl)-1,3,4,5-tetrahydrothiopyrano[4,3-b]indole-6-carboxamide (35.9 mg) as an off-white solid. ¹H NMR (DMSO-d₆) δ: 10.95 (s, 1H), 8.00 (br s, 1H), 7.58 (d, J=7.7 Hz, 1H), 7.33 (br s, 1H), 6.73-6.99 (m, 2H), 6.04-6.23 (m, 1H), 5.60-5.82 (m, 2H), 4.22-4.38 (m, 2H), 3.64-3.84 (m, 4H), 3.02 (br d, J=5.0 Hz, 2H), 2.81-2.96 (m, 2H), 2.31 (br d, J=14.5 Hz, 2H).

### Example 9

### Preparation of 9-(2-acryloyl-1,2,3,4-tetrahydroisoquinolin-5-yl)-8-fluoro-1,3,4,5-tetrahydrothiopyrano[4,3-b]indole-6-carboxamide 2-oxide (Compound 9-1 and 9-2)

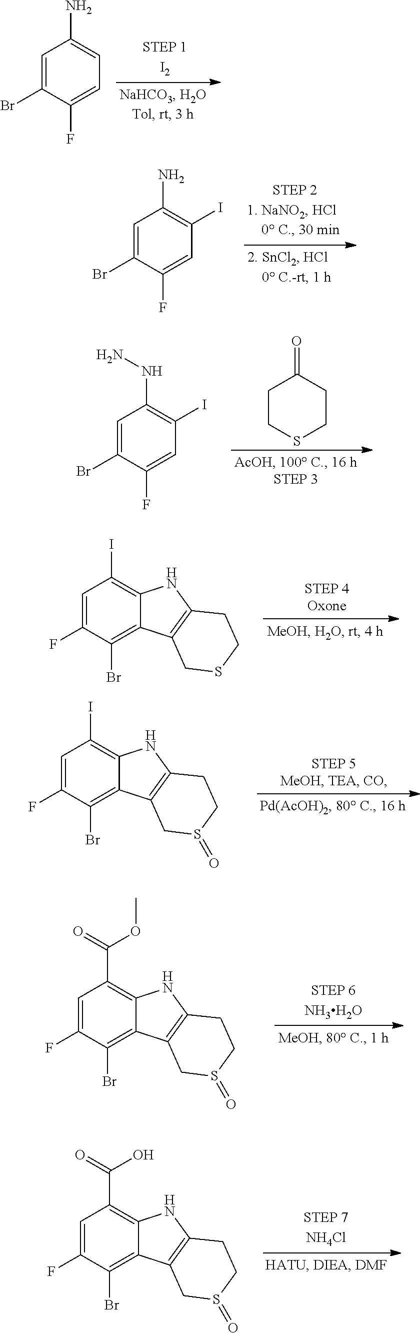

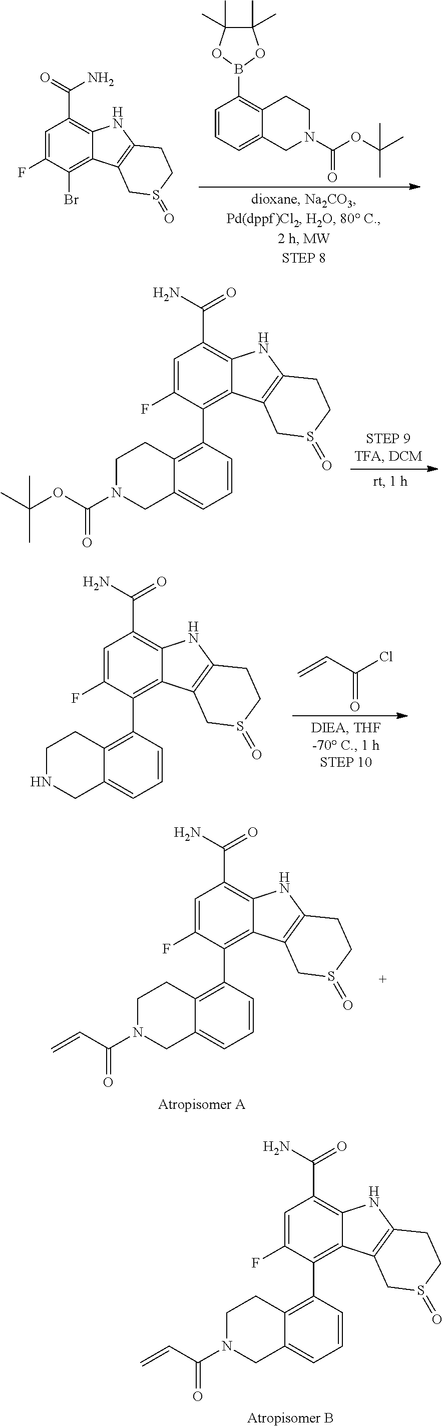

### Step 1: 5-bromo-4-fluoro-2-iodoaniline

To a solution of 3-bromo-4-fluoroaniline (50 g, 0.26 mol) in toluene (500 mL) and H₂O (100 mL) was added 12 (66 g, 0.26 mol) and NaHCO₃ (43 g, 0.52 mmol), the reaction mixture was stirred at rt for 16 h. LCMS found the desired product. The mixture was diluted with water (200 mL), extracted with EA (3×300 mL). The combined organic layer was washed with brine, dried over Na₂SO₄, filtered and concentrated. The residue was purified by silica chromatography (PE/EA=20:1) to afford the desired product 5-bromo-4-fluoro-2-iodoaniline (20 g) as a red solid.

¹H NMR (CDCl₃) δ 7.39 (d, J=7.6 Hz, 2H), 6.90 (d, J=7.6 Hz, 2H), 3.98 (bs, 2H).

### Step 2: (5-bromo-4-fluoro-2-iodophenyl)hydrazine

To a solution of 5-bromo-4-fluoro-2-iodoaniline (20 g, 0.06 mol) in AcOH (50 mL) was added slowly conc. HCl (200 mL), the reaction mixture was cooled to 0° C. and treated slowly with a solution NaNO₂ (4.55 g, 0.066 mol) in H₂O (50 mL). The reaction mixture was stirred at rt for 1 h, then a solution of SnCl₂.2H₂O (28 g, 0.126 mol) in conc. HCl (50 mL) was added slowly. The reaction mixture came to warm to rt and stirred for 2 h. LCMS showed the reaction was completed. The suspension was filtered, washed with water and dried under vacuum to give the desired product (12 g) as a yellow solid. ¹H NMR (DMSO-d₆) δ 10.3 (bs, 2H), 7.85 (d, J=7.6 Hz, 2H), 7.76 (bs, 1H), 7.31 (d, J=7.6 Hz, 2H).

### Step 3: 9-bromo-8-fluoro-6-iodo-1,3,4,5-tetrahydrothiopyrano[4,3-b]indole

To a solution of (5-bromo-4-fluoro-2-iodophenyl)hydrazine (1.00 g, 3.02 mmol) in AcOH (50 mL) was added tetrahydro-4H-thiopyran-4-one (350 mg, 3.02 mmol), the reaction mixture was stirred at 100° C. for 16 h. LCMS found the desired product. The mixture was concentrated and the residue was purified by silica chromatography (PE/EA=5:1) to afford the desire product 9-bromo-8-fluoro-6-iodo-1,3,4,5-tetrahydrothiopyrano[4,3-b]indole (900 mg) as a red solid. HRMS (ESI) calculated for C₁₁H₈BrFINS⁺ [M+H]⁺ m/z 410.86, found 411.8.

### Step 4: 9-bromo-8-fluoro-6-iodo-1,3,4,5-tetrahydrothiopyrano[4,3-b]indole 2-oxide

To a solution of 9-bromo-8-fluoro-6-iodo-1,3,4,5-tetrahydrothiopyrano[4,3-b]indole (300 mg, 0.73 mmol) in MeOH (30 mL) was added Oxone (448 mg, 0.73 mmol) in H₂O (6 mL), the reaction mixture was stirred at rt for 4 h. LCMS showed the reaction was completed. The mixture was diluted with water (10 mL), extracted with EA (3×50 mL). The combined organic layer was washed with brine, dried over Na₂SO₄, filtered and concentrated to afford the desire product 9-bromo-8-fluoro-6-iodo-1,3,4,5-tetrahydrothiopyrano[4,3-b]indole 2-oxide (260 mg, crude) as a white solid. HRMS (ESI) calculated for C₁₁H₈BrFINOS⁺ [M+H]⁺ m/z 426.85, found 427.9.

### Step 5: methyl 9-bromo-8-fluoro-1,3,4,5-tetrahydrothiopyrano[4,3-b]indole-6-carboxylate 2-oxide

To a 100 mL round bottom flask equipped with a stirring bar was added 9-bromo-8-fluoro-6-iodo-1,3,4,5-tetrahydrothiopyrano[4,3-b]indole 2-oxide (260 mg, 0.607 mmol), MeOH (10 mL), TEA (61 mg, 0.61 mmol) and Pd(OAc)₂ (100 mg). The flask was well evaluated and refilled with CO (3 times) and equipped with a CO balloon. The reaction mixture was stirred at 80° C. for 16 h. LCMS showed the reaction was completed. The mixture was filtered, the filtrate was concentrated. The residue was purified by silica chromatography (DCM/MeOH=10:1) to afford the desired product methyl 9-bromo-8-fluoro-1,3,4,5-tetrahydrothiopyrano[4,3-b]indole-6-carboxylate 2-oxide (130 mg, 49% two step yield) as a white solid. HRMS (ESI) calculated for C₁₃H₁₁BrFNO₃S⁺ [M+H]⁺ m/z 358.96, found 359.9.

### Step 6: 9-bromo-8-fluoro-1,3,4,5-tetrahydrothiopyrano[4,3-b]indole-6-carboxylic acid 2-oxide

To a solution of methyl 9-bromo-8-fluoro-1,3,4,5-tetrahydrothiopyrano[4,3-b]indole-6-carboxylate 2-oxide (130 mg, 0.36 mmol) in MeOH (2 mL) was added ammonium hydroxide (2 mL), the reaction mixture was stirred at 80° C. for 16 h. LCMS showed the reaction was completed. The mixture was concentrated to give the desired product 9-bromo-8-fluoro-1,3,4,5-tetrahydrothiopyrano[4,3-b]indole-6-carboxylic acid 2-oxide (120 mg, crude) as a white solid. HRMS (ESI) calculated for C₁₂H₉BrFNO₃S⁺ [M+H]⁺ m/z 344.95, found 345.9.

### Step 7: 9-bromo-8-fluoro-1,3,4,5-tetrahydrothiopyrano[4,3-b]indole-6-carboxamide 2-oxide

To a solution of 9-bromo-8-fluoro-1,3,4,5-tetrahydrothiopyrano[4,3-b]indole-6-carboxylic acid 2-oxide (120 mg, 0.35 mmol) in DMF (2 mL) was added ammonium chloride (56.16 mg, 1.05 mmol) and DIEA (135 mg, 1.05 mmol), the reaction mixture was stirred at rt for 1 h. LCMS showed the reaction was completed. The mixture was diluted with water (10 mL), extracted with EA (3×10 mL). The combined organic layer was washed with brine, dried over Na₂SO₄, filtered and concentrated. The residue was purified by prep-TLC (DCM/MeOH=10:1) to give the desired product 9-bromo-8-fluoro-1,3,4,5-tetrahydrothiopyrano[4,3-b]indole-6-carboxamide 2-oxide (30 mg, 25% two step yield) as a colorless oil. HRMS (ESI) calculated for C₁₂H₁₀BrFN₂O₂S⁺ [M+H]⁺ m/z 343.96, found 345.0.

### Step 8: tert-butyl 5-(6-carbamoyl-8-fluoro-2-oxido-1,3,4,5-tetrahydrothiopyrano[4,3-b]indol-9-yl)-3,4-dihydroisoquinoline-2(1H)-carboxylate

To a 10 mL microwave tube equipped with a stirring bar was added 9-bromo-8-fluoro-1,3,4,5-tetrahydrothiopyrano[4,3-b]indole-6-carboxamide 2-oxide (20 mg, 0.06 mmol), tert-butyl 5-(4,4,5,5-tetramethyl-1,3,2-dioxaborolan-2-yl)-3,4-dihydroisoquinoline-2(1H)-carboxylate 10 (41 mg, 0.115 mmol), Pd(dppf)Cl₂ (5 mg, 0.0057 mmol), sodium carbonate (18 mg, 0.174 mmol) followed up with dioxane (2 mL). The tube was evacuated, refilled with N₂ and capped, and then heated to 80° C. under microwave with stirring for 2 h. Then the reaction mixture was filtered, the residue was washed with MeOH (5 mL). The combined filtrate was concentrated. The residue was purified by prep-TLC (DCM/MeOH=10:1) to afford the tert-butyl 5-(6-carbamoyl-8-fluoro-2-oxido-1,3,4,5-tetrahydrothiopyrano[4,3-b]indol-9-yl)-3,4-dihydroisoquinoline-2(1H)-carboxylate (25 mg, 86% yield) as a colorless oil. HRMS (ESI) calculated for C₂₆H₂₈FN₃O₄S⁺ [M-Boc+H]⁺ m/z 497.18, found 398.2.

### Step 9: 8-fluoro-9-(1,2,3,4-tetrahydroisoquinolin-5-yl)-1,3,4,5-tetrahydrothiopyrano[4,3-b]indole-6-carboxamide 2-oxide

To a solution of tert-butyl 5-(6-carbamoyl-8-fluoro-2-oxido-1,3,4,5-tetrahydrothiopyrano[4,3-b]indol-9-yl)-3,4-dihydroisoquinoline-2(1H)-carboxylate (20 mg, 0.04 mmol) in DCM (2 mL) was added TFA (0.5 mL), the reaction mixture was stirred at rt for 1 h. LCMS showed the reaction was completed. The reaction mixture was concentrated to give the crude product 8-fluoro-9-(1,2,3,4-tetrahydroisoquinolin-5-yl)-1,3,4,5-tetrahydrothiopyrano[4,3-b]indole-6-carboxamide-2-oxide (16 mg, crude) as a red oil. HRMS (ESI) calculated for C₂₁H₂₀FN₃O₂S⁺ [M+H]⁺ m/z 397.13, found 398.2.

### Step 10: 9-(2-acryloyl-1,2,3,4-tetrahydroisoquinolin-5-yl)-8-fluoro-1,3,4,5-tetrahydrothiopyrano[4,3-b]indole-6-carboxamide 2-oxide (Compounds 9-1 and 9-2)

To a solution of 8-fluoro-9-(1,2,3,4-tetrahydroisoquinolin-5-yl)-1,3,4,5-tetrahydrothiopyrano[4,3-b]indole-6-carboxamide 2-oxide (16 mg, crude) in THF (3 mL) was added DIEA (26 mg, 0.2 mmol), then acryloyl chloride (4 mg, 0.04 mmol) was added slowly at −70° C., the reaction mixture was stirred at −70° C. for 1 h. LCMS showed the reaction was completed. The reaction mixture was concentrated at room temperature carefully. The residue was purified by prep-HPLC (mobile phase: 0.1% FA/ACN/H₂O) to give the desired products (peak A; 1.8 mg, 10% two step yield) as a white solid and (peak B; 1.3 mg, 7% two step yield) as a white solid.

Peak A:

¹H NMR (400 MHz, DMSO) δ 11.29 (s, 1H), 8.13 (s, 1H), 7.62 (d, J=10.4 Hz, 1H), 7.55 (s, 1H), 7.36-7.34 (m, 2H), 7.14-7.12 (m, 1H), 6.80-6.74 (m, 1H), 6.14-6.10 (m, 1H), 5.73-5.65 (m, 1H), 4.88-4.69 (m, 2H), 3.70 (br s, 2H), 3.23-3.14 (m, 4H), 2.99-2.90 (m, 2H), 2.88-2.31 (m, 2H).

HRMS (ESI) calculated for C₂₄H₃₂N₆O_{2S}⁺ [M+H]⁺ m/z 451.14, found 452.1.

Peak B:

¹H NMR (400 MHz, DMSO) δ 11.28 (s, 1H), 8.12 (s, 1H), 7.61 (d, J=10.8 Hz, 1H), 7.54 (s, 1H), 7.36-7.30 (m, 2H), 7.13-7.10 (m, 1H), 6.79-6.72 (m, 1H), 6.13-6.08 (m, 1H), 5.72-5.62 (m, 1H), 4.87-4.69 (m, 2H), 3.81-3.60 (m, 2H), 3.17-3.11 (m, 3H), 2.94 (br s, 1H), 2.42-2.30 (m, 2H).

HRMS (ESI) calculated for C₂₄H₃₂N₆O₂S⁺ [M+H]⁺ m/z 451.14, found 452.1.

Compounds 9-3 through 9-8, shown in Table 10, were prepared in a manner similar to that described for Example 9.

TABLE 10
COMPOUNDS 9-3 THROUGH 9-8

| Compound | | |
|---|---|---|
| No. | Structure | ¹H NMR |
| 9-3 | 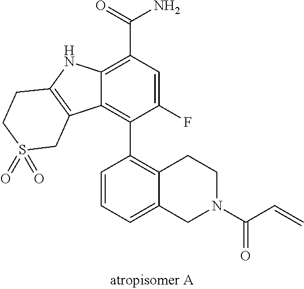 [SMILES: C=CC(=O)N1CCc2c(cccc2-c2c(F)cc(C(N)=O)c3[nH]c4c(c23)CS(=O)(=O)CC4)C1] | (400 MHz, DMSO) δ 11.44 (s, 1H), 8.18 (s, 1H), 7.67 (d, J = 10.7 Hz, 1H), 7.61 (s, 1H), 7.44-7.33 (m, 2H), 7.14 (d, J = 8.4 Hz, 1H), 7.00-6.73 (m, 1H), 6.14 (d, J = 16.8 Hz, 1H), 5.78-5.58 (m, 1H), 4.97-4.68 (m, 2H), 3.83-3.54 (m, 2H), 3.41- 3.37 (m, 2H), 3.32-3.24 (m, 4H), 2.44-2.29 (m, 2H). |
| 9-4 | 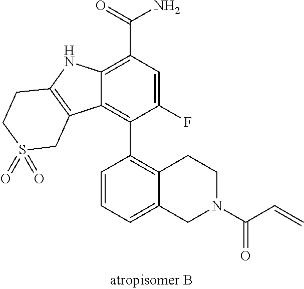 [SMILES: C=CC(=O)N1CCc2c(cccc2-c2c(F)cc(C(N)=O)c3[nH]c4c(c23)CS(=O)(=O)CC4)C1] | (400 MHz, DMSO) δ 11.44 (s, 1H), 8.18 (s, 1H), 7.67 (d, J = 10.7 Hz, 1H), 7.61 (s, 1H), 7.44-7.33 (m, 2H), 7.14 (d, J = 8.4 Hz, 1H), 7.00-6.73 (m, 1H), 6.14 (d, J = 16.8 Hz, 1H), 5.78-5.58 (m, 1H), 4.97-4.68 (m, 2H), 3.83-3.54 (m, 2H), 3.41- 3.37 (m, 2H), 3.32-3.24 (m, 4H), 2.44-2.29 (m, 2H). |
| 9-5 | 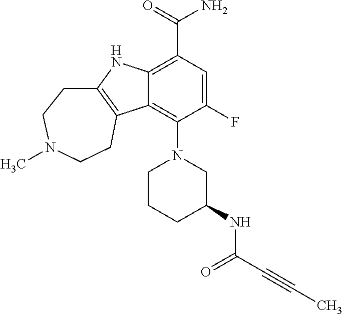 [SMILES: CC#CC(=O)N[C@H]1CCCN(c2c(F)cc(C(N)=O)c3[nH]c4c(c23)CCN(C)CC4)C1] | (400 MHz, CD₃OD) δ 7.33 (d, J = 13.9 Hz, 1H), 4.07-3.98 (m, 1H), 3.65-3.60 (m, 2H), 3.45- 3.41 (m, 2H), 3.12-3.08 (m, 4H), 3.04-2.98 (m, 2H), 2.97-2.84 (m, 4H), 2.61 (s, 3H), 2.07-1.98 (m, 2H), 1.95 (s, 3H), 1.85-1.78 (m, 2H). |
| 9-6 | 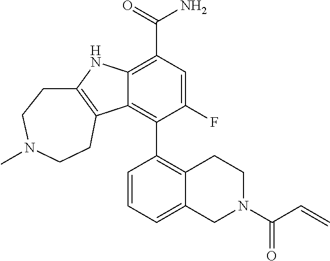 [SMILES: C=CC(=O)N1CCc2c(cccc2-c2c(F)cc(C(N)=O)c3[nH]c4c(c23)CCN(C)CC4)C1] | (400 MHz, CD₃OD) δ 7.45 (d, J = 10.3 Hz, 1H), 7.41-7.26 (m, 2H), 7.22-7.12 (m, 1H), 6.86- 6.74 (m, 1H), 6.27-6.22 (m, 1H), 5.81-5.73 (m, 1H), 4.81 (d, J = 17.0 Hz, 2H), 3.78-3.75 (m, 2H), 3.11-3.00 (m, 2H), 2.84-2.71 (m, 2H), 2.60- 2.40 (m, 4H), 2.38 (s, 3H), 2.22-2.04 (m, 2H). ¹⁹F NMR (376.5 MHz, DMSO) δ −130.2. |
| 9-7 | 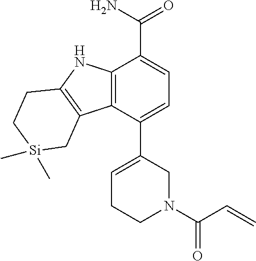 [SMILES: C=CC(=O)N1CCC=C(c2ccc(C(N)=O)c3[nH]c4c(c23)C[Si](C)(C)CC4)C1] | (400 MHz, DMSO-d₆) δ 10.63 (s, 1H), 8.02 (s, 1H), 7.47-7.44 (m, 2H), 6.91-6.81 (m, 1H), 6.80-6.74 (m, 1H), 6.13 (d, J = 16.5 Hz, 1H), 5.77-5.65 (m, 2H), 4.23-4.19 (m, 2H), 3.84-3.61 (m, 2H), 3.00-2.90 (m, 2H), 2.41-1.95 (m, 4H), 1.85-1.26 (m, 2H), 0.83 (t, J = 7.6 Hz, 3H), 0.05 (s, 3H), 0.03 (s, 3H). ¹⁹F NMR (376.5 MHz, DMSO) δ −131.0 |

### Example 10-1 & 10-2

### Preparation of: (R)-8-(1-acryloylpiperidin-3-yl)-7-fluoro-1,2,3,4-tetrahydrocyclopenta[b]-indole-5-carboxamide (Compound 10-1); and (S)-8-(1-acryloylpiperidin-3-yl)-7-fluoro-1,2,3,4-tetrahydrocyclo-penta[b]indole-5-carboxamide (Compound 10-2)

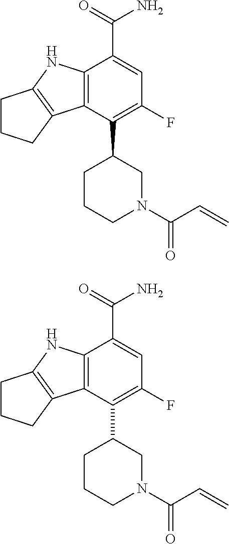

### Step 1. (5-bromo-4-fluoro-2-iodo-phenyl)hydrazine

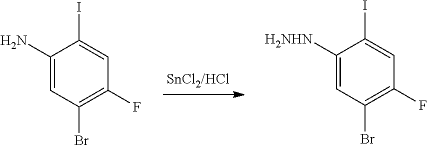

An aqueous solution of sodium nitrite (3M, 129 mL) was added dropwise to a cold suspension of 5-bromo-4-fluoro-2-iodoaniline (102 g, 323 mmol) in 37% aqueous HCl (500 mL) which was stirred at −10° C. on a NaCl ice bath, at such rate that the temperature did not exceed 0° C. The resulting suspension was stirred at 0° C. for 1 h then was treated with a solution of SnCl₂.H₂O (7M, 138 mL) in 37% aqueous HCl. The temperature increased to 10° C. and the mixture was stirred in the ice bath for 1 h. The resulting precipitate was collected by vacuum filtration, washed with concentrated HCl (3×100 mL) and EtOAc (3×500 mL) then washed with EtOAc until filtrate wash was colorless. The obtained solid was dried overnight under reduced pressure to give (5-bromo-4-fluoro-2-iodo-phenyl)hydrazine (96.8 g) as an off-white solid.

### Step 2. 8-bromo-7-fluoro-5-iodo-1,2,3,4-tetrahydrocyclopenta[b]indole

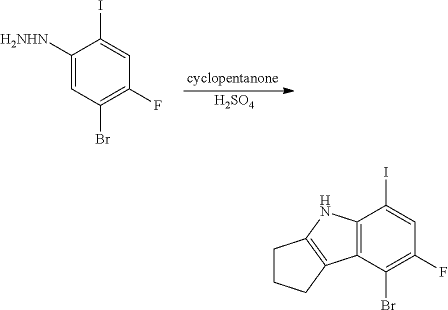

To a mixture of (5-bromo-4-fluoro-2-iodo-phenyl)hydrazine (96.65 g, 263.1 mmol) and cyclopentanone (23.24 g, 276.2 mmol, 24.5 mL) in methanol (450 mL) at rt was added a catalytic amount (2.5% by volume) of sulfuric acid (25.80 g, 263.08 mmol, 11.25 mL). The reaction was slightly exothermic and the mixture was allowed to stir at rt for 30 min. The intermediate 5-bromo-N-(cyclopentylideneamino)-4-fluoro-2-iodoaniline was observed by LC/MS but was not isolated. The reaction mixture was heated for 16 h at 80° C. then cooled and the methanol was removed under reduced pressure. The residue was treated with saturated aqueous sodium bicarbonate and extracted with ethyl acetate (3×). The combined organic layer was washed with brine, dried over anhydrous MgSO₄, filtered and concentrated under vacuum. The residue obtained was purified by silica plug chromatography eluting with 2% ethyl acetate in heptane to elute top spot, followed by 5% ethyl acetate in heptane to elute the product. The product fractions were combined and concentrated under reduced pressure give 8-bromo-7-fluoro-5-iodo-1,2,3,4-tetrahydrocyclopenta[b]indole (50.7 g) as an off-white solid. Fractions that were not pure were combined and concentrated under reduced pressure. The residue obtained was purified by silica gel chromatography using a gradient of 2%-10% EtOAc in heptane to give 8-bromo-7-fluoro-5-iodo-1,2,3,4-tetrahydrocyclopenta[b]indole (9.78 g) as an off-white solid

### Step 3. 8-bromo-7-fluoro-1,2,3,4-tetrahydrocyclopenta[b]indole-5-carbonitrile

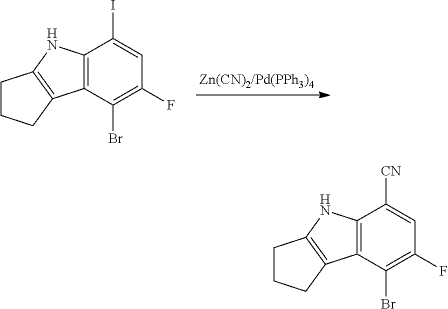 [SMILES: Fc1cc(I)c2[nH]c3c(c2c1Br)CCC3 ; N#Cc1cc(F)c(Br)c2c3c([nH]c12)CCC3]

To a mixture of 8-bromo-7-fluoro-5-iodo-1,2,3,4-tetrahydrocyclopenta[b]indole (50.5 g, 133 mmol) and Zn(CN)₂ (7.80 g, 66.45 mmol) in DMF (300 mL) purged with nitrogen was added tetrakis(triphenylphosphine)palladium (15.36 g, 13.29 mmol).

Reaction mixture was then heated at 90° C. for 2 h. The crude reaction mixture was cooled to room temperature and diluted with MTBE and water. Solids were filtered off through Celite and the layers were separated. The organic phase was washed with water (2×), brine, dried over (MgSO₄), filtered and concentrated under reduced pressure. The residue obtained was triturated in a solvent mixture of 1:4 EtOAc:heptane (200 mL).

The resulting solid was collected by vacuum filtration and washed (4×) with a solvent mixture of 1:4 EtOAc:heptane (50 mL). The solid was dried overnight under vacuum to give 8-bromo-7-fluoro-1,2,3,4-tetrahydrocyclopenta[b]indole-5-carbonitrile (24.9 g) as a brown solid.

### Step 4. tert-butyl 5-(5-cyano-7-fluoro-1,2,3,4-tetrahydrocyclopenta[b]indol-8-yl)-3,6-dihydro-2H-pyridine-1-carboxylate

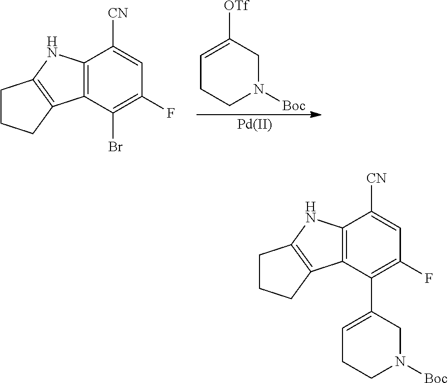

To a solution of 8-bromo-7-fluoro-1,2,3,4-tetrahydrocyclopenta[b]indole-5-carbonitrile (24.91 g, 89.25 mmol) in THF (240 mL) and water (60 mL) was added tert-butyl 5-(4,4,5,5-tetramethyl-1,3,2-dioxaborolan-2-yl)-3,6-dihydro-2H-pyridine-1-carboxylate (33.12 g, 107.1 mmol) and K₃PO₄ (56.8 g, 267.7 mmol). The mixture was then purged with N₂ for 5 min. To this was added 1,1′-bis(diphenylphosphino)ferrocene palladium(II)chloride dichloromethane complex (7.29 g, 8.92 mmol) under N₂ and the mixture was heated at 60° C. for 16 h. The reaction was cooled to rt and diluted with EtOAc and water, filter through Celite. The organic layer was separated and the aq layer was extracted with EtOAc twice more. The combined organic layer was washed with brine, dried over MgSO₄, filtered and concentrated under reduced pressure. The residue was taken up in 200 mL of 20% EtOAc in heptane. The solid was collected by vacuum filtration washed 4× with 50 mL of 20% EtOAc in heptane and dried under high vac to give tert-butyl 5-(5-cyano-7-fluoro-1,2,3,4-tetrahydrocyclopenta[b]indol-8-yl)-3,6-dihydro-2H-pyridine-1-carboxylate (27.40 g) as a brown solid.

### Step 5. tert-butyl 5-(5-carbamoyl-7-fluoro-1,2,3,4-tetrahydrocyclopenta [b]indol-8-yl)-3,6-dihydro-2H-pyridine-1-carboxylate

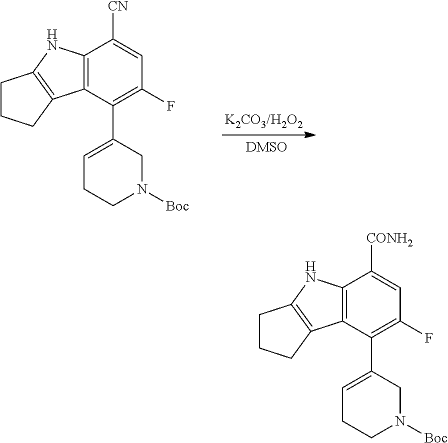 [SMILES: CC(C)(C)OC(=O)N1CCC=C(c2c(F)cc(C#N)c3[nH]c4c(c23)CCC4)C1 ; CC(C)(C)OC(=O)N1CCC=C(c2c(F)cc(C(N)=O)c3[nH]c4c(c23)CCC4)C1]

To a stirred mixture of tert-butyl 5-(5-cyano-7-fluoro-1,2,3,4-tetrahydrocyclopenta[b]indol-8-yl)-3,6-dihydro-2H-pyridine-1-carboxylate (11 g, 28.84 mmol) and anhydrous potassium carbonate (11.96 g, 86.51 mmol) in DMSO (110 mL) was added hydrogen peroxide (14.01 g, 144.19 mmol, 12.74 mL, 35% purity) at rt. The reaction was exothermic with vigorous gas formation occurring after 10 minutes. The reaction mixture stirred for 2 h at rt then quenched by the addition of water and extracted with ethyl acetate (3×). The combined organic extracts were washed with brine, dried over anhydrous sodium sulfate and concentrated under reduced pressure. To the residue obtained was added water and the resulting precipitate was collected by vacuum filtration to give tert-butyl 5-(5-carbamoyl-7-fluoro-1,2,3,4-tetrahydrocyclopenta[b]indol-8-yl)-3,6-dihydro-2H-pyridine-1-carboxylate (8.06 g) as a light brown solid.

### Step 6. tert-butyl 3-(5-carbamoyl-7-fluoro-1,2,3,4-tetrahydrocyclopenta [b]indol-8-yl)piperidine-1-carboxylate

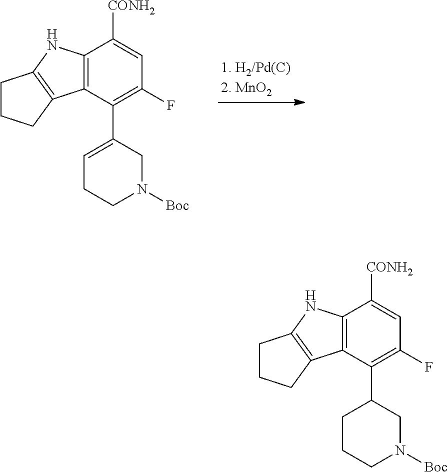 [SMILES: CC(C)(C)OC(=O)N1CCC=C(c2c(F)cc(C(N)=O)c3[nH]c4c(c23)CCC4)C1 ; CC(C)(C)OC(=O)N1CCCC(c2c(F)cc(C(N)=O)c3[nH]c4c(c23)CCC4)C1]

A mixture of tert-butyl 5-(5-carbamoyl-7-fluoro-1,2,3,4-tetrahydrocyclopenta[b]indol-8-yl)-3,6-dihydro-2H-pyridine-1-carboxylate (4 g, 10.01 mmol) and 10% palladium on carbon (1.07 g, 10.01 mmol) in ethanol (30 mL) and tetrahydrofuran (30 mL) was stirred for 12 h at 50° C. under hydrogen (2-3 atm). The mixture was filtered, the filtrate was concentrated under vacuum to give tert-butyl 3-(5-carbamoyl-7-fluoro-1,2,3,3a,4,8b-hexahydrocyclopenta[b]indol-8-yl)piperidine-1-carboxylate (3.8 g, crude) as a yellow solid. To the crude compound (3.8 g, 9.42 mmol) in THF (40 mL) was added MnO₂ (8.19 g, 94.18 mmol) at 25° C. The reaction mixture was stirred at 60° C. for 2 h. After completed, the cooled mixture was filtered, the filtrate was concentrated under vacuum. The residue was purified by column chromatography on silica gel eluting with ethyl acetate in petroleum ether (0 to 50%) to give tert-butyl 3-(5-carbamoyl-7-fluoro-1,2,3,4-tetrahydrocyclopenta[b]indol-8-yl)piperidine-1-carboxylate (1.4 g, crude) as a yellow solid. The solid was dissolved in THF (20 mL) at 70° C., followed by the addition of petroleum ether (30 mL) was added and stirred for 1 hour. The precipitated solid was collected by filtration, washed with petroleum ether (30 mL×2) and dried under vacuum to give tert-butyl 3-(5-carbamoyl-7-fluoro-1,2,3,4-tetrahydrocyclopenta[b]indol-8-yl)piperidine-1-carboxylate (800 mg) as a yellow solid.

### Step 7. 7-fluoro-8-(3-piperidyl)-1,2,3,4-tetrahydrocyclopenta[b]indole-5-carboxamide

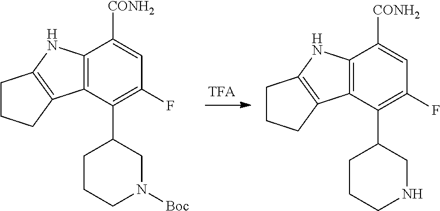 [SMILES: CC(C)(C)OC(=O)N1CCCC(c2c(F)cc(C(N)=O)c3[nH]c4c(c23)CCC4)C1 ; NC(=O)c1cc(F)c(C2CCCNC2)c2c3c([nH]c12)CCC3]

To a solution of tert-butyl 3-(5-carbamoyl-7-fluoro-1,2,3,4-tetrahydrocyclopenta[b]indol-8-yl)piperidine-1-carboxylate (4.6 g, 11.46 mmol) in dichloromethane (50 mL) was added trifluoroacetic acid (10 mL). The reaction mixture was stirred for 1 hour at 25° C. The resulting mixture was concentrated under vacuum to give 7-fluoro-8-(3-piperidyl)-1,2,3,4-tetrahydrocyclopenta[b]indole-5-carboxamide (7.2 g, crude) as a brown oil.

### Step 8. 7-fluoro-8-(1-prop-2-enoyl-3-piperidyl)-1,2,3,4-tetrahydrocyclopenta[b]indole-5-carboxamide

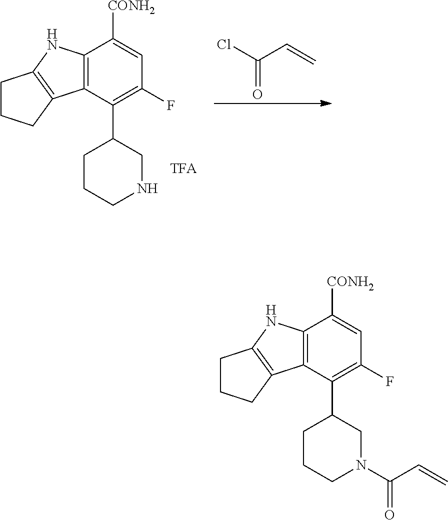

To a mixture of 7-fluoro-8-(3-piperidyl)-1,2,3,4-tetrahydrocyclopenta[b]indole-5-carboxamide (7.2 g, 17.33 mmol) in tetrahydrofuran (70 mL) was added N-ethyl-N-isopropyl-propan-2-amine (11.20 g, 86.67 mmol, 15.10 mL). After 10 min, prop-2-enoyl chloride (1.88 g, 20.80 mmol) was added to the stirred solution at −78° C. The reaction mixture was stirred at −78° C. for 1 hour. The reaction mixture was quenched with water (100 mL) and extracted with ethyl acetate (100 ml×3). The combined organic extracts were washed with brine (100 ml), dried over anhydrous sodium sulfate, filtered and concentrated under vacuum. The residue was purified by column chromatography on silica gel eluting with ethyl acetate in dichloromethane (0 to 15%) to give 7-fluoro-8-(1-prop-2-enoyl-3-piperidyl)-1,2,3,4-tetrahydrocyclopenta[b]indole-5-carboxamide (1.75 g) as an off-white.

### Step 9. 7-fluoro-8-[(3S)-1-prop-2-enoyl-3-piperidyl]-1,2,3,4-tetrahydrocyclopenta-[b]indole-5-carboxamide and 7-fluoro-8-[(3R)-1-prop-2-enoyl-3-piperidyl]-1,2,3,4-tetrahydrocyclopenta-[b]indole-5-carboxamide

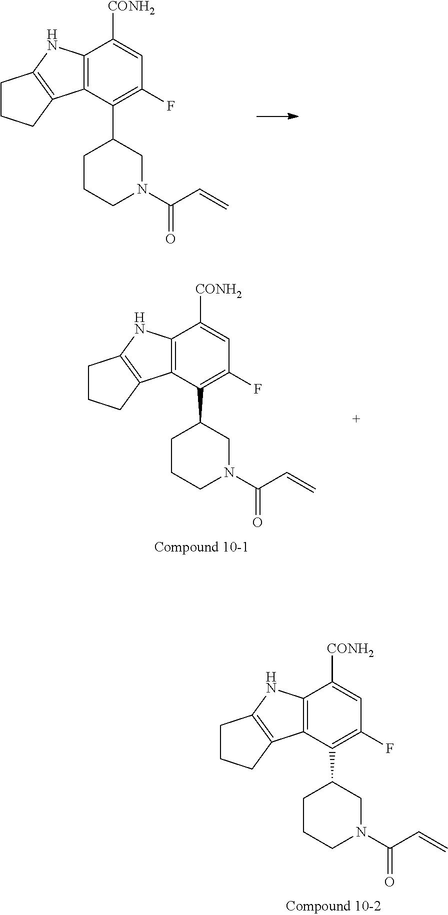 [SMILES: C=CC(=O)N1CCCC(c2c(F)cc(C(N)=O)c3[nH]c4c(c23)CCC4)C1 ; C=CC(=O)N1CCC[C@@H](c2c(F)cc(C(N)=O)c3[nH]c4c(c23)CCC4)C1 ; C=CC(=O)N1CCC[C@H](c2c(F)cc(C(N)=O)c3[nH]c4c(c23)CCC4)C1]

Racemic 7-fluoro-8-(1-prop-2-enoyl-3-piperidyl)-1,2,3,4-tetrahydrocyclopenta[b]indole-5-carboxamide (1.7 g, 4.78 mmol) was separated by Prep-SFC with the following conditions: Column: Lux 5 um Cellulose-2, 2.12×25 cm, 5 μm; Mobile Phase A:CO₂, Mobile Phase B:MeOH (0.1% 2M NH₃-MeOH); Flow rate: 40 mL/min; Gradient: 50% B; Column Temperature: 33° C.; Back Pressure: 100 bar; 220 nm.

Compound 10-1:

7-fluoro-8-[(3S)-1-prop-2-enoyl-3-piperidyl]-1,2,3,4-tetrahydrocyclopenta-[b]indole-5-carboxamide (650 mg) as a white solid. ¹H NMR (300 MHz, DMSO-d₆) δ 10.97 (s, 1H), 8.01 (s, 1H), 7.64-7.26 (m, 2H), 7.00-6.71 (m, 1H), 6.26-5.98 (m, 1H), 5.79-5.54 (m, 1H), 4.75-4.44 (m, 1H), 4.28-4.02 (m, 1H), 3.69-3.43 (m, 0.5H), 3.25-2.93 (m, 2H), 2.92-2.58 (m, 4.5H), 2.48-2.33 (m, 2H), 2.22-1.98 (m, 1H), 1.97-1.70 (m, 2H), 1.63-1.34 (m, 1H).

Compound 10-2:

7-fluoro-8-[(3R)-1-prop-2-enoyl-3-piperidyl]-1,2,3,4-tetrahydrocyclopenta-[b]indole-5-carboxamide (640 mg) as a white solid. ¹H NMR (300 MHz, DMSO-d₆) δ 10.97 (s, 1H), 8.01 (s, 1H), 7.64-7.26 (m, 2H), 7.00-6.71 (m, 1H), 6.26-5.98 (m, 1H), 5.79-5.54 (m, 1H), 4.75-4.44 (m, 1H), 4.28-4.02 (m, 1H), 3.69-3.43 (m, 0.5H), 3.25-2.93 (m, 2H), 2.92-2.58 (m, 4.5H), 2.48-2.33 (m, 2H), 2.22-1.98 (m, 1H), 1.97-1.70 (m, 2H), 1.63-1.34 (m, 1H).

### Examples 10-3 & 10-4

### Preparation of: 8-[(3 S)-3-(but-2-ynoylamino)cyclohexen-1-yl]-7-fluoro-1,2,3,4-tetrahydrocyclopenta[b]indole-5-carboxamide (Compound 10-3); and 8-[(5S)-5-(but-2-ynoylamino)cyclohexen-1-yl]-7-fluoro-1,2,3,4-tetrahydrocyclopenta[b]indole-5-carboxamide (Compound 10-4)

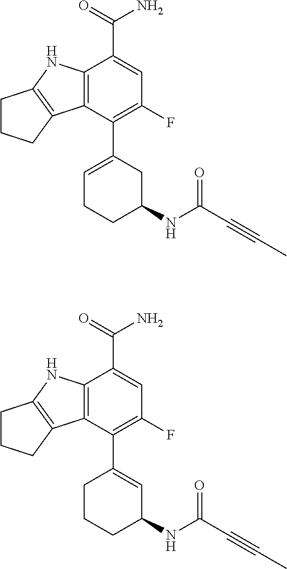 [SMILES: CC#CC(=O)N[C@H]1CCC=C(c2c(F)cc(C(N)=O)c3[nH]c4c(c23)CCC4)C1 ; CC#CC(=O)N[C@@H]1C=C(c2c(F)cc(C(N)=O)c3[nH]c4c(c23)CCC4)CCC1]

### Step 1: [(5S)-5-(tert-butoxycarbonylamino)cyclohexen-1-yl] trifluoromethanesulfonate

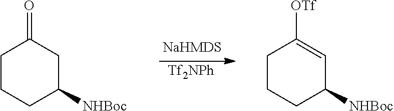 [SMILES: "Tf2NPh" ; CC(C)(C)OC(=O)N[C@H]1CCCC(=O)C1 ; CC(C)(C)OC(=O)N[C@@H]1C=C(OS(=O)(=O)C(F)(F)F)CCC1]

A solution of tert-butyl N-[(1S)-3-oxocyclohexyl]carbamate (5 g, 23.44 mmol) and 1,1,1-trifluoro-N-phenyl-N-(trifluoromethylsulfonyl)methanesulfonamide (10.89 g, 30.48 mmol) in THF (50 mL) was evacuated and flushed three times with nitrogen atmosphere. Sodium bis(trimethylsilyl)amide (2 M, 25.79 mL) was dropped into the solution at −70° C. under nitrogen atmosphere and the reaction mixture was stirred at −50 C for 2 h. The reaction mixture was quenched with saturated sodium bicarbonate (50 mL), extracted with ethyl acetate (3×50 mL). The combined organic extracts were washed with brine (100 mL), dried over anhydrous sodium sulfate and filtered. The filtrate was concentrated under reduced pressure to afford the crude product which was purified by column chromatography on silica gel, eluting with ethyl acetate in petroleum ether (0-7%) to afford [(5S)-5-(tert-butoxy-carbonylamino)cyclohexen-1-yl] trifluoromethanesulfonate (3.2 g) as a white solid

### Step 2: tert-butyl N-[(1S)-3-(4,4,5,5-tetramethyl-1,3,2-dioxaborolan-2-yl)cyclohex-3-en-1-yl]carbamate

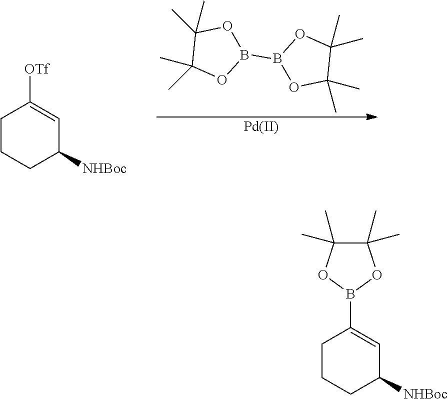

To a mixture of [(5S)-5-(tert-butoxycarbonylamino)cyclohexen-1-yl] trifluoromethanesulfonate (3.2 g, 9.27 mmol), 4,4,5,5-tetramethyl-2-(4,4,5,5-tetramethyl-1,3,2-dioxaborolan-2-yl)-1,3,2-dioxaborolane (2.59 g, 10.19 mmol) and KOAc (2.73 g, 27.80 mmol, 1.74 mL) in dioxane (30 mL) was added 1,1-bis(diphenylphosphino)ferrocenedichloro-palladium(II) (756 mg, 926 μmol) under nitrogen atmosphere. The reaction mixture was evacuated and flushed three times with N₂ atmosphere and then stirred at 100° C. for 2 h. The reaction mixture was cooled and quenched with water (50 mL), extracted with ethyl acetate (50 ml×3) and the combined organic extracts were washed with brine (100 mL), dried over anhydrous sodium sulfate and filtered. The filtrate was concentrated under reduced pressure to afford the crude product which was purified by column chromatography onto silica gel, eluting with ethyl acetate in petroleum ether (0-15%) to afford tert-butyl N-[(1S)-3-(4,4,5,5-tetramethyl-1,3,2-dioxaborolan-2-yl)cyclohex-3-en-1-yl]carbamate (2.2 g, 6.81 mmol, 73.45% yield) as colorless oil.

### Step 3: tert-butyl N-[(1S)-3-(5-cyano-7-fluoro-1,2,3,4-tetrahydro-cyclopenta[b]indol-8-yl)cyclohex-3-en-1-yl]carbamate

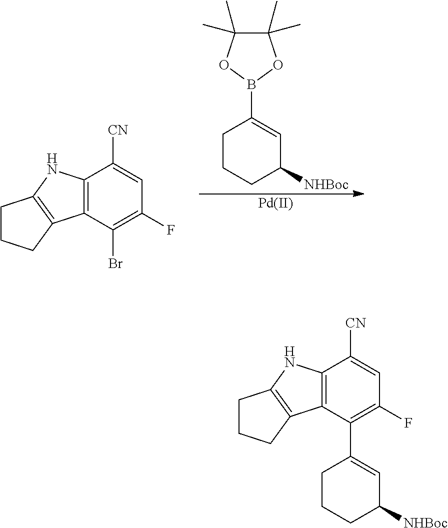

To a mixture of 8-bromo-7-fluoro-1,2,3,4-tetrahydrocyclopenta[b]indole-5-carbonitrile (1 g, 3.58 mmol), tert-butyl N-[(1S)-3-(4,4,5,5-tetramethyl-1,3,2-dioxaborolan-2-yl)cyclohex-3-en-1-yl]carbamate (1.39 g, 4.30 mmol) and K₃PO₄ (2.28 g, 10.75 mmol) in THF (8 mL) and water (2 mL) was added 1,1 bis(diphenylphosphino) ferrocenedichloropalladium(II) (262 mg, 358 μmol) under a nitrogen atmosphere. The reaction mixture was evacuated and flushed three times with nitrogen then stirred at 70° C. for 8 h. The reaction mixture was quenched with water (30 mL), extracted with ethyl acetate (20 ml×3) and the combined organic extracts were washed with brine (50 mL), dried over anhydrous sodium sulfate and filtered. The filtrate was concentrated under reduced pressure to afford the crude product which was purified by column chromatography using silica gel, eluting with ethyl acetate in petroleum ether (0-20%) to afford the desired tert-butyl N-[(1S)-3-(5-cyano-7-fluoro-1,2,3,4-tetrahydrocyclopenta[b]indol-8-yl)cyclohex-3-en-1-yl]carbamate (1 g) as a yellow solid.

### Step 4: tert-butyl N-[(1S)-3-(5-carbamoyl-7-fluoro-1,2,3,4-tetrahydro-cyclopenta[b]indol-8-yl)cyclohex-3-en-1-yl]carbamate

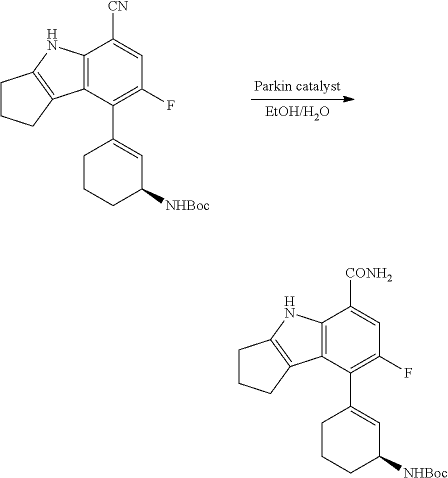 [SMILES: CC(C)(C)OC(=O)N[C@@H]1C=C(c2c(F)cc(C#N)c3[nH]c4c(c23)CCC4)CCC1 ; CC(C)(C)OC(=O)N[C@@H]1C=C(c2c(F)cc(C(N)=O)c3[nH]c4c(c23)CCC4)CCC1]

To a mixture of tert-butyl N-[(1S)-3-(5-cyano-7-fluoro-1,2,3,4-tetrahydro-cyclopenta[b]indol-8-yl)cyclohex-3-en-1-yl]carbamate (1.0 g, 2.53 mmol) in ethanol (30 mL) and water (8 mL) was added Parkin's catalyst (54.0 mg, 126 μmol). The reaction mixture was stirred at 90° C. for 12 h. The reaction mixture was cooled and quenched with water (50 mL) and extracted with ethyl acetate (30 ml×3). The combined organic extracts were washed with brine (50 mL), dried over anhydrous sodium sulfate, filtered and concentrated under vacuum. The residue was purified by column chromatography using silica gel, eluting with ethyl acetate in petroleum ether (0-60%) to afford tert-butyl N-[(1S)-3-(5-carbamoyl-7-fluoro-1,2,3,4-tetrahydro-cyclopenta[b]indol-8-yl)cyclohex-3-en-1-yl]carbamate (860 mg) as a yellow solid.

### Step 5: 8-[(5S)-5-aminocyclohexen-1-yl]-7-fluoro-1,2,3,4-tetrahydro-cyclopenta[b]indole-5-carboxamide hydrochloride

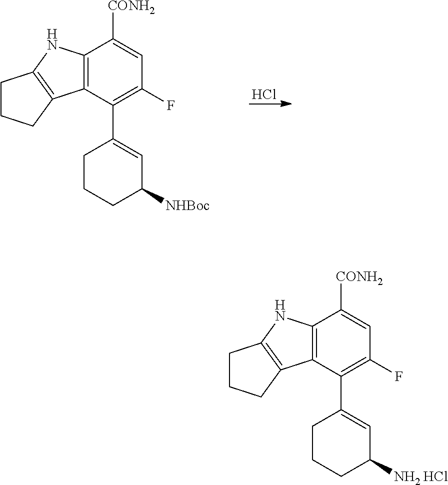 [SMILES: CC(C)(C)OC(=O)N[C@@H]1C=C(c2c(F)cc(C(N)=O)c3[nH]c4c(c23)CCC4)CCC1 ; Cl ; NC(=O)c1cc(F)c(C2=C[C@@H](N)CCC2)c2c3c([nH]c12)CCC3 ; Cl]

A solution of tert-butyl N-[(1S)-3-(5-carbamoyl-7-fluoro-1,2,3,4-tetrahydrocyclo-penta[b]indol-8-yl)cyclohex-3-en-1-yl]carbamate (200 mg, 483 μmol) and HCl (4 M in dioxane, 1.91 mL) was stirred at 20° C. for 2 h. The reaction mixture was concentrated under vacuum to give 8-[(5S)-5-aminocyclohexen-1-yl]-7-fluoro-1,2,3,4-tetrahydrocyclopenta[b]indole-5-carboxamide (180 mg) as a yellow solid which was used directly without further purification

### Step 6: 8-[(3S)-3-(but-2-ynoylamino)cyclohexen-1-yl]-7-fluoro-1,2,3,4-tetrahydrocyclopenta[b]-indole-5-carboxamide and 8-[(5S)-5-(but-2-ynoylamino)cyclohexen-1-yl]-7-fluoro-1,2,3,4-tetra-hydrocyclopenta[b]indole-5-carboxamide

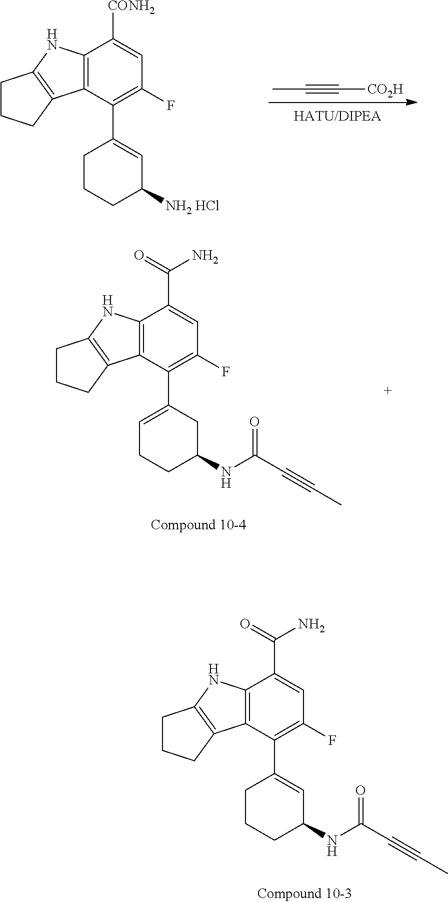

To a solution of the amine from step C above (150 mg, 428 μmol) and but-2-ynoic acid (43.2 mg, 514 μmol) in DMF (5 mL) was added HATU (228 mg, 600 μmol) then DIPEA (166 mg, 1.29 mmol, 224 μL). The reaction solution was stirred at 20° C. for 2 h. The reaction was quenched with water (15 mL), extracted with ethyl acetate (20 ml×3) and the organic phase was washed with brine (40 mL), dried over anhydrous sodium sulfate, filtered and concentrated under vacuum. The residue was purified using prep-achiral-SFC with the flowing condition: Column: GreenSep Basic, 30*150 mm 5 um; Mobile Phase A: CO₂, Mobile Phase B: IPA (0.5% 2M NH₃-MeOH); Flow rate: 60 mL/min; Gradient: 40% B; 254 nm to afford:

Compound 10-4 RT1: (1.98 min)

8-[(5S)-5-(but-2-ynoylamino)cyclohexen-1-yl]-7-fluoro-1,2,3,4-tetra-hydrocyclopenta[b]indole-5-carboxamide (34.1 mg) as a light-yellow solid. ¹H NMR (400 MHz, DMSO-d₆) δ 10.92 (s, 1H), 8.54 (d, J=7.6 Hz, 1H), 8.00 (s, 1H), 7.44-7.40 (m, 2H), 5.68 (s, 1H), 4.00-3.88 (m, 1H), 2.80 (t, J=7.2 Hz, 2H), 2.66 (t, J=7.2 Hz, 2H), 2.41-2.26 (m, 6H), 1.95 (s, 3H), 1.88-1.84 (m, 1H), 1.57-1.54 (m, 1H). ESI-MS [M+H]⁺ calculated for (C₂₂H₁₂FN₃O₂) 380.17 found: 380.10; and

Compound 10-3 RT2: (3.23 min)

8-[(3 S)-3-(but-2-ynoylamino)cyclohexen-1-yl]-7-fluoro-1,2,3,4-tetra-hydrocyclopenta[b]indole-5-carboxamide (12.2 mg) as a light-yellow solid. ¹H NMR (400 MHz, DMSO-d₆) δ 10.93 (s, 1H), 8.71 (d, J=8.0 Hz, 1H), 8.00 (s, 1H), 7.44-7.40 (m, 2H), 5.52 (s, 1H), 4.50-4.44 (m, 1H), 2.80 (t, J=7.2 Hz, 2H), 2.69 (t, J=7.2 Hz, 2H), 2.43-2.32 (m, 3H), 2.19-2.12 (m, 1H), 1.95 (s, 3H), 1.91-1.83 (m, 2H), 1.74-1.53 (m, 2H). ESI-MS [M+H]⁺ calculated for (C₂₂H₁₂FN₃O₂) 380.17 found: 380.15.

### Example 10-5 & 10-6

### Preparation of: 8-[(1S,3S)-3-(but-2-ynoylamino)cyclohexyl]-7-fluoro-1,2,3,4-tetrahydrocyclopenta[b]indole-5-carboxamide (Compound 10-5); and 8-[(1R,3S)-3-(but-2-ynoylamino)cyclohexyl]-7-fluoro-1,2,3,4-tetrahydro-cyclopenta[b]indole-5-carboxamide (Compound 10-6)

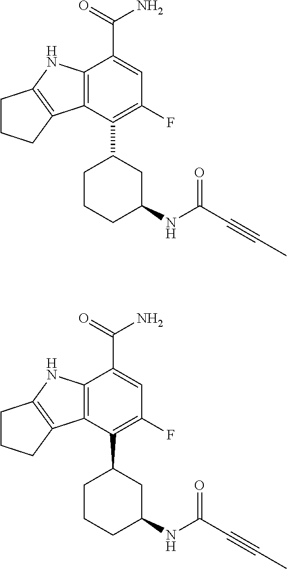

### Step 1. 8-[(3S)-3-aminocyclohexyl]-7-fluoro-1,2,3,4-tetrahydro-cyclopenta [b]indole-5-carboxamide

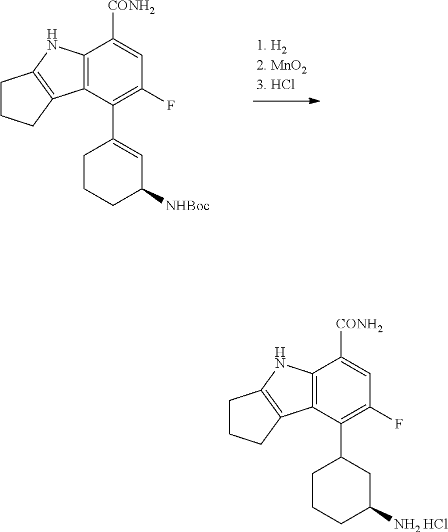 [SMILES: CC(C)(C)OC(=O)N[C@@H]1C=C(c2c(F)cc(C(N)=O)c3[nH]c4c(c23)CCC4)CCC1 ; NC(=O)c1cc(F)c(C2CCC[C@H](N)C2)c2c3c([nH]c12)CCC3 ; Cl]

To a solution of tert-butyl N-[(1S)-3-(5-carbamoyl-7-fluoro-1,2,3,4-tetrahydrocyclo-penta[b]indol-8-yl)cyclohex-3-en-1-yl]carbamate (960 mg, 2.32 mmol) in THF (10 mL) and ethanol (10 mL) was added 10% Pd/C (900 mg, 845 μmol) and the reaction mixture was stirred at 50° C. for 16 under an hydrogen atmosphere (20 psi). The reaction was cooled to room temperature and was filtered through Celite. The filter cake was washed with THF and the filtrate was concentrated to afford the crude product. The residue was purified by column chromatography on silica gel, eluting with ethyl acetate in petroleum ether (0-40%) to afford tert-butyl N-[(1S)-3-(5-carbamoyl-7-fluoro-1,2,3,3a,4,8b-hexahydrocyclopenta[b]indol-8-yl) cyclohexyl]carbamate (700 mg, 1.68 mmol) as a yellow solid which was re-suspended in THF (8 mL) and treated with MnO₂ (1.46 g, 16.7 mmol). The reaction mixture was stirred at 60° C. for 3 h then cooled to room temperature and filtered through Celite. The filter cake was washed with THF and filtrate was concentrated to afford the crude product. The residue was purified by column chromatography on silica gel, eluting with ethyl acetate in petroleum ether (0-40%) to afford tert-butyl N-[(1S)-3-(5-carbamoyl-7-fluoro-1,2,3,4-tetrahydrocyclopenta[b]indol-8-yl)cyclohexyl]carbamate (255 mg) as a yellow solid. The material was then dissolved in 4.0M HCl in dioxane (10 mL) and the reaction mixture was stirred at 25° C. for 2 h. The mixture was evaporated to dryness under reduced pressure to give 8-[(3S)-3-aminocyclohexyl]-7-fluoro-1,2,3,4-tetrahydrocyclopenta[b]indole-5-carboxamide (230 mg) as a yellow solid which was used in the next reaction without further purification.

### Step 2. 8-[(1R,3S)-3-(but-2-ynoylamino)cyclohexyl]-7-fluoro-1,2,3,4-tetrahydro-cyclopenta[b]indole-5-carboxamide and 8-[(1S,3S)-3-(but-2-ynoylamino)cyclohexyl]-7-fluoro-1,2,3,4-tetrahydrocyclopenta[b]indole-5-carboxamide

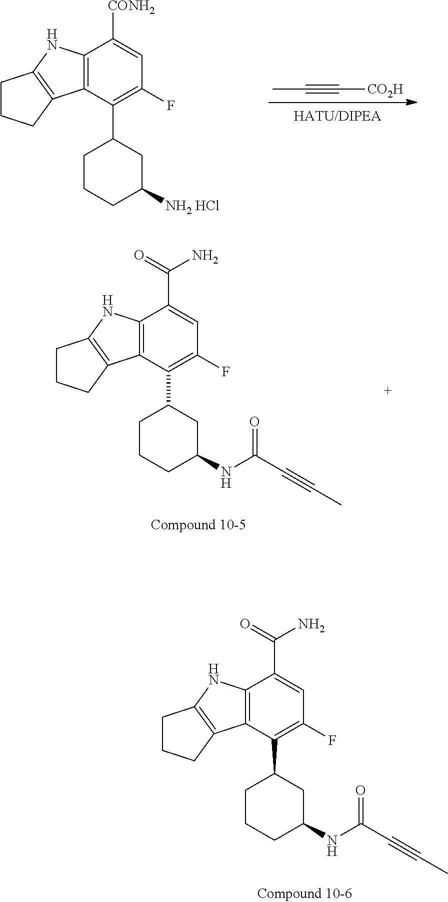

To a solution of 8-[(3S)-3-aminocyclohexyl]-7-fluoro-1,2,3,4-tetrahydrocyclopenta[b]indole-5-carboxamide (169.34 mg, 536.93 μmol), but-2-ynoic acid (54.17 mg, 644.32 μmol) and HATU (265 mg, 698 μmol) in DMF (15 mL) was added DIPEA (347 mg, 2.68 mmol, 467 μL). The reaction mixture was stirred at 25° C. for 2 h. The reaction mixture was quenched with water (50 mL) and extracted with EtOAc (50 mL×3). The organic extracts were combined and washed with brine (40 mL×3), dried over anhydrous sodium sulfate and evaporated in vacuo to give the crude product. The residue was purified by Prep-HPLC with the following conditions: (Column: Kinetex EVO C18 Column, 30*150, 5 um; Mobile Phase A:Water (50 mM NH₄HCO₃), Mobile Phase B: ACN; Flow rate: 25 mL/min; Gradient: 40 B to 50 B in 12 min; 254 nm.

Compound 10-5

RT2: (8.97 min) to give assumed 8-[(1S,3S)-3-(but-2-ynoylamino)cyclohexyl]-7-fluoro-1,2,3,4-tetrahydrocyclopenta[b]indole-5-carboxamide (23.8 mg) as a white solid. ¹H NMR (400 MHz, DMSO-d₆) δ 10.91 (s, 1H), 8.51 (d, J=6.4 Hz, 1H), 7.97 (bs, 1H), 7.38 (d, J=12.8 Hz, 2H), 3.71 (m, 1H), 3.05 (m, 1H), 2.96-2.92 (m, 2H), 2.80 (t, J=7.0 Hz, 2H), 2.54-2.40 (m, 2H), 1.91-1.75 (m, 4H), 1.70-1.63 (m, 2H), 1.55-1.44 (m, 2H). ESI-MS [M+H]⁺=382.35.

Compound 10-6

RT1: (8.13 min) assumed 8-[(1R,3S)-3-(but-2-ynoylamino)cyclohexyl]-7-fluoro-1,2,3,4-tetrahydrocyclopenta[b]indole-5-carboxamide (43.5 mg) as a white solid. ¹H NMR (400 MHz, DMSO-d₆) δ 10.88 (s, 1H), 8.66 (d, J=6.4 Hz, 1H), 7.95 (bs, 1H), 7.36 (d, J=12.8 Hz, 2H), 4.06 (m, 1H), 2.96-2.92 (m, 2H), 2.80 (t, J=7.0 Hz, 2H), 2.54-2.40 (m, 2H), 1.91-1.75 (m, 4H), 1.70-1.63 (m, 2H), 1.55-1.48 (m, 2H). ESI-MS [M+H]⁺=382.35.

### Example 10-7

### Preparation of 8-[(3S)-3-[but-2-ynoyl(methyl)amino]-1-piperidyl]-7-fluoro-1,2,3,4-tetrahydrocyclopenta[b]indole-5-carboxamide (Compound 10-7)

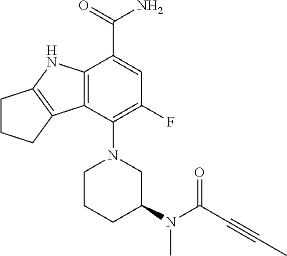 [SMILES: CC#CC(=O)N(C)[C@H]1CCCN(c2c(F)cc(C(N)=O)c3[nH]c4c(c23)CCC4)C1]

### Step 1. tert-butyl N-[(3S)-1-(5-cyano-7-fluoro-1,2,3,4-tetrahydrocyclopenta [b]indol-8-yl)-3-piperidyl]-N-methyl-carbamate

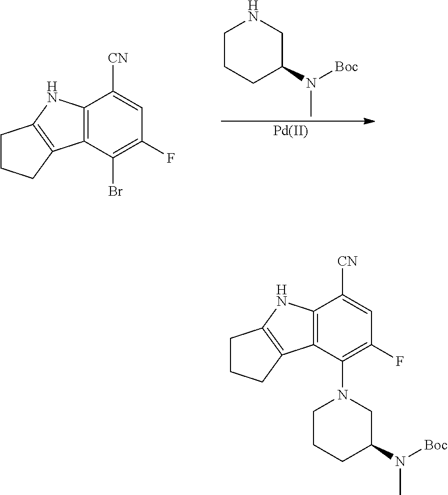

To a mixture of 8-bromo-7-fluoro-1,2,3,4-tetrahydrocyclopenta[b]indole-5-carbonitrile (400 mg, 1.43 mmol) and tert-butyl N-methyl-N-[(3S)-3-piperidyl]carbamate (368.55 mg, 1.72 mmol) in dioxane (8 mL) were added tris(dibenzylidenacetone)dipalladium(0) (131.24 mg, 143.31 μmol), dicyclohexyl-[2-(2,6-diisopropoxyphenyl)phenyl]phosphane (133.75 mg, 286.63 μmol) and cesium carbonate (1.40 g, 4.30 mmol). The reaction mixture was evacuated and flushed three times with nitrogen atmosphere and stirred at 100° C. for 16 h. The reaction mixture was cooled and quenched with water (10 ml), extracted with ethyl acetate (15 ml×3) and the combined organic extracts were washed with brine (20 ml), dried over anhydrous sodium sulfate, filtered and concentrated under vacuum to afford tert-butyl N-[(3S)-1-(5-cyano-7-fluoro-1,2,3,4-tetrahydrocyclopenta[b]indol-8-yl)-3-piperidyl]-N-methylcarbamate (600 mg, crude) as a brown solid.

### Step 2. 7-fluoro-8-[(3S)-3-(methylamino)-1-piperidyl]-1,2,3,4-tetrahydrocyclopenta[b]indole-5-carboxamide hydrochloride

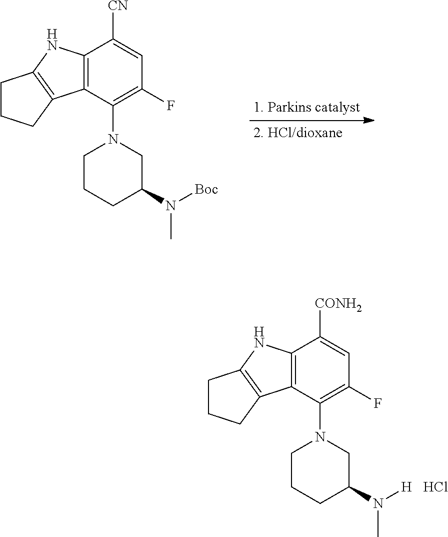 [SMILES: CN(C(=O)OC(C)(C)C)[C@H]1CCCN(c2c(F)cc(C#N)c3[nH]c4c(c23)CCC4)C1 ; CN[C@H]1CCCN(c2c(F)cc(C(N)=O)c3[nH]c4c(c23)CCC4)C1 ; Cl]

To a mixture of tert-butyl N-[(3S)-1-(5-cyano-7-fluoro-1,2,3,4-tetrahydrocyclopenta[b]indol-8-yl)-3-piperidyl]-N-methylcarbamate (600 mg, 1.45 mmol) in EtOH (10 mL) and H₂O (3 mL) was added Parkin's catalyst (62.14 mg, 145.45 μmol). The reaction mixture was stirred at 90° C. for 2 h. The reaction mixture was cooled and quenched with water (30 mL) and extracted with ethyl acetate (30 mL×3). The combined organic extracts were washed with brine (50 ml), dried over anhydrous sodium sulfate, filtered and concentrated under vacuum. The residue was purified by column chromatography onto silica gel, eluting with ethyl acetate in petroleum ether (0-40%) to afford 300 mg of tert-butyl N-[(35)-1-(5-carbamoyl-7-fluoro-1,2,3,4-tetrahydrocyclopenta[b]indol-8-yl)-3-piperidyl]-N-methylcarbamate as a yellow solid which was covered with and 4 mL of 4M HCl in dioxane and stirred at 20° C. for 2 h. The reaction mixture was concentrated under vacuum to give 7-fluoro-8-[(3 S)-3-(methylamino)-1-piperidyl]-1,2,3,4-tetrahydrocyclopenta[b]indole-5-carboxamide (260 mg) as a yellow solid which was used directly without further purification.

### Step 3. 8-[(3S)-3-[but-2-ynoyl(methyl)amino]-1-piperidyl]-7-fluoro-1,2,3,4-tetrahydrocyclopenta[b]indole-5-carboxamide (Compound 10-7)

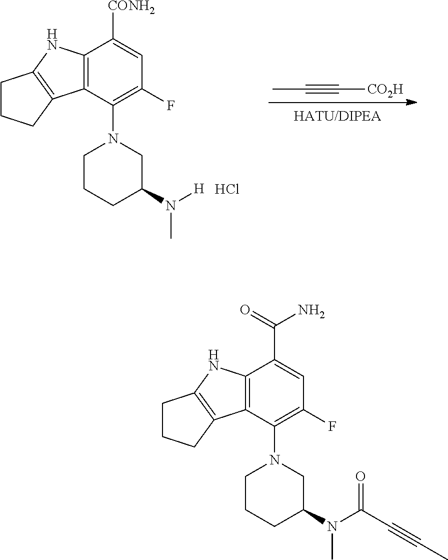

To a solution of but-2-ynoic acid (79 mg, 944 μmol) in DMF (5 mL) was added HATU (418 mg, 1.10 mmol) followed by DIPEA (305 mg, 2.36 mmol, 411 μL). The reaction solution was stirred at 20° C. for 2 h. The reaction was quenched with water (15 mL), extracted with EtOAc (20 mL×3). The organic extracts were washed with brine (40 mL), dried over anhydrous Na₂SO₄ and filtered. The filtrate was concentrated under reduced pressure and purified by prep-HPLC with the flowing condition: Column: Xselect CSH OBD Column 30*150 mm 5 um; Mobile Phase A: Water (10 mM/NH₄HCO₃+0.1% NH₄OH), Mobile Phase B: ACN; Flow rate: 60 mL/min; Gradient: 37 B to 57 B in 7 min; 220 nm to afford 8-[(35)-3-[but-2-ynoyl(methyl)amino]-1-piperidyl]-7-fluoro-1,2,3,4-tetrahydrocyclopenta[b]indole-5-carboxamide (70.9 mg) as a white solid. ¹H NMR (400 MHz, DMSO-d₆) δ 10.87 (d, J=9.2 Hz, 1H), 7.91 (s, 1H), 7.45-7.39 (m, 1H), 7.30 (s, 1H), 4.567-4.38 (m, 1H), 3.25-3.06 (m, 5H), 3.00-2.92 (m, 2H), 2.88-2.78 (m, 4H), 2.47-2.38 (m, 2H), 2.02 (d, J=6.8 Hz, 3H), 1.87-1.65 (m, 4H). ESI-MS [M+H]⁺ calculated for (C₂₂H₂₅FN₄O₂) 397.20 found: 397.15.

### Examples 10-8 & 10-9

Compound 10-8,8-((3S,5S)-3-(but-2-ynamido)-5-fluoropiperidin-1-yl)-7-fluoro-1,2,3,4-tetrahydrocyclopenta[b]indole-5-carboxamide, and 10-9,8-((3S,5R)-3-(but-2-ynamido)-5-fluoro-piperidin-1-yl)-7-fluoro-1,2,3,4-tetrahydrocyclopenta[b]indole-5-carboxamide, were prepared in a manner similar to that described above using the specified amine in Table 11.

TABLE 11
COMPOUNDS 10-8 AND 10-9

| Compound | | | | |
|---|---|---|---|---|
| No. | Structure/Name | Amine | ¹H NMR | LCMS |
| 10-8 | 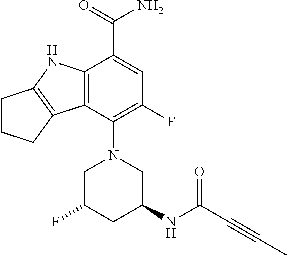 [SMILES: CC#CC(=O)N[C@H]1C[C@H](F)CN(c2c(F)cc(C(N)=O)c3[nH]c4c(c23)CCC4)C1] | 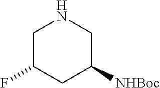 [SMILES: CC(C)(C)OC(=O)N[C@@H]1CNC[C@@H](F)C1] | (400 MHz, DMSO-d₆) δ 10.89 (s, 1H), 8.53 (d, J = 9.2 Hz, 1H), 7.92 (s, 1H), 7.46-7.40 (m, 2H), 5.06 (s, 1H), 4.90 (s, 1H), 4.27- 4.18 (m, 1H), 3.25-3.10 (m, 3H), 3.00-2.92 (m, 5H), 2.44-2.34 (m, 2H), 2.20-2.15 (m, 1H), 1.95 (s, 3H), 1.75 (t, J = 6.8 Hz, 1H), 1.66 (t, J = 6.8 Hz, 1H), | 401.1 |
| 10-9 | 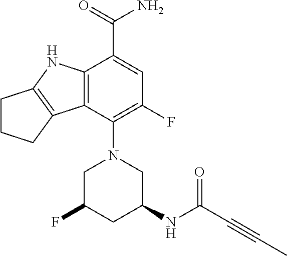 [SMILES: CC#CC(=O)N[C@H]1C[C@@H](F)CN(c2c(F)cc(C(N)=O)c3[nH]c4c(c23)CCC4)C1] | 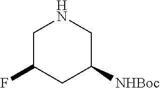 [SMILES: CC(C)(C)OC(=O)N[C@@H]1CNC[C@H](F)C1] | (400 MHz, DMSO-d6) δ 10.88 (s, 1H), 8.52 (d, J = 8.0 Hz, 1H), 7.91 (s, 1H), 7.46-7.26 (m, 2H), 5.11- 4.85 (m, 1H), 4.30-4.19 (m, 1H) 3.31-3.12 (m, 2H), 3.08-2.65 (m, 6H), 2.45-2.31 (m, 2H), 2.20-2.05 (m, 1H), 1.94 (s, 3H), 1.84-1.56 (m, 1H) | 401.1 |

### Example 11

### Assay to Determine Activity Against BTK

Solutions of compounds (test or control) in DMSO were prepared at the desired concentrations, and serially diluted to 11 concentrations by 3-fold dilution in 384pp-plate using TECAN EVO200. 20 nL of stock were transferred to 384 plate using Echo550. DMSO was used as vehicle control.

Two separate solutions were prepared—an ATP solution containing MgCl₂ (10 mM), Brij-35 (0.01%), DTT (2 mM), BSA (0.05%), EGTA (1 mM), HEPE (pH7.5) (50 mM), FLPeptide (6 uM) and ATP (4 mM); and a BTK solution containing MgCl₂ (10 mM), Brij-35 (0.01%), DTT (2 mM), BSA (0.05%), EGTA (1 mM), HEPE (pH7.5) (50 mM) and BTK (2.67 nM). (BTK was obtained from Cama; FLPeptide2 was obtained from PerkinElmer and Ibrutinib was obtained from Selleck.) 5 μL of ATP solution were added to each well, followed by addition of 15 μL of BTK solution to initiate the reaction. (Note the final volume of each well was 20 μL containing MgCl₂ (10 mM), Brij-35 (0.01%), DTT (2 mM), BSA (0.05%), EGTA (1 mM), HEPE (pH7.5) (50 mM), FLPeptide (1.5 uM), ATP (1 mM) and BTK (2 nM).

The plates were incubated at room temperature for 90 minutes and then stopping buffer added (75 μL, containing 0.5 M EDTA) to terminate the reaction. Samples from each well were analyzed using EZ reader. The % remaining activity was calculated using read conversion ratio (CR) according to the equation:

$$Remaining Activity ( \% ) = 10 \; 0 \times \frac{C {R}_{Compound}}{C {R}_{Vehicle}}$$

XLFit (equation 201) was used to calculate IC₅₀'s by floating both bottom and top.

BTK IC₅₀ values are provided for the compounds of the present invention in Table 12, below. With respect to BTK activity, Table 7 lists activity as follows:

“A” denotes an IC₅₀ of less than 10 nM;

“B” denotes and IC₅₀ of from 10 nM to less than 100 nM; and

“C” denotes and IC₅₀ of 100 nM or more.

### Example 12

### Assay to Determine BTK Activity in RAMOS B Cells

On the day before assay, Ramos B cells were plated in plating medium (RPMI1640 medium containing 1% FBS and 1× pencillin-streptomycin). On the day of the assay, 2× dye solution was prepared following the manual of the FLIRP Calcium 6 Assay Kit: Dilute the dye with assay buffer (20 mM HEPES in IX HBSS, pH7.4); Add probenecid to the final concentration of 5 mM; vortex vigorously for 1-2 minutes. Cells were collected by centrifuging, and the pellet was re-suspended in plating medium. After counting, cells were re-suspended at a density of 3×10⁶/ml in plating medium. Equal volume of 2× dye solution was added to the cell suspension. Cells were then plated at 20 μl/well into a 384-well poly-D-lysine coated plate. Plate was centrifuged at 1000 rpm for 3 minutes and then incubated at 37° C. for 2 hours followed by an additional 15-minute incubation at 25° C. Compounds were prepared at 3× concentration in dilution buffer (20 mM HEPES and 0.1% BSA in 1×HBSS, pH 7.4). Serially diluted compound was transferred from source plate to a 384-well compound plate by using an Echo 550 (Labcyte). 20 μl/well compound dilution buffer was added to the compound plate and mixed on plate shaker for 2 mins. 4× EC80 of Anti-IgM (Jackson ImmunoResearch) was prepared in dilution buffer and 20 μl/well was added to a new 384-well compound plate. After 60 mins of incubation at 25° C. in the dark; cell plate, compound plate containing 4×EC₈₀ of anti-IgM and FLIPR tips were placed into FLIPR (Molecular Devices). 10 ul/well of 4×EC₈₀ anti-IgM was transferred to the cell plate by FLIPR. Plates were read for 160 sec with 1 sec interval.

IC₅₀ values are provided for representative compounds of the present invention in Table 12, below. With respect to Ramos activity, Table 12 lists activity as follows:

“A” denotes and IC₅₀ of less than 10 nM;

“B” denotes and IC₅₀ of from 10 nM to less than 100 nM; and

“C” denotes and IC₅₀ of 100 nM or more.

TABLE 12
ACTIVITY OF REPRESENTATIVE COMPOUNDS

| | BTK | Ramos |
|---|---|---|
| Compound | IC₅₀ | IC₅₀ |
| 1-1 | B | B |
| 2-1 | B | B |
| 2-2 | B | B |
| 2-3 | C | C |
| 2-4 | A | A |
| 2-5 | B | C |
| 2-6 | A | B |
| 2-7 | C | C |
| 2-8 | B | C |
| 2-9 | B | B |
| 3-1 | A | A |
| 3-2 | A | A |
| 3-3 | A | A |
| 3-4 | C | C |
| 4-1 | B | B |
| 4-2 | C | C |
| 4-3 | B | B |
| 4-4 | B | C |
| 5-1 | B | B |
| 5-2 | C | C |
| 5-3 | B | B |
| 5-4 | A | B |
| 5-5 | B | B |
| 5-6 | A | A |
| 5-7 | C | C |
| 5-8 | B | C |
| 5-9 | B | B |
| 6-1 | B | B |
| 6-2 | A | A |
| 7-1 | B | B |
| 7-2 | B | C |
| 7-3 | C | C |
| 7-4 | C | C |
| 7-5 | B | B |
| 7-6 | C | C |
| 7-11 | A | A |
| 7-12 | A | B |
| 7-13 | B | C |
| 7-14 | C | C |
| 7-15 | B | C |
| 7-16 | A | B |
| 7-17 | B | C |
| 7-18 | B | C |
| 7-19 | B | C |
| 7-20 | C | C |
| 7-21 | A | B |
| 7-22 | B | B |
| 7-23 | A | A |
| 7-24 | A | B |
| 7-25 | C | C |
| 7-26 | C | C |
| 7-29 | B | B |
| 7-30 | A | B |
| 8-1 | A | A |
| 9-1 | A | C |
| 9-2 | B | C |
| 9-3 | A | B |
| 9-4 | C | C |
| 9-5 | A | B |
| 9-6 | B | B |
| 9-7 | A | B |
| 10-1 | A | A |
| 10-2 | A | A |
| 10-3 | A | A |
| 10-4 | A | A |
| 10-5 | A | A |
| 10-6 | B | C |
| 10-7 | A | B |
| 10-8 | A | A |
| 10-9 | B | B |

### Example 13

### Large Panel Kinase Profiling

Large panel kinase profiling was performed at AssayQuant Technologies, Inc. 260 Cedar Hill Street, Marlborough, Mass. 01752 using the PhosphoSens® CSox-Sensor platform using 1 μM test article concentration and 1 mM ATP concentration (www.assayquant.com). Reactions were run in Corning, half-area 96-well, white flat round bottom polystyrene NBS microplates (Cat. #3642) after sealing using optically-clear adhesive film (TopSealA-Plus plate seal, PerkinElmer), applied with either a roller or a paddle to eliminate evaporation and resulting drift. The standard total reaction volume was 50 μL. All experiments were ran at 30° C. to control for fluctuations in ambient temperature. Plates were read from the top in kinetic mode (readings taken every 1 min), monitoring fluorescence intensity with filters using Ex 360 nm and Em 485 nm and a band width of 40 and 20 nm, respectively, and a gain of 80. Data analysis was performed using either Graphpad Prism or Gene Data Screener software.

Results from the large panel kinase screen are shown in the table below. Compounds 5-6, 6-2 and 10-2 are highly selective and demonstrate the greatest inhibition of the target kinase (BTK) of the 288 kinases assayed.

TABLE 13
KINASE PANEL ACTIVITY

| | % Inhibition at 1 μM Test Article concentration | | |
|---|---|---|---|
| | Compound | Compound | Compound |
| Kinase | 5-6 | 6-2 | 10-2 |
| AAK1 | −20.3 | −15.7 | −14.3 |
| ABL1 | −14.6 | 2.5 | 3.1 |
| ABL2 | −29.7 | −1.3 | 1.9 |
| AKT1 | 20.0 | −17.0 | −14.8 |
| AKT2 | 0.5 | 0.4 | 11.9 |
| AKT3 | −14.4 | −16.3 | −19.4 |
| ALK | −26.4 | 11.7 | −2.7 |
| AURKA/AURA | −42.0 | −24.7 | −12.1 |
| AURKB/AURB | −19.0 | −11.5 | −14.9 |
| AURKC/AURC | −8.9 | −2.0 | 16.7 |
| AXL | −20.3 | 1.5 | −1.7 |
| BLK | 30.3 | 65.7 | −2.3 |
| BMP2K/BIKE | −18.6 | −12.3 | −6.6 |
| BMX | 42.0 | 86.0 | 65.2 |
| BRAF(V599E) | −13.2 | −4.9 | 4.3 |
| BRSK1 | −30.9 | −13.0 | 10.1 |
| BRSK2 | −31.2 | −5.7 | 1.8 |
| BTK | 92.8 | 97.4 | 93.8 |
| BUB1 | 8.2 | −11.5 | −2.3 |
| CAMK1A | 24.4 | 34.5 | 7.9 |
| CAMK1D | 19.6 | 26.1 | 10.5 |
| CAMK1G | 15.6 | 19.6 | 9.7 |
| CAMK2A | 4.9 | 35.8 | 14.7 |
| CAMK2D | 1.1 | 8.4 | −10.7 |
| CAMK2G | 15.7 | −7.1 | −37.5 |
| CAMK4 | 15.7 | 26.6 | 15.3 |
| CAMKK1 | 22.2 | 5.7 | 3.7 |
| CAMKK2 | 25.8 | 0.4 | 2.6 |
| CDC42BPA/MRCKA | −30.9 | −16.2 | 1.8 |
| CDC42BPB/MRCKB | −31.0 | −12.6 | −10.2 |
| CDK1/B1 | −13.7 | −9.5 | −3.8 |
| CDK1/E1 | −3.6 | −7.0 | −10.6 |
| CDK16/T | 19.7 | −3.1 | −8.1 |
| CDK18 | 19.0 | −15.0 | 2.0 |
| CDK2/A2 | −6.3 | 5.6 | 2.3 |
| CDK2/E1 | −7.3 | −12.9 | 7.0 |
| CDK3/E1 | −15.3 | 13.7 | −7.7 |
| CDK4/D3 | −4.9 | −13.0 | −19.5 |
| CDK5/P25 | −11.1 | −13.0 | 1.6 |
| CDK5/P35 | −13.2 | −37.5 | 2.9 |
| CDK6/D1 | −5.8 | −4.3 | 12.8 |
| CDK6/D3 | −8.0 | −6.1 | −18.4 |
| CDK7 | −10.1 | −6.0 | 14.1 |
| CDK9/T1 | −11.0 | −9.9 | −26.7 |
| CHEK1/CHK1 | −34.8 | −23.1 | 6.8 |
| CHEK2/CHK2 | 0.6 | −14.1 | −9.2 |
| CHUK/IKKA | 4.0 | −36.4 | −14.5 |
| CRIK/CIT | −9.9 | 9.1 | 11.1 |
| CLK1 | −26.4 | −10.4 | −11.1 |
| CLK2 | 0.7 | −4.6 | −3.2 |
| CLK3 | −29.3 | −0.1 | −19.2 |
| CLK4 | 12.3 | 6.5 | 2.7 |
| CSF1R/FMS | 21.3 | 24.9 | 20.2 |
| CSK | −9.4 | −6.4 | −8.5 |
| CSNK1A1/CK1A1 | 9.6 | 11.2 | −2.8 |
| CSNK1D/CK1D | 4.2 | 11.7 | 16.5 |
| CSNK1E/CK1E | −4.2 | −7.4 | 15.1 |
| CSNK1G1/CK1G1 | 17.2 | 14.0 | −2.8 |
| CSNK2A1/CK2A1 | 0.4 | −12.0 | −32.0 |
| CSNK2A2/CK2A2 | 11.4 | −140.9 | −144.3 |
| DAPK1 | 8.1 | 22.1 | −20.3 |
| DAPK2 | −19.1 | 13.4 | 13.9 |
| DAPK3 | 25.9 | 10.0 | −4.9 |
| DDR1 | 1.4 | 24.5 | −41.1 |
| DDR2 | −6.5 | −15.9 | −26.7 |
| DYRK1A | 20.4 | −2.2 | 7.2 |
| DYRK1B | 16.1 | −0.2 | 6.6 |
| DYRK2 | 6.1 | 13.3 | 0.3 |
| DYRK3 | 2.8 | −4.9 | −3.3 |
| DYRK4 | 23.2 | 15.4 | −6.6 |
| EGFR | −7.8 | −7.7 | 1.6 |
| EIF2AK1/HRI | −6.1 | −34.9 | −89.5 |
| EIF2AK2/PKR | −28.0 | −1.6 | 65.5 |
| EIF2AK3/PEK | −9.2 | −18.5 | −3.5 |
| EIF2AK4/GCN2 | −20.4 | 25.9 | 29.1 |
| EPHA1 | 3.7 | 22.0 | 8.5 |
| EPHA2 | −1.9 | 9.5 | −9.6 |
| EPHA3 | 0.7 | 10.1 | 1.6 |
| EPHA4 | 4.2 | 4.9 | −44.2 |
| EPHA5 | 8.1 | 17.0 | −13.5 |
| EPHA6 | 6.1 | −14.6 | −17.7 |
| EPHA7 | −0.1 | 5.1 | −4.7 |
| EPHA8 | 10.0 | −15.3 | −19.1 |
| EPHB1 | 14.5 | 12.4 | −3.1 |
| EPHB2 | 21.3 | 11.8 | 0.9 |
| EPHB3 | 17.6 | −24.4 | −10.1 |
| EPHB4 | 7.0 | 7.4 | −8.5 |
| ERBB2/HER2 | −2.2 | 11.9 | 14.5 |
| ERBB4/HER4 | −19.7 | 2.4 | 17.1 |
| ERN1 | −5.4 | 3.1 | −2.9 |
| ERN2 | −8.0 | −14.6 | −7.1 |
| FER | 32.0 | 2.1 | 29.3 |
| FES | 24.5 | −33.0 | −31.2 |
| FGFR1 | 18.4 | 6.6 | 8.7 |
| FGFR2 | 17.7 | −15.3 | −3.5 |
| FGFR3 | 24.2 | 10.5 | 18.3 |
| FGFR4 | 24.5 | 4.9 | 9.8 |
| FGR | 17.1 | −25.9 | 3.6 |
| FLT1 | 12.1 | 20.7 | 8.6 |
| FLT3 | 2.2 | −9.4 | −4.6 |
| FLT4 | 19.7 | 20.4 | 1.8 |
| FRK | 4.2 | −2.8 | −4.1 |
| FYNA | 12.0 | −5.2 | −24.6 |
| FYNB | 21.0 | −20.0 | −36.2 |
| GAK | 17.9 | −3.7 | −28.4 |
| GRK1 | 16.4 | −46.1 | −1.4 |
| GRK2 | −14.8 | −10.5 | −1.6 |
| GRK3 | −25.6 | −48.3 | −42.7 |
| GRK4 | 3.4 | −53.0 | −36.1 |
| GRK5 | −8.8 | −113.6 | −2.2 |
| GRK6 | 8.0 | −3.1 | −1.9 |
| GRK7 | 1.3 | 10.9 | −33.0 |
| GSK3A | 2.9 | −5.1 | −37.7 |
| GSK3B | −6.0 | 18.7 | −12.5 |
| HCK | 6.8 | 2.8 | −5.8 |
| HIPK1 | 6.6 | 41.1 | 33.6 |
| HIPK2 | −18.6 | −55.4 | −66.1 |
| HIPK3 | −11.2 | 30.6 | 34.7 |
| HIPK4 | −1.6 | −1.9 | 24.3 |
| IGF1R | −16.7 | −11.6 | −26.1 |
| IKBKB/IKKB | 2.5 | −7.6 | −17.0 |
| IKBKE/IKKE | 3.4 | −18.0 | −12.8 |
| INSR | 10.3 | −6.6 | −6.0 |
| INSRR | 10.7 | −54.1 | −5.7 |
| IRAK1 | −28.6 | 5.6 | 7.8 |
| IRAK4 | −4.7 | 4.1 | −9.2 |
| ITK | 44.8 | 29.2 | 12.2 |
| JAK1(JH1) | 1.3 | 25.6 | 1.9 |
| JAK1(JH1JH2) | 8.5 | 29.4 | 14.8 |
| JAK2(JH1) | 11.4 | 10.3 | −0.8 |
| JAK2(JH1JH2) | 1.0 | −9.4 | −9.8 |
| JAK3(JH1) | 2.0 | 12.5 | 2.4 |
| KDR | 10.3 | 31.6 | 32.7 |
| KIT | 5.5 | 26.9 | 20.8 |
| LATS1 | −24.7 | 10.1 | 22.9 |
| LATS2 | 6.3 | 1.5 | −0.7 |
| LCK | 0.2 | 15.9 | 18.3 |
| LIMK1 | −7.2 | −14.7 | −10.7 |
| LRRK2 | −8.9 | −2.7 | −7.2 |
| LTK | 0.6 | 18.3 | 24.4 |
| LYNA | −2.9 | 36.4 | 22.8 |
| LYNB | 8.9 | 28.9 | 23.2 |
| MAP3K1 | 6.4 | −7.9 | 10.3 |
| MAP3K10 | −8.6 | 18.9 | −17.7 |
| MAP3K11 | 16.0 | 17.6 | −3.2 |
| MAP3K12 | 5.0 | −18.8 | −27.7 |
| MAP3K13 | 3.6 | 16.1 | 3.2 |
| MAP3K14 | 10.5 | −11.5 | −0.2 |
| MAP3K2 | −19.0 | −6.6 | 15.7 |
| MAP3K3 | −7.0 | −3.6 | 19.9 |
| MAP3K5 | −10.4 | 20.0 | 16.7 |
| MAP3K7 | −19.6 | 10.0 | 13.2 |
| MAP3K8 | 7.4 | 10.1 | 4.5 |
| MAP3K9 | −0.9 | 28.8 | 12.3 |
| MAP4K1/HPK1 | −3.2 | 16.3 | 0.8 |
| MAP4K2/GCK | −46.6 | −1.3 | −1.3 |
| MAP4K3/GLK | −7.3 | 6.2 | 5.0 |
| MAP4K4/HGK | −22.7 | 2.9 | −10.5 |
| MAP4K5/KSH1 | −1.1 | 4.1 | −4.9 |
| MAPK1/ERK2 | −4.4 | −3.8 | −8.8 |
| MAPK10/JNK3 | −13.0 | 32.1 | −4.0 |
| MAPK11/P38B | −3.3 | 32.9 | 5.7 |
| MAPK12/P38G | −1.9 | 31.3 | 4.2 |
| MAPK13/P38D | 6.7 | 26.0 | 9.0 |
| MAPK14/P38A | −8.3 | 28.2 | 5.0 |
| MAPK15/ERK7 | −3.4 | 15.3 | −2.0 |
| MAPK3/ERK1 | −22.1 | 22.7 | 5.5 |
| MAPK7/ERK5 | −0.7 | 9.4 | −4.3 |
| MAPK8/JNK1 | −0.6 | 21.7 | 13.6 |
| MAPK9/JNK2 | −6.3 | 19.7 | 3.8 |
| MAPKAPK2/MK2 | −5.9 | 24.1 | −7.1 |
| MAPKAPK3/MK3 | 2.1 | 1.5 | −42.3 |
| MAPKAPK5/MK5 | 3.7 | 8.6 | −12.6 |
| MARK1 | 15.4 | −63.9 | −12.9 |
| MARK2 | 11.8 | −33.3 | −8.8 |
| MARK3 | 1.6 | −10.6 | −90.5 |
| MARK4 | 8.1 | −10.2 | 1.7 |
| MASTL | 2.2 | −9.0 | −10.2 |
| MELK | −27.6 | −14.5 | 11.1 |
| MERTK/MER | −0.8 | 12.4 | 14.8 |
| MET | 12.0 | 18.9 | 28.1 |
| MINK1 | −2.8 | −22.7 | −18.4 |
| MST1R/RON | −2.4 | −0.6 | −2.5 |
| MUSK | 10.9 | −3.1 | 3.0 |
| MYLK/MLCK | 19.7 | −24.0 | −27.4 |
| MYLK2 | 16.8 | −28.4 | −33.6 |
| MYLK3 | 21.5 | 9.9 | 14.9 |
| MYLK4 | 29.7 | −30.5 | −21.1 |
| MYO3B | −36.2 | −16.5 | −11.0 |
| NEK1 | −3.1 | 7.9 | 11.8 |
| NEK2 | −26.5 | −1.4 | 10.6 |
| NEK3 | −21.2 | −8.1 | −5.0 |
| NEK4 | −5.3 | −6.5 | −11.1 |
| NEK6 | −13.5 | −5.8 | −13.6 |
| NEK7 | 14.6 | 13.8 | −22.6 |
| NEK9 | −9.3 | 18.0 | 2.4 |
| NTRK1/TRKA | −5.0 | 19.6 | 21.6 |
| NTRK2/TRKB | −20.9 | 13.1 | 19.7 |
| NTRK3/TRKC | −25.7 | 5.7 | 2.9 |
| NUAK1 | 3.8 | 13.2 | −9.3 |
| PAK1 | 9.3 | 11.4 | 6.8 |
| PAK2 | 13.9 | 1.3 | 7.0 |
| PAK3 | 5.4 | 10.2 | 21.7 |
| PASK | 1.8 | −8.0 | −8.5 |
| PDGFRA | −26.6 | 23.9 | 2.1 |
| PDGFRB | −52.9 | 23.2 | 4.7 |
| PDK1 | 22.8 | −7.8 | −28.3 |
| PHKG2 | 10.1 | 64.5 | 75.0 |
| PIM1 | 7.0 | −43.0 | −7.1 |
| PIM2 | 2.5 | 10.9 | 3.3 |
| PIM3 | −14.1 | −56.4 | −16.8 |
| PKN1 | 5.2 | −10.2 | 12.0 |
| PKN2 | 0.7 | 16.2 | 7.5 |
| PKN3 | 5.7 | −9.4 | 3.5 |
| PLK1 | −6.3 | −4.3 | 13.8 |
| PRKAA1/AMPK111 | −13.5 | 13.2 | 2.2 |
| PRKAA1/AMPK121 | −7.9 | 31.3 | 34.2 |
| PRKAA2/AMPK211 | −24.4 | 28.8 | 36.6 |
| PRKAA2/AMPK221 | −17.9 | 11.7 | 32.1 |
| PRKACA | 11.4 | 12.2 | 1.0 |
| PRKACB | 10.6 | 13.2 | 1.6 |
| PRKACG | 4.2 | −27.2 | −19.2 |
| PRKCA/PKCA | 2.3 | 14.7 | −3.5 |
| PRKCB1/PKCB1 | 8.6 | 8.9 | −9.4 |
| PRKCB2/PKCB2 | −32.3 | −9.4 | −28.2 |
| PRKCD/CPKCD | −42.2 | −0.1 | −0.6 |
| PRKCE/PKCE | −3.2 | 0.0 | −3.5 |
| PRKCG/PKCG | −7.3 | 8.6 | 1.3 |
| PRKCH/PKCH | −3.6 | 5.3 | −7.9 |
| PRKCI/PKCI | −0.6 | 9.0 | −3.3 |
| PRKCQ/PKCQ | −3.7 | 0.7 | −72.5 |
| PRKCZ/PKCZ | −4.0 | 8.5 | −4.2 |
| PRKD1 | −6.6 | −16.0 | −24.2 |
| PRKD2 | −14.2 | −32.9 | −12.1 |
| PRKD3 | 4.4 | −62.4 | −65.4 |
| PRKDC/DNA-PK | −40.6 | 35.0 | 14.6 |
| PRKG1 | −2.8 | 0.0 | −5.8 |
| PTK2 | −69.1 | 16.5 | −14.6 |
| PTK2B | −16.1 | −15.4 | −8.8 |
| PTK6 | −14.1 | 22.2 | 10.1 |
| RAF1 | 3.7 | 1.0 | −3.1 |
| RET | −24.0 | 23.0 | 6.0 |
| RIPK2 | 7.1 | −31.3 | −45.4 |
| ROCK1 | 3.5 | 2.5 | 13.7 |
| ROCK2 | −28.3 | 0.8 | 21.2 |
| ROS | −35.7 | −3.6 | −22.0 |
| RPS6KA1/RSK1 | 6.7 | −49.1 | −29.2 |
| RPS6KA2/RSK3 | 1.7 | −47.9 | −38.0 |
| RPS6KA3/RSK2 | 5.8 | −34.6 | −27.7 |
| RPS6KA4/MSK2 | −6.1 | −23.2 | −21.2 |
| RPS6KA5/MSK1 | −20.7 | −4.2 | −14.5 |
| RPS6KA6/RSK4 | −5.8 | −14.8 | 0.3 |
| SGK1 | 4.3 | 10.7 | 14.8 |
| SGK2 | −46.7 | −26.2 | −34.9 |
| SGK3 | −28.1 | −19.9 | −32.5 |
| SK1 | −13.6 | 4.6 | 13.5 |
| SK2 | −0.1 | 7.7 | 23.9 |
| SK3 | 5.3 | 14.7 | 11.6 |
| SLK | −3.7 | 8.9 | −48.3 |
| SRC | −28.3 | −7.0 | −1.8 |
| SRMS | −10.0 | −35.5 | −32.0 |
| SRPK2 | 24.9 | −60.6 | −29.1 |
| STK10/LOK | 3.1 | 12.2 | −8.7 |
| STK11/LKB1 | 23.5 | −4.3 | −6.0 |
| STK17A/DRAK1 | 22.4 | −33.9 | −28.0 |
| STK17B/DRAK2 | 0.3 | −26.9 | −23.7 |
| STK3/MST2 | −13.7 | −25.3 | −14.4 |
| STK33 | −7.9 | −27.1 | 20.5 |
| STK4/MST1 | 0.9 | −9.5 | 0.4 |
| SYK | 2.8 | −5.3 | −14.1 |
| TAOK1 | 11.9 | 9.5 | 11.1 |
| TAOK2 | −14.5 | −24.8 | 11.4 |
| TAOK3 | 14.1 | 1.6 | −2.9 |
| TBK1 | 10.9 | −34.6 | −31.3 |
| TEC | 80.3 | 84.5 | 76.1 |
| TEK/TIE2 | −11.2 | 4.6 | −15.2 |
| TNIK | 9.9 | −29.3 | −22.9 |
| TNK2/ACK | −8.3 | 13.4 | 2.0 |
| TSSK1 | 7.3 | −3.0 | 14.0 |
| TSSK2 | 10.2 | 22.9 | 25.3 |
| TXK | 74.7 | 59.1 | 71.0 |
| TYK2(JH1) | −2.7 | −1.1 | 3.5 |
| TYK2(JH1JH2) | 18.9 | −27.1 | 28.6 |
| TYRO3 | 2.4 | 14.7 | 10.1 |
| ULK1 | −3.1 | −23.6 | −6.1 |
| ULK2 | −17.5 | −6.2 | −5.8 |
| YES | 8.8 | 27.0 | 23.9 |
| ZAP70 | −22.5 | 1.7 | −9.5 |

### Example 14

### Determination of Plasma and Total Brain Concentrations at 1 h Post a Single Oral Administration Test Compound to Female C57BL/6 Mice

Test Article Preparation

The appropriate amount of test article was dissolved 10% dimethylacetamide (DMA)/90% (20% hydroxypropyl-β-cyclodextrin (HP-B-CD) w/v in water to obtain a final concentration of 1 mg/mL for oral dosing. Sonication, vortex, and homogenization was used as needed. Three female C57BL/6 mice aged 7-9 weeks (20-30 grams) were dosed by oral gavage 10 mg/kg solution of the test article.

Sample Collection and Processing

| | |
|---|---|
| Collection Site: | Cardiac puncture vein |
| Volume Collected: | −0.10 mL |
| Anticoagulant: | EDTA-K₂ |
| Blood Samples | Approximately 0.10 mL blood is collected at each time point. |
| Processing and | Blood of each sample is transferred into plastic micro centrifuge |
| Storage: | tubes containing anticoagulant of EDTA-K₂. Collection tubes with |
| | blood samples and anticoagulant are inverted several times for |
| | proper mixing of the tube contents and then placed on wet ice |
| | prior to centrifugation for plasma. |
| | Blood samples are centrifuged at 4,000 g, 5 mins, 4° C. to obtain |
| | plasma. |
| | Samples were stored at −75 ± 15° C. prior to analysis. |
| Brain Sample | The animals were fully exsanguinated with a rising concentration |
| Processing and | of CO₂ gas prior to tissue collection. Procedure: open chest cavity, |
| Storage: | cut ventricle and perform a gentle iv saline flush (saline flush |
| | volume ~20 mL) with the animal placed head down at a 45° angle |
| | to facilitate blood removal. |
| | For brain collection. Tissue samples are collected at adopted time |
| | point, dried with clean gauze and placed into weighed tube. Brain |
| | samples are weighed and frozen in liquid nitrogen and kept at −75 ± 15° |
| | C. before analysis. |

Sample Analysis

Concentrations of test compounds in the plasma and brain samples (use plasma and brain homogenate standard curve for all samples appropriately) were analyzed using LC-MS/MS. Compounds having a brain-to-plasma ratio of greater than 0.2 were considered brain penetrant. Known BTK inhibitors are listed in Table 14 for reference.

TABLE 14
MOUSE BRAIN TO PLASMA RATIO

| | Mean [plasma], | Mean [brain], | |
|---|---|---|---|
| Compound No. | ng/mL | ng/g | ratio |
| 2-4 | 163 | 39 | 0.24 |
| 5-4 | 18 | 4 | 0.22 |
| 5-6 | 42 | 16 | 0.40 |
| 6-2 | 78 | 18 | 0.24 |
| 7-2 | 109 | 43 | 0.39 |
| 10-2 | 191 | 72 | 0.37 |
| Acalabrutinib | 120 | 4 | 0.04 |
| Evobrutinib | 177 | 11 | 0.06 |
| Branebrutinib | 6823 | 15 | 0.002 |
| Ibrutinib | 306 | 15 | 0.001 |

### Example 15

### Determination of Plasma and Total Brain Concentrations at 1 h Post a Single Oral Administration Test Compound to Male Wistar Han Rats

Test Article Preparation

The appropriate amount of test article was dissolved 10% dimethylacetamide (DMA)/90% (20% hydroxypropyl-β-cyclodextrin (HP-B-CD) w/v in water to obtain a final concentration of 1 mg/mL for oral dosing. Sonication, vortex, and homogenization was used as needed. Three male Wistar Han rats aged 6-8 weeks (200-300 grams) were dosed by oral gavage 10 mg/kg solution of the test article.

Dosing and Sample Collection

At 1 h, blood and whole brains were collected. Blood was collected via cardiac puncture into tubes containing potassium EDTA and kept on ice until plasma collection. Plasma was obtained via centrifugation at 4000×g for five minutes at 4° C. Following exsanguination, animals were transcardially perfused with approximately 20 mL of saline, and brains were collected and immediately frozen. Plasma and brains were at −75±15° C. until analysis.

LC-MS/MS Analysis of Plasma and Brain Samples

The desired serial concentrations of working solutions were achieved by diluting stock solution of analyte with 50% acetonitrile in water solution. 5 μL of working solutions (2, 5, 10, 50, 100, 500, 1000, 5000, 10000 ng/mL) were added to 50 μL of the male blank Wistar Han rats plasma, brain homogenate to achieve calibration standards of 0.2˜1000 ng/mL (0.2, 0.5, 1, 5, 10, 50, 100, 500, 1000 ng/mL) in a total volume of 55 μL. Four quality control samples at 0.5 ng/mL, 1 ng/mL, 50 ng/mL and 800 ng/mL for plasma, brain homogenate were prepared independently of those used for the calibration curves. These QC samples were prepared on the day of analysis in the same way as calibration standards.

55 μL of standards, 55 μL of QC samples and 55 μL of unknown samples (50 μL of plasma, brain homogenate with 5 μL of blank solution) were added to 200 μL of acetonitrile containing IS mixture for precipitating protein respectively. Then the samples were vortexed for 30 s. After centrifugation at 4° C., 4700 rpm for 15 min, the supernatant was diluted 3 times with water. 10 μL of diluted supernatant was injected into the LC/MS/MS system for quantitative analysis.

The results from select compounds are shown in Table 15 below. Compounds having a brain-to-plasma ratio of greater than 0.2 were considered brain penetrant.

TABLE 15
RAT BRAIN TO PLASMA RATIO

| | Mean [plasma], | Mean [brain], | |
|---|---|---|---|
| Compound No. | ng/mL | ng/mL | ratio |
| 5-6 | 196 | 84 | 0.43 |
| 6-2 | 395 | 108 | 0.28 |
| 10-2 | 421 | 116 | 0.29 |

The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet are incorporated herein by reference, in their entirety, to the same extent as if each individual publication, patent, or patent application was specifically and individually indicated to be incorporated by reference. Aspects of the embodiments can be modified, if necessary to employ concepts of the various patents, applications and publications to provide yet further embodiments.

This application claims the benefit of priority to U.S. Provisional Application No. 63/008,531, filed Apr. 10, 2020, and U.S. Provisional Application No. 63/142,411, filed Jan. 27, 2021, which applications are hereby incorporated by reference in their entireties.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

1. A compound having the structure of formula (I): 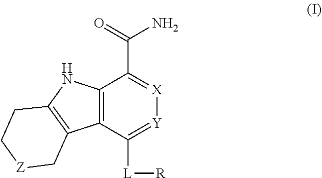
or a pharmaceutically acceptable salt, solvate, hydrate, isomer, tautomer, racemate, or isotope thereof, wherein:
X is CR¹ or N;
Y is CR² or N;
R¹ is H, halo, or C₁₋₃ alkyl;
R² is H, halo, or C₁₋₃ alkyl;
Z is a bond, C(R³R⁴), C(R⁵R⁶)—C(R⁷R⁸), C(R⁹R¹⁰)—N(R¹¹), N(R¹¹)—C(R⁹R¹⁰), O, S, Si(R¹²R¹³),
R³ and R⁴ are each, independently, H, halo, or C₁₋₃ alkyl, or R³ and R⁴, together with the carbon to which they are attached, form ring A: 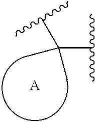
wherein ring A is an optionally substituted carbocycle or an optionally substituted heterocycle;
R⁵, R⁶, R⁷, R⁸, R⁹, and R¹⁰ are each, independently, H, halo, or C₁₋₃ alkyl;
R¹¹, R¹², and R¹³ are each, independently, H or C₁₋₃ alkyl;
L is 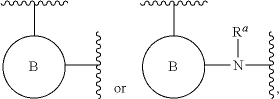
ring B is an optionally substituted carbocycle or an optionally substituted heterocycle;
R^{a} is H or C₁₋₃ alkyl;
R is C(O)R¹⁴; and
R¹⁴ is C₂₋₆ alkenyl substituted with 0-3 R′ or C₂₋₆ alkynyl substituted with 0-3 R′;
R′ is at each occurrence, independently, halo, —OR^{b}, —NR^{b}R^{c}, or optionally substituted carbocycle; and
R^{b} and R^{c} are at each occurrence, independently, H, C₁₋₆ alkyl, or C₁₋₆ haloalkyl;
with the proviso that if Z is CH₂ or CF₂, then at least one of X and Y is N.

2. The compound of claim 1, or a pharmaceutically acceptable salt, solvate, hydrate, isomer, tautomer, racemate, or isotope thereof, wherein
ring B is substituted with 0-5 R¹⁵, and
R¹⁵ is at each occurrence, independently, —OH, —CN, halo, C₁₋₆ alkyl, C₁₋₆ haloalkyl, C₁₋₆ alkoxy, or C₁₋₆ haloalkoxy.

3. The compound of claim 2, or a pharmaceutically acceptable salt, solvate, hydrate, isomer, tautomer, racemate, or isotope thereof, wherein
ring B is substituted with 1-5 R¹⁵; and
at least one R¹⁵ is —OH, F, or —CH₃.

4. The compound of claim 1, having the structure of any one of Formula (I-A-iii-a), Formula (I-A-iii-b) or Formula (I-A-iv): 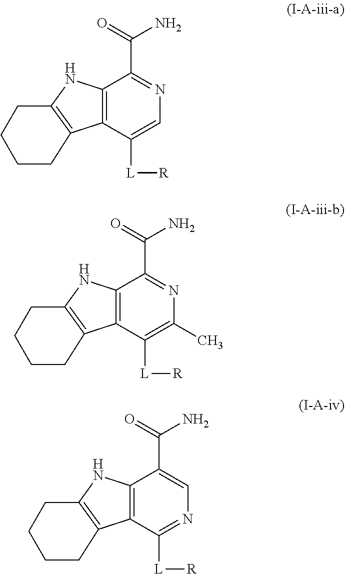
or a pharmaceutically acceptable salt, solvate, hydrate, isomer, tautomer, racemate, or isotope thereof.

5. The compound of claim 1, having the structure of Formula (I-B): 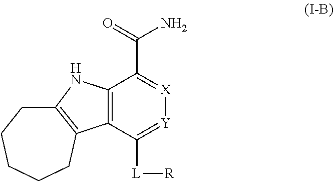
or a pharmaceutically acceptable salt, solvate, hydrate, isomer, tautomer, racemate, or isotope thereof.

6. The compound of claim 5, having the structure of any one of Formula (I-B-i), Formula (I-B-ii), Formula (I-B-iii-a), Formula (I-B-iii-b) or Formula (I-B-iv): 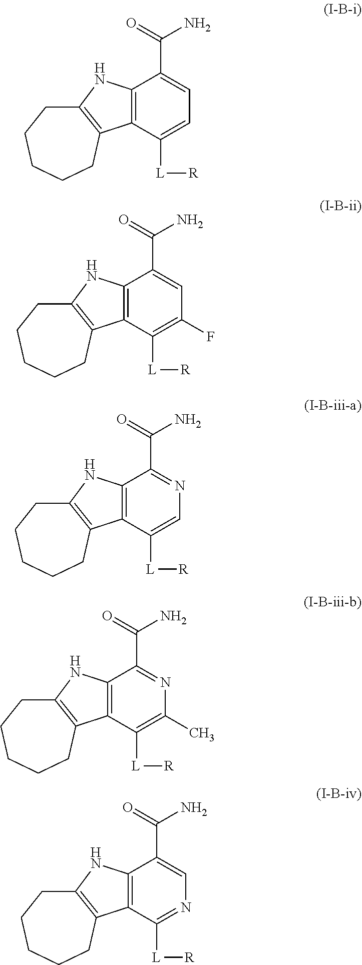
or a pharmaceutically acceptable salt, solvate, hydrate, isomer, tautomer, racemate, or isotope thereof.

7. The compound of claim 1, having the structure of Formula (I-C): 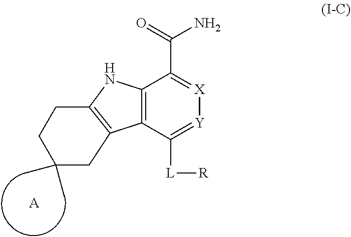
or a pharmaceutically acceptable salt, solvate, hydrate, isomer, tautomer, racemate, or isotope thereof.

8. The compound of claim 7, having the structure of any one of Formula (I-C-i), Formula (I-C-ii), Formula (I-C-iii-a), Formula (I-C-iii-b) or Formula (I-C-iv): 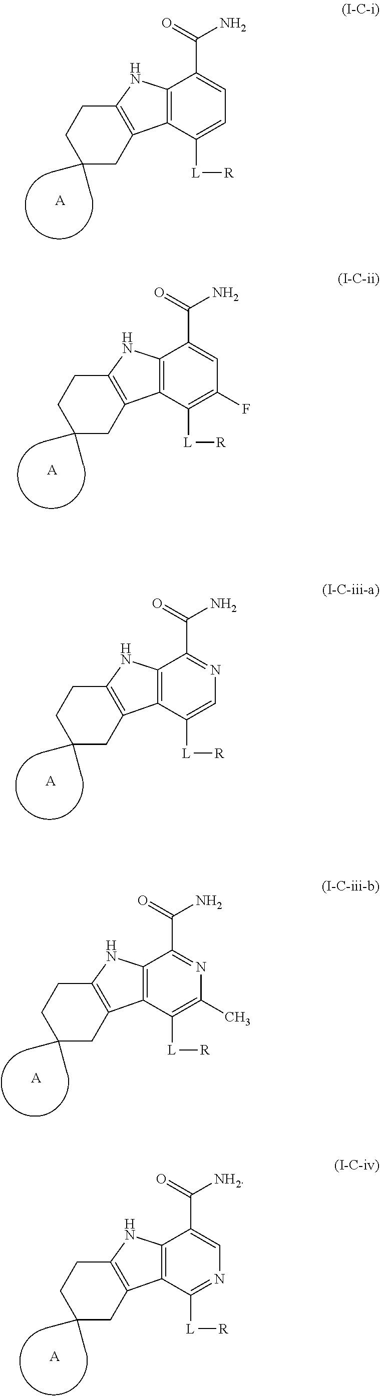
or a pharmaceutically acceptable salt, solvate, hydrate, isomer, tautomer, racemate, or isotope thereof.

9. The compound of claim 1, having the structure of Formula (I-D): 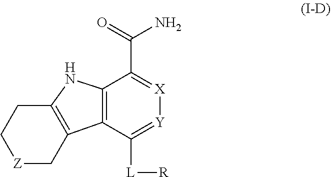
or a pharmaceutically acceptable salt, solvate, hydrate, isomer, tautomer, racemate, or isotope thereof, wherein:
Z is a bond, O, S, Si(CH₃)₂ or CH₂N(CH₃).

10. The compound of claim 9, having the structure of any one of Formula (I-D-i), Formula (I-D-ii), Formula (I-D-iii-a), Formula (I-D-iii-b) or Formula (I-D-iv): 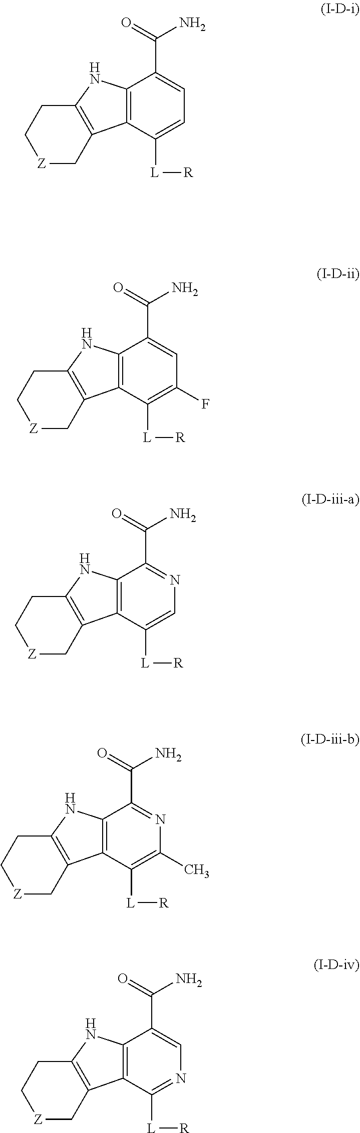
or a pharmaceutically acceptable salt, solvate, hydrate, isomer, tautomer, racemate, or isotope thereof.

11. The compound of claim 9, having the structure of any one of Formula (I-D-v), Formula (I-D-vi), Formula (I-D-vii), Formula (I-D-viii) or Formula (I-D-ix):

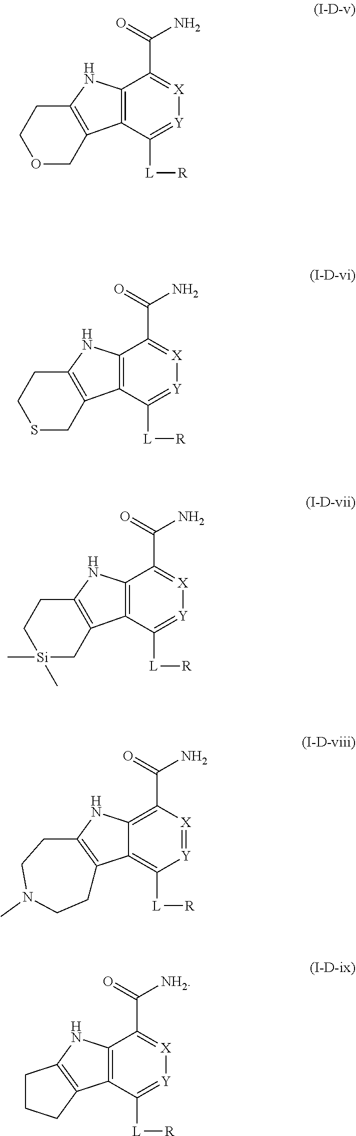

12. The compound of claim 8, or a pharmaceutically acceptable salt, solvate, hydrate, isomer, tautomer, racemate, or isotope thereof, wherein A is:

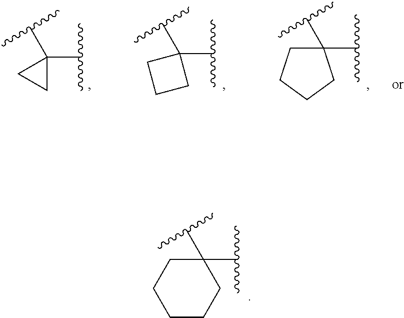 [SMILES: *C1(*)CC1 ; *C1(*)CCC1 ; *C1(*)CCCC1 ; *C1(*)CCCCC1]

13. The compound of claim 8, or a pharmaceutically acceptable salt, solvate, hydrate, isomer, tautomer, racemate, or isotope thereof, wherein A is:

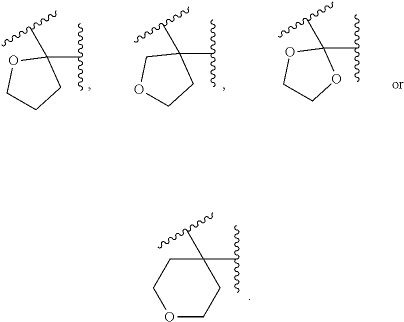 [SMILES: *C1(*)CCCO1 ; *C1(*)CCOC1 ; *C1(*)OCCO1 ; *C1(*)CCOCC1]

14. The compound of claim 8, or a pharmaceutically acceptable salt, solvate, hydrate, isomer, tautomer, racemate, or isotope thereof, wherein A is a 5-membered carbocyclic ring containing 1 or 2 nitrogen atoms in place of 1 or 2 ring carbon atoms, once or twice substituted with oxo, and having one of the following structures:

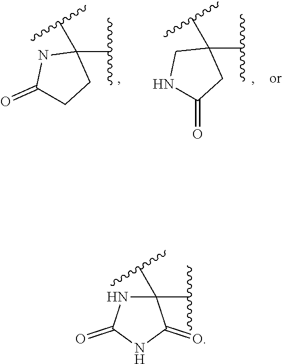 [SMILES: *C1(*)CCC(=O)N1 ; *C1(*)CNC(=O)C1 ; *C1(*)NC(=O)NC1=O]

15. The compound of claim 1, or a pharmaceutically acceptable salt, solvate, hydrate, isomer, tautomer, racemate, or isotope thereof, wherein ring B is an optionally substituted carbocycle.

16. The compound of claim 1, or a pharmaceutically acceptable salt, solvate, hydrate, isomer, tautomer, racemate, or isotope thereof, wherein L is:

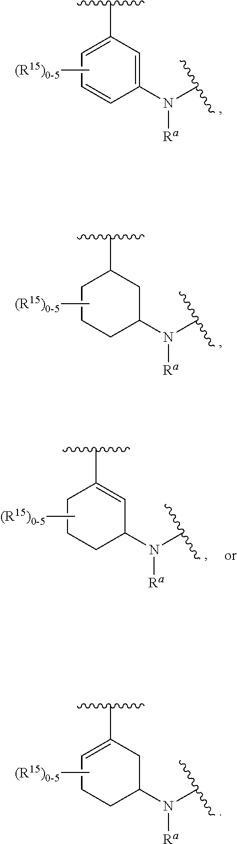

17. The compound of claim 16, or a pharmaceutically acceptable salt, solvate, hydrate, isomer, tautomer, racemate, or isotope thereof, wherein L is:

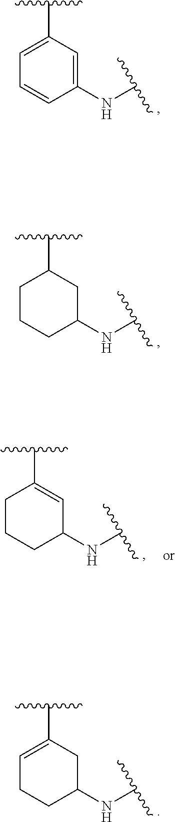 [SMILES: *Nc1cccc(*)c1 ; *NC1CCCC(*)C1 ; *NC1C=C(*)CCC1 ; *NC1CCC=C(*)C1]

18. The compound of claim 1, or a pharmaceutically acceptable salt, solvate, hydrate, isomer, tautomer, racemate, or isotope thereof, wherein ring B is an optionally substituted heterocycle.

19. The compound of claim 1, or a pharmaceutically acceptable salt, solvate, hydrate, isomer, tautomer, racemate, or isotope thereof, wherein L is:

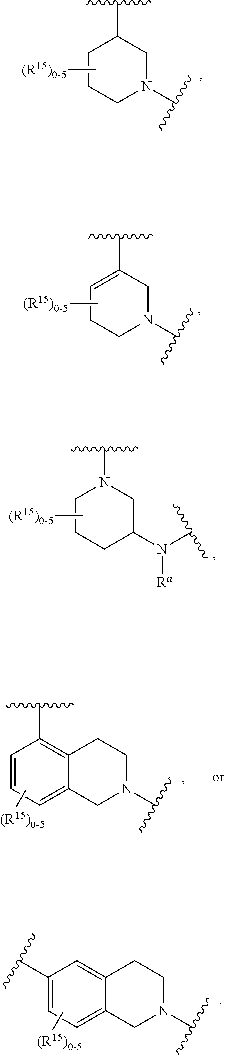

20. The compound of claim 19, or a pharmaceutically acceptable salt, solvate, hydrate, isomer, tautomer, racemate, or isotope thereof, wherein L is:

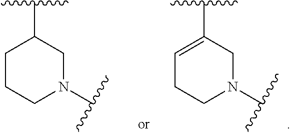 [SMILES: *C1CCCN(*)C1 ; *C1=CCCN(*)C1]

21. The compound of claim 19, or a pharmaceutically acceptable salt, solvate, hydrate, isomer, tautomer, racemate, or isotope thereof, wherein L is:

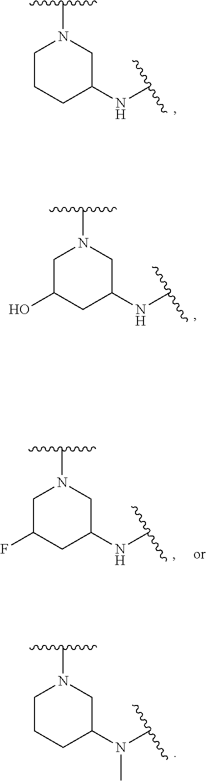

22. The compound of claim 19, or a pharmaceutically acceptable salt, solvate, hydrate, isomer, tautomer, racemate, or isotope thereof, wherein L is:

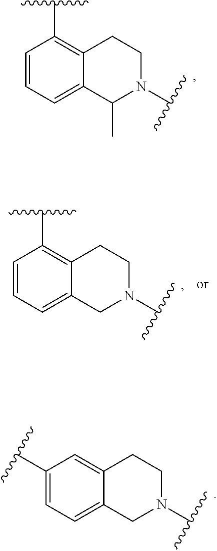 [SMILES: *c1cccc2c1CCN(*)C2C ; *c1cccc2c1CCN(*)C2 ; *c1ccc2c(c1)CCN(*)C2]

23. The compound of claim 1, or a pharmaceutically acceptable salt, solvate, hydrate, isomer, tautomer, racemate, or isotope thereof, wherein R¹⁴ is C₂₋₆ alkenyl substituted with 0-3 R′.

24. The compound of claim 1, or a pharmaceutically acceptable salt, solvate, hydrate, isomer, tautomer, racemate, or isotope thereof, wherein R is:

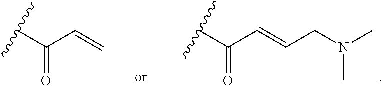

25. The compound of claim 1, or a pharmaceutically acceptable salt, solvate, hydrate, isomer, tautomer, racemate, or isotope thereof, wherein R¹⁴ is C₂₋₆ alkynyl substituted with 0-3 R′.

26. The compound of claim 1, or a pharmaceutically acceptable salt, solvate, hydrate, isomer, tautomer, racemate, or isotope thereof, wherein R is:

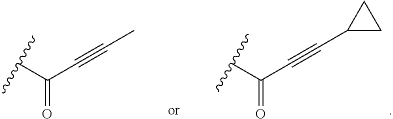

27. A compound having any one of the structures listed in Table 1, or a pharmaceutically acceptable salt, solvate, hydrate, isomer, tautomer, racemate, or isotope thereof.

28. A pharmaceutically acceptable salt of a compound of claim 1.

29. A pharmaceutical composition comprising a compound claim 1, or a pharmaceutically acceptable salt, solvate, hydrate, isomer, tautomer, racemate, or isotope thereof, and at least one pharmaceutically acceptable excipient.

30. A method of inhibiting a protein kinase comprising contacting the protein kinase with an effective amount of a compound of claim 1, or a pharmaceutically acceptable salt, solvate, hydrate, isomer, tautomer, racemate, isotope, or pharmaceutical composition thereof.

31. The method of claim 30, wherein the protein kinase is BTK.

32. A method for treating a BTK dependent condition, comprising administering to a subject in need thereof, an effective amount of a compound of claim 1, or a pharmaceutically acceptable salt, solvate, hydrate, isomer, tautomer, racemate, isotope, or pharmaceutical composition thereof.

33. The method of claim 32, wherein, wherein the BTK dependent condition is cancer, an autoimmune disease, an inflammatory disease, or a thromboembolic disease.

34-41. (canceled)
